SEQUENCE LISTING (42 sequences)

SEQ ID NO: 1 - RNA, 1975 nt, Homo sapiens
acuuuccccccucggcgccccaccggcucccgcgcgccuccccucgcgcccgagcuucgagccaagcagcguccuggggagcgcgucauggccuuaccagugaccgccuugcuccugccgcuggccuugcugcuccacgccgccaggccgagccaguuccgggugucgccgcuggaucggaccuggaaccugggcgagacaguggagcugaagugccaggugcugcuguccaacccgacgucgggcugcucguggcucuuccagccgcgcggcgccgccgccagucccaccuuccuccuauaccucucccaaaacaagcccaaggcggccgaggggcuggacacccagcgguucucgggcaagagguugggggacaccuucguccucacccugagcgacuuccgccgagagaacgagggcuacuauuucugcucggcccugagcaacuccaucauguacuucagccacuucgugccggucuuccugccagcgaagcccaccacgacgccagcgccgcgaccaccaacaccggcgcccaccaucgcgucgcagccccugucccugcgcccagaggcgugccggccagcggcggggggcgcagugcacacgagggggcuggacuucgccugugauaucuacaucugggcgccccuggccgggacuugugggguccuucuccugucacugguuaucacccuuuacugcaaccacaggaaccgaagacguguuugcaaauguccccggccuguggucaaaucgggagacaagcccagccuuucggcgagauacgucuaacccugugcaacagccacuacauuacuucaaacugagauccuuccuuuugagggagcaaguccuucccuuucauuuuuuccagucuuccucccuguguauucauuuucaugauuauuauuuuagugggggcggggugggaaagauuacuuuuucuuuauguguuugacgggaaacaaaacuagguaaaaucuacaguacaccacaagggucacaauacuguugugcgcacaucgcgguagggcguggaaaggggcaggccagagcuacccgcagaguucucagaaucaugcugagagagcuggaggcacccaugccaucucaaccucuuccccgcccguuuuacaaagggggaggcuaaagcccagagacagcuugaucaaaggcacacagcaagucaggguuggagcaguagcuggagggaccuugucucccagcucagggcucuuuccuccacaccauucaggucuuucuuuccgaggccccugucucagggugaggugcuugagucuccaacggcaagggaacaaguacuucuugauaccugggauacugugcccagagccucgaggagguaaugaauuaaagaagagaacugccuuuggcagaguucuauaauguaaacaauaucagacuuuuuuuuuuuauaaucaagccuaaaauuguauagaccuaaaauaaaaugaaguggugagcuuaacccuggaaaaugaaucccucuaucucuaaagaaaaucucugugaaaccccuacguggaggcggaauugcucucccagcccuugcauugcagaggggcccaugaaagaggacaggcuaccccuuuacaaauagaauuugagcaucagugagguuaaacuaaggcccucuugaaucucugaauuugagauacaaacauguuccugggaucacugaugacuuuuuauacuuuguaaagacaauuguuggagagccccucacacagcccuggccuccgcucaacuagcagauacagggaugaggcagaccugacucucuuaaggaggcugagagcccaaacugcugucccaaacaugcacuuccuugcuuaagguaugguacaagcaaugccugcccauuggagagaaaaaacuuaaguagauaaggaaauaagaaccacucauaauucuucaccuuaggaauaaucuccuguuaauaugguguaca

SEQ ID NO: 2 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 3 - RNA, 934 nt, Homo sapiens
ugagaagaugcaaccaauccugcuucugcuggccuuccuccugcugcccagggcagaugcaggggagaucaucgggggacaugaggccaagccccacucccgccccuacauggcuuaucuuaugaucugggaucagaagucucugaagaggugcgguggcuuccugauacaagacgacuucgugcugacagcugcucacuguuggggaagcuccauaaaugucaccuugggggcccacaauaucaaagaacaggagccgacccagcaguuuaucccugugaaaagacccaucccccauccagccuauaauccuaagaacuucuccaacgacaucaugcuacugcagcuggagagaaaggccaagcggaccagagcugugcagccccucaggcuaccuagcaacaaggcccaggugaagccagggcagacaugcaguguggccggcugggggcagacggccccccugggaaaacacucacacacacuacaagaggugaagaugacagugcaggaagaucgaaagugcgaaucugacuuacgccauuauuacgacaguaccauugaguugugcgugggggacccagagauuaaaaagacuuccuuuaagggggacucuggaggcccucuuguguguaacaagguggcccagggcauugucuccuauggacgaaacaauggcaugccuccacgagccugcaccaaagucucaagcuuuguacacuggauaaagaaaaccaugaaacgcuacuaacuacaggaagcaaacuaagcccccgcuguaaugaaacaccuucucuggagccaaguccagauuuacacugggagaggugccagcaacugaauaaauaccucucccaguguaaaucuggagccaaguccagauuuacacugggagaggugccagcaacugaauaaauaccucuuagcugagugg

SEQ ID NO: 4 - protein, 247 aa, Homo sapiens
MQPILLLLAFLLLPRADAGEIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 5 - RNA, 1193 nt, Homo sapiens
ugaagaucagcuauuagaagagaaagaucaguuaaguccuuuggaccugaucagcuugauacaagaacuacugauuucaacuucuuuggcuuaauucucucggaaacgaugaaauauacaaguuauaucuuggcuuuucagcucugcaucguuuuggguucucuuggcuguuacugccaggacccauauguaaaagaagcagaaaaccuuaagaaauauuuuaaugcaggucauucagauguagcggauaauggaacucuuuucuuaggcauuuugaagaauuggaaagaggagagugacagaaaaauaaugcagagccaaauugucuccuuuuacuucaaacuuuuuaaaaacuuuaaagaugaccagagcauccaaaagaguguggagaccaucaaggaagacaugaaugucaaguuuuucaauagcaacaaaaagaaacgagaugacuucgaaaagcugacuaauuauucgguaacugacuugaauguccaacgcaaagcaauacaugaacucauccaagugauggcugaacugucgccagcagcuaaaacagggaagcgaaaaaggagucagaugcuguuucaaggucgaagagcaucccaguaaugguuguccugccugcaauauuugaauuuuaaaucuaaaucuauuuauuaauauuuaacauuauuuauauggggaauauauuuuuagacucaucaaucaaauaaguauuuauaauagcaacuuuuguguaaugaaaaugaauaucuauuaauauauguauuauuuauaauuccuauauccugugacugucucacuuaauccuuuguuuucugacuaauuaggcaaggcuaugugauuacaaggcuuuaucucaggggccaacuaggcagccaaccuaagcaagaucccauggguuguguguuuauuucacuugaugauacaaugaacacuuauaagugaagugauacuauccaguuacugccgguuugaaaauaugccugcaaucugagccagugcuuuaauggcaugucagacagaacuugaaugugucaggugacccugaugaaaacauagcaucucaggagauuucaugccuggugcuuccaaauauuguugacaacugugacuguacccaaauggaaaguaacucauuuguuaaaauuaucaauaucuaauauauaugaauaaaguguaaguucacaacu

SEQ ID NO: 6 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 7 - RNA, 2545 nt, Homo sapiens
auccaauacaggagugacuuggaacuccauucuaucacuaugaagaaaagugguguucuuuuccucuugggcaucaucuugcugguucugauuggagugcaaggaaccccaguagugagaaagggucgcuguuccugcaucagcaccaaccaagggacuauccaccuacaauccuugaaagaccuuaaacaauuugccccaagcccuuccugcgagaaaauugaaaucauugcuacacugaagaauggaguucaaacaugucuaaacccagauucagcagaugugaaggaacugauuaaaaagugggagaaacaggucagccaaaagaaaaagcaaaagaaugggaaaaaacaucaaaaaaagaaaguucugaaaguucgaaaaucucaacguucucgucaaaagaagacuacauaagagaccacuucaccaauaaguauucuguguuaaaaauguucuauuuuaauuauaccgcuaucauuccaaaggaggauggcauauaauacaaaggcuuauuaauuugacuagaaaauuuaaaacauuacucugaaauuguaacuaaaguuagaaaguugauuuuaagaauccaaacguuaagaauuguuaaaggcuaugauugucuuuguucuucuaccacccaccaguugaauuucaucaugcuuaaggccaugauuuuagcaauacccaugucuacacagauguucacccaaccacaucccacucacaacagcugccuggaagagcagcccuaggcuuccacguacugcagccuccagagaguaucugaggcacaugucagcaaguccuaagccuguuagcaugcuggugagccaagcaguuugaaauugagcuggaccucaccaagcugcuguggccaucaaccucuguauuugaaucagccuacaggccucacacacaaugugucugagagauucaugcugauuguuauuggguaucaccacuggagaucaccaguguguggcuuucagagccuccuuucuggcuuuggaagccaugugauuccaucuugcccgcucaggcugaccacuuuauuucuuuuuguuccccuuugcuucauucaagucagcucuucuccauccuaccacaaugcagugccuuucuucucuccagugcaccugucauaugcucugauuuaucugagucaacuccuuucucaucuuguccccaacaccccacagaagugcuuucuucucccaauucauccucacucaguccagcuuaguucaaguccugccucuuaaauaaaccuuuuuggacacacaaauuaucuuaaaacuccuguuucacuugguucaguaccacaugggugaacacucaaugguuaacuaauucuuggguguuuauccuaucucuccaaccagauugucagcuccuugagggcaagagccacaguauauuucccuguuucuuccacagugccuaauaauacuguggaacuagguuuuaauaauuuuuuaauugauguuguuaugggcaggauggcaaccagaccauugucucagagcaggugcuggcucuuuccuggcuacuccauguuggcuagccucugguaaccucuuacuuauuaucuucaggacacucacuacagggaccagggaugaugcaacauccuugucuuuuuaugacaggauguuugcucagcuucuccaacaauaagaagcacgugguaaaacacuugcggauauucuggacuguuuuuaaaaaauauacaguuuaccgaaaaucauauaaucuuacaaugaaaaggacuuuauagaucagccagugaccaaccuuuucccaaccauacaaaaauuccuuuucccgaaggaaaagggcuuucucaauaagccucagcuuucuaagaucuaacaagauagccaccgagauccuuaucgaaacucauuuuaggcaaauaugaguuuuauuguccguuuacuuguuucagaguuuguauugugauuaucaauuaccacaccaucucccaugaagaaagggaacggugaaguacuaagcgcuagaggaagcagccaagucgguuaguggaagcaugauuggugcccaguuagccucugcaggauguggaaaccuccuuccaggggagguucagugaauuguguaggagagguugucuguggccagaauuuaaaccuauacucacuuucccaaauugaaucacugcucacacugcugaugauuuagagugcuguccgguggagaucccacccgaacgucuuaucuaaucaugaaacucccuaguuccuucauguaacuucccugaaaaaucuaaguguuucauaaauuugagagucugugacccacuuaccuugcaucucacagguagacaguauauaacuaacaaccaaagacuacauauugucacugacacacacguuauaaucauuuaucauauauauacauacaugcauacacucucaaagcaaauaauuuuucacuucaaaacaguauugacuuguauaccuuguaauuugaaauauuuucuuuguuaaaauagaaugguaucaauaaauagaccauuaaucag

SEQ ID NO: 8 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 9 - protein, 10 aa, Artificial Sequence
  Synthetic Construct
GFTFSDSWIH

SEQ ID NO: 10 - protein, 18 aa, Artificial Sequence
  Synthetic Construct
AWISPYGGSTYYADSVKG

SEQ ID NO: 11 - protein, 9 aa, Artificial Sequence
  Synthetic Construct
RHWPGGFDY

SEQ ID NO: 12 - protein, 11 aa, Artificial Sequence
  Synthetic Construct
RASQDVSTAVA

SEQ ID NO: 13 - protein, 7 aa, Artificial Sequence
  Synthetic Construct
SASFLYS

SEQ ID NO: 14 - protein, 9 aa, Artificial Sequence
  Synthetic Construct
QQYLYHPAT

SEQ ID NO: 15 - protein, 118 aa, Artificial Sequence [sequence omitted]

SEQ ID NO: 16 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 17 - protein, 447 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 214 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 440 aa, Artificial Sequence
  Synthethic Construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 214 aa, Artificial Sequence
  Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - RNA, 1204 nt, Homo sapiens
ggggugcaaagaagagacagcagcgcccagcuuggaggugcuaacuccagaggccagcaucagcaacugggcacagaaaggagccgccugggcagggaccauggcacggccacaucccugguggcugugcguucuggggacccugguggggcucucagcuacuccagcccccaagagcugcccagagaggcacuacugggcucagggaaagcugugcugccagaugugugagccaggaacauuccucgugaaggacugugaccagcauagaaaggcugcucagugugauccuugcauaccgggggucuccuucucuccugaccaccacacccggccccacugugagagcugucggcacuguaacucuggucuucucguucgcaacugcaccaucacugccaaugcugagugugccugucgcaauggcuggcagugcagggacaaggagugcaccgagugugauccucuuccaaacccuucgcugaccgcucggucgucucaggcccugagcccacacccucagcccacccacuuaccuuaugucagugagaugcuggaggccaggacagcugggcacaugcagacucuggcugacuucaggcagcugccugcccggacucucucuacccacuggccaccccaaagaucccugugcagcuccgauuuuauucgcauccuugugaucuucucuggaauguuccuuguuuucacccuggccggggcccuguuccuccaucaacgaaggaaauauagaucaaacaaaggagaaaguccuguggagccugcagagccuugucguuacagcugccccagggaggaggagggcagcaccauccccauccaggaggauuaccgaaaaccggagccugccugcucccccugagccagcaccugcgguagcugcacuacagcccuggccuccacccccaccccgccgaccauccaagggagagugagaccuggcagccacaacugcagucccauccucuugucagggcccuuuccuguguacacgugacagagugccuuuucgagacuggcagggacgaggacaaauauggaugagguggagagugggaagcaggagcccagccagcugcgccugcgcugcaggagggcgggggcucugguuguaaaacacacuuccugcugcgaaagacccacaugcuacaagacgggcaaaauaaagugacagaugaccacc

SEQ ID NO: 22 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 23 - RNA, 2397 nt, Homo sapiens
gcacacacucaucgaaaaaaauuuggauuauuagaagagagaggucugcggcuuccacaccguacagcgugguuuuucuucucgguauaaaagcaaaguuguuuuugauacgugacaguuucccacaagccaggcugauccuuuucugucaguccacuucaccaagccugcccuuggacaaggacccgaugcccaaccccaggccuggcaagcccucggccccuuccuuggcccuuggcccauccccaggagccucgcccagcuggagggcugcacccaaagccucagaccugcugggggcccggggcccagggggaaccuuccagggccgagaucuucgaggcggggcccaugccuccucuucuuccuugaaccccaugccaccaucgcagcugcagcugcccacacugccccuagucaugguggcacccuccggggcacggcugggccccuugccccacuuacaggcacuccuccaggacaggccacauuucaugcaccagcucucaacgguggaugcccacgcccggaccccugugcugcaggugcacccccuggagagcccagccaugaucagccucacaccacccaccaccgccacuggggucuucucccucaaggcccggccuggccucccaccugggaucaacguggccagccuggaauggguguccagggagccggcacugcucugcaccuucccaaaucccagugcacccaggaaggacagcacccuuucggcugugccccagagcuccuacccacugcuggcaaauggugucugcaaguggcccggaugugagaaggucuucgaagagccagaggacuuccucaagcacugccaggcggaccaucuucuggaugagaagggcagggcacaaugucuccuccagagagagaugguacagucucuggagcagcagcuggugcuggagaaggagaagcugagugccaugcaggcccaccuggcugggaaaauggcacugaccaaggcuucaucuguggcaucauccgacaagggcuccugcugcaucguagcugcuggcagccaaggcccugucgucccagccuggucuggcccccgggaggccccugacagccuguuugcuguccggaggcaccugugggguagccauggaaacagcacauucccagaguuccuccacaacauggacuacuucaaguuccacaacaugcgacccccuuucaccuacgccacgcucauccgcugggccauccuggaggcuccagagaagcagcggacacucaaugagaucuaccacugguucacacgcauguuugccuucuucagaaaccauccugccaccuggaagaacgccauccgccacaaccugagucugcacaagugcuuugugcggguggagagcgagaagggggcuguguggaccguggaugagcuggaguuccgcaagaaacggagccagaggcccagcagguguuccaacccuacaccuggccccugaccucaagaucaaggaaaggaggauggacgaacaggggccaaacuggugggaggcagagguggugggggcagggaugauaggcccuggaugugcccacagggaccaagaagugagguuuccacugucuugccugccagggccccuguucccccgcuggcagccacccccucccccaucauauccuuugccccaaggcugcucagaggggccccgguccuggccccagcccccaccuccgccccagacacaccccccagucgagcccugcagccaaacagagccuucacaaccagccacacagagccugccucagcugcucgcacagauuacuucagggcuggaaaagucacacagacacacaaaaugucacaauccugucccucacucaacacaaaccccaaaacacagagagccugccucaguacacucaaacaaccucaaagcugcaucaucacacaaucacacacaagcacagcccugacaacccacacaccccaaggcacgcacccacagccagccucagggcccacaggggcacugucaacacaggggugugcccagaggccuacacagaagcagcgucaguacccucaggaucugaggucccaacacgugcucgcucacacacacggccuguuagaauucaccuguguaucucacgcauaugcacacgcacagccccccagugggucucuugagucccgugcagacacacacagccacacacacugccuugccaaaaauaccccgugucuccccugccacucaccucacucccauucccugagcccugauccaugccucagcuuagacugcagaggaacuacucauuuauuugggauccaaggcccccaacccacaguaccguccccaauaaacugcagccgagcuccccacaaaaaaaaaaaaaaaa

SEQ ID NO: 24 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 25 - RNA, 2033 nt, Homo sapiens
cuucugugugugcacauguguaauacauaucugggaucaaagcuaucuauauaaaguccuugauucuguguggguucaaacacauuucaaagcuucaggauccugaaagguuuugcucuacuuccugaagaccugaacaccgcucccauaaagccauggcuugccuuggauuucagcggcacaaggcucagcugaaccuggcuaccaggaccuggcccugcacucuccuguuuuuucuucucuucaucccugucuucugcaaagcaaugcacguggcccagccugcugugguacuggccagcagccgaggcaucgccagcuuugugugugaguaugcaucuccaggcaaagccacugagguccgggugacagugcuucggcaggcugacagccaggugacugaagucugugcggcaaccuacaugauggggaaugaguugaccuuccuagaugauuccaucugcacgggcaccuccaguggaaaucaagugaaccucacuauccaaggacugagggccauggacacgggacucuacaucugcaagguggagcucauguacccaccgccauacuaccugggcauaggcaacggaacccagauuuauguaauugauccagaaccgugcccagauucugacuuccuccucuggauccuugcagcaguuaguucgggguuguuuuuuuauagcuuucuccucacagcuguuucuuugagcaaaaugcuaaagaaaagaagcccucuuacaacaggggucuaugugaaaaugcccccaacagagccagaaugugaaaagcaauuucagccuuauuuuauucccaucaauugagaaaccauuaugaagaagagaguccauauuucaauuuccaagagcugaggcaauucuaacuuuuuugcuauccagcuauuuuuauuuguuugugcauuuggggggaauucaucucucuuuaauauaaaguuggaugcggaacccaaauuacguguacuacaauuuaaagcaaaggaguagaaagacagagcugggauguuucugucacaucagcuccacuuucagugaaagcaucacuugggauuaauauggggaugcagcauuaugaugugggucaaggaauuaaguuagggaauggcacagcccaaagaaggaaaaggcagggagcgagggagaagacuauauuguacacaccuuauauuuacguaugagacguuuauagccgaaaugaucuuuucaaguuaaauuuuaugccuuuuauuucuuaaacaaauguaugauuacaucaaggcuucaaaaauacucacauggcuauguuuuagccagugaugcuaaagguuguauugcauauauacauauauauauauauauauauauauauauauauauauauauauauauauauauauauuuuaauuugauaguauugugcauagagccacguauguuuuuguguauuuguuaaugguuugaauauaaacacuauauggcagugucuuuccaccuugggucccagggaaguuuuguggaggagcucaggacacuaauacaccagguagaacacaaggucauuugcuaacuagcuuggaaacuggaugaggucauagcagugcuugauugcguggaauugugcugaguugguguugacaugugcuuuggggcuuuuacaccaguuccuuucaaugguuugcaaggaagccacagcuggugguaucugaguugacuugacagaacacugucuugaagacaauggcuuacuccaggagacccacagguaugaccuucuaggaagcuccaguucgaugggcccaauucuuacaaacaugugguuaaugccauggacagaagaaggcagcagguggcagaauggggugcaugaagguuucugaaaauuaacacugcuuguguuuuuaacucaauauuuuccaugaaaaugcaacaacauguauaauauuuuuaauuaaauaaaaaucugugguggucguuuuaaaaaaaaaaaaaaaaaaaaaaaaaaaaaaaaaaaaaaaaaaaaaaaaaaaaaaaaaa

SEQ ID NO: 26 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 27 - RNA, 2978 nt, Homo sapiens
cguccuaucugcagucggcuacuuucaguggcagaagaggccacaucugcuuccuguaggcccucugggcagaagcaugcgcuggugucuccuccugaucugggcccaggggcugaggcaggcuccccucgccucaggaaugaugacaggcacaauagaaacaacggggaacauuucugcagagaaagguggcucuaucaucuuacaaugucaccucuccuccaccacggcacaagugacccaggucaacugggagcagcaggaccagcuucuggccauuuguaaugcugacuugggguggcacaucuccccauccuucaaggaucgaguggccccaggucccggccugggccucacccuccagucgcugaccgugaacgauacaggggaguacuucugcaucuaucacaccuacccugaugggacguacacugggagaaucuuccuggagguccuagaaagcucaguggcugagcacggugccagguuccagauuccauugcuuggagccauggccgcgacgcugguggucaucugcacagcagucaucgugguggucgcguugacuagaaagaagaaagcccucagaauccauucuguggaaggugaccucaggagaaaaucagcuggacaggaggaauggagccccagugcucccucacccccaggaagcuguguccaggcagaagcugcaccugcugggcucuguggagagcagcggggagaggacugugccgagcugcaugacuacuucaauguccugaguuacagaagccuggguaacugcagcuucuucacagagacugguuagcaaccagaggcaucuucuggaagauacacuuuugucuuugcuauuauagaugaauauauaagcagcuguacucuccaucagugcugcguguguguguguguguguauguguguguguguucaguugagugaauaaaugucauccucuucuccaucuucauuuccuuggccuuuucguucuauuccauuuugcauuauggcaggccuagggugaguaacguggaucuugaucauaaaugcaaaauuaaaaaauaucuugaccugguuuuaaaucuggcaguuugagcagauccuaugucucugagagacacauuccucauaauggccagcauuuugggcuacaagguuuugugguugaugaugaggauggcaugacugcagagccauccucaucucauuuuuucacgucauuuucaguaacuuucacucauucaaaggcagguuauaaguaaguccugguagcagccucuauggggagauuugagagugacuaaaucuugguaucugcccucaagaacuuacaguuaaauggggagacaauguugucaugaaaagguauuauaguaaggagagaaggagacauacacaggccuucaggaagagacgacaguuuggggugagguaguuggcauaggcuuaucugugaugaaguggccugggagcaccaaggggauguugaggcuagucugggaggagcaggaguuuugucuagggaacuuguaggaaauucuuggagcugaaagucccacaaagaaggcccuggcaccaagggagucagcaaacuucagauuuuauucucugggcaggcauuucaaguuuccuuuugcugugacauacucauccauuagacagccugauacaggccuguagccucuuccggccgugugugcuggggaagccccaggaaacgcacaugcccacacagggagccaagucguagcauuugggccuugaucuaccuuuucugcaucaauacacucuugagccuuugaaaaaagaacguuucccacuaaaaagaaaauguggauuuuuaaaauagggacucuuccuaggggaaaaaggggggcugggagugauagaggguuuaaaaaauaaacaccuucaaacuaacuucuucgaacccuuuuauucacucccugacgacuuugugcugggguugggguaacugaaccgcuuauuucuguuuaauugcauucaggcuggaucuuagaagacuuuuauccuuccaccaucucucucagaggaaugagcggggagguuggauuuacuggugacugauuuucuuucaugggccaaggaacugaaagagaaugugaagcaagguugugucuugcgcaugguuaaaaauaaagcauuguccugcuuccuaagacuuagacugggguugacaauuguuuuagcaacaagacaauucaacuauuucuccuaggauuuuuauuauuauuauuuuuucacuuuucuaccaaauggguuacauaggaagaaugaacugaaaucuguccagagcuccaaguccuuuggaagaaagauuagaugaacguaaaaauguuguuguuugcuguggcaguuuacagcauuuuucuugcaaaauuagugcaaaucuguuggaaauagaacacaauucacaaauuggaagugaacuaaaauguaaugacgaaaagggaguaguguuuugauuuggaggagguguauauucggcagagguuggacugagaguuggguguuauuuaacauaauuaugguaauugggaaacauuuauaaacacuauugggauggugauaaaauacaaaagggccuauagauguuagaaaugggucagguuacugaaaugggauucaauuugaaaaaaauuuuuuuaaauagaacucacugaacuagauucuccucugagaaccagagaagaccauuucauaguuggauuccuggagacaugcgcuauccaccacguagccacuuuccacauguggccaucaaccacuuaagaugggguuaguuuaaaucaagaugugcuguuauaauugguauaagcauaaaaucacacuagauucuggagauuuaauaugaauaauaagaauacuauuucaguaguuuugguauauugugugucaaaaaugauaauauuuuggauguauugggugaaauaaaauauuaacauuaaaaaaaaaaa

SEQ ID NO: 28 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 29 - RNA, 1944 nt, Homo sapiens
aauuucucacugccccugugauaaacuguggucacuggcuguggcagcaacuauuauaagaugcucugaaaacucuucagacacugaggggcaccagaggagcagacuacaagaauggcacacgcuauggaaaacuccuggacaaucaguaaagaguaccauauugaugaagaagugggcuuugcucugccaaauccacaggaaaaucuaccugauuuuuauaaugacuggauguucauugcuaaacaucugccugaucucauagagucuggccagcuucgagaaagaguugagaaguuaaacaugcucagcauugaucaucucacagaccacaagucacagcgccuugcacgucuaguucugggaugcaucaccauggcauauguguggggcaaaggucauggagauguccguaaggucuugccaagaaauauugcuguuccuuacugccaacucuccaagaaacuggaacugccuccuauuuugguuuaugcagacugugucuuggcaaacuggaagaaaaaggauccuaauaagccccugacuuaugagaacauggacguuuuguucucauuucgugauggagacugcaguaaaggauucuuccuggucucucuauugguggaaauagcagcugcuucugcaaucaaaguaauuccuacuguauucaaggcaaugcaaaugcaagaacgggacacuuugcuaaaggcgcuguuggaaauagcuucuugcuuggagaaagcccuucaaguguuucaccaaauccacgaucaugugaacccaaaagcauuuuucaguguucuucgcauauauuugucuggcuggaaaggcaacccccagcuaucagacggucugguguaugaaggguucugggaagacccaaaggaguuugcagggggcagugcaggccaaagcagcgucuuucagugcuuugacguccugcugggcauccagcagacugcugguggaggacaugcugcucaguuccuccaggacaugagaagauauaugccaccagcucacaggaacuuccugugcucauuagagucaaaucccucaguccgugaguuuguccuuucaaaaggugaugcuggccugcgggaagcuuaugacgccugugugaaagcucuggucucccugaggagcuaccaucugcaaaucgugacuaaguacauccugauuccugcaagccagcagccaaaggagaauaagaccucugaagacccuucaaaacuggaagccaaaggaacuggaggcacugauuuaaugaauuuccugaagacuguaagaaguacaacugagaaaucccuuuugaaggaagguuaauguaacccaacaagagcacauuuuaucauagcagagacaucuguaugcauuccugucauuacccauuguaacagagccacaaacuaauacuaugcaauguuuuaccaauaaugcaauacaaaagaccucaaaauaccugugcauuucuuguaggaaaacaacaaaagguaauuauguguaauuauacuagaaguuuuguaaucuguaucuuaucauuggaauaaaaugacauucaauaaauaaaaaugcauaagauauauucugucggcugggcgcgguggcucacgccuguaaucccagcacuuugggaggccgaggcgggcggaucacaaggucaggagaucgagaccaucuuggcuaacacggugaaaccccgucucuacuaaaaauacaaaaaauuagccgggcgcgguggcgggcaccuguagucccagcuacucgggaggcugaggcaggagaauggcgugaaccugggaggcggagcuugcagugagccaagauugugccacugcaauccggccugggcuaaagagcgggacuccgucucaaaaaaaaaaaaaaaaagauauauucugucauaauaaauaaaaaugcauaagauauaaaaaaaaaaaaaaa

SEQ ID NO: 30 - protein, 403 aa, Homo sapiens [sequence omitted]

SEQ ID NO: 31 - RNA, 1227 nt, Homo sapiens
cuuugcagauaaauauggcacacuagccccacguuuucugagacauuccucaauugcuuagacauauucugagccuacagcagaggaaccuccagucucagcaccaugaaucaaacugccauucugauuugcugccuuaucuuucugacucuaaguggcauucaaggaguaccucucucuagaacuguacgcuguaccugcaucagcauuaguaaucaaccuguuaauccaaggucuuuagaaaaacuugaaauuauuccugcaagccaauuuuguccacguguugagaucauugcuacaaugaaaaagaagggugagaagagaugucugaauccagaaucgaaggccaucaagaauuuacugaaagcaguuagcaaggaaaggucuaaaagaucuccuuaaaaccagaggggagcaaaaucgaugcagugcuuccaaggauggaccacacagaggcugccucucccaucacuucccuacauggaguauaugucaagccauaauuguucuuaguuugcaguuacacuaaaaggugaccaaugauggucaccaaaucagcugcuacuacuccuguaggaagguuaauguucaucauccuaagcuauucaguaauaacucuacccuggcacuauaauguaagcucuacugaggugcuauguucuuaguggauguucugacccugcuucaaauauuucccucaccuuucccaucuuccaaggguacuaaggaaucuuucugcuuugggguuuaucagaauucucagaaucucaaauaacuaaaagguaugcaaucaaaucugcuuuuuaaagaaugcucuuuacuucauggacuuccacugccauccucccaaggggcccaaauucuuucaguggcuaccuacauacaauuccaaacacauacaggaagguagaaauaucugaaaauguauguguaaguauucuuauuuaaugaaagacuguacaaaguagaagucuuagauguauauauuuccuauauuguuuucaguguacauggaauaacauguaauuaaguacuauguaucaaugaguaacaggaaaauuuuaaaaauacagauagauauaugcucugcauguuacauaagauaaaugugcugaaugguuuucaaaauaaaaaugagguacucuccuggaaauauuaagaaagacuaucuaaauguugaaagaucaaaagguuaauaaaguaauuauaacuaagaaaaaaaaaaaa

SEQ ID NO: 32 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 33 - RNA, 1610 nt, Homo sapiens
agagaacaaaacagaaacucuuggaagcaggaaaggugcaugacucaaagagggaaauuccugugccauaaaaggauugcugguguauaaaaugcucuauauaugccaauuaucaauuuccuuucauguucagcauuucuacuccuuccaagaagagcagcaaagcugaaguagcagcagcagcaccagcagcaacagcaaaaaacaaacaugagugugaagggcauggcuauagccuuggcugugauauugugugcuacaguuguucaaggcuuccccauguucaaaagaggacgcugucuuugcauaggcccugggguaaaagcagugaaaguggcagauauugagaaagccuccauaauguacccaaguaacaacugugacaaaauagaagugauuauuacccugaaagaaaauaaaggacaacgaugccuaaaucccaaaucgaagcaagcaaggcuuauaaucaaaaaaguugaaagaaagaauuuuuaaaaauaucaaaacauaugaaguccuggaaaagagcaucugaaaaaccuagaacaaguuuaacugugacuacugaaaugacaagaauucuacaguaggaaacugagacuuuucuaugguuuugugacuuucaacuuuuguacaguuaugugaaggaugaaaggugggugaaaggaccaaaaacagaaauacagucuuccugaaugaaugacaaucagaauuccacugcccaaaggaguccaacaauuaaauggauuucuaggaaaagcuaccuuaagaaaggcugguuaccaucggaguuuacaaagugcuuucacguucuuacuuguugcauuauacauucaugcauuucuaggcuagagaaccuucuagauuugaugcuuacaacuauucuguugugacuaugagaacauuucugucucuagaagucaucugucuguauugaucuuuaugcuauauuacuaucugugguuacgguggagacauugacauuauuacuggagucaagcccuuauaagucaaaagcaucuaugugucguaaaacauuccucaaacauuuuuucaugcaaauacacacuucuuuccccaaacaucauguagcacaucaauauguagggagacauucuuaugcaucauuugguuuguuuuauaaccaauucauuaaauguaauucauaaaauguacuaugaaaaaaauuauacgcuaugggauacuggcaaaagugcacauauuucauaaccaaauuaguagcaccagucuuaauuugauguuuuucaacuuuuauucauugagauguuuugaagcaauuaggauauguguguuuacuguacuuuuuguuuugauccguuuguauaaaugauagcaauaucuuggacacaucugaaauacaaaauguuuuugucuaccaaagaaaaauguugaaaaauaagcaaauguauaccuagcaaucacuuuuacuuuuuguaauucugucucuuagaaaaauacauaaucuaaucaauuucuuuguucaugccuauauacuguaaaauuuagguauacucaagacuaguuuaaagaaucaaagucauuuuuuucucuaauaaacuaccacaaccuuucuuuuuuaaaaaaaaaaa

SEQ ID NO: 34 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 35 - RNA, 1135 nt, Homo sapiens
uuccuccuccgagagcggacagaucucugggugcugggcggucauggcgcuacuagauguaugcggagccccccgagggcagcggccggaaucggcucucccgguugcgggaagcgggcgucgcucggacccaggacacuacaguuucucuaugcgaucuccagagcucgcuuuaccccggggaaugcagcccacagaauucuuccagucccuggguggggacggagaaaggaacguucagauugagauggcccauggcaccaccacgcucgccuucaaguuccagcauggagugauugcagcaguggauucucgggccucagcuggguccuacauuagugccuuacgggugaacaaggugauugagauuaacccuuaccugcuuggcaccaugucuggcugugcagcagacugucaguacugggagcgccugcuggccaaggaaugcaggcuguacuaucugcgaaauggagaacguauuucagugucggcagccuccaagcugcuguccaacaugaugugccaguaccggggcaugggccucucuaugggcaguaugaucuguggcugggauaagaaggguccuggacucuacuacguggaugaacaugggacucggcucucaggaaauauguucuccacggguagugggaacacuuaugccuacggggucauggacaguggcuaucggccuaaucuuagcccugaagaggccuaugaccuuggccgcagggcuauugcuuaugccacucacagagacagcuauucuggaggcguugucaauauguaccacaugaaggaagaugguugggugaaaguagaaaguacagaugucagugaccugcugcaccaguaccgggaagccaaucaauaaugguggugguggcagcugggcaggucuccucugggaggucuuggccgacucagggaccuaagccacguuaaguccaaggagaagaagaggccuagccugagccaaagagagaguacgggcucagcagccagaggaggccggugaagugcaucuucugcguguucucuauuugaacaagcauuucccccagggaaguuucugggugccccacuaaguagaauaaagaaaaacgguuauaaauaaaaaaaaaaaaaaaaaaaa

SEQ ID NO: 36 - protein, 276 aa, Homo sapiens
MALLDVCGAPRGQRPESALPVAGSGRRSDPGHYSFSMRSPELALPRGMQPTEFFQSLGGDGERNVQIEMAHGTTTLAFKFQHGVIAAVDSRASAGSYISALRVNKVIEINPYLLGTMSGCAADCQYWERLLAKECRLYYLRNGERISVSAASKLLSNMMCQYRGMGLSMGSMICGWDKKGPGLYYVDEHGTRLSGNMFSTGSGNTYAYGVMDSGYRPNLSPEEAYDLGRRAIAYATHRDSYSGGVVNMYHMKEDGWVKVESTDVSDLLHQYREANQ

SEQ ID NO: 37 - RNA, 1048 nt, Homo sapiens
gcgcguugugcgcugucccagguuggaaaccagugccccaggcggcgaggagagcggugccuugcagggaugcugcgggcgggagcaccaaccggggacuuaccccgggcgggagaaguccacaccgggaccaccaucauggcaguggaguuugacgggggcguugugauggguucugauucccgagugucugcaggcgaggcgguggugaaccgaguguuugacaagcuguccccgcugcacgagcgcaucuacugugcacucucugguucagcugcugaugcccaagccguggccgacauggccgccuaccagcuggagcuccaugggauagaacuggaggaaccuccacuuguuuuggcugcugcaaauguggugagaaauaucagcuauaaauaucgagaggacuugucugcacaucucaugguagcuggcugggaccaacgugaaggaggucagguauauggaacccugggaggaaugcugacucgacagccuuuugccauugguggcuccggcagcaccuuuaucuaugguuauguggaugcagcauauaagccaggcaugucucccgaggagugcaggcgcuucaccacagacgcuauugcucuggccaugagccgggauggcucaagcgggggugucaucuaccuggucacuauuacagcugccgguguggaccaucgagucaucuugggcaaugaacugccaaaauucuaugaugagugaaccuuccccagacuucucuuucuuauuuuguaauaaacucucuagggccaaaaccugguauggucauugggaaaugagugcucagggagauggagcuuaggggaggugggugcuucccuccuagaugucagcauacacucuuucuucuuuugucccaggucuaaaacaucuuuccuagagaaaacaaaagggacuaaacuagaaauauaaagagcccuauacaugacaggugaucacguacugaaugauuuugaaguaguacaaacaauaaaaauucucauuccgcaucaucaugcgguccaugaugaugaggccgcaa

SEQ ID NO: 38 - protein, 219 aa, Homo sapiens
MLRAGAPTGDLPRAGEVHTGTTIMAVEFDGGVVMGSDSRVSAGEAVVNRVFDKLSPLHERIYCALSGSAADAQAVADMAAYQLELHGIELEEPPLVLAAANVVRNISYKYREDLSAHLMVAGWDQREGGQVYGTLGGMLTRQPFAIGGSGSTFIYGYVDAAYKPGMSPEECRRFTTDAIALAMSRDGSSGGVIYLVTITAAGVDHRVILGNELPKFYDE

SEQ ID NO: 39 - RNA, 2974 nt, Homo sapiens
gugugcgugauggagaaaauugggcaccagggcugcucccgagauucucagaucugauuuccacgcuugcuaccaaaauagucugggcaggccacuuuuggaaguaggcguuaucuagugagcaggcggccgcuuucgauuucgcuuuccccuaaauggcugagcuucucgccagcgcaggaucagccuguuccugggacuuuccgagagccccgcccucguucccucccccagccgccaguaggggaggacucggcgguacccggagcuucaggccccaccggggcgcggagagucccaggcccggccgggaccgggacggcguccgagugccaauggcuagcucuaggugucccgcuccccgcgggugccgcugccuccccggagcuucucucgcauggcuggggacaguacugcuacuucucgccgacugggugcugcuccggaccgcgcugccccgcauauucucccugcuggugcccaccgcgcugccacugcuccgggucugggcggugggccugagccgcugggccgugcucuggcugggggccugcgggguccucagggcaacgguuggcuccaagagcgaaaacgcaggugcccagggcuggcuggcugcuuugaagccauuagcugcggcacugggcuuggcccugccgggacuugccuuguuccgagagcugaucucauggggagcccccggguccgcggauagcaccaggcuacugcacuggggaagucacccuaccgccuucguugucaguuaugcagcggcacugcccgcagcagcccuguggcacaaacucgggagccucugggugcccggcggucagggcggcucuggaaacccugugcgucggcuucuaggcugccugggcucggagacgcgccgccucucgcuguuccugguccuggugguccucuccucucuuggggagauggccauuccauucuuuacgggccgccucacugacuggauucuacaagauggcucagccgauaccuucacucgaaacuuaacucucauguccauucucaccauagccagugcagugcuggaguucgugggugacgggaucuauaacaacaccaugggccacgugcacagccacuugcagggagagguguuuggggcuguccugcgccaggagacggaguuuuuccaacagaaccagacagguaacaucaugucucggguaacagaggacacguccacccugagugauucucugagugagaaucugagcuuauuucugugguaccuggugcgaggccuaugucucuuggggaucaugcucuggggaucagugucccucaccauggucacccugaucacccugccucugcuuuuccuucugcccaagaaggugggaaaaugguaccaguugcuggaagugcaggugcgggaaucucuggcaaaguccagccagguggccauugaggcucugucggccaugccuacaguucgaagcuuugccaacgaggagggcgaagcccagaaguuuagggaaaagcugcaagaaauaaagacacucaaccagaaggaggcuguggccuaugcagucaacuccuggaccacuaguauuucagguaugcugcugaaagugggaauccucuacauuggugggcagcuggugaccaguggggcuguaagcagugggaaccuugucacauuuguucucuaccagaugcaguucacccaggcuguggagguacugcucuccaucuaccccagaguacagaaggcugugggcuccucagagaaaauauuugaguaccuggaccgcaccccucgcugcccacccaguggucuguugacucccuuacacuuggagggccuuguccaguuccaagaugucuccuuugccuacccaaaccgcccagaugucuuagugcuacaggggcugacauucacccuacgcccuggcgaggugacggcgcuggugggacccaaugggucugggaagagcacaguggcugcccugcugcagaaucuguaccagcccaccgggggacagcugcuguuggaugggaagccccuuccccaauaugagcaccgcuaccugcacaggcagguggcugcagugggacaagagccacagguauuuggaagaagucuucaagaaaauauugccuauggccugacccagaagccaacuauggaggaaaucacagcugcugcaguaaagucuggggcccauaguuucaucucuggacucccucagggcuaugacacagagguagacgaggcugggagccagcugucagggggucagcgacaggcaguggcguuggcccgagcauugauccggaaaccguguguacuuauccuggaugaugccaccagugcccuggaugcaaacagccaguuacagguggagcagcuccuguacgaaagcccugagcgguacucccgcucagugcuucucaucacccagcaccucagccugguggagcaggcugaccacauccucuuucuggaaggaggcgcuauccgggaggggggaacccaccagcagcucauggagaaaaaggggugcuacugggccauggugcaggcuccugcagaugcuccagaaugaaagccuucucagaccugcgcacuccaucucccucccuuuucuucucucugugguggagaaccacagcugcagaguaggcagcugccuccaggaugaguuacuugaaauuugccuugaguguguuaccuccuuuccaagcuccucgugauaaugcagacuuccuggaguacaaacacaggauuuguaauuccuuacuguaacggaguuuagagccagggcugaugcuuugguguggccagcacucugaaacugagaaauguucagaauguacggaaagaugaucagcuauuuucaacauaacugaaggcauaugcuggcccauaaacacccuguagguucuugauauuuauaauaaaauugguguuuuguaaaaaaaaaaaaaaaaaaaaaaa

SEQ ID NO: 40 - protein, 808 aa, Homo sapiens
MAELLASAGSACSWDFPRAPPSFPPPAASRGGLGGTRSFRPHRGAESPRPGRDRDGVRVPMASSRCPAPRGCRCLPGASLAWLGTVLLLLADWVLLRTALPRIFSLLVPTALPLLRVWAVGLSRWAVLWLGACGVLRATVGSKSENAGAQGWLAALKPLAAALGLALPGLALFRELISWGAPGSADSTRLLHWGSHPTAFVVSYAAALPAAALWHKLGSLWVPGGQGGSGNPVRRLLGCLGSETRRLSLFLVLVVLSSLGEMAIPFFTGRLTDWILQDGSADTFTRNLTLMSILTIASAVLEFVGDGIYNNTMGHVHSHLQGEVFGAVLRQETEFFQQNQTGNIMSRVTEDTSTLSDSLSENLSLFLWYLVRGLCLLGIMLWGSVSLTMVTLITLPLLFLLPKKVGKWYQLLEVQVRESLAKSSQVAIEALSAMPTVRSFANEEGEAQKFREKLQEIKTLNQKEAVAYAVNSWTTSISGMLLKVGILYIGGQLVTSGAVSSGNLVTFVLYQMQFTQAVEVLLSIYPRVQKAVGSSEKIFEYLDRTPRCPPSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPCVLILDDATSALDANSQLQVEQLLYESPERYSRSVLLITQHLSLVEQADHILFLEGGAIREGGTHQQLMEKKGCYWAMVQAPADAPE

SEQ ID NO: 41 - RNA, 5732 nt, Homo sapiens
agaaugaaggccuuggcuggggaagcgaaagcgaaagcugcccgagcccugacgcccgcccuggccgagcguagcuggcggaccagagccgguagcgagguugggagagacggagcggaccucagcgcugaagcagaaguccccggagcugcggucuccccgccgcggcugagccaugcggcucccugaccugagacccuggaccucccugcugcugguggacgcggcuuuacuguggcugcuucagggcccucuggggacuuugcuuccucaagggcugccaggacuauggcuggaggggacccugcggcugggagggcugugggggcugcuaaagcuaagagggcugcugggauuuguggggacacugcugcucccgcucugucuggccaccccccugacugucucccugagagcccuggucgcgggggccucacgugcucccccagccagagucgcuucagccccuuggagcuggcugcuggugggguacggggcugcggggcucagcuggucacugugggcuguucugagcccuccuggagcccaggagaaggagcaggaccaggugaacaacaaagucuugauguggaggcugcugaagcucuccaggccggaccugccucuccucguugccgccuucuucuuccuuguccuugcuguuuugggugagacauuaaucccucacuauucuggucgugugauugacauccugggaggugauuuugacccccaugccuuugccagugccaucuucuucaugugccucuucuccuuuggcagcucacugucugcaggcugccgaggaggcugcuucaccuacaccaugucucgaaucaacuugcggauccgggagcagcuuuucuccucccugcugcgccaggaccucgguuucuuccaggagacuaagacaggggagcugaacucacggcugagcucggauaccacccugaugaguaacuggcuuccuuuaaaugccaaugugcucuugcgaagccuggugaaagugguggggcuguauggcuucaugcucagcauaucgccucgacucacccuccuuucucugcugcacaugcccuucacaauagcagcggagaagguguacaacacccgccaucaggaagugcuucgggagauccaggaugcaguggccagggcggggcagguggugcgggaagccguuggagggcugcagaccguucgcaguuuuggggccgaggagcaugaagucugucgcuauaaagaggcccuugaacaaugucggcagcuguauuggcggagagaccuggaacgcgccuuguaccugcucguaaggagggugcugcacuugggggugcagaugcugaugcugagcugugggcugcagcagaugcaggauggggagcucacccagggcagccugcuuuccuuuaugaucuaccaggagagcguggggagcuaugugcagacccugguauacauauauggggauaugcucagcaacgugggagcugcagagaagguuuucuccuacauggaccgacagccaaaucugccuucaccuggcacgcuugcccccaccacucugcaggggguugugaaauuccaagacgucuccuuugcauaucccaaucgcccugacaggccugugcucaaggggcugacguuuacccuacguccuggugaggugacggcgcuggugggacccaaugggucugggaagagcacaguggcugcccugcugcagaaucuguaccagcccacagggggacaggugcugcuggaugaaaagcccaucucacaguaugaacacugcuaccugcacagccaggugguuucaguugggcaggagccugugcuguucuccgguucugugaggaacaacauugcuuaugggcugcagagcugcgaagaugauaaggugauggcggcugcccaggcugcccacgcagaugacuucauccaggaaauggagcauggaauauacacagauguaggggagaagggaagccagcuggcugcgggacagaaacaacgucuggccauugcccgggcccuuguacgagacccgcggguccucauccuggaugaggcuacuagugcccuagaugugcagugcgagcaggcccugcaggacuggaauucccguggggaucgcacagugcuggugauugcucacaggcugcagacaguucagcgcgcccaccagauccuggugcuccaggagggcaagcugcagaagcuugcccagcucuaggagggacaggaccucuauucccgccuggugcagcagcggcugauggacugaggccccagggauacugggcccucuucucaggggcgucuccaggacccagagcuguuccugcuuugaguuucccuagagcugugcggccagauagcuguuccugaguugcaggcacgauggagauuuggacacugugugcuuuuggugggguagagagguggggugggguggggugggggcugucuguguccaggaaacuuaauucccuggugacuagagcuuugccuggugaugaggaguauuuuguggcauaauacauauauuuuaaaauauuuuccuucuuacaugaacuguauacauucauauagaaaauuuagacaauauaaaaaaguacaaagaagaaaaguaaaaguacccauuguuucacuuccuggagauaaccauaguugcuauuuugcugccugucccaucagucguuuaucuguuguuugagauagaaauuaaccaaaaaugacauaaauauucaugagauugccuuccuauauccuuccuuguuccuaccagugucugcuauuuugaagaagcuagggucuggagggacagagaacaguucccugauuaacaguauuaauagcgacauugguaacagcuaccauuuauagaguuuuaaugggaguaggagcuaugcuaaguguuuuucauguauuaucguuuuuaaucauuauccccaacccuaugagguugguuauuauccccauuuuacagaugaggaaacugaagcucaaagaggcucaaugacuuucccaagguggucguagugguggaguuggaguuugaacacaggccugacccuagaguccacacccugacccaaucaauuauauugcaucuuggguccauaaacccuaauccauaaucccaucaagaaaagcucugcugcucuuagcucuaaauaauucagaaucuauucucuucucuccagucccguuguuauagucuucacucauagacuuaagaugaucccaucaccagagagguuucucuaccauuagcuucccucuuccggccauucuucacaaagucauuuuucuaaauucugugucacauacgaugauggcauuucuggaaauuccuucaggugcucucaagcccugcugcagagauccuuuucagagcacacacuguuccagcccaucugucucacccucuccuguuguauccagcuccacgacaaacuucugccuuccccaacaccuuugugccuuugcauaugguguuuucuugcccauuuucugcucgacucgccccugauuuucaaguucaagacuuaacucaggguucaggucuuccaggaggccuuacuuaugucgucagucuggggaacucuccaugugcuucuaucacugugcgguuaccucuuucacagcccuuuuaaaguucuaucuucccuuucccaccuuuuuugaccuuccacuagaccaugagcaccugggcggaaagccauauaucuuauuaagcuuuauaucugcuaccuggccgagggccuaauucauaguggagaauaaauagucaauugaauaaaugaauaaauaucuccaccaucguacuaaucuuaauccucccugcccacucccaccacugaaaaugcaacauuguacacaucacugguuguugggagggacuuaccuuggaaaguugcuauucuaggaaagagaaaccuucauauuccuggaaacagcagguaguuuccagugcuggcaaugaauuccccagaacugcuguuuuggauuuuuucuugccuggcagcuguugggagcagggugcagugaggauggggugagagugggcaguuucuugugcagauuugccuuucuuucauccuggggcugacuugcagcuccacacccauccaucucucaaauuucacagaggguaaaauaggcauuuggagagaaagaacucuggccugauuccuuucucucccacaaauguccuuuauucauaaaacaggaauaauaauuccuguaucucccaacuacauggaagcugcagcccucacagaagaagaugaucugagaaauucuuugauuuccucaguacaguuauacccaugcaucauaauacuuuaagccuggaaggcaucuuaaaaauaaugcaacagucaaaccuaauuuuacagagaaacugacaugaaaucacgcagcuaaucaugauaaagcuggguggaaaacuuaucuugaugggcaguacaggaagaugcaguagaccuuaagauguccugaaaguuucuuaucucaggggaaacucccagguaggcuuuaugucagggacacagaaaaaugcucccugaaagucaaaauauucgggcuagacagacaaauuccuguaagugugguuugucugggaaccacagaugucacuaauccugguuugcuccagaguucuuuuuguucacuccuaccccccaucaccauuugauugaucuccuuacccuguaauuuccccuucuugucgcuuaccugcaguaucuuucccacccaggcaugccuuauucuuucuaaaggaaaguaugaauggagaggggaaagcuugggaaacugauagauuuccuuggaugccaaaacaccuccauagccugucugcccggcccuauguggaaacagcauugaguuucaaguccuuuaugccuccacccagggauagccacuuguaauccacauggcaauugugaaacaagcaggaaaugcguaauugucagaauuuuguggggaaaggacuagggaauaaggaaaacaaagaucuuccuuguguuuuagagcugucagcuagaggagcaccugcuugagucugaugccaucuaauggucccagaagaaacuggguuuugaaccuagaguuccauggacucuuaggaauuagacuacuacuacuacuaagcauucacuggugcuuacuaugugcuauugcugugccaaguaucugaaaccugucuucuuaccuuauuuuucaagauaauucuauguggcagguauuacuaucucaauucuaagagugagaaaauggaguuuuagaaacauuuacuaacuugccugggucacauagcuaaggaagagguggacuugcccagcuuugcauaaaacuccucaaaagaguugccuauacucccugacuccacuuaucuuccuacuauccucuuuuuaaaauauauuauuuauuuauuuaaauaagcaauauaugaaugugguuugaaauucaaaagacacaaagaaguauacagaggaaagccucacucucaauccuucucaagguuugcuaauuccucuugcauaggcaauccguucuuccagcuuuguguuuaucuuuccagagaaguuuacuguguauuaagcaaauauguauaucuuuauucuugcucaguauuuucgcaaacagcagcugucuaaguucacuguucugaacuuuauuuuuuaaauuaaaaauauauggcuauguaguauucuauuuua

SEQ ID NO: 42 - protein, 686 aa, Homo sapiens
MRLPDLRPWTSLLLVDAALLWLLQGPLGTLLPQGLPGLWLEGTLRLGGLWGLLKLRGLLGFVGTLLLPLCLATPLTVSLRALVAGASRAPPARVASAPWSWLLVGYGAAGLSWSLWAVLSPPGAQEKEQDQVNNKVLMWRLLKLSRPDLPLLVAAFFFLVLAVLGETLIPHYSGRVIDILGGDFDPHAFASAIFFMCLFSFGSSLSAGCRGGCFTYTMSRINLRIREQLFSSLLRQDLGFFQETKTGELNSRLSSDTTLMSNWLPLNANVLLRSLVKVVGLYGFMLSISPRLTLLSLLHMPFTIAAEKVYNTRHQEVLREIQDAVARAGQVVREAVGGLQTVRSFGAEEHEVCRYKEALEQCRQLYWRRDLERALYLLVRRVLHLGVQMLMLSCGLQQMQDGELTQGSLLSFMIYQESVGSYVQTLVYIYGDMLSNVGAAEKVFSYMDRQPNLPSPGTLAPTTLQGVVKFQDVSFAYPNRPDRPVLKGLTFTLRPGEVTALVGPNGSGKSTVAALLQNLYQPTGGQVLLDEKPISQYEHCYLHSQVVSVGQEPVLFSGSVRNNIAYGLQSCEDDKVMAAAQAAHADDFIQEMEHGIYTDVGEKGSQLAAGQKQRLAIARALVRDPRVLILDEATSALDVQCEQALQDWNSRGDRTVLVIAHRLQTVQRAHQILVLQEGKLQKLAQL

(19) United States
(12) Patent Application Publication
(10) Pub. No.: US 2020/0157635 A1
(43) Pub. Date: May 21, 2020

(54) DIAGNOSTIC AND THERAPEUTIC METHODS FOR CANCER

(71) Applicant: Genentech, Inc., South San Francisco, CA (US)

(72) Inventors: Marcin KOWANETZ, San Francisco, CA (US); Mahrukh HUSENI, Union City, CA (US); Wei ZOU, San Carlos, CA (US)

(21) Appl. No.: 16/591,813

(22) Filed: Oct. 3, 2019

Related U.S. Application Data
(63) Continuation of application No. PCT/US2018/027561, filed on Apr. 13, 2018.
(60) Provisional application No. 62/628,227, filed on Feb. 8, 2018.
(60) Provisional application No. 62/485,874, filed on Apr. 14, 2017.

(51) Int. Cl. C12Q 1/6886; G01N 33/50; C07K 16/28

(52) U.S. Cl. CPC C12Q 1/6886; G01N 33/5011; C07K 16/2827; G01N 2333/70532; A61K 31/337; G01N 2333/96436; G01N 2333/70517; G01N 2333/7158; G01N 2333/57

(57) ABSTRACT

The present invention provides diagnostic methods, therapeutic methods, and compositions for the treatment of cancer. The invention is based, at least in part, on the discovery that an immune-score expression level based on one or more of PD-L1, CXCL9, IFNG, GZMB, CD8A, and PD-1 in a sample obtained from an individual having cancer can be used in methods of predicting the therapeutic efficacy of treatment with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)).

### SEQUENCE LISTING

The instant application contains a Sequence Listing which has been submitted electronically in ASCII format and is hereby incorporated by reference in its entirety. Said ASCII copy, created on Sep. 25, 2019 is named 50474-158003_Sequence_Listing_9.25.19_ST25 and is 96,586 bytes in size.

### FIELD OF THE INVENTION

The present invention is directed to diagnostic and therapeutic methods for the treatment of cancer using PD-L1 axis binding antagonists. Also provided are related assays and kits.

### BACKGROUND OF THE INVENTION

Cancer remains to be one of the most deadly threats to human health. In the U.S., cancer affects nearly 1.3 million new patients each year and is the second leading cause of death after heart disease, accounting for approximately 1 in 4 deaths. It is also predicted that cancer may surpass cardiovascular diseases as the number one cause of death within 5 years. Solid tumors are responsible for most of those deaths.

Studies in humans with immune checkpoint inhibitors have demonstrated the promise of harnessing the immune system to control and eradicate tumor growth. The programmed death 1 (PD-1) receptor and its ligand programmed death-ligand 1 (PD-L1) are immune checkpoint proteins that have been implicated in the suppression of immune system responses during chronic infections, pregnancy, tissue allografts, autoimmune diseases, and cancer. PD-L1 regulates the immune response by binding to the inhibitory receptor PD-1, which is expressed on the surface of T-cells, B-cells, and monocytes. PD-L1 negatively regulates T-cell function also through interaction with another receptor, B7-1. Formation of the PD-L1/PD-1 and PD-L1/B7-1 complexes negatively regulates T-cell receptor signaling, resulting in the subsequent downregulation of T-cell activation and suppression of anti-tumor immune activity.

Although there have been significant advances in the medical treatment of certain cancers, the overall 5-year survival rate for all cancers has improved only by about 10% in the past 20 years. Malignant solid tumors, in particular, metastasize and grow rapidly in an uncontrolled manner, making their timely detection and treatment extremely difficult.

Despite the significant advancement in the treatment of cancer, improved diagnostic methods and cancer therapies and are still being sought.

### SUMMARY OF THE INVENTION

The present invention provides therapeutic and diagnostic methods and compositions for treating an individual having a cancer.

In one aspect, provided herein is a method of identifying an individual having a cancer who may benefit from a treatment comprising a PD-L1 binding antagonist, the method comprising determining the expression level of PD-L1, CXCL9, and IFNG in a sample from the individual, wherein an immune-score expression level of PD-L1, CXCL9, and IFNG in the sample that is above a reference immune-score expression level identifies the individual as one who may benefit from a treatment comprising a PD-L1 binding antagonist, wherein the reference immune-score expression level is an immune-score expression level of PD-L1, CXCL9, and IFNG in a reference population.

In another aspect, provided herein is a method for selecting a therapy for an individual having a cancer, the method comprising determining the expression level of PD-L1, CXCL9, and IFNG in a sample from the individual, wherein an immune-score expression level of PD-L1, CXCL9, and IFNG in the sample that is above a reference immune-score expression level identifies the individual as one who may benefit from a treatment comprising a PD-L1 binding antagonist, wherein the reference immune-score expression level is an immune-score expression level of PD-L1, CXCL9, and IFNG in a reference population.

In some embodiments, the immune-score expression level of PD-L1, CXCL9, and IFNG in the sample is above the reference immune-score expression level and the method further comprises administering to the individual an effective amount of a PD-L1 binding antagonist. In some embodiments, an immune-score expression level of PD-L1, CXCL9, and IFNG in the sample that is below the reference immune-score expression level identifies the individual as one who is less likely to benefit from a treatment comprising a PD-L1 binding antagonist. In some embodiments, the immune-score expression level of PD-L1, CXCL9, and IFNG in the sample is below the reference immune-score expression level and the method further comprises administering to the individual an effective amount of an anti-cancer therapy other than, or in addition to, a PD-L1 binding antagonist (e.g., the anti-cancer therapy other than, or in addition to, a PD-L1 binding antagonist may include a cytotoxic agent, a growth-inhibitory agent, a radiation therapy, an anti-angiogenic agent, as described herein, or a combination thereof, alone, or in addition to a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) and/or any additional therapeutic agent described herein).

In another aspect, provided herein is a method of treating an individual having a cancer, the method comprising (a) determining the expression level of PD-L1, CXCL9, and IFNG in a sample from the individual, wherein an immune-score expression level of PD-L1, CXCL9, and IFNG in the sample has been determined to be above a reference immune-score expression level, wherein the reference immune-score expression level is an immune-score expression level of PD-L1, CXCL9, and IFNG in a reference population, and (b) administering an effective amount of a PD-L1 binding antagonist to the individual based on the immune-score expression level of PD-L1, CXCL9, and IFNG determined in step (a).

In another aspect, provided herein is a method of treating an individual having a cancer, the method comprising administering to the individual an effective amount of a PD-L1 binding antagonist, wherein prior to treatment the expression level of PD-L1, CXCL9, and IFNG in a sample from the individual has been determined and an immune-score expression level of PD-L1, CXCL9, and IFNG in the sample that is above a reference immune-score expression level has been determined, wherein the reference immune-score expression level is an immune-score expression level of PD-L1, CXCL9, and IFNG in a reference population.

In some embodiments, the immune-score expression level of PD-L1, CXCL9, and IFNG in the sample is in the top 80^{th} percentile of the immune-score expression level of PD-L1, CXCL9, and IFNG in the reference population. In some embodiments, the immune-score expression level of PD-L1, CXCL9, and IFNG in the sample is in the top 50^{th} percentile of the immune-score expression level of PD-L1, CXCL9, and IFNG in the reference population. In some embodiments, the immune-score expression level of PD-L1, CXCL9, and IFNG in the sample is in the top 20^{th} percentile of the immune-score expression level of PD-L1, CXCL9, and IFNG in the reference population.

In some embodiments, the reference population is a population of individuals having the cancer, the population of individuals consisting of a first subset of individuals who have been treated with a PD-L1 binding antagonist therapy and a second subset of individuals who have been treated with a non-PD-L1 binding antagonist therapy, wherein the non-PD-L1 binding antagonist therapy does not comprise a PD-L1 binding antagonist.

In some embodiments, the immune-score expression level of PD-L1, CXCL9, and IFNG is an average of the expression level of each of PD-L1, CXCL9, and IFNG. In some embodiments, the average of the expression level of each of PD-L1, CXCL9, and IFNG is an average of a normalized expression level of each of PD-L1, CXCL9, and IFNG. In some embodiments, the immune-score expression level of PD-L1, CXCL9, and IFNG is a median of the expression level of each of PD-L1, CXCL9, and IFNG. In some embodiments, the immune-score expression level of PD-L1, CXCL9, and IFNG is a median of a normalized expression level of each of PD-L1, CXCL9, and IFNG. In some embodiments, the normalized expression level of each of PD-L1, CXCL9, and IFNG is the expression level of each of PD-L1, CXCL9, and IFNG normalized to a reference gene. In some embodiments, the reference immune-score expression level is a pre-assigned expression level of PD-L1, CXCL9, and IFNG.

In another aspect, provided herein is a method of identifying an individual having a cancer who may benefit from a treatment comprising a PD-L1 binding antagonist, the method comprising determining the expression level of PD-L1, IFNG, GZMB, and CD8A in a sample from the individual, wherein an immune-score expression level of PD-L1, IFNG, GZMB, and CD8A in the sample that is above a reference immune-score expression level identifies the individual as one who may benefit from a treatment comprising a PD-L1 binding antagonist, wherein the reference immune-score expression level is an immune-score expression level of PD-L1, IFNG, GZMB, and CD8A in a reference population.

In another aspect, provided herein is a method for selecting a therapy for an individual having a cancer, the method comprising determining the expression level of PD-L1, IFNG, GZMB, and CD8A in a sample from the individual, wherein an immune-score expression level of PD-L1, IFNG, GZMB, and CD8A in the sample that is above a reference immune-score expression level identifies the individual as one who may benefit from a treatment comprising a PD-L1 binding antagonist, wherein the reference immune-score expression level is an immune-score expression level of PD-L1, IFNG, GZMB, and CD8A in a reference population.

In some embodiments, the immune-score expression level of PD-L1, IFNG, GZMB, and CD8A in the sample is above the reference immune-score expression level and the method further comprises administering to the individual an effective amount of a PD-L1 binding antagonist.

In some embodiments, the immune-score expression level of PD-L1, IFNG, GZMB, and CD8A in the sample that is below the reference immune-score expression level identifies the individual as one who is less likely to benefit from a treatment comprising a PD-L1 binding antagonist.

In some embodiments, the immune-score expression level of PD-L1, IFNG, GZMB, and CD8A in the sample is below the reference immune-score expression level and the method further comprises administering to the individual an effective amount of an anti-cancer therapy other than, or in addition to, a PD-L1 binding antagonist (e.g., the anti-cancer therapy other than, or in addition to, a PD-L1 binding antagonist may include a cytotoxic agent, a growth-inhibitory agent, a radiation therapy, an anti-angiogenic agent, as described herein, or a combination thereof, alone, or in addition to a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) and/or any additional therapeutic agent described herein).

In another aspect, provided herein is a method of treating an individual having a cancer, the method comprising (a) determining the expression level of PD-L1, IFNG, GZMB, and CD8A in a sample from the individual, wherein an immune-score expression level of PD-L1, IFNG, GZMB, and CD8A in the sample has been determined to be above a reference immune-score expression level, wherein the reference immune-score expression level is an immune-score expression level of PD-L1, IFNG, GZMB, and CD8A in a reference population, and (b) administering an effective amount of a PD-L1 binding antagonist to the individual based on the immune-score expression level of PD-L1, IFNG, GZMB, and CD8A determined in step (a).

In another aspect, provided herein is a method of treating an individual having a cancer, the method comprising administering to the individual an effective amount of a PD-L1 binding antagonist, wherein prior to treatment the expression level of PD-L1, IFNG, GZMB, and CD8A in a sample from the individual has been determined and an immune-score expression level of PD-L1, IFNG, GZMB, and CD8A in the sample that is above a reference immune-score expression level has been determined, wherein the reference immune-score expression level is an immune-score expression level of PD-L1, IFNG, GZMB, and CD8A in a reference population.

In some embodiments, the immune-score expression level of PD-L1, IFNG, GZMB, and CD8A in the sample is in the top 80^{th} percentile of the immune-score expression level of PD-L1, IFNG, GZMB, and CD8A in the reference population. In some embodiments, the immune-score expression level of PD-L1, IFNG, GZMB, and CD8A in the sample is in the top 50^{th} percentile of the immune-score expression level of PD-L1, IFNG, GZMB, and CD8A in the reference population. In some embodiments, the immune-score expression level of PD-L1, IFNG, GZMB, and CD8A in the sample is in the top 20^{th} percentile of the immune-score expression level of PD-L1, IFNG, GZMB, and CD8A in the reference population.

In some embodiments, the reference population is a population of individuals having the cancer, the population of individuals consisting of a first subset of individuals who have been treated with a PD-L1 binding antagonist therapy and a second subset of individuals who have been treated with a non-PD-L1 binding antagonist therapy, wherein the non-PD-L1 binding antagonist therapy does not comprise a PD-L1 binding antagonist.

In some embodiments, the immune-score expression level of PD-L1, IFNG, GZMB, and CD8A is an average of the expression level of each of PD-L1, IFNG, GZMB, and CD8A. In some embodiments, the average expression level of each of PD-L1, IFNG, GZMB, and CD8A is an average of a normalized expression level of each of PD-L1, IFNG, GZMB, and CD8A. In some embodiments, the immune-score expression level of PD-L1, IFNG, GZMB, and CD8A is a median of the expression level of each of PD-L1, IFNG, GZMB, and CD8A. In some embodiments, the immune-score expression level of PD-L1, IFNG, GZMB, and CD8A is a median of a normalized expression level of each of PD-L1, IFNG, GZMB, and CD8A. In some embodiments, the normalized expression level of each of PD-L1, IFNG, GZMB, and CD8A is the expression level of each of PD-L1, IFNG, GZMB, and CD8A normalized to a reference gene. In some embodiments, the reference immune-score expression level is a pre-assigned expression level of PD-L1, IFNG, GZMB, and CD8A.

In another aspect, provided herein is a method of identifying an individual having a cancer who may benefit from a treatment comprising a PD-L1 binding antagonist, the method comprising determining the expression level of PD-L1, IFNG, GZMB, CD8A, and PD-1 in a sample from the individual, wherein an immune-score expression level of PD-L1, IFNG, GZMB, CD8A, and PD-1 in the sample that is above a reference immune-score expression level identifies the individual as one who may benefit from a treatment comprising a PD-L1 binding antagonist, wherein the reference immune-score expression level is an immune-score expression level of PD-L1, IFNG, GZMB, CD8A, and PD-1 in a reference population.

In another aspect, provided herein method for selecting a therapy for an individual having a cancer, the method comprising determining the expression level of PD-L1, IFNG, GZMB, CD8A, and PD-1 in a sample from the individual, wherein an immune-score expression level of PD-L1, IFNG, GZMB, CD8A, and PD-1 in the sample that is above a reference immune-score expression level identifies the individual as one who may benefit from a treatment comprising a PD-L1 binding antagonist, wherein the reference immune-score expression level is an immune-score expression level of PD-L1, IFNG, GZMB, CD8A, and PD-1 in a reference population.

In some embodiments, the immune-score expression level of PD-L1, IFNG, GZMB, CD8A, and PD-1 in the sample is above the reference immune-score expression level and the method further comprises administering to the individual an effective amount of a PD-L1 binding antagonist. In some embodiments, the immune-score expression level of PD-L1, IFNG, GZMB, CD8A, and PD-1 in the sample that is below the reference immune-score expression level identifies the individual as one who is less likely to benefit from a treatment comprising a PD-L1 binding antagonist. In some embodiments, the immune-score expression level of PD-L1, IFNG, GZMB, CD8A, and PD-1 in the sample is below the reference immune-score expression level and the method further comprises administering to the individual an effective amount of an anti-cancer therapy other than, or in addition to, a PD-L1 binding antagonist (e.g., the anti-cancer therapy other than, or in addition to, a PD-L1 binding antagonist may include a cytotoxic agent, a growth-inhibitory agent, a radiation therapy, an anti-angiogenic agent, as described herein, or a combination thereof, alone, or in addition to a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) and/or any additional therapeutic agent described herein).

In another aspect, provided herein is a method of treating an individual having a cancer, the method comprising (a) determining the expression level of PD-L1, IFNG, GZMB, CD8A, and PD-1 in a sample from the individual, wherein an immune-score expression level of PD-L1, IFNG, GZMB, CD8A, and PD-1 in the sample relative to a reference immune-score expression level has been determined, wherein the reference immune-score expression level is an immune-score expression level of PD-L1, IFNG, GZMB, CD8A, and PD-1 in a reference population, and (b) administering an effective amount of a PD-L1 binding antagonist to the individual based on the immune-score expression level of PD-L1, IFNG, GZMB, CD8A, and PD-1 determined in step (a).

In another aspect, provided herein is a method of treating an individual having a cancer, the method comprising administering to the individual an effective amount of a PD-L1 binding antagonist, wherein prior to treatment the expression level of PD-L1, IFNG, GZMB, CD8A, and PD-1 in a sample from the individual has been determined and an immune-score expression level of PD-L1, IFNG, GZMB, CD8A, and PD-1 in the sample that is above a reference immune-score expression level has been determined, wherein the reference immune-score expression level is an immune-score expression level of PD-L1, IFNG, GZMB, CD8A, and PD-1 in a reference population.

In some embodiments, the immune-score expression level of PD-L1, IFNG, GZMB, CD8A, and PD-1 in the sample is in the top 80^{th} percentile of the immune-score expression level of PD-L1, IFNG, GZMB, CD8A, and PD-1 in the reference population. In some embodiments, the immune-score expression level of PD-L1, IFNG, GZMB, CD8A, and PD-1 in the sample is in the top 50^{th} percentile of the immune-score expression level of PD-L1, IFNG, GZMB, CD8A, and PD-1 in the reference population. In some embodiments, the immune-score expression level of PD-L1, IFNG, GZMB, CD8A, and PD-1 in the sample is in the top 20^{th} percentile of the immune-score expression level of PD-L1, IFNG, GZMB, CD8A, and PD-1 in the reference population.

In some embodiments, the immune-score expression level of PD-L1, IFNG, GZMB, CD8A, and PD-1 is an average of the expression level of each of PD-L1, IFNG, GZMB, CD8A, and PD-1. In some embodiments, the average of the expression level of each of PD-L1, IFNG, GZMB, CD8A, and PD-1 is an average of a normalized expression level of each of PD-L1, IFNG, GZMB, CD8A, and PD-1.

In some embodiments, the immune-score expression level of PD-L1, IFNG, GZMB, CD8A, and PD-1 is a median of the expression level of each of PD-L1, IFNG, GZMB, CD8A, and PD-1.

In some embodiments, the immune-score expression level of PD-L1, IFNG, GZMB, CD8A, and PD-1 is a median of a normalized expression level of each of PD-L1, IFNG, GZMB, CD8A, and PD-1. In some embodiments, the normalized expression level of each of PD-L1, IFNG, GZMB, CD8A, and PD-1 is the expression level of each of PD-L1, IFNG, GZMB, CD8A, and PD-1 normalized to a reference gene. In some embodiments, reference immune-score expression level is a pre-assigned expression level of PD-L1, IFNG, GZMB, CD8A, and PD-1.

In some embodiments of any of the above aspects, the reference population is a population of individuals having the cancer, the population of individuals consisting of a first subset of individuals who have been treated with a PD-L1 binding antagonist therapy and a second subset of individuals who have been treated with a non-PD-L1 binding antagonist therapy, wherein the non-PD-L1 binding antagonist therapy does not comprise a PD-L1 binding antagonist.

In some embodiments of any of the above aspects, the reference immune-score expression level significantly separates each of the first and second subsets of individuals based on a significant difference between an individual's responsiveness to treatment with the PD-L1 binding antagonist therapy and an individual's responsiveness to treatment with the non-PD-L1 binding antagonist therapy above the reference immune-score expression level, wherein the individual's responsiveness to treatment with the PD-L1 binding antagonist therapy is significantly improved relative to the individual's responsiveness to treatment with the non-PD-L1 binding antagonist therapy.

In some embodiments of any of the above aspects, the reference immune-score expression level significantly separates each of the first and second subsets of individuals based on a significant difference between an individual's responsiveness to treatment with the PD-L1 binding antagonist therapy and an individual's responsiveness to treatment with the non-PD-L1 binding antagonist therapy below the reference immune-score expression level, wherein the individual's responsiveness to treatment with the non-PD-L1 binding antagonist therapy is significantly improved relative to the individual's responsiveness to treatment with the PD-L1 binding antagonist therapy.

In some embodiments of any of the above aspects, the responsiveness to treatment is an increase in PFS.

In some embodiments of any of the above aspects, the responsiveness to treatment is an increase in OS.

In some embodiments of any of the above aspects, the reference gene is a housekeeping gene. In some embodiments, the housekeeping gene is TMEM55B.

In some embodiments of any of the above aspects, benefit from the treatment comprising a PD-L1 binding antagonist is an increase in OS.

In some embodiments of any of the above aspects, benefit from the treatment comprising a PD-L1 binding antagonist is an increase in PFS.

In some embodiments of any of the above aspects, benefit from the treatment comprising a PD-L1 binding antagonist is an increase in OS and PFS.

In some embodiments of any of the above aspects, the expression level is a nucleic acid expression level. In some embodiments, the nucleic acid expression level is an mRNA expression level. In some embodiments, the mRNA expression level is determined by RNA-seq, RT-qPCR, qPCR, multiplex qPCR or RT-qPCR, microarray analysis, SAGE, MassARRAY technique, ISH, or a combination thereof. In some embodiments, the mRNA expression level is detected using RNA-seq. In some embodiments, the mRNA expression level is detected using RT-qPCR. In some embodiments, the expression level is detected in tumor cells, tumor infiltrating immune cells, stromal cells, or a combination thereof.

In some embodiments of any of the above aspects, the sample is a tissue sample, a cell sample, a whole blood sample, a plasma sample, a serum sample, or a combination thereof. In some embodiments, the tissue sample is a tumor tissue sample. In some embodiments, the tumor tissue sample comprises tumor cells, tumor-infiltrating immune cells, stromal cells, or a combination thereof. In some embodiments, the tumor tissue sample is a formalin-fixed and paraffin-embedded (FFPE) sample, an archival sample, a fresh sample, or a frozen sample. In some embodiments, the tumor tissue sample is a FFPE sample.

In some embodiments of any of the above aspects, the cancer is selected from the group consisting of a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a merkel cell cancer, or a hematologic malignancy. In some embodiments, the cancer is a lung cancer, a kidney cancer, a bladder cancer, or a breast cancer. In some embodiments, the lung cancer is a non-small cell lung cancer (NSCLC). In some embodiments, the kidney cancer is a renal cell carcinoma (RCC). In some embodiments, the bladder cancer is a urothelial bladder cancer (UBC). In some embodiments, the breast cancer is a triple negative breast cancer (TNBC).

In some embodiments of any of the above aspects, the PD-L1 binding antagonist inhibits the binding of PD-L1 to PD-1, the binding of PD-L1 to B7-1, or the binding of PD-L1 to both PD-1 and B7-1. In some embodiments, the PD-L1 binding antagonist is an anti-PD-L1 antibody.

In some embodiments of any of the above aspects, the anti-PD-L1 antibody is selected from the group consisting of atezolizumab (MPDL3280A), YW243.55.S70, MSB0010718C, MDX-1105, and MEDI4736. In some embodiments, the anti-PD-L1 antibody comprises the following hypervariable regions: (a) an HVR-H1 sequence of GFTFSDSWIH (SEQ ID NO: 9); (b) an HVR-H2 sequence of AWISPYGGSTYYADSVKG (SEQ ID NO: 10); (c) an HVR-H3 sequence of RHWPGGFDY (SEQ ID NO: 11); (d) an HVR-L1 sequence of RASQDVSTAVA (SEQ ID NO: 12); (e) an HVR-L2 sequence of SASFLYS (SEQ ID NO: 13); and (f) an HVR-L3 sequence of QQYLYHPAT (SEQ ID NO: 14). In some embodiments, the anti-PD-L1 antibody comprises (a) a heavy chain variable (VH) domain comprising an amino acid sequence having at least 90% sequence identity to the amino acid sequence of SEQ ID NO: 16; (b) a light chain variable (VL) domain comprising an amino acid sequence having at least 90% sequence identity to the amino acid sequence of SEQ ID NO: 17; or (c) a VH domain as in (a) and a VL domain as in (b). In some embodiments, the anti-PD-L1 antibody comprises: (a) a heavy chain variable (VH) domain comprising an amino acid sequence having at least 95% sequence identity to the amino acid sequence of SEQ ID NO: 16; (b) a light chain variable (VL) domain comprising an amino acid sequence having at least 95% sequence identity to the amino acid sequence of SEQ ID NO: 17; or (c) a VH domain as in (a) and a VL domain as in (b). In some embodiments, the anti-PD-L1 antibody comprises: (a) a heavy chain variable (VH) domain comprising an amino acid sequence having at least 96% sequence identity to the amino acid sequence of SEQ ID NO: 16; (b) a light chain variable (VL) domain comprising an amino acid sequence having at least 96% sequence identity to the amino acid sequence of SEQ ID NO: 17; or (c) a VH domain as in (a) and a VL domain as in (b). In some embodiments, the anti-PD-L1 antibody comprises: (a) a heavy chain variable (VH) domain comprising an amino acid sequence having at least 97% sequence identity to the amino acid sequence of SEQ ID NO: 16; (b) a light chain variable (VL) domain comprising an amino acid sequence having at least 97% sequence identity to the amino acid sequence of SEQ ID NO: 17; or (c) a VH domain as in (a) and a VL domain as in (b). In some embodiments, the anti-PD-L1 antibody comprises: (a) a heavy chain variable (VH) domain comprising an amino acid sequence having at least 98% sequence identity to the amino acid sequence of SEQ ID NO: 16; (b) a light chain variable (VL) domain comprising an amino acid sequence having at least 98% sequence identity to the amino acid sequence of SEQ ID NO: 17; or (c) a VH domain as in (a) and a VL domain as in (b). In some embodiments, the anti-PD-L1 antibody comprises: (a) a heavy chain variable (VH) domain comprising an amino acid sequence having at least 99% sequence identity to the amino acid sequence of SEQ ID NO: 16; (b) a light chain variable (VL) domain comprising an amino acid sequence having at least 99% sequence identity to the amino acid sequence of SEQ ID NO: 17; or (c) a VH domain as in (a) and a VL domain as in (b). In some embodiments, the anti-PD-L1 antibody comprises: (a) a VH domain comprising the amino acid sequence of SEQ ID NO: 16; (b) a VL domain comprising the amino acid sequence of SEQ ID NO: 17; or (c) a VH domain as in (a) and a VL domain as in (b). In some embodiments the anti-PD-L1 antibody comprises: (a) a VH domain comprising the amino acid sequence of SEQ ID NO: 16; and (b) a VL domain comprising the amino acid sequence of SEQ ID NO: 17. In some embodiments, the anti-PD-L1 antibody is atezolizumab.

In some embodiments of any of the above aspects, the non-PD-L1 binding antagonist is an anti-neoplastic agent, a chemotherapeutic agent, a growth inhibitory agent, an anti-angiogenic agent, a radiation therapy, or a cytotoxic agent.

In some embodiments of any of the above aspects, the anti-cancer therapy is an anti-neoplastic agent, a chemotherapeutic agent, a growth inhibitory agent, an anti-angiogenic agent, a radiation therapy, or a cytotoxic agent.

In some embodiments of any of the above aspects, the individual has not been previously treated for the cancer. In some embodiments of any of the above aspects, the individual has not been previously administered a PD-L1 binding antagonist.

In some embodiments of any of the above aspects, the treatment comprising a PD-L1 binding antagonist is a monotherapy.

In some embodiments of any of the above aspects, the method further comprises administering to the individual an effective amount of an additional therapeutic agent. In some embodiments, the additional therapeutic agent is an anti-neoplastic agent, a chemotherapeutic agent, a growth inhibitory agent, an anti-angiogenic agent, a radiation therapy, or a cytotoxic agent.

In some embodiments of any of the above aspects, the individual is a human.

In another aspect, provided herein is a kit for identifying an individual having a cancer who may benefit from a treatment comprising a PD-L1 binding antagonist, the kit comprising (a) reagents for determining the expression level of PD-L1, CXCL9, and IFNG in a sample from the individual; and, optionally, (b) instructions for using the reagents to identify an individual having a cancer who may benefit from a treatment comprising a PD-L1 binding antagonist.

In another aspect, provided herein is a kit for identifying an individual having a cancer who may benefit from a treatment comprising a PD-L1 binding antagonist, the kit comprising (a) reagents for determining the expression level of PD-L1, IFNG, GZMB, and CD8A in a sample from the individual; and, optionally, (b) instructions for using the reagents to identify an individual having a cancer who may benefit from a treatment comprising a PD-L1 binding antagonist.

In another aspect, provided herein is a kit for identifying an individual having a cancer who may benefit from a treatment comprising a PD-L1 binding antagonist, the kit comprising reagents for determining the expression level of PD-L1, IFNG, GZMB, CD8A, and PD-1 in a sample from the individual; and, optionally, instructions for using the reagents to identify an individual having a cancer who may benefit from a treatment comprising a PD-L1 binding antagonist.

In another aspect, provided herein is an assay for identifying an individual having a cancer who is a candidate for a treatment comprising a PD-L1 binding antagonist, the assay comprising determining the expression level of PD-L1, CXCL9, and IFNG in a sample from the individual, wherein an immune-score expression level of PD-L1, CXCL9, and IFNG in the sample that is above a reference immune-score expression level identifies the individual as one who may benefit from the treatment comprising a PD-L1 binding antagonist, and wherein the reference immune-score expression level is an immune-score expression level of PD-L1, CXCL9, and IFNG in a reference population.

In another aspect, provided herein is an assay for identifying an individual having a cancer who is a candidate for a treatment comprising a PD-L1 binding antagonist, the assay comprising determining the expression level of PD-L1, IFNG, GZMB, and CD8A in a sample from the individual, wherein an immune-score expression level of PD-L1, IFNG, GZMB, and CD8A in the sample that is above a reference immune-score expression level identifies the individual as one who may benefit from the treatment comprising a PD-L1 binding antagonist, and wherein the reference immune-score expression level is an immune-score expression level of PD-L1, IFNG, GZMB, and CD8A in a reference population.

In another aspect, provided herein is an assay for identifying an individual having a cancer who is a candidate for a treatment comprising a PD-L1 binding antagonist, the assay comprising determining the expression level of PD-L1, IFNG, GZMB, CD8A, and PD-1 in a sample from the individual, wherein an immune-score expression level of PD-L1, IFNG, GZMB, CD8A, and PD-1 in the sample that is above a reference immune-score expression level identifies the individual as one who may benefit from the treatment comprising a PD-L1 binding antagonist, and wherein the reference immune-score expression level is an immune-score expression level of PD-L1, IFNG, GZMB, CD8A, and PD-1 in a reference population.

In another aspect, provided herein is a method of identifying an individual having a cancer who may benefit from a treatment comprising a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the method comprising determining the expression level of PD-L1, CXCL9, and IFNG in a sample from the individual, wherein an immune-score expression level of PD-L1, CXCL9, and IFNG in the sample that is above a reference immune-score expression level identifies the individual as one who may benefit from a treatment comprising a PD-L1 axis binding antagonist, wherein the reference immune-score expression level is an immune-score expression level of PD-L1, CXCL9, and IFNG in a reference population.

In another aspect, provided herein is a method for selecting a therapy for an individual having a cancer, the method comprising determining the expression level of PD-L1, CXCL9, and IFNG in a sample from the individual, wherein an immune-score expression level of PD-L1, CXCL9, and IFNG in the sample that is above a reference immune-score expression level identifies the individual as one who may benefit from a treatment comprising a PD-L1 axis binding antagonist, wherein the reference immune-score expression level is an immune-score expression level of PD-L1, CXCL9, and IFNG in a reference population.

In some embodiments, the immune-score expression level of PD-L1, CXCL9, and IFNG in the sample is above the reference immune-score expression level and the method further comprises administering to the individual an effective amount of a PD-L1 axis binding antagonist. In some embodiments, an immune-score expression level of PD-L1, CXCL9, and IFNG in the sample that is below the reference immune-score expression level identifies the individual as one who is less likely to benefit from a treatment comprising a PD-L1 axis binding antagonist. In some embodiments, the immune-score expression level of PD-L1, CXCL9, and IFNG in the sample is below the reference immune-score expression level and the method further comprises administering to the individual an effective amount of an anti-cancer therapy other than, or in addition to, a PD-L1 axis binding antagonist (e.g., the anti-cancer therapy other than, or in addition to, a PD-L1 axis binding antagonist may include a cytotoxic agent, a growth-inhibitory agent, a radiation therapy, an anti-angiogenic agent, as described herein, or a combination thereof, alone, or in addition to a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) and/or any additional therapeutic agent described herein).

In another aspect, provided herein is a method of treating an individual having a cancer, the method comprising (a) determining the expression level of PD-L1, CXCL9, and IFNG in a sample from the individual, wherein an immune-score expression level of PD-L1, CXCL9, and IFNG in the sample has been determined to be above a reference immune-score expression level, wherein the reference immune-score expression level is an immune-score expression level of PD-L1, CXCL9, and IFNG in a reference population, and (b) administering an effective amount of a PD-L1 axis binding antagonist to the individual based on the immune-score expression level of PD-L1, CXCL9, and IFNG determined in step (a).

In another aspect, provided herein is a method of treating an individual having a cancer, the method comprising administering to the individual an effective amount of a PD-L1 axis binding antagonist, wherein prior to treatment the expression level of PD-L1, CXCL9, and IFNG in a sample from the individual has been determined and an immune-score expression level of PD-L1, CXCL9, and IFNG in the sample that is above a reference immune-score expression level has been determined, wherein the reference immune-score expression level is an immune-score expression level of PD-L1, CXCL9, and IFNG in a reference population.

In some embodiments, the immune-score expression level of PD-L1, CXCL9, and IFNG in the sample is in the top 80^{th} percentile of the immune-score expression level of PD-L1, CXCL9, and IFNG in the reference population. In some embodiments, the immune-score expression level of PD-L1, CXCL9, and IFNG in the sample is in the top 50^{th} percentile of the immune-score expression level of PD-L1, CXCL9, and IFNG in the reference population. In some embodiments, the immune-score expression level of PD-L1, CXCL9, and IFNG in the sample is in the top 20^{th} percentile of the immune-score expression level of PD-L1, CXCL9, and IFNG in the reference population.

In some embodiments, the reference population is a population of individuals having the cancer, the population of individuals consisting of a first subset of individuals who have been treated with a PD-L1 axis binding antagonist therapy and a second subset of individuals who have been treated with a non-PD-L1 axis binding antagonist therapy, wherein the non-PD-L1 axis binding antagonist therapy does not comprise a PD-L1 axis binding antagonist.

In some embodiments, the immune-score expression level of PD-L1, CXCL9, and IFNG is an average of the expression level of each of PD-L1, CXCL9, and IFNG. In some embodiments, the average of the expression level of each of PD-L1, CXCL9, and IFNG is an average of a normalized expression level of each of PD-L1, CXCL9, and IFNG. In some embodiments, the immune-score expression level of PD-L1, CXCL9, and IFNG is a median of the expression level of each of PD-L1, CXCL9, and IFNG. In some embodiments, the immune-score expression level of PD-L1, CXCL9, and IFNG is a median of a normalized expression level of each of PD-L1, CXCL9, and IFNG. In some embodiments, the normalized expression level of each of PD-L1, CXCL9, and IFNG is the expression level of each of PD-L1, CXCL9, and IFNG normalized to a reference gene. In some embodiments, the reference immune-score expression level is a pre-assigned expression level of PD-L1, CXCL9, and IFNG.

In another aspect, provided herein is a method of identifying an individual having a cancer who may benefit from a treatment comprising a PD-L1 axis binding antagonist, the method comprising determining the expression level of PD-L1, IFNG, GZMB, and CD8A in a sample from the individual, wherein an immune-score expression level of PD-L1, IFNG, GZMB, and CD8A in the sample that is above a reference immune-score expression level identifies the individual as one who may benefit from a treatment comprising a PD-L1 axis binding antagonist, wherein the reference immune-score expression level is an immune-score expression level of PD-L1, IFNG, GZMB, and CD8A in a reference population.

In another aspect, provided herein is a method for selecting a therapy for an individual having a cancer, the method comprising determining the expression level of PD-L1, IFNG, GZMB, and CD8A in a sample from the individual, wherein an immune-score expression level of PD-L1, IFNG, GZMB, and CD8A in the sample that is above a reference immune-score expression level identifies the individual as one who may benefit from a treatment comprising a PD-L1 axis binding antagonist, wherein the reference immune-score expression level is an immune-score expression level of PD-L1, IFNG, GZMB, and CD8A in a reference population.

In some embodiments, the immune-score expression level of PD-L1, IFNG, GZMB, and CD8A in the sample is above the reference immune-score expression level and the method further comprises administering to the individual an effective amount of a PD-L1 axis binding antagonist.

In some embodiments, the immune-score expression level of PD-L1, IFNG, GZMB, and CD8A in the sample that is below the reference immune-score expression level identifies the individual as one who is less likely to benefit from a treatment comprising a PD-L1 axis binding antagonist.

In some embodiments, the immune-score expression level of PD-L1, IFNG, GZMB, and CD8A in the sample is below the reference immune-score expression level and the method further comprises administering to the individual an effective amount of an anti-cancer therapy other than, or in addition to, a PD-L1 axis binding antagonist (e.g., the anti-cancer therapy other than, or in addition to, a PD-L1 axis binding antagonist may include a cytotoxic agent, a growth-inhibitory agent, a radiation therapy, an anti-angiogenic agent, as described herein, or a combination thereof, alone, or in addition to a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) and/or any additional therapeutic agent described herein).

In another aspect, provided herein is a method of treating an individual having a cancer, the method comprising (a) determining the expression level of PD-L1, IFNG, GZMB, and CD8A in a sample from the individual, wherein an immune-score expression level of PD-L1, IFNG, GZMB, and CD8A in the sample has been determined to be above a reference immune-score expression level, wherein the reference immune-score expression level is an immune-score expression level of PD-L1, IFNG, GZMB, and CD8A in a reference population, and (b) administering an effective amount of a PD-L1 axis binding antagonist to the individual based on the immune-score expression level of PD-L1, IFNG, GZMB, and CD8A determined in step (a).

In another aspect, provided herein is a method of treating an individual having a cancer, the method comprising administering to the individual an effective amount of a PD-L1 axis binding antagonist, wherein prior to treatment the expression level of PD-L1, IFNG, GZMB, and CD8A in a sample from the individual has been determined and an immune-score expression level of PD-L1, IFNG, GZMB, and CD8A in the sample that is above a reference immune-score expression level has been determined, wherein the reference immune-score expression level is an immune-score expression level of PD-L1, IFNG, GZMB, and CD8A in a reference population.

In some embodiments, the immune-score expression level of PD-L1, IFNG, GZMB, and CD8A in the sample is in the top 80^{th} percentile of the immune-score expression level of PD-L1, IFNG, GZMB, and CD8A in the reference population. In some embodiments, the immune-score expression level of PD-L1, IFNG, GZMB, and CD8A in the sample is in the top 50^{th} percentile of the immune-score expression level of PD-L1, IFNG, GZMB, and CD8A in the reference population. In some embodiments, the immune-score expression level of PD-L1, IFNG, GZMB, and CD8A in the sample is in the top 20^{th} percentile of the immune-score expression level of PD-L1, IFNG, GZMB, and CD8A in the reference population.

In some embodiments, the reference population is a population of individuals having the cancer, the population of individuals consisting of a first subset of individuals who have been treated with a PD-L1 axis binding antagonist therapy and a second subset of individuals who have been treated with a non-PD-L1 axis binding antagonist therapy, wherein the non-PD-L1 axis binding antagonist therapy does not comprise a PD-L1 axis binding antagonist.

In some embodiments, the immune-score expression level of PD-L1, IFNG, GZMB, and CD8A is an average of the expression level of each of PD-L1, IFNG, GZMB, and CD8A. In some embodiments, the average expression level of each of PD-L1, IFNG, GZMB, and CD8A is an average of a normalized expression level of each of PD-L1, IFNG, GZMB, and CD8A. In some embodiments, the immune-score expression level of PD-L1, IFNG, GZMB, and CD8A is a median of the expression level of each of PD-L1, IFNG, GZMB, and CD8A. In some embodiments, the immune-score expression level of PD-L1, IFNG, GZMB, and CD8A is a median of a normalized expression level of each of PD-L1, IFNG, GZMB, and CD8A. In some embodiments, the normalized expression level of each of PD-L1, IFNG, GZMB, and CD8A is the expression level of each of PD-L1, IFNG, GZMB, and CD8A normalized to a reference gene. In some embodiments, the reference immune-score expression level is a pre-assigned expression level of PD-L1, IFNG, GZMB, and CD8A.

In another aspect, provided herein is a method of identifying an individual having a cancer who may benefit from a treatment comprising a PD-L1 axis binding antagonist, the method comprising determining the expression level of PD-L1, IFNG, GZMB, CD8A, and PD-1 in a sample from the individual, wherein an immune-score expression level of PD-L1, IFNG, GZMB, CD8A, and PD-1 in the sample that is above a reference immune-score expression level identifies the individual as one who may benefit from a treatment comprising a PD-L1 axis binding antagonist, wherein the reference immune-score expression level is an immune-score expression level of PD-L1, IFNG, GZMB, CD8A, and PD-1 in a reference population.

In another aspect, provided herein method for selecting a therapy for an individual having a cancer, the method comprising determining the expression level of PD-L1, IFNG, GZMB, CD8A, and PD-1 in a sample from the individual, wherein an immune-score expression level of PD-L1, IFNG, GZMB, CD8A, and PD-1 in the sample that is above a reference immune-score expression level identifies the individual as one who may benefit from a treatment comprising a PD-L1 axis binding antagonist, wherein the reference immune-score expression level is an immune-score expression level of PD-L1, IFNG, GZMB, CD8A, and PD-1 in a reference population.

In some embodiments, the immune-score expression level of PD-L1, IFNG, GZMB, CD8A, and PD-1 in the sample is above the reference immune-score expression level and the method further comprises administering to the individual an effective amount of a PD-L1 axis binding antagonist. In some embodiments, the immune-score expression level of PD-L1, IFNG, GZMB, CD8A, and PD-1 in the sample that is below the reference immune-score expression level identifies the individual as one who is less likely to benefit from a treatment comprising a PD-L1 axis binding antagonist. In some embodiments, the immune-score expression level of PD-L1, IFNG, GZMB, CD8A, and PD-1 in the sample is below the reference immune-score expression level and the method further comprises administering to the individual an effective amount of an anti-cancer therapy other than, or in addition to, a PD-L1 axis binding antagonist (e.g., the anti-cancer therapy other than, or in addition to, a PD-L1 axis binding antagonist may include a cytotoxic agent, a growth-inhibitory agent, a radiation therapy, an anti-angiogenic agent, as described herein, or a combination thereof, alone, or in addition to a PD-L1 axis binding antagonist (e.g., PD-L1 axis binding antagonist (e.g., anti-PD-L1 antibody, e.g., atezolizumab (MPDL3280A)) or PD-1 binding antagonist (e.g., anti-PD-1 antibody)) and/or any additional therapeutic agent described herein).

In another aspect, provided herein is a method of treating an individual having a cancer, the method comprising (a) determining the expression level of PD-L1, IFNG, GZMB, CD8A, and PD-1 in a sample from the individual, wherein an immune-score expression level of PD-L1, IFNG, GZMB, CD8A, and PD-1 in the sample relative to a reference immune-score expression level has been determined, wherein the reference immune-score expression level is an immune-score expression level of PD-L1, IFNG, GZMB, CD8A, and PD-1 in a reference population, and (b) administering an effective amount of a PD-L1 axis binding antagonist to the individual based on the immune-score expression level of PD-L1, IFNG, GZMB, CD8A, and PD-1 determined in step (a).

In another aspect, provided herein is a method of treating an individual having a cancer, the method comprising administering to the individual an effective amount of a PD-L1 axis binding antagonist, wherein prior to treatment the expression level of PD-L1, IFNG, GZMB, CD8A, and PD-1 in a sample from the individual has been determined and an immune-score expression level of PD-L1, IFNG, GZMB, CD8A, and PD-1 in the sample that is above a reference immune-score expression level has been determined, wherein the reference immune-score expression level is an immune-score expression level of PD-L1, IFNG, GZMB, CD8A, and PD-1 in a reference population.

In some embodiments, the immune-score expression level of PD-L1, IFNG, GZMB, CD8A, and PD-1 in the sample is in the top 80^{th} percentile of the immune-score expression level of PD-L1, IFNG, GZMB, CD8A, and PD-1 in the reference population. In some embodiments, the immune-score expression level of PD-L1, IFNG, GZMB, CD8A, and PD-1 in the sample is in the top 50^{th} percentile of the immune-score expression level of PD-L1, IFNG, GZMB, CD8A, and PD-1 in the reference population. In some embodiments, the immune-score expression level of PD-L1, IFNG, GZMB, CD8A, and PD-1 in the sample is in the top 20^{th} percentile of the immune-score expression level of PD-L1, IFNG, GZMB, CD8A, and PD-1 in the reference population.

In some embodiments, the immune-score expression level of PD-L1, IFNG, GZMB, CD8A, and PD-1 is an average of the expression level of each of PD-L1, IFNG, GZMB, CD8A, and PD-1. In some embodiments, the average of the expression level of each of PD-L1, IFNG, GZMB, CD8A, and PD-1 is an average of a normalized expression level of each of PD-L1, IFNG, GZMB, CD8A, and PD-1.

In some embodiments, the immune-score expression level of PD-L1, IFNG, GZMB, CD8A, and PD-1 is a median of the expression level of each of PD-L1, IFNG, GZMB, CD8A, and PD-1.

In some embodiments, the immune-score expression level of PD-L1, IFNG, GZMB, CD8A, and PD-1 is a median of a normalized expression level of each of PD-L1, IFNG, GZMB, CD8A, and PD-1. In some embodiments, the normalized expression level of each of PD-L1, IFNG, GZMB, CD8A, and PD-1 is the expression level of each of PD-L1, IFNG, GZMB, CD8A, and PD-1 normalized to a reference gene. In some embodiments, reference immune-score expression level is a pre-assigned expression level of PD-L1, IFNG, GZMB, CD8A, and PD-1.

In some embodiments of any of the above aspects, the reference population is a population of individuals having the cancer, the population of individuals consisting of a first subset of individuals who have been treated with a PD-L1 axis binding antagonist therapy and a second subset of individuals who have been treated with a non-PD-L1 axis binding antagonist therapy, wherein the non-PD-L1 axis binding antagonist therapy does not comprise a PD-L1 axis binding antagonist.

In some embodiments of any of the above aspects, the reference immune-score expression level significantly separates each of the first and second subsets of individuals based on a significant difference between an individual's responsiveness to treatment with the PD-L1 axis binding antagonist therapy and an individual's responsiveness to treatment with the non-PD-L1 axis binding antagonist therapy above the reference immune-score expression level, wherein the individual's responsiveness to treatment with the PD-L1 axis binding antagonist therapy is significantly improved relative to the individual's responsiveness to treatment with the non-PD-L1 axis binding antagonist therapy.

In some embodiments of any of the above aspects, the reference immune-score expression level significantly separates each of the first and second subsets of individuals based on a significant difference between an individual's responsiveness to treatment with the PD-L1 axis binding antagonist therapy and an individual's responsiveness to treatment with the non-PD-L1 axis binding antagonist therapy below the reference immune-score expression level, wherein the individual's responsiveness to treatment with the non-PD-L1 axis binding antagonist therapy is significantly improved relative to the individual's responsiveness to treatment with the PD-L1 axis binding antagonist therapy.

In some embodiments of any of the above aspects, the responsiveness to treatment is an increase in PFS.

In some embodiments of any of the above aspects, the responsiveness to treatment is an increase in OS.

In some embodiments of any of the above aspects, the reference gene is a housekeeping gene. In some embodiments, the housekeeping gene is TMEM55B.

In some embodiments of any of the above aspects, benefit from the treatment comprising a PD-L1 axis binding antagonist is an increase in OS.

In some embodiments of any of the above aspects, benefit from the treatment comprising a PD-L1 axis binding antagonist is an increase in PFS.

In some embodiments of any of the above aspects, benefit from the treatment comprising a PD-L1 axis binding antagonist is an increase in OS and PFS.

In some embodiments of any of the above aspects, the expression level is a nucleic acid expression level. In some embodiments, the nucleic acid expression level is an mRNA expression level. In some embodiments, the mRNA expression level is determined by RNA-seq, RT-qPCR, qPCR, multiplex qPCR or RT-qPCR, microarray analysis, SAGE, MassARRAY technique, ISH, or a combination thereof. In some embodiments, the mRNA expression level is detected using RNA-seq. In some embodiments, the mRNA expression level is detected using RT-qPCR. In some embodiments, the expression level is detected in tumor cells, tumor infiltrating immune cells, stromal cells, or a combination thereof.

In some embodiments of any of the above aspects, the sample is a tissue sample, a cell sample, a whole blood sample, a plasma sample, a serum sample, or a combination thereof. In some embodiments, the tissue sample is a tumor tissue sample. In some embodiments, the tumor tissue sample comprises tumor cells, tumor-infiltrating immune cells, stromal cells, or a combination thereof. In some embodiments, the tumor tissue sample is a formalin-fixed and paraffin-embedded (FFPE) sample, an archival sample, a fresh sample, or a frozen sample. In some embodiments, the tumor tissue sample is a FFPE sample.

In some embodiments of any of the above aspects, the cancer is selected from the group consisting of a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a merkel cell cancer, or a hematologic malignancy. In some embodiments, the cancer is a lung cancer, a kidney cancer, a bladder cancer, or a breast cancer. In some embodiments, the lung cancer is a non-small cell lung cancer (NSCLC). In some embodiments, the kidney cancer is a renal cell carcinoma (RCC). In some embodiments, the bladder cancer is a urothelial bladder cancer (UBC). In some embodiments, the breast cancer is a triple negative breast cancer (TNBC).

In some embodiments of any of the above aspects, the PD-L1 axis binding antagonist inhibits the binding of PD-L1 to PD-1, the binding of PD-L1 to B7-1, or the binding of PD-L1 to both PD-1 and B7-1. In some embodiments, the PD-L1 axis binding antagonist is a PD-L1 binding antagonist. In other embodiments, the PD-L1 axis binding antagonist is a PD-1 binding antagonist.

In some embodiments, the PD-L1 binding antagonist is an anti-PD-L1 antibody (e.g., atezolizumab (MPDL3280A), YW243.55.S70, MSB0010718C (avelumab), MDX-1105, or MEDI4736 (durvalumab)). In some embodiments, the PD-1 binding antagonist is an anti-PD-1 antibody (e.g., MDX 1106 (nivolumab), MK-3475 (pembrolizumab), CT-011 (pidilizumab), MEDI-0680 (AMP-514), PDR001, REGN2810, or BGB-108).

In some embodiments of any of the above aspects, the anti-PD-L1 antibody is selected from the group consisting of atezolizumab (MPDL3280A), YW243.55.S70, MSB0010718C, MDX-1105, and MEDI4736. In some embodiments, the anti-PD-L1 antibody comprises the following hypervariable regions: (a) an HVR-H1 sequence of GFTFSDSWIH (SEQ ID NO: 9); (b) an HVR-H2 sequence of AWISPYGGSTYYADSVKG (SEQ ID NO: 10); (c) an HVR-H3 sequence of RHWPGGFDY (SEQ ID NO: 11); (d) an HVR-L1 sequence of RASQDVSTAVA (SEQ ID NO: 12); (e) an HVR-L2 sequence of SASFLYS (SEQ ID NO: 13); and (f) an HVR-L3 sequence of QQYLYHPAT (SEQ ID NO: 14). In some embodiments, the anti-PD-L1 antibody comprises (a) a heavy chain variable (VH) domain comprising an amino acid sequence having at least 90% sequence identity to the amino acid sequence of SEQ ID NO: 16; (b) a light chain variable (VL) domain comprising an amino acid sequence having at least 90% sequence identity to the amino acid sequence of SEQ ID NO: 17; or (c) a VH domain as in (a) and a VL domain as in (b). In some embodiments, the anti-PD-L1 antibody comprises: (a) a heavy chain variable (VH) domain comprising an amino acid sequence having at least 95% sequence identity to the amino acid sequence of SEQ ID NO: 16; (b) a light chain variable (VL) domain comprising an amino acid sequence having at least 95% sequence identity to the amino acid sequence of SEQ ID NO: 17; or (c) a VH domain as in (a) and a VL domain as in (b). In some embodiments, the anti-PD-L1 antibody comprises: (a) a heavy chain variable (VH) domain comprising an amino acid sequence having at least 96% sequence identity to the amino acid sequence of SEQ ID NO: 16; (b) a light chain variable (VL) domain comprising an amino acid sequence having at least 96% sequence identity to the amino acid sequence of SEQ ID NO: 17; or (c) a VH domain as in (a) and a VL domain as in (b). In some embodiments, the anti-PD-L1 antibody comprises: (a) a heavy chain variable (VH) domain comprising an amino acid sequence having at least 97% sequence identity to the amino acid sequence of SEQ ID NO: 16; (b) a light chain variable (VL) domain comprising an amino acid sequence having at least 97% sequence identity to the amino acid sequence of SEQ ID NO: 17; or (c) a VH domain as in (a) and a VL domain as in (b). In some embodiments, the anti-PD-L1 antibody comprises: (a) a heavy chain variable (VH) domain comprising an amino acid sequence having at least 98% sequence identity to the amino acid sequence of SEQ ID NO: 16; (b) a light chain variable (VL) domain comprising an amino acid sequence having at least 98% sequence identity to the amino acid sequence of SEQ ID NO: 17; or (c) a VH domain as in (a) and a VL domain as in (b). In some embodiments, the anti-PD-L1 antibody comprises: (a) a heavy chain variable (VH) domain comprising an amino acid sequence having at least 99% sequence identity to the amino acid sequence of SEQ ID NO: 16; (b) a light chain variable (VL) domain comprising an amino acid sequence having at least 99% sequence identity to the amino acid sequence of SEQ ID NO: 17; or (c) a VH domain as in (a) and a VL domain as in (b). In some embodiments, the anti-PD-L1 antibody comprises: (a) a VH domain comprising the amino acid sequence of SEQ ID NO: 16; (b) a VL domain comprising the amino acid sequence of SEQ ID NO: 17; or (c) a VH domain as in (a) and a VL domain as in (b). In some embodiments the anti-PD-L1 antibody comprises: (a) a VH domain comprising the amino acid sequence of SEQ ID NO: 16; and (b) a VL domain comprising the amino acid sequence of SEQ ID NO: 17. In some embodiments, the anti-PD-L1 antibody is atezolizumab. In some embodiments, the anti-PD-L1 antibody is

In some embodiments, the PD-L1 axis binding antagonist is an anti-PD-1 antibody.

In some embodiments of any of the above aspects, the non-PD-L1 axis binding antagonist is an anti-neoplastic agent, a chemotherapeutic agent, a growth inhibitory agent, an anti-angiogenic agent, a radiation therapy, or a cytotoxic agent.

In some embodiments of any of the above aspects, the anti-cancer therapy is an anti-neoplastic agent, a chemotherapeutic agent, a growth inhibitory agent, an anti-angiogenic agent, a radiation therapy, or a cytotoxic agent.

In some embodiments of any of the above aspects, the individual has not been previously treated for the cancer. In some embodiments of any of the above aspects, the individual has not been previously administered a PD-L1 axis binding antagonist.

In some embodiments of any of the above aspects, the treatment comprising a PD-L1 axis binding antagonist is a monotherapy.

In some embodiments of any of the above aspects, the treatment comprising a PD-L1 binding antagonist is a combination therapy.

In some embodiments of any of the above aspects, the method further comprises administering to the individual an effective amount of an additional therapeutic agent. In some embodiments, the additional therapeutic agent is an anti-neoplastic agent, a chemotherapeutic agent, a growth inhibitory agent, an anti-angiogenic agent, a radiation therapy, a cytotoxic agent, or a combination thereof.

In some embodiments, the additional therapeutic agent is a chemotherapeutic agent. In some embodiments, the chemotherapeutic agent is carboplatin; paclitaxel; or carboplatin and paclitaxel. In certain embodiments, the chemotherapeutic agent is carboplatin and paclitaxel.

In some embodiments, the additional therapeutic agent is an anti-angiogenic agent. In some embodiments, the anti-angiogenic agent is an anti-VEGF antibody (e.g., bevacizumab).

In some embodiments, the additional therapeutic agent is a combination of an anti-angiogenic agent and a chemotherapeutic agent. In some embodiments, the chemotherapeutic agent is carboplatin; paclitaxel; or carboplatin and paclitaxel. In some embodiments, the chemotherapeutic is carboplatin and paclitaxel. In some embodiments, the anti-angiogenic agent is an anti-VEGF antibody (e.g., bevacizumab).

In some embodiments of any of the above aspects, the individual is a human.

In another aspect, provided herein is a kit for identifying an individual having a cancer who may benefit from a treatment comprising a PD-L1 axis binding antagonist, the kit comprising (a) reagents for determining the expression level of PD-L1, CXCL9, and IFNG in a sample from the individual; and, optionally, (b) instructions for using the reagents to identify an individual having a cancer who may benefit from a treatment comprising a PD-L1 axis binding antagonist.

In another aspect, provided herein is a kit for identifying an individual having a cancer who may benefit from a treatment comprising a PD-L1 axis binding antagonist, the kit comprising (a) reagents for determining the expression level of PD-L1, IFNG, GZMB, and CD8A in a sample from the individual; and, optionally, (b) instructions for using the reagents to identify an individual having a cancer who may benefit from a treatment comprising a PD-L1 axis binding antagonist.

In another aspect, provided herein is a kit for identifying an individual having a cancer who may benefit from a treatment comprising a PD-L1 axis binding antagonist, the kit comprising reagents for determining the expression level of PD-L1, IFNG, GZMB, CD8A, and PD-1 in a sample from the individual; and, optionally, instructions for using the reagents to identify an individual having a cancer who may benefit from a treatment comprising a PD-L1 axis binding antagonist.

In another aspect, provided herein is an assay for identifying an individual having a cancer who is a candidate for a treatment comprising a PD-L1 axis binding antagonist, the assay comprising determining the expression level of PD-L1, CXCL9, and IFNG in a sample from the individual, wherein an immune-score expression level of PD-L1, CXCL9, and IFNG in the sample that is above a reference immune-score expression level identifies the individual as one who may benefit from the treatment comprising a PD-L1 axis binding antagonist, and wherein the reference immune-score expression level is an immune-score expression level of PD-L1, CXCL9, and IFNG in a reference population.

In another aspect, provided herein is an assay for identifying an individual having a cancer who is a candidate for a treatment comprising a PD-L1 axis binding antagonist, the assay comprising determining the expression level of PD-L1, IFNG, GZMB, and CD8A in a sample from the individual, wherein an immune-score expression level of PD-L1, IFNG, GZMB, and CD8A in the sample that is above a reference immune-score expression level identifies the individual as one who may benefit from the treatment comprising a PD-L1 axis binding antagonist, and wherein the reference immune-score expression level is an immune-score expression level of PD-L1, IFNG, GZMB, and CD8A in a reference population.

In another aspect, provided herein is an assay for identifying an individual having a cancer who is a candidate for a treatment comprising a PD-L1 axis binding antagonist, the assay comprising determining the expression level of PD-L1, IFNG, GZMB, CD8A, and PD-1 in a sample from the individual, wherein an immune-score expression level of PD-L1, IFNG, GZMB, CD8A, and PD-1 in the sample that is above a reference immune-score expression level identifies the individual as one who may benefit from the treatment comprising a PD-L1 axis binding antagonist, and wherein the reference immune-score expression level is an immune-score expression level of PD-L1, IFNG, GZMB, CD8A, and PD-1 in a reference population.

### BRIEF DESCRIPTION OF THE DRAWINGS

FIG. 1 is a graph showing the Kaplan-Meier Curve of progression-free survival (PFS) of the biomarker evaluable population (BEP) of patients (nBEP=753 patients) in the atezolizumab (MPDL3280A) treatment (black) arm and docetaxel control (gray) arm of the OAK Trial (Clinical Trial ID No.: NCT02008227), each arm stratified according to an immune-score expression level of PD-L1, CXCL9, and IFNG. Patients with an immune-score expression level of PD-L1, CXCL9, and IFNG that is higher than approximately 50% of the total BEP (cut-off value: averaged normalized dCt≥−1.9) are indicated by solid lines and patients with an immune-score expression level of PD-L1, CXCL9, and IFNG that is lower than approximately 50% of the total BEP (cut-off value: averaged normalized dCt<−1.9) are indicated by dashed lines. Also shown is a table listing the number of patients who did not have a PFS event within each subgroup of the BEP at a given time point. The time point for each column corresponds to the times shown along the x-axis of the above graph. Averaged normalized dCt is the average of the normalized dCt values for each of PD-L1, CXCL9, and IFNG.

dCt(target gene)=Ct(control gene)−Ct(target gene).

FIG. 2 is a table with forest plots showing hazard ratios (HRs) for PFS in patients in the OAK Trial (Clinical Trial ID No.: NCT02008227) treated with atezolizumab (MPDL3280A) compared to docetaxel (control). The HRs are listed across subgroups of patients defined by different cut-off values (averaged normalized dCt values at different percentile cut-offs of the BEP) for the immune-score expression level of PD-L1, CXCL9, and IFNG. Averaged normalized dCt is the average of the normalized dCt values for each of PD-L1, CXCL9, and IFNG.

dCt(target gene)=Ct(control gene)−Ct(target gene).

FIG. 3 is a graph showing the Kaplan-Meier Curve of overall survival (OS) of the BEP of patients in the atezolizumab (MPDL3280A) treatment (black) arm and docetaxel control (gray) arm of the OAK Trial (Clinical Trial ID No.: NCT02008227), each arm stratified according to an immune-score expression level of PD-L1, CXCL9, and IFNG. Patients with an immune-score expression level of PD-L1, CXCL9, and IFNG that is higher than approximately 50% of the total BEP (cut-off value: averaged normalized dCt≥−1.9) are indicated by solid lines and patients with an immune-score expression level of PD-L1, CXCL9, and IFNG that is lower than approximately 50% of the total BEP (cut-off value: averaged normalized dCt<−1.9) are indicated by dashed lines. Also shown is a table listing the number of surviving patients within each subgroup of the BEP at a given time point. The time point for each column corresponds to the times shown along the x-axis of the above graph. Averaged normalized dCt is the average of the normalized dCt values for each of PD-L1, CXCL9, and IFNG. dCt(target gene)=Ct(control gene)−Ct(target gene).

FIG. 4 is a table with forest plots showing HRs for OS in patients in the OAK Trial treated with atezolizumab (MPDL3280A) compared to docetaxel (control). The HRs are listed across subgroups of patients defined by different cut-off values (averaged normalized dCt values at different percentile cut-offs of the BEP) for the immune-score expression level of PD-L1, CXCL9, and IFNG. Averaged normalized dCt is the average of the normalized dCt values for each of PD-L1, CXCL9, and IFNG.

dCt(target gene)=Ct(control gene)−Ct(target gene).

FIG. 5 is a table with forest plots showing HRs for PFS in patients in the OAK Trial treated with atezolizumab (MPDL3280A) compared to docetaxel (control). The HRs are listed across subgroups of patients defined by different cut-off values (averaged normalized dCt values at different percentile cut-offs of the BEP) for the immune-score expression level of PD-L1, IFNG, GZMB, and CD8A. Averaged normalized dCt is the average of the normalized dCt values for each of PD-L1, IFNG, GZMB, and CD8A.

dCt(target gene)=Ct(control gene)−Ct(target gene).

FIG. 6 is a table with forest plots showing HRs for OS in patients in the OAK Trial treated with atezolizumab (MPDL3280A) compared to docetaxel (control). The HRs are listed across subgroups of patients defined by different cut-off values (averaged normalized dCt values at different percentile cut-offs of the BEP) for the immune-score expression level of PD-L1, IFNG, GZMB, and CD8A. Averaged normalized dCt is the average of the normalized dCt values for each of PD-L1, IFNG, GZMB, and CD8A.

dCt(target gene)=Ct(control gene)−Ct(target gene).

FIG. 7 is a table showing the prevalence, HRs for PFS, and HRs for OS in patients in the OAK Trial treated with atezolizumab (MPDL3280A) compared to docetaxel (control). The HRs are listed across subgroups of patients defined by different cut-off values (averaged normalized dCt values at different quantile cut-offs of the BEP) for the immune-score expression level of (i) CXCL9; (ii) IFNG; (ii) PD-L1 (CD274) and PD-1; (iii) PD-L1 (CD274) and IFNG; (iv) CD8A, GZMB, PD-L1 (CD274), IFNG, and CXCL9; and (v) GZMB, PD-L1 (CD274), IFNG, CXCL9, and PD-1. dCt=Ct(control gene)−Ct(target gene), where a higher dCt indicates higher expression of level of the target gene.

FIG. 8A is a table with forest plots showing HRs for PFS in patients in the POPLAR Trial (Clinical Trial ID No.: NCT01903993) treated with atezolizumab (MPDL3280A) compared to docetaxel (control). The HRs are listed across subgroups of patients defined by different cut-off values (averaged normalized dCt values at different percentile cut-offs of the BEP) for the immune-score expression level of PD-L1, CXCL9, and IFNG. Averaged normalized dCt is the average of the normalized dCt values for each of PD-L1, CXCL9, and IFNG.

dCt(target gene)=Ct(control gene)−Ct(target gene).

FIG. 8B is a table indicating the objective response rates (ORRs) for the corresponding patient populations in FIG. 8A.

FIG. 9 is a table with forest plots showing HRs for OS in patients in the POPLAR Trial (Clinical Trial ID No.: NCT01903993) treated with atezolizumab (MPDL3280A) compared to docetaxel (control). The HRs are listed across subgroups of patients defined by different cut-off values (averaged normalized dCt values at different percentile cut-offs of the BEP) for the immune-score expression level of PD-L1, CXCL9, and IFNG. Averaged normalized dCt is the average of the normalized dCt values for each of PD-L1, CXCL9, and IFNG.

dCt(target gene)=Ct(control gene)−Ct(target gene).

FIG. 10 is a graph showing the Kaplan-Meier Curve of OS of the BEP of patients with urothelial bladder cancer treated with atezolizumab in cohort 2 the IMvigor210 Trial (Clinical Trial ID No.: NCT02108652), stratified according to an immune-score expression level of PD-L1, CXCL9, and IFNG. Patients with an immune-score expression level of PD-L1, CXCL9, and IFNG that is higher than approximately 66% of the total BEP (cut-off value: ≥66^{th} percentile cut-off of the BEP) are indicated by a solid line and patients with an immune-score expression level of PD-L1, CXCL9, and IFNG that is lower than approximately 66% of the total BEP (cut-off value: <66^{th} percentile cut-off of the BEP) are indicated by a dashed line. Also shown is a table listing the number of surviving patients within each subgroup of the BEP at a given time point. The time point for each column corresponds to the times shown along the x-axis of the above graph.

FIG. 11 is a graph showing the Kaplan-Meier Curve of PFS of the BEP of patients with renal cell carcinoma in the atezolizumab (MPDL3280A) and bevacizumab combination treatment (black) arm and sunitinib (gray) arm of the IMMotion150 Trial (Clinical Trial ID No.: NCT01984242), each arm stratified according to an immune-score expression level of PD-L1, IFNG, GZMB, CD8A, and PD-1. Patients with an immune-score expression level of PD-L1, IFNG, GZMB, CD8A, and PD-1 that is higher than approximately 50% of the total BEP (cut-off value: ≥50^{th} percentile cut-off of the BEP) are indicated by solid lines and patients with an immune-score expression level of PD-L1, IFNG, GZMB, CD8A, and PD-1 that is lower than approximately 50% of the total BEP (cut-off value: <50^{th} percentile cut-off of the BEP) are indicated by dashed lines. Also shown is a table listing the number of patients who did not have a PFS event within each subgroup of the BEP at a given time point. The time point for each column corresponds to the times shown along the x-axis of the above graph.

FIG. 12 is a graph showing the Kaplan-Meier Curve of OS of the BEP of patients who were treated with atezolizumab in the PCD4989g Trial, stratified according to an immune-score expression level of PD-L1, CXCL9, and IFNG. Patients with an immune-score expression level of PD-L1, CXCL9, and IFNG that is higher than approximately 50% of the total BEP (cut-off value: ≥50^{th} percentile cut-off of the BEP) are indicated by a solid line and patients with an immune-score expression level of PD-L1, CXCL9, and IFNG that is lower than approximately 50% of the total BEP (cut-off value: <50^{th} percentile cut-off of the BEP) are indicated by a dashed line. Also shown is a table listing the number of surviving patients within each subgroup of the BEP at a given time point. The time point for each column corresponds to the times shown along the x-axis of the above graph.

FIG. 13 is a boxplot showing the association between the averaged normalized expression of PD-L1 (CD274), IFNG, and CXCL9 and complete response or partial response (CR/PR), stable disease (SD), and progressive disease (PD) in patients with TNBC treated with atezolizumab (MPDL3280A) in the PCD4989g Trial (Clinical Trial ID No.: NCT01375842).

FIG. 14 is a hierarchical diagram showing the study design of the Phase III IMpower150 Trial (Clinical Trial ID No. NCT02366143).

FIG. 15 is a CONSORT diagram for the IMpower150 Trial.

FIG. 16 is a Kaplan-Meier Curve of PFS in the intention-to-treat (ITT)-WT population of the atezolizumab, bevacizumab, carboplatin, and paclitaxel arm (ABCP; Arm B) or the bevacizumab, carboplatin, and paclitaxel arm (BCP, Arm C) of the IMpower150 Trial. Stratified (by randomization factors for ITT-WT) HRs are given.

FIGS. 17A and 17B show Kaplan-Meier Curves of independent review facility (IRF)-assessed PFS in the ITT-WT population (FIG. 17A) or the ITT ISEL^{high}-WT (FIG. 17B) of the ABCP arm (Arm B) or the BCP arm (Arm C) of the IMpower150 Trial. Stratified HRs are given for the ITT-WT (FIG. 17A; stratification by randomization factors for ITT-WT) and ISEL^{high}-WT (FIG. 17B; stratification by sex and liver metastases for ISEL^{high}-WT).

FIGS. 18A and 18B show Kaplan-Meier Curves of PFS in the ISEL^{high}-WT population (FIG. 18A) and the ISEL^{low}-WT population (FIG. 18B) of the ABCP arm (Arm B) or the BCP arm (Arm C) of the IMpower150 Trial. Stratified (by sex and liver metastases for ISEL^{high}-WT) HR for immune-score expression level-high WT; unstratified HR for ISEL^{low}-WT.

FIG. 19 is a table with forest plots showing HRs for PFS in patients in the IMpower150 Trial treated with ABCP (Arm B) or BCP (Arm C). The HRs are listed across subgroups of patients defined by different cut-off values (averaged normalized dCt values at different percentile cut-offs of the BEP) for the immune-score expression level of PD-L1, CXCL9, and IFNG. Averaged normalized dCt is the average of the normalized dCt values for each of PD-L1, CXCL9, and IFNG.

dCt(target gene)=Ct(control gene)−Ct(target gene).

FIG. 20 is a Kaplan-Meier Curve of PFS in patients with EGFR or ALK genomic alterations in the ABCP arm (Arm B) or the BCP arm (Arm C) of the IMpower150 Trial.

FIG. 21 is a Kaplan-Meier Curve of PFS in the ITT population, including patient with EGFR mutation or ALK translocation, in the ABCP arm (Arm B) or the BCP arm (Arm C) of the IMpower150 Trial. Stratified (by randomization factors) HR.

FIG. 22 is a table with forest plots showing HRs and 95% confidence intervals (Cls) for PFS in clinical subgroups of the ITT-WT population.

FIG. 23 is a Kaplan-Meier Curve of an interim OS analysis in the ITT-WT population of the ABCP arm (Arm B) or the BCP arm (Arm C) of the IMpower150 Trial. Stratified (per randomization factors) HR.

FIG. 24 is a table with forest plots showing HRs for PFS in patients in the IMpower150 Trial treated with atezolizumab, carboplatin, and paclitaxel (ACP; Arm A) or BCP (Arm C). The HRs are listed across subgroups of patients defined by different cut-off values (averaged normalized dCt values at different percentile cut-offs of the BEP) for the immune-score expression level of PD-L1, CXCL9, and IFNG. Averaged normalized dCt is the average of the normalized dCt values for each of PD-L1, CXCL9, and IFNG.

dCt(target gene)=Ct(control gene)−Ct(target gene).

FIGS. 25A and 25B show Kaplan-Meier Curves of PFS at different immune-score expression level cut-offs (approximately 44% prevalence (FIG. 25A) and approximately 25% prevalence (FIG. 25B)) in the ISEL^{high}-WT population and the ISEL^{low}-WT population of the ACP arm (Arm A) or BCP arm (Arm C) of the IMpower150 Trial.

FIG. 26 is a Kaplan-Meier Curve of OS in the intention-to-treat (ITT) population of the atezolizumab arm or the chemotherapy arm of the IMvigor211 Trial.

FIGS. 27A and 27B show Kaplan-Meier Curves of OS in the ISEL^{high}-WT population (FIG. 27A) and the ISEL^{low}-WT population (FIG. 27B) of the atezolizumab arm or the chemotherapy arm of the IMvigor211 Trial.

### DETAILED DESCRIPTION

The present invention provides diagnostic methods, therapeutic methods, and compositions for the treatment of cancer (e.g., lung cancer (e.g., non-small cell lung cancer (NSCLC)), bladder cancer (e.g., urothelial bladder cancer (UBC)), kidney cancer (e.g., renal cell carcinoma (RCC)), and breast cancer (e.g., triple-negative breast cancer (TNBC))). The invention is based, at least in part, on the discovery that an immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4) in a sample obtained from an individual having cancer can be used as a biomarker (e.g., predictive biomarker) in methods of identifying whether the individual is likely to respond to treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)); selecting a therapy for treating the individual; optimizing therapeutic efficacy of a treatment that includes a PD-L1 axis binding antagonist; and/or monitoring the response of the individual to a treatment that includes a PD-L1 axis binding antagonist.

### I. DEFINITIONS

The term “about” as used herein refers to the usual error range for the respective value readily known to the skilled person in this technical field. Reference to “about” a value or parameter herein includes (and describes) embodiments that are directed to that value or parameter per se.

As used herein, “administering” is meant a method of giving a dosage of a compound (e.g., a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) or a composition (e.g., a pharmaceutical composition, e.g., a pharmaceutical composition including a PD-L1 axis binding antagonist) to a subject. The compounds and/or compositions utilized in the methods described herein can be administered, for example, intravenously (e.g., by intravenous infusion), subcutaneously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The term “antibody” herein is used in the broadest sense and encompasses various antibody structures, including, but not limited to, monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g., scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The terms “anti-PD-L1 antibody” and “an antibody that binds to PD-L1” refer to an antibody that is capable of binding PD-L1 with sufficient affinity such that the antibody is useful as a diagnostic and/or therapeutic agent in targeting PD-L1. In one embodiment, the extent of binding of an anti-PD-L1 antibody to an unrelated, non-PD-L1 protein is less than about 10% of the binding of the antibody to PD-L1 as measured, e.g., by a radioimmunoassay (RIA). In certain embodiments, an anti-PD-L1 antibody binds to an epitope of PD-L1 that is conserved among PD-L1 from different species. In certain embodiments, the anti-PD-L1 antibody is atezolizumab (MPDL3280A). PD-L1 (programmed death ligand 1) is also referred to in the art as “programmed cell death 1 ligand 1,” “PDCD1LG1,” “CD274,” “B7-H,” and “PDL1.” An exemplary human PD-L1 is shown in UniProtKB/Swiss-Prot Accession No. Q9NZQ7.1.

The term “anti-cancer therapy” refers to a therapy useful for treating a cancer (e.g., a lung cancer (e.g., non-small cell lung cancer (NSCLC)), a bladder cancer (e.g., a urothelial bladder cancer (UBC)), a kidney cancer (e.g., a renal cell carcinoma (RCC)), or a breast cancer (e.g., a triple-negative breast cancer (TNBC))). Examples of anti-cancer therapeutic agents include, but are limited to, e.g., chemotherapeutic agents, growth inhibitory agents, cytotoxic agents, agents used in radiation therapy, anti-angiogenesis agents, apoptotic agents, anti-tubulin agents, and other agents to treat cancer, for example, anti-CD20 antibodies, platelet derived growth factor inhibitors (e.g., GLEEVEC™ (imatinib mesylate)), a COX-2 inhibitor (e.g., celecoxib), interferons, cytokines, antagonists (e.g., neutralizing antibodies) that bind to one or more of the following targets: PDGFR-β, BlγS, APRIL, BCMA receptor(s), TRAIL/Apo2, other bioactive and organic chemical agents, and the like. Combinations thereof are also included in the invention.

An “article of manufacture” is any manufacture (e.g., a package or container) or kit comprising at least one reagent, e.g., a medicament for treatment of a disease or disorder (e.g., a cancer, e.g., a lung cancer (e.g., NSCLC), a bladder cancer (e.g., UBC), a kidney cancer (e.g., RCC), or a breast cancer (e.g., TNBC)), or a probe for specifically detecting a biomarker described herein. In certain embodiments, the manufacture or kit is promoted, distributed, or sold as a unit for performing the methods described herein.

The phrase “based on” when used herein means that the information about one or more biomarkers is used to inform a treatment decision, information provided on a package insert, or marketing/promotional guidance, etc.

A “blocking” antibody or an “antagonist” antibody is one which inhibits or reduces biological activity of the antigen it binds. Preferred blocking antibodies or antagonist antibodies substantially or completely inhibit the biological activity of the antigen.

By “binding domain” is meant a part of a compound or a molecule that specifically binds to a target epitope, antigen, ligand, or receptor. Binding domains include, but are not limited to, antibodies (e.g., monoclonal, polyclonal, recombinant, humanized, and chimeric antibodies), antibody fragments or portions thereof (e.g., Fab fragments, Fab′2, scFv antibodies, SMIP, domain antibodies, diabodies, minibodies, scFv-Fc, affibodies, nanobodies, and VH and/or VL domains of antibodies), receptors, ligands, aptamers, and other molecules having an identified binding partner.

The term “biomarker” as used herein refers to an indicator, e.g., predictive, diagnostic, and/or prognostic, which can be detected in a sample (e.g., PD-L1, CXCL9, IFNG, GZMB, CD8A, PD-1, or a combination thereof, including, for example, PD-L1, CXCL9, and IFNG; PD-L1, IFNG, GZMB, and CD8A; or PD-L1, IFNG, GZMB, CD8A, and PD-1). The biomarker may serve as an indicator of a particular subtype of a disease or disorder (e.g., a cancer, e.g., a lung cancer (e.g., NSCLC), a bladder cancer (e.g., UBC), a kidney cancer (e.g., RCC), or a breast cancer (e.g., TNBC)) characterized by certain molecular, pathological, histological, and/or clinical features. In some embodiments, a biomarker is a gene. Biomarkers include, but are not limited to, polynucleotides (e.g., DNA, and/or RNA), polynucleotide copy number alterations (e.g., DNA copy numbers), polypeptides, polypeptide and polynucleotide modifications (e.g., posttranslational modifications), carbohydrates, and/or glycolipid-based molecular markers.

The terms “biomarker signature,” “signature,” “biomarker expression signature,” or “expression signature” are used interchangeably herein and refer to one or a combination of biomarkers whose expression is an indicator, e.g., predictive, diagnostic, and/or prognostic (e.g., the immune-score expression level of PD-L1, CXCL9, and IFNG; PD-L1, IFNG, GZMB, and CD8A; or PD-L1, IFNG, GZMB, CD8A, and PD-1). The biomarker signature may serve as an indicator of a particular subtype of a disease or disorder (e.g., a cancer, e.g., a lung cancer (e.g., NSCLC), a bladder cancer (e.g., UBC), a kidney cancer (e.g., RCC), or a breast cancer (e.g., TNBC)) characterized by certain molecular, pathological, histological, and/or clinical features. In some embodiments, the biomarker signature is a “gene signature.” The term “gene signature” is used interchangeably with “gene expression signature” and refers to one or a combination of polynucleotides whose expression is an indicator, e.g., predictive, diagnostic, and/or prognostic. In some embodiments, the biomarker signature is a “protein signature.” The term “protein signature” is used interchangeably with “protein expression signature” and refers to one or a combination of polypeptides whose expression is an indicator, e.g., predictive, diagnostic, and/or prognostic.

The term “CD8A” as used herein, refers to any native CD8A from any vertebrate source, including mammals such as primates (e.g., humans) and rodents (e.g., mice and rats), unless otherwise indicated. The term encompasses “full-length,” unprocessed CD8A as well as any form of CD8A that results from processing in the cell. The term also encompasses naturally occurring variants of CD8A e.g., splice variants or allelic variants. The nucleic acid sequence of an exemplary human CD8A is listed in SEQ ID NO: 1. The amino acid sequence of an exemplary protein encoded by human CD8A is shown in SEQ ID NO: 2.

The term “GZMB” as used herein, refers to any native GZMB (Granzyme B) from any vertebrate source, including mammals such as primates (e.g., humans) and rodents (e.g., mice and rats), unless otherwise indicated. The term encompasses “full-length,” unprocessed GZMB as well as any form of GZMB that results from processing in the cell. The term also encompasses naturally occurring variants of GZMB, e.g., splice variants or allelic variants. The nucleic acid sequence of an exemplary human GZMB is listed in SEQ ID NO: 3. The amino acid sequence of an exemplary protein encoded by human GZMB is shown in SEQ ID NO: 4.

The term “IFNG” as used herein, refers to any native IFNG (Interferon-γ) from any vertebrate source, including mammals such as primates (e.g., humans) and rodents (e.g., mice and rats), unless otherwise indicated. The term encompasses “full-length,” unprocessed IFNG as well as any form of IFNG that results from processing in the cell. The term also encompasses naturally occurring variants of IFNG, e.g., splice variants or allelic variants. The nucleic acid sequence of an exemplary human IFNG is listed in SEQ ID NO: 5. The amino acid sequence of an exemplary protein encoded by human IFNG is shown in SEQ ID NO: 6.

The term “CXCL9” as used herein, refers to any native CXCL9 (Chemokine (C-X-C Motif) Ligand 9) from any vertebrate source, including mammals such as primates (e.g., humans) and rodents (e.g., mice and rats), unless otherwise indicated. The term encompasses “full-length,” unprocessed CXCL9 as well as any form of CXCL9 that results from processing in the cell. The term also encompasses naturally occurring variants of CXCL9, e.g., splice variants or allelic variants. The nucleic acid sequence of an exemplary human CXCL9 is listed in SEQ ID NO: 7. The amino acid sequence of an exemplary protein encoded by human CXCL9 is shown in SEQ ID NO: 8.

The term “CD27” as used herein, refers to any native CD27 (also known in the art as CD27L receptor or TNFRSF7) from any vertebrate source, including mammals such as primates (e.g., humans) and rodents (e.g., mice and rats), unless otherwise indicated. The term encompasses “full-length,” unprocessed CD27 as well as any form of CD27 that results from processing in the cell. The term also encompasses naturally occurring variants of CD27, e.g., splice variants or allelic variants. The nucleic acid sequence of an exemplary human CD27 is listed in SEQ ID NO: 21. The amino acid sequence of an exemplary protein encoded by human CD27 is shown in SEQ ID NO: 22.

The term “FOXP3” as used herein, refers to any native FOXP3 (forkhead box P3, also known in the art as scurfin) from any vertebrate source, including mammals such as primates (e.g., humans) and rodents (e.g., mice and rats), unless otherwise indicated. The term encompasses “full-length,” unprocessed FOXP3 as well as any form of FOXP3 that results from processing in the cell. The term also encompasses naturally occurring variants of FOXP3, e.g., splice variants or allelic variants. The nucleic acid sequence of an exemplary human FOXP3 is listed in SEQ ID NO: 23. The amino acid sequence of an exemplary protein encoded by human FOXP3 is shown in SEQ ID NO: 24.

The term “CTLA4” as used herein, refers to any native CTLA4 (cytotoxic T-lymphocyte-associated protein 4, also known in the art as CD152) from any vertebrate source, including mammals such as primates (e.g., humans) and rodents (e.g., mice and rats), unless otherwise indicated. The term encompasses “full-length,” unprocessed CTLA4 as well as any form of CTLA4 that results from processing in the cell. The term also encompasses naturally occurring variants of CTLA4, e.g., splice variants or allelic variants. The nucleic acid sequence of an exemplary human CTLA4 is listed in SEQ ID NO: 25. The amino acid sequence of an exemplary protein encoded by human CTLA4 is shown in SEQ ID NO: 26.

The term “TIGIT” as used herein, refers to any native TIGIT (T cell immunoreceptor with Ig and ITIM domains) from any vertebrate source, including mammals such as primates (e.g., humans) and rodents (e.g., mice and rats), unless otherwise indicated. The term encompasses “full-length,” unprocessed TIGIT as well as any form of TIGIT that results from processing in the cell. The term also encompasses naturally occurring variants of TIGIT, e.g., splice variants or allelic variants. The nucleic acid sequence of an exemplary human TIGIT is listed in SEQ ID NO: 27. The amino acid sequence of an exemplary protein encoded by human TIGIT is shown in SEQ ID NO: 28.

The term “IDO1” as used herein, refers to any native IDO1 (indoleamine 2,3-dioxygenase 1) from any vertebrate source, including mammals such as primates (e.g., humans) and rodents (e.g., mice and rats), unless otherwise indicated. The term encompasses “full-length,” unprocessed IDO1 as well as any form of IDO1 that results from processing in the cell. The term also encompasses naturally occurring variants of IDO1, e.g., splice variants or allelic variants. The nucleic acid sequence of an exemplary human IDO1 is listed in SEQ ID NO: 29. The amino acid sequence of an exemplary protein encoded by human IDO1 is shown in SEQ ID NO: 30.

The term “CXCL10” as used herein, refers to any native CXCL10 (C-X-C motif chemokine 10; also known in the art as interferon gamma-induced protein 10 or small-inducible cytokine B10) from any vertebrate source, including mammals such as primates (e.g., humans) and rodents (e.g., mice and rats), unless otherwise indicated. The term encompasses “full-length,” unprocessed CXCL10 as well as any form of CXCL10 that results from processing in the cell. The term also encompasses naturally occurring variants of CXCL10, e.g., splice variants or allelic variants. The nucleic acid sequence of an exemplary human CXCL10 is listed in SEQ ID NO: 31. The amino acid sequence of an exemplary protein encoded by human CXCL10 is shown in SEQ ID NO: 32.

The term “CXCL11” as used herein, refers to any native CXCL11 (C-X-C motif chemokine 11) from any vertebrate source, including mammals such as primates (e.g., humans) and rodents (e.g., mice and rats), unless otherwise indicated. The term encompasses “full-length,” unprocessed CXCL11 as well as any form of CXCL11 that results from processing in the cell. The term also encompasses naturally occurring variants of CXCL11, e.g., splice variants or allelic variants. The nucleic acid sequence of an exemplary human CXCL11 is listed in SEQ ID NO: 33. The amino acid sequence of an exemplary protein encoded by human CXCL11 is shown in SEQ ID NO: 34.

The term “PSMB8” as used herein, refers to any native PSMB8 (proteasome subunit beta type-8) from any vertebrate source, including mammals such as primates (e.g., humans) and rodents (e.g., mice and rats), unless otherwise indicated. The term encompasses “full-length,” unprocessed PSMB8 as well as any form of PSMB8 that results from processing in the cell. The term also encompasses naturally occurring variants of PSMB8, e.g., splice variants or allelic variants. The nucleic acid sequence of an exemplary human PSMB8 is listed in SEQ ID NO: 35. The amino acid sequence of an exemplary protein encoded by human PSMB8 is shown in SEQ ID NO: 36.

The term “PSMB9” as used herein, refers to any native PSMB9 (proteasome subunit beta type-9) from any vertebrate source, including mammals such as primates (e.g., humans) and rodents (e.g., mice and rats), unless otherwise indicated. The term encompasses “full-length,” unprocessed PSMB9 as well as any form of PSMB9 that results from processing in the cell. The term also encompasses naturally occurring variants of PSMB9, e.g., splice variants or allelic variants. The nucleic acid sequence of an exemplary human PSMB9 is listed in SEQ ID NO: 37. The amino acid sequence of an exemplary protein encoded by human PSMB9 is shown in SEQ ID NO: 38.

The term “TAP1” as used herein, refers to any native TAP1 (transporter associated with antigen processing 1; also known in the art as antigen peptide transporter 1) from any vertebrate source, including mammals such as primates (e.g., humans) and rodents (e.g., mice and rats), unless otherwise indicated. The term encompasses “full-length,” unprocessed TAP1 as well as any form of TAP1 that results from processing in the cell. The term also encompasses naturally occurring variants of TAP1, e.g., splice variants or allelic variants. The nucleic acid sequence of an exemplary human TAP1 is listed in SEQ ID NO: 39. The amino acid sequence of an exemplary protein encoded by human TAP1 is shown in SEQ ID NO: 40.

The term “TAP2” as used herein, refers to any native TAP2 (antigen peptide transporter 2) from any vertebrate source, including mammals such as primates (e.g., humans) and rodents (e.g., mice and rats), unless otherwise indicated. The term encompasses “full-length,” unprocessed TAP2 as well as any form of TAP2 that results from processing in the cell. The term also encompasses naturally occurring variants of TAP2, e.g., splice variants or allelic variants. The nucleic acid sequence of an exemplary human TAP2 is listed in SEQ ID NO: 41. The amino acid sequence of an exemplary protein encoded by human TAP2 is shown in SEQ ID NO: 42.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia or lymphoid malignancies. More particular examples of such cancers include, but are not limited to, lung cancer, including small-cell lung cancer, non-small cell lung cancer, adenocarcinoma of the lung, and squamous carcinoma of the lung; bladder cancer (e.g., urothelial bladder cancer (UBC), muscle invasive bladder cancer (MIBC), and BCG-refractory non-muscle invasive bladder cancer (NMIBC)); kidney or renal cancer (e.g., renal cell carcinoma (RCC)); cancer of the urinary tract; breast cancer (e.g., HER2+ breast cancer and triple-negative breast cancer (TNBC), which are estrogen receptors (ER−), progesterone receptors (PR−), and HER2 (HER2−) negative); prostate cancer, such as castration-resistant prostate cancer (CRPC); cancer of the peritoneum; hepatocellular cancer; gastric or stomach cancer, including gastrointestinal cancer and gastrointestinal stromal cancer; pancreatic cancer; glioblastoma; cervical cancer; ovarian cancer; liver cancer; hepatoma; colon cancer; rectal cancer; colorectal cancer; endometrial or uterine carcinoma; salivary gland carcinoma; prostate cancer; vulval cancer; thyroid cancer; hepatic carcinoma; anal carcinoma; penile carcinoma; melanoma, including superficial spreading melanoma, lentigo maligna melanoma, acral lentiginous melanomas, and nodular melanomas; multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases.

The terms “cell proliferative disorder” and “proliferative disorder” refer to disorders that are associated with some degree of abnormal cell proliferation. In one embodiment, the cell proliferative disorder is a cancer (e.g., a lung cancer (e.g., NSCLC), a bladder cancer (e.g., UBC), a kidney cancer (e.g., RCC), or a breast cancer (e.g., TNBC)). In another embodiment, the cell proliferative disorder is a tumor.

A “chemotherapeutic agent” is a chemical compound useful in the treatment of a cancer (e.g., cancer, e.g., a lung cancer (e.g., NSCLC), a bladder cancer (e.g., UBC), a kidney cancer (e.g., RCC), or a breast cancer (e.g., TNBC)). Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ₁^{l} and calicheamicin omegall (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®, Bristol-Myers Squibb Oncology, Princeton, N.J.), albumin-engineered nanoparticle formulation of paclitaxel (ABRAXANE™), and docetaxel (TAXOTERE®, Rhome-Poulene Rorer, Antony, France); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (TARCEVA™)); and VEGF-A that reduce cell proliferation; vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g., celecoxib or etoricoxib), proteosome inhibitor (e.g., PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors; tyrosine kinase inhibitors; serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin, and pharmaceutically acceptable salts, acids or derivatives of any of the above; as well as combinations of two or more of the above.

Chemotherapeutic agents as defined herein also include “anti-hormonal agents” or “endocrine therapeutics” which act to regulate, reduce, block, or inhibit the effects of hormones that can promote the growth of cancer (e.g., a lung cancer (e.g., NSCLC), a bladder cancer (e.g., UBC), a kidney cancer (e.g., RCC), or a breast cancer (e.g., TNBC)). They may be hormones themselves, including, but not limited to: anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON.cndot.toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; Vinorelbine and Esperamicins (see U.S. Pat. No. 4,675,187), and pharmaceutically acceptable salts, acids or derivatives of any of the above; as well as combinations of two or more of the above.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

As used herein, “delaying progression” of a disorder or disease means to defer, hinder, slow, retard, stabilize, and/or postpone development of the disease or disorder (e.g., a cancer, e.g., a lung cancer (e.g., NSCLC), a bladder cancer (e.g., UBC), a kidney cancer (e.g., RCC), or a breast cancer (e.g., TNBC)). This delay can be of varying lengths of time, depending on the history of the disease and/or subject being treated. As is evident to one skilled in the art, a sufficient or significant delay can, in effect, encompass prevention, in that the subject does not develop the disease.

The terms “determination,” “determining,” “detection,” “detecting,” and grammatical variations thereof include any means of determining or detecting, including direct and indirect determination or detection.

A “disorder” or “disease” is any condition that would benefit from treatment including, but not limited to, chronic and acute disorders or diseases including those pathological conditions which predispose the mammal to the disorder in question (e.g., cancer, e.g., a lung cancer (e.g., NSCLC), a bladder cancer (e.g., UBC), a kidney cancer (e.g., RCC), or a breast cancer (e.g., TNBC)).

The term “diagnosis” is used herein to refer to the identification or classification of a molecular or pathological state, disease or condition (e.g., cancer, e.g., a lung cancer (e.g., NSCLC), a bladder cancer (e.g., UBC), a kidney cancer (e.g., RCC), or a breast cancer (e.g., TNBC)). For example, “diagnosis” may refer to identification of a particular type of cancer. “Diagnosis” may also refer to the classification of a particular subtype of cancer, e.g., by histopathological criteria, or by molecular features (e.g., a subtype characterized by expression of one or a combination of biomarkers (e.g., particular genes or proteins encoded by said genes)).

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., PD-L1); and B cell activation.

An “effective amount” of a compound, for example, an PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody))PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), or a composition (e.g., pharmaceutical composition) thereof, is at least the minimum amount required to achieve the desired therapeutic or prophylactic result, such as a measurable increase in overall survival (OS) or progression-free survival (PFS) of a particular disease or disorder (e.g., a cancer, e.g., a lung cancer (e.g., NSCLC), a bladder cancer (e.g., UBC), a kidney cancer (e.g., RCC), or a breast cancer (e.g., TNBC)). An effective amount herein may vary according to factors such as the disease state, age, sex, and weight of the individual, and the ability of the antibody to elicit a desired response in the subject. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved. For example, an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) as a cancer treatment may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5^{th} Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full-length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5^{th} Ed. Public Health Service, National Institutes of Health, Bethesda, Md. (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The word “label” when used herein refers to a detectable compound or composition. The label is typically conjugated or fused directly or indirectly to a reagent, such as a polynucleotide probe or an antibody, and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which results in a detectable product.

The terms “level of expression” or “expression level” in general are used interchangeably and generally refer to the amount of a biomarker in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). Expression level can be measured by methods known to one skilled in the art and also disclosed herein, including, for example, RT-qPCR and RNA-seq. The expression level assessed can be used to determine the response to the treatment.

The term “immune-score expression level” refers to a numerical value that reflects the expression level (e.g., a normalized expression level) of a single gene of interest, or an aggregated expression level for more than one gene of interest (e.g., at least two, at least three, at least four, at least five, or at least six genes of interest), related to immune response. An immune-score expression level for more than one gene of interest may be determined by aggregation methods known to one skilled in the art and also disclosed herein, including, for example, by calculating the median or mean of all the expression levels of the genes of interest. Before aggregation, the expression level of each gene of interest may be normalized by using statistical methods known to one skilled in the art and also disclosed herein, including, for example, normalized to the expression level of one or more housekeeping genes, or normalized to a total library size, or normalized to the median or mean expression level value across all genes measured. In some instances, before aggregation across multiple genes of interest, the normalized expression level of each gene of interest may be standardized by using statistical methods known to one skilled in the art and also disclosed herein, including, for example, by calculating the Z-score of the normalized expression level of each gene of interest. In some instances, each gene of interest may have an assigned weight score and the immune-score expression level of multiple genes of interest may be calculated by incorporating the weight score to determine the mean of all the weighted expression level of the genes of interest. For example, an immune-score expression level may refer to a numerical value that reflects the normalized expression level of a single gene selected from the group consisting of PD-L1, CXCL9, IFNG, GZMB, CD8A, and PD-1. Alternatively, an immune-score expression level may, for example, refer to a numerical value that reflects the aggregated normalized expression level (e.g., median of the normalized expression levels, or mean of the normalized expression levels) for at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4), and optionally further reflects the expression level of other genes associated with T-effector cells, including, for example, one or more genes (e.g., one, two, three, four, five, six, seven, eight, nine, ten, eleven, twelve, thirteen, fourteen, fifteen, sixteen, seventeen, eighteen, or nineteen genes) selected from the group consisting of CD8A, GZMA, GZMB, IFNG, EOMES, PRF1, PD-L1, PD-1, CXCL9, CD27, FOXP3, CTLA4, TIGIT, IDO1, CXCL10, CXCL11, PSMB8, PSMB9, TAP1, and TAP2, or combinations thereof, wherein the one or more biomarkers correlated with T-effector cells are different from the one or more genes selected from the group consisting of PD-L1, CXCL9, IFNG, GZMB, CD8A, and PD-1. In some instances, an immune-score expression level may, for example, refer to a numerical value that reflects the aggregated Z-score expression level (e.g., mean of the Z-score normalized expression level, or median of the Z-score normalized expression level) for at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4), and optionally further reflects the expression level of other genes associated with T-effector cells, including, for example, one or more genes (e.g., one, two, three, four, five, six, seven, eight, nine, ten, eleven, twelve, thirteen, fourteen, fifteen, sixteen, seventeen, eighteen, or nineteen genes) selected from the group consisting of CD8A, GZMA, GZMB, IFNG, EOMES, PRF1, PD-L1, PD-1, CXCL9, CD27, FOXP3, CTLA4, TIGIT, IDO1, CXCL10, CXCL11, PSMB8, PSMB9, TAP1, and TAP2, or combinations thereof, wherein the one or more genes associated with T-effector cells are different from the one or more genes selected from the group consisting of PD-L1, CXCL9, IFNG, GZMB, CD8A, and PD-1.

As used herein, the term “reference immune-score expression level” refers to an immune-score expression level against which another immune-score expression level (e.g., for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) is compared, e.g., to make a diagnostic, predictive, prognostic, and/or therapeutic determination. For example, the reference immune-score expression level may be derived from expression levels (e.g., for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference sample, a reference population, and/or a pre-assigned value (e.g., a cut-off value which was previously determined to significantly (e.g., statistically significantly) separate a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a significant difference between an individual's responsiveness to treatment with the PD-L1 axis binding antagonist therapy and an individual's responsiveness to treatment with the non-PD-L1 axis binding antagonist therapy above the cut-off value and/or below the cut-off value, wherein the individual's responsiveness to treatment with the PD-L1 axis binding antagonist therapy is significantly (e.g., statistically significantly) improved relative to the individual's responsiveness to treatment with the non-PD-L1 axis binding antagonist therapy above the cut-off value and/or the individual's responsiveness to treatment with the non-PD-L1 axis binding antagonist therapy is significantly (e.g., statistically significantly) improved relative to the individual's responsiveness to treatment with the PD-L1 axis binding antagonist therapy below the cut-off value). It will be appreciated by one skilled in the art that the numerical value for the reference immune-score expression level may vary depending on the indication (e.g., a cancer (e.g., a breast cancer, a lung cancer, a kidney cancer, or a bladder cancer), the methodology used to detect expression levels (e.g., RNA-seq or RT-qPCR), the statistical methods used to generate an immune-score, and/or the specific combinations of genes examined (e.g., for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)).

“Elevated expression,” “elevated expression levels,” or “elevated levels” refers to an increased expression or increased levels of a gene or combination of genes (e.g., for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a subject relative to a control, such as a subject or subjects who are not suffering from the disease or disorder (e.g., a cancer, e.g., a lung cancer (e.g., NSCLC), a bladder cancer (e.g., UBC), a kidney cancer (e.g., RCC), or a breast cancer (e.g., TNBC)) or an internal control (e.g., housekeeping gene, e.g., TMEM55B), or a reference level, such as a reference immune-score expression level.

“Reduced expression,” “reduced expression levels,” or “reduced levels” refers to a decrease expression or decreased levels of a gene or combination of genes (e.g., for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a subject relative to a control, such as a subject or subjects who are not suffering from the disease or disorder (e.g., a cancer, e.g., a lung cancer (e.g., NSCLC), a bladder cancer (e.g., UBC), a kidney cancer (e.g., RCC), or a breast cancer (e.g., TNBC)) or an internal control (e.g., housekeeping gene, e.g., TMEM55B), or a reference level, such as a reference immune-score expression level. In some embodiments, reduced expression is little or no expression.

A “reference gene” as used herein, refers to a gene or group of genes (e.g., one, two, three, four, five, or six or more genes) that is used for comparison purposes, such as a housekeeping gene. A “housekeeping gene” refers herein to a gene or group of genes (e.g., one, two, three, four, five, or six or more genes) which encode proteins whose activities are essential for the maintenance of cell function and which are typically similarly present in all cell types (e.g., TMEM55B).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “oligonucleotide” refers to a relatively short polynucleotide (e.g., less than about 250 nucleotides in length), including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The term “package insert” is used to refer to instructions customarily included in commercial packages of therapeutic products, that contain information about the indications, usage, dosage, administration, combination therapy, contraindications and/or warnings concerning the use of such therapeutic products.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The terms “Programmed Death Ligand 1” and “PD-L1” refer herein to a native sequence PD-L1 polypeptide, polypeptide variants, and fragments of a native sequence polypeptide and polypeptide variants (which are further defined herein). The PD-L1 polypeptide described herein may be that which is isolated from a variety of sources, such as from human tissue types or from another source, or prepared by recombinant or synthetic methods.

“PD-L1 polypeptide variant”, or variations thereof, means a PD-L1 polypeptide, generally an active PD-L1 polypeptide, as defined herein having at least about 80% amino acid sequence identity with any of the native sequence PD-L1 polypeptide sequences as disclosed herein. Such PD-L1 polypeptide variants include, for instance, PD-L1 polypeptides wherein one or more amino acid residues are added, or deleted, at the N- or C-terminus of a native amino acid sequence. Ordinarily, a PD-L1 polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a native sequence PD-L1 polypeptide sequence as disclosed herein. Ordinarily, PD-L1 variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 281, 282, 283, 284, 285, 286, 287, 288, 289 amino acids in length, or more. Optionally, PD-L1 variant polypeptides will have no more than one conservative amino acid substitution as compared to a native PD-L1 polypeptide sequence, alternatively no more than 2, 3, 4, 5, 6, 7, 8, 9, or 10 conservative amino acid substitution as compared to the native PD-L1 polypeptide sequence.

A “native sequence PD-L1 polypeptide” comprises a polypeptide having the same amino acid sequence as the corresponding PD-L1 polypeptide derived from nature.

The term “PD-L1 axis binding antagonist” refers to a molecule that inhibits the interaction of a PD-L1 axis binding partner with one or more of its binding partners, so as to remove T cell dysfunction resulting from signaling on the PD-1 signaling axis, with a result being restored or enhanced T cell function. As used herein, a PD-L1 axis binding antagonist includes a PD-L1 binding antagonist and a PD-1 binding antagonist, as well as molecules that interfere with the interaction between PD-L1 and PD-1 (e.g., a PD-L2-Fc fusion).

The term “PD-L1 binding antagonist” refers to a molecule that decreases, blocks, inhibits, abrogates, or interferes with signal transduction resulting from the interaction of PD-L1 with either one or more of its binding partners, such as PD-1 or B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen-binding fragments thereof, immunoadhesins, fusion proteins, oligopeptides, and other molecules that decrease, block, inhibit, abrogate, or interfere with signal transduction resulting from the interaction of PD-L1 with one or more of its binding partners, such as PD-1 or B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. In a specific embodiment, the anti-PD-L1 antibody is atezolizumab (CAS Registry Number: 1422185-06-5), also known as MPDL3280A, and described herein. In another specific embodiment, the anti-PD-L1 antibody is YW243.55.S70, described herein. In another specific embodiment, the anti-PD-L1 antibody is MDX-1105, described herein. In still another specific aspect, the anti-PD-L1 antibody is MEDI4736 (durvalumab), described herein. In still another specific aspect, the anti-PD-L1 antibody is MSB0010718C (avelumab), described herein.

As used herein, a “PD-1 binding antagonist” is a molecule that decreases, blocks, inhibits, abrogates or interferes with signal transduction resulting from the interaction of PD-1 with one or more of its binding partners, such as PD-L1 and/or PD-L2. In some embodiments, the PD-1 binding antagonist is a molecule that inhibits the binding of PD-1 to its binding partners. In a specific aspect, the PD-1 binding antagonist inhibits the binding of PD-1 to PD-L1 and/or PD-L2. For example, PD-1 binding antagonists include anti PD-1 antibodies and antigen-binding fragments thereof, immunoadhesins, fusion proteins, oligopeptides, small molecule antagonists, polynucleotide antagonists, and other molecules that decrease, block, inhibit, abrogate or interfere with signal transduction resulting from the interaction of PD-1 with PD-L1 and/or PD-L2. In one embodiment, a PD-1 binding antagonist reduces the negative signal mediated by or through cell surface proteins expressed on T lymphocytes, and other cells, mediated signaling through PD-1 or PD-L1 so as render a dysfunctional T cell less dysfunctional. In some embodiments, the PD-1 binding antagonist is an anti-PD-1 antibody. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is CT-011 (pidilizumab). In another specific aspect, a PD-1 binding antagonist is MEDI-0680 (AMP-514). In another specific aspect, a PD-1 binding antagonist is PDR001. In another specific aspect, a PD-1 binding antagonist is REGN2810. In another specific aspect, a PD-1 binding antagonist is BGB-108. In another specific aspect, a PD-1 binding antagonist is AMP-224.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The technique of “polymerase chain reaction” or “PCR” as used herein generally refers to a procedure wherein minute amounts of a specific piece of nucleic acid, RNA and/or DNA, are amplified as described in U.S. Pat. No. 4,683,195 issued 28 Jul. 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51: 263 (1987); Erlich, ed., PCR Technology, (Stockton Press, N Y, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

As used herein, the term “reverse transcriptase polymerase chain reaction” or “RT-PCR” refers to the replication and amplification of RNA sequences. In this method, reverse transcription is coupled to PCR, e.g., as described in U.S. Pat. No. 5,322,770, herein incorporated by reference in its entirety. In RT-PCR, the RNA template is converted to cDNA due to the reverse transcriptase activity of an enzyme, and then amplified using the polymerizing activity of the same or a different enzyme. Both thermostable and thermolabile reverse transcriptase and polymerase can be used. The “reverse transcriptase” (RT) may include reverse transcriptases from retroviruses, other viruses, as well as a DNA polymerase exhibiting reverse transcriptase activity.

As used herein, the term “reverse transcriptase quantitative polymerase chain reaction” or “RT-qPCR” is a form of PCR wherein the nucleic acid to be amplified is RNA that is first reverse transcribed into cDNA and the amount of PCR product is measured at each step in a PCR reaction.

“Quantitative real time polymerase chain reaction” or “qRT-PCR” refers to a form of PCR wherein the amount of PCR product is measured at each step in a PCR reaction. This technique has been described in various publications including Cronin et al., Am. J. Pathol. 164(1):35-42 (2004); and Ma et al., Cancer Cell 5:607-616 (2004).

The term “multiplex-PCR” refers to a single PCR reaction carried out on nucleic acid obtained from a single source (e.g., an individual) using more than one primer set for the purpose of amplifying two or more DNA sequences in a single reaction.

The term “RNA-seq,” also called “Whole Transcriptome Shotgun Sequencing (WTSS),” refers to the use of high-throughput sequencing technologies to sequence and/or quantify cDNA to obtain information about a sample's RNA content. Publications describing RNA-seq include: Wang et al. “RNA-Seq: a revolutionary tool for transcriptomics” Nature Reviews Genetics 10 (1): 57-63 (January 2009); Ryan et al. BioTechniques 45 (1): 81-94 (2008); and Maher et al. “Transcriptome sequencing to detect gene fusions in cancer”. Nature 458 (7234): 97-101 (January 2009).

The term “polynucleotide,” when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

“Response to a treatment,” “responsiveness to treatment,” or “benefit from a treatment” can be assessed using any endpoint indicating a benefit to the individual, including, without limitation, (1) inhibition, to some extent, of disease progression (e.g., cancer progression), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e. reduction, slowing down or complete stopping) of metastasis; (5) relief, to some extent, of one or more symptoms associated with the disease or disorder (e.g., cancer); (6) increase or extend in the length of survival, including overall survival (OS HR<1) and progression free survival (PFS HR<1); and/or (9) decreased mortality at a given point of time following treatment (e.g., a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

As used herein, “progression-free survival” or “PFS” refers to the length of time during and after treatment during which the disease being treated (e.g., cancer, e.g., a lung cancer (e.g., NSCLC), a bladder cancer (e.g., UBC), a kidney cancer (e.g., RCC), or a breast cancer (e.g., TNBC)) does not progress or get worse. Progression-free survival may include the amount of time individuals have experienced a complete response or a partial response, as well as the amount of time individuals have experienced stable disease.

As used herein, “overall survival” or “OS” refers to the percentage of subjects in a group who are likely to be alive after a particular duration of time (e.g., 6 months, 1 year, 2 years, 3 years, 4 years, 5 years, 10 years, 15 years, 20 years, or more than 20 years from the time of diagnosis or treatment).

As used herein, “complete response” or “CR” refers to disappearance of all signs of cancer in response to treatment. This does not necessarily mean the cancer has been cured.

As used herein, “partial response” or “PR” refers to a decrease in the size of one or more tumors or lesions, or in the extent of cancer in the body, in response to treatment.

As used herein, “hazard ratio” or “HR” is a statistical definition for rates of events. For the purpose of the invention, hazard ratio is defined as representing the probability of an event (e.g., PFS or OS) in the experimental (e.g., treatment) group/arm divided by the probability of an event in the control group/arm at any specific point in time. An HR with a value of 1 indicates that the relative risk of an endpoint (e.g., death) is equal in both the “treatment” and “control” groups; a value greater than 1 indicates that the risk is greater in the treatment group relative to the control group; and a value less than 1 indicates that the risk is greater in the control group relative to the treatment group. “Hazard ratio” in progression-free survival analysis (i.e., PFS HR) is a summary of the difference between two progression-free survival curves, representing the reduction in the risk of death on treatment compared to control, over a period of follow-up. “Hazard ratio” in overall survival analysis (i.e., OS HR) is a summary of the difference between two overall survival curves, representing the reduction in the risk of death on treatment compared to control, over a period of follow-up.

By “extending survival” is meant increasing overall survival or progression free survival in a treated individual relative to an untreated individual (i.e. relative to an individual not treated with the medicament), or relative to an individual who does not express a biomarker at the designated level, and/or relative to an individual treated with an approved anti-tumor agent. An objective response refers to a measurable response, including complete response (CR) or partial response (PR).

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated (e.g., a cancer, e.g., a lung cancer (e.g., NSCLC), a bladder cancer (e.g., UBC), a kidney cancer (e.g., RCC), or a breast cancer (e.g., TNBC)), the presence or size of metastases, or the size of the primary tumor.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from the same subject or individual. In another embodiment, a reference sample is obtained from one or more individuals who are not the subject or individual. In either of the preceding embodiments, the one or more individuals from which the reference sample, reference cell, reference tissue, control sample, control cell, or control tissue is obtained has a cancer. In certain embodiments, the one or more individuals from which the reference sample, reference cell, reference tissue, control sample, control cell, or control tissue is obtained has a cancer and has been previously treated with an anti-cancer therapy (e.g., one or more doses of a PD-L1 axis binding antagonist). In other embodiments, the one or more individuals from which the reference sample, reference cell, reference tissue, control sample, control cell, or control tissue is obtained has a cancer and is treatment naïve. In any of the preceding embodiments, the subject/individual and the one or more individuals who are not the subject or individual have the same cancer. In yet another embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

As used herein, the terms “individual,” “patient,” and “subject” are used interchangeably and refer to any single animal, more preferably a mammal (including such non-human animals as, for example, dogs, cats, horses, rabbits, zoo animals, cows, pigs, sheep, and non-human primates) for which treatment is desired. In certain embodiments, the individual, patient, or subject is a human.

As used herein, “treatment” (and grammatical variations thereof, such as “treat” or “treating”) refers to clinical intervention in an attempt to alter the natural course of the subject being treated, and can be performed either for prophylaxis or during the course of clinical pathology. Desirable effects of treatment include, but are not limited to, preventing occurrence or recurrence of a disease (e.g., a cancer, e.g., a lung cancer (e.g., NSCLC), a bladder cancer (e.g., UBC), a kidney cancer (e.g., RCC), or a breast cancer (e.g., TNBC)), alleviation of symptoms, diminishment of any direct or indirect pathological consequences of the disease, preventing metastasis, decreasing the rate of disease progression, amelioration or palliation of the disease state, and remission or improved prognosis. In some embodiments, the treatments described herein are used to delay development of a disease or to slow the progression of a disease (e.g., a cancer, e.g., a lung cancer (e.g., NSCLC), a bladder cancer (e.g., UBC), a kidney cancer (e.g., RCC), or a breast cancer (e.g., TNBC)). In some instances, the treatment may increase overall survival (OS) (e.g., by about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, about 96% or greater, about 97% or greater, about 98% or greater, or about 99% or greater). In some instances, the treatment may increase OS, e.g., by about 5% to about 500%, e.g., from about 10% to about 450%, e.g., from about 20% to about 400%, e.g., from about 25% to about 350%, e.g., from about 30% to about 400%, e.g., from about 35% to about 350%, e.g., from about 40% to about 300%, e.g., from about 45% to about 250%, e.g., from about 50% to about 200%, e.g., from about 55% to about 150%, e.g., from about 60% to about 100%, e.g., from about 65% to about 100%, e.g., from about 70% to about 100%, e.g., from about 75% to about 100%, e.g., from about 80% to about 100%, e.g., from about 85% to about 100%, e.g., from about 90% to about 100%, e.g., from about 95% to about 100%, e.g., from about 98% to about 100%. In some instances, the treatment may increase the progression-free survival (PFS) (e.g., by about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, about 96% or greater, about 97% or greater, about 98% or greater, or about 99% or greater). In some instances, the treatment may increase PFS, e.g., by about 5% to about 500%, e.g., from about 10% to about 450%, e.g., from about 20% to about 400%, e.g., from about 25% to about 350%, e.g., from about 30% to about 400%, e.g., from about 35% to about 350%, e.g., from about 40% to about 300%, e.g., from about 45% to about 250%, e.g., from about 50% to about 200%, e.g., from about 55% to about 150%, e.g., from about 60% to about 100%, e.g., from about 65% to about 100%, e.g., from about 70% to about 100%, e.g., from about 75% to about 100%, e.g., from about 80% to about 100%, e.g., from about 85% to about 100%, e.g., from about 90% to about 100%, e.g., from about 95% to about 100%, e.g., from about 98% to about 100%.

By “tissue sample” or “cell sample” is meant a collection of similar cells obtained from a tissue of a subject or individual. The source of the tissue or cell sample may be solid tissue as from a fresh, frozen, and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease (e.g., prostate cancer, e.g., CRPC, e.g., mCRPC or locally confined, inoperable CRPC) tissue/organ. The tissue sample may contain compounds which are not naturally intermixed with the tissue in nature such as preservatives, anticoagulants, buffers, fixatives, nutrients, antibiotics, or the like.

For the purposes herein a “section” of a tissue sample is meant a single part or piece of a tissue sample, e.g. a thin slice of tissue or cells cut from a tissue sample. It is understood that multiple sections of tissue samples may be taken and subjected to analysis, provided that it is understood that the same section of tissue sample may be analyzed at both morphological and molecular levels, or analyzed with respect to both polypeptides and polynucleotides.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

### II. DIAGNOSTIC METHODS AND ASSAYS

Provided herein are methods and assays for identifying an individual having a cancer (e.g., a lung cancer (e.g., non-small cell lung cancer (NSCLC)), a bladder cancer (e.g., a urothelial bladder cancer (UBC)), a kidney cancer (e.g., a renal cell carcinoma (RCC)), or a breast cancer (e.g., triple-negative breast cancer (TNBC))) who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). The methods and assays described herein are based on the finding that the immune-score expression level of at least one, at least two, at least three, at least four, at least five, or all six of PD-L1, CXCL9, IFNG, GZMB, CD8A, and PD-1 (e.g., PD-L1, CXCL9, and IFNG; PD-L1, IFNG, GZMB, and CD8A; PD-L1, IFNG GZMB, CD8A, and PD-1; or any combination of gene(s) listed in Tables 1-4) in a sample from the individual may be used to predict the therapeutic efficacy of a PD-L1 axis binding antagonist therapy, e.g., a PD-L1 axis binding antagonist monotherapy or combination therapy including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

Further provided herein are methods and assays for selecting a therapy for an individual having a cancer (e.g., lung cancer (e.g., NSCLC), bladder cancer (e.g., UBC), kidney cancer (e.g., RCC), or breast cancer (e.g., TNBC)); methods for determining whether an individual having a cancer is likely to respond to treatment including a PD-L1 axis binding antagonist; methods for predicting the responsiveness of an individual having a cancer to treatment comprising a PD-L1 axis binding antagonist; and methods for monitoring the response of an individual having a cancer to treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Any of the methods provided herein may further include administering to the individual a PD-L1 axis binding antagonist (e.g., as described below in Section III) to the individual.

### A. One-Gene Immune-Scores and Two-Gene Immune-Score Combinations

In particular instances, the methods and assays provided herein may be used to determine an immune-score expression level of a single gene selected from PD-L1, CXCL9, IFNG, GZMB, CD8A, and PD-1. For example, the determination step may include determining the expression level of any one gene selected from PD-L1, CXCL9, IFNG, GZMB, CD8A, and PD-1.

In some instances, the determination step includes determining the expression levels of any one gene selected from PD-L1, CXCL9, IFNG, GZMB, CD8A, and PD-1 and one or more additional genes associated with T-effector cells, e.g., determining the expression level of (i) one gene selected from the group consisting of PD-L1, CXCL9, IFNG, GZMB, CD8A, and PD-1 and (ii) one or more genes associated with T-effector cells (e.g., at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, or nineteen of CD8A, GZMA, GZMB, IFNG, EOMES, PRF1, PD-L1, PD-1, CXCL9, CD27, FOXP3, CTLA4, TIGIT, IDO1, CXCL10, CXCL11, PSMB8, PSMB9, TAP1, and/or TAP2), wherein the one or more genes associated with T-effector cells are different from the one gene selected from the group consisting of PD-L1, CXCL9, IFNG, GZMB, CD8A, and PD-1.

In one aspect, provided herein are methods for identifying an individual having a cancer (e.g., lung cancer (e.g., NSCLC), bladder cancer (e.g., UBC), kidney cancer (e.g., RCC), or breast cancer (e.g., TNBC)) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of any one gene selected from PD-L1, CXCL9, IFNG, GZMB, CD8A, and PD-1 in a sample from the individual (e.g., a tumor tissue sample), wherein an immune-score expression level of the gene selected from PD-L1, CXCL9, IFNG, GZMB, CD8A, and PD-1 in the sample that is above a reference immune-score expression level (e.g., an immune-score expression level of the same selected gene in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of any one gene selected from PD-L1, CXCL9, IFNG, GZMB, CD8A, and PD-1 in the sample that is below a reference immune-score expression level (e.g., an immune-score expression level of the same selected gene in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In another aspect, also provided herein are methods for selecting a therapy for an individual having a cancer (e.g., lung cancer (e.g., NSCLC), bladder cancer (e.g., UBC), kidney cancer (e.g., RCC), or breast cancer (e.g., TNBC)), the methods including determining the expression level of any one gene selected from PD-L1, CXCL9, IFNG, GZMB, CD8A, and PD-1 in a sample from the individual, wherein an immune-score expression level of the gene selected from PD-L1, CXCL9, IFNG, GZMB, CD8A, and PD-1 in the sample that is above a reference immune-score expression level (e.g., an immune-score expression level of the same selected gene in a reference population) identities an individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of any one gene selected from PD-L1, CXCL9, IFNG, GZMB, CD8A, and PD-1 in the sample that is below a reference immune-score expression level (e.g., an immune-score expression level of the same selected gene in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

The examples and embodiments described in Sections II.B (i-vi), II.C (i-vi), II.D (i-vi), and II.E (i-vi), below, are also specifically contemplated to apply to the one-gene immune-score expression level for any one gene selected from PD-L1, CXCL9, IFNG, GZMB, CD8A, and PD-1.

In particular instances, the methods and assays provided herein may be used to determine an immune-score expression level of two genes selected from PD-L1, CXCL9, IFNG, GZMB, CD8A, and PD-1. For example, the determination step may include determining the expression levels of any of the two-gene combinations listed in Table 1.

In some instances, the determination step includes determining the expression levels of a particular combination of the two genes listed in Table 1 and one or more additional genes associated with T-effector cells, e.g., determining the expression level of (i) two genes selected from the group consisting of PD-L1, CXCL9, IFNG, GZMB, CD8A, and PD-1 (e.g., any one of the combinations of genes listed in Table 1) and (ii) one or more genes associated with T-effector cells (e.g., at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, or eighteen of CD8A, GZMA, GZMB, IFNG, EOMES, PRF1, PD-L1, PD-1, CXCL9, CD27, FOXP3, CTLA4, TIGIT, IDO1, CXCL10, CXCL11, PSMB8, PSMB9, TAP1, and/or TAP2), wherein the one or more genes associated with T-effector cells are different from the two genes selected from the group consisting of PD-L1, CXCL9, IFNG, GZMB, CD8A, and PD-1.

TABLE 1
Exemplary two-gene immune-score combinations

PD-L1 and CXCL9
PD-L1 and IFNG
PD-L1 and GZMB
PD-L1 and CD8A
PD-L1 and PD-1
CXCL9 and IFNG
CXCL9 and GZMB
CXCL9 and CD8A
CXCL9 and PD-1
IFNG and GZMB
IFNG and CD8A
IFNG and PD-1
GZMB and CD8A
GZMB and PD-1
CD8A and PD-1

In one aspect, provided herein are methods for identifying an individual having a cancer (e.g., lung cancer (e.g., NSCLC), bladder cancer (e.g., UBC), kidney cancer (e.g., RCC), or breast cancer (e.g., TNBC)) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of a combination of two genes listed in Table 1 in a sample from the individual (e.g., a tumor tissue sample), wherein an immune-score expression level of the combination of two genes listed in Table 1 in the sample that is above a reference immune-score expression level (e.g., an immune-score expression level of the same combination of two genes listed in Table 1 in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of a combination of two genes listed in Table 1 in the sample that is below a reference immune-score expression level (e.g., an immune-score expression level of the same combination of two genes listed in Table 1 in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In another aspect, also provided herein are methods for selecting a therapy for an individual having a cancer (e.g., lung cancer (e.g., NSCLC), bladder cancer (e.g., UBC), kidney cancer (e.g., RCC), or breast cancer (e.g., TNBC)), the methods including determining the expression level of a combination of two genes listed in Table 1 in a sample from the individual, wherein an immune-score expression level of a combination of two genes listed in Table 1 in the sample that is above a reference immune-score expression level (e.g., an immune-score expression level of the same combination of two genes listed in Table 1 in a reference population) identities an individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of a combination of two genes listed in Table 1 in the sample that is below a reference immune-score expression level (e.g., an immune-score expression level of the same combination of two genes listed in Table 1 in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

The examples and embodiments described in Sections II.B (i-vi), II.C (i-vi), II.D (i-vi), and II.E (i-vi), below, are also specifically contemplated to apply to the two-gene immune-score expression level for any combination of two genes selected from PD-L1, CXCL9, IFNG, GZMB, CD8A, and PD-1, as described in Table 1 above.

### B. Three-Gene Immune-Score Combinations

In particular instances, the methods and assays provided herein may be used to determine an immune-score expression level of three genes selected from PD-L1, CXCL9, IFNG, GZMB, CD8A, and PD-1. For example, the determination step may include determining the expression levels of any of the three-gene combinations listed in Table 2.

In some instances, the determination step includes determining the expression levels of a particular combination of the three genes listed in Table 2 and one or more additional genes associated with T-effector cells, e.g., determining the expression level of (i) three genes selected from the group consisting of PD-L1, CXCL9, IFNG, GZMB, CD8A, and PD-1 (e.g., any one of the combinations of genes listed in Table 2) and (ii) one or more genes associated with T-effector cells (e.g., at least one, at least two, at least three, at least four, at least five, at least six, at least seven, at least eight, at least nine, at least ten, at least eleven, at least twelve, at least thirteen, at least fourteen, at least fifteen, at least sixteen, or seventeen of CD8A, GZMA, GZMB, IFNG, EOMES, PRF1, PD-L1, PD-1, CXCL9, CD27, FOXP3, CTLA4, TIGIT, IDO1, CXCL10, CXCL11, PSMB8, PSMB9, TAP1, and/or TAP2), wherein the one or more genes associated with T-effector cells are different from the three genes selected from the group consisting of PD-L1, CXCL9, IFNG, GZMB, CD8A, and PD-1.

TABLE 2
Exemplary three-gene immune-score combinations

PD-L1; CXCL9; and IFNG
PD-L1; CXCL9; and GZMB
PD-L1; CXCL9; and CD8A
PD-L1; CXCL9; and PD-1
PD-L1; IFNG; and GZMB
PD-L1; IFNG; and CD8A
PD-L1; IFNG; and PD-1
PD-L1; GZMB; and CD8A
PD-L1; GZMB; and PD-1
PD-L1; CD8A; and PD-1
CXCL9; IFNG; and GZMB
CXCL9; IFNG; and CD8A
CXCL9; IFNG; and PD-1
CXCL9; GZMB; and CD8A
CXCL9; GZMB; and PD-1
CXCL9; CD8A; and PD-1
IFNG; GZMB; and CD8A
IFNG; GZMB; and PD-1
IFNG; CD8A; and PD-1
GZMB; CD8A; and PD-1

In one aspect, provided herein are methods for identifying an individual having a cancer (e.g., lung cancer (e.g., NSCLC), bladder cancer (e.g., UBC), kidney cancer (e.g., RCC), or breast cancer (e.g., TNBC)) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of a combination of three genes listed in Table 2 in a sample from the individual (e.g., a tumor tissue sample), wherein an immune-score expression level of the combination of three genes listed in Table 2 in the sample that is above a reference immune-score expression level (e.g., an immune-score expression level of the same combination of three genes listed in Table 2 in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of a combination of three genes listed in Table 2 in the sample that is below a reference immune-score expression level (e.g., an immune-score expression level of the same combination of three genes listed in Table 2 in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In another aspect, also provided herein are methods for selecting a therapy for an individual having a cancer (e.g., lung cancer (e.g., NSCLC), bladder cancer (e.g., UBC), kidney cancer (e.g., RCC), or breast cancer (e.g., TNBC)), the methods including determining the expression level of a combination of three genes listed in Table 2 in a sample from the individual, wherein an immune-score expression level of a combination of three genes listed in Table 2 in the sample that is above a reference immune-score expression level (e.g., an immune-score expression level of the same combination of three genes listed in Table 2 in a reference population) identities an individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of a combination of three genes listed in Table 2 in the sample that is below a reference immune-score expression level (e.g., an immune-score expression level of the same combination of three genes listed in Table 2 in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

The examples and embodiments described below for the combination of the genes PD-L1, CXCL9, and IFNG may also apply to any one of the three-gene combinations listed in Table 2.

(i) Expression of PD-L1, CXCL9, and IFNG

In particular instances, the methods and assays provided herein may be used to determine the immune-score expression level of PD-L1, CXCL9, and IFNG. Various diagnostic methods based on a determination of the immune-score expression level of PD-L1, CXCL9, and IFNG are further described below.

In one aspect, provided herein are methods for identifying an individual having a cancer (e.g., lung cancer (e.g., NSCLC), bladder cancer (e.g., UBC), kidney cancer (e.g., RCC), or breast cancer (e.g., TNBC)) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of PD-L1, CXCL9, and IFNG in a sample from the individual (e.g., a tumor tissue sample), wherein an immune-score expression level of at least one, at least two, or all three of PD-L1, CXCL9, and IFNG in the sample that is above a reference immune-score expression level (e.g., an immune-score expression level of PD-L1, CXCL9, and IFNG in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of at least one, at least two, or all three of PD-L1, CXCL9, and IFNG in the sample that is below a reference immune-score expression level (e.g., an immune-score expression level of PD-L1, CXCL9, and IFNG in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In another aspect, provided herein are methods for selecting a therapy for an individual having a cancer (e.g., lung cancer (e.g., NSCLC), bladder cancer (e.g., UBC), kidney cancer (e.g., RCC), or breast cancer (e.g., TNBC)), the methods including determining the expression level of PD-L1, CXCL9, and IFNG in a sample from the individual, wherein an immune-score expression level of at least one, at least two, or all three of PD-L1, CXCL9, and IFNG in the sample that is above a reference immune-score expression level (e.g., an immune-score expression level of PD-L1, CXCL9, and IFNG in a reference population) identities an individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of at least one, at least two, or all three of PD-L1, CXCL9, and IFNG in the sample that is below a reference immune-score expression level (e.g., an immune-score expression level of PD-L1, CXCL9, and IFNG in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

Further provided herein are methods for determining whether an individual with a cancer (e.g., lung cancer (e.g., NSCLC), bladder cancer (e.g., UBC), kidney cancer (e.g., RCC), or breast cancer (e.g., TNBC)) is likely to respond to treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of PD-L1, CXCL9, and IFNG in a sample from the individual (e.g., a tumor tissue sample), wherein an immune-score expression level of at least one, at least two, or all three of PD-L1, CXCL9, and IFNG in the sample that is above a reference immune-score expression level (e.g., an immune-score expression level of PD-L1, CXCL9, and IFNG in a reference population) indicates that the individual is likely to respond to treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of at least one, at least two, or all three of PD-L1, CXCL9, and IFNG in the sample that is below a reference immune-score expression level (e.g., an immune-score expression level of PD-L1, CXCL9, and IFNG in a reference population) indicates that the individual is less likely to respond to treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

Further provided herein are methods for predicting the responsiveness of an individual with a cancer (e.g., lung cancer (e.g., NSCLC), bladder cancer (e.g., UBC), kidney cancer (e.g., RCC), or breast cancer (e.g., TNBC)) to treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of PD-L1, CXCL9, and IFNG in a sample from the individual (e.g., tumor tissue), wherein an immune-score expression level of at least one, at least two, or all three of PD-L1, CXCL9, and IFNG in the sample that is above a reference immune-score expression level (e.g., an immune-score expression level of PD-L1, CXCL9, and IFNG in a reference population) indicates that the individual is more likely to be responsive to treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of at least one, at least two, or all three of PD-L1, CXCL9, and IFNG in the sample that is below a reference immune-score expression level (e.g., an immune-score expression level of PD-L1, CXCL9, and IFNG in a reference population) indicates that the individual is less likely to be responsive to treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

Further provided herein are methods for determining the likelihood that an individual with a cancer (e.g., lung cancer (e.g., NSCLC), bladder cancer (e.g., UBC), kidney cancer (e.g., RCC), or breast cancer (e.g., TNBC)) will exhibit benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of PD-L1, CXCL9, and IFNG in a sample from the individual (e.g., tumor tissue), wherein an immune-score expression level of at least one, at least two, or all three of PD-L1, CXCL9, and IFNG in the sample that is above a reference immune-score expression level (e.g., an immune-score expression level of PD-L1, CXCL9, and IFNG in a reference population) indicates that the individual will have an increased likelihood of benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of at least one, at least two, or all three of PD-L1, CXCL9, and IFNG in the sample that is below a reference immune-score expression level (e.g., an immune-score expression level of PD-L1, CXCL9, and IFNG in a reference population) indicates that the individual will have a decreased likelihood of benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In any of the preceding methods, the individual having a cancer (e.g., lung cancer (e.g., NSCLC), bladder cancer (e.g., UBC), kidney cancer (e.g., RCC), or breast cancer (e.g., TNBC)) may be provided a recommendation prior to administration of the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), based on the immune-score expression level of PD-L1, CXCL9, and/or IFNG determined in accordance with any of the above methods. In some instances, the methods further include providing a recommendation that the individual will be likely to respond to, or benefit from, treatment with a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, the methods include providing a recommendation that the therapy selected for the individual includes treatment with a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In any of the preceding methods, the methods may further include administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) to the individual. In some instances, the methods further include administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein the immune-score expression level of at least one, at least two, or all three of PD-L1, CXCL9, and IFNG in the sample from the individual is above a reference immune-score expression level and (e.g., a reference immune-score expression level is an immune-score expression level of PD-L1, CXCL9, and IFNG in a reference population). The PD-L1 axis binding antagonist may be any PD-L1 axis binding antagonist known in the art or described herein, for example, in Section III.F, below. For example, in some instances, the PD-L1 axis binding antagonist is a PD-L1 binding antagonist. In some instances, the PD-L1 binding antagonist is an antibody. In some instances, the antibody is selected from the group consisting of: YW243.55.S70, MPDL3280A (atezolizumab), MDX-1105, MEDI4736 (durvalumab), and MSB0010718C (avelumab). In some instances, the antibody comprises a heavy chain comprising HVR-H1 sequence of SEQ ID NO: 9, HVR-H2 sequence of SEQ ID NO: 10, and HVR-H3 sequence of SEQ ID NO: 11; and a light chain comprising HVR-L1 sequence of SEQ ID NO: 12, HVR-L2 sequence of SEQ ID NO: 13, and HVR-L3 sequence of SEQ ID NO: 14. In some instances, the antibody comprises a heavy chain variable region comprising the amino acid sequence of SEQ ID NO: 15 and a light chain variable region comprising the amino acid sequence of SEQ ID NO: 16.

In some instances, the methods further include administering to the individual an effective amount of an additional therapeutic agent. In some instances, the additional therapeutic agent is selected from the group consisting of a cytotoxic agent, a growth-inhibitory agent, a radiation therapy, an anti-angiogenic agent, as described herein, or a combination thereof.

Alternatively, in cases for which an individual is determined to have a decreased immune-score expression level of at least one, at least two, or all three of PD-L1, CXCL9, and IFNG relative to a reference immune-score expression level, the methods may further include administering to the individual an effective amount of an anti-cancer therapy other than, or in addition to, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). For example, the anti-cancer therapy other than, or in addition to, a PD-L1 axis binding antagonist may include a cytotoxic agent, a growth-inhibitory agent, a radiation therapy, an anti-angiogenic agent, as described herein, or a combination thereof, alone, or in addition to a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) and/or any additional therapeutic agent described herein.

(ii) Increased Immune-Score Expression Level of PD-L1, CXCL9, and IFNG

An immune-score expression level of PD-L1, CXCL9, and IFNG in a sample from the individual having cancer that is above or higher than a reference immune-score expression level of PD-L1, CXCL9, and IFNG may indicate that the individual is more likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein the reference immune-score expression level is an immune-score expression level of PD-L1, CXCL9, and IFNG in a reference population.

For example, in some instances, an immune-score expression level of PD-L1, CXCL9, and IFNG in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, CXCL9, and IFNG in the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level of PD-L1, CXCL9, and IFNG in the sample that is in about the top 10^{th} to about the top 90^{th} percentile, about the top 20^{th} to about the top 80^{th} percentile, about the top 30^{th} to about the top 70^{th} percentile, about the top 40^{th} to about the top 60^{th} percentile, about the top 45^{th} to about the top 55^{th} percentile, about the top 48^{th} to about the top 52^{th} percentile, about the top 49.5^{th} to about the top 50.5^{th} percentile, about the top 49.9^{th} to about the top 50.1^{th} percentile, or about the top 50^{th} percentile of the immune-score expression level of PD-L1, CXCL9, and IFNG in the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). For example, in some instances, an immune-score expression level of PD-L1, CXCL9, and IFNG in the sample that is between about 10% to about 90% prevalence, about 15% to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level of PD-L1, CXCL9, and IFNG in the sample that is in about the top 80^{th} percentile (i.e., equal to, or higher than, the 20% prevalence level) of the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, an immune-score expression level of PD-L1, CXCL9, and IFNG in the sample that is in about the top 75^{th} percentile (i.e., equal to, or higher than, the 25% prevalence level) of the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, an immune-score expression level of PD-L1, CXCL9, and IFNG in the sample that is in about the top 50^{th} percentile (i.e., equal to, or higher than, the 50% prevalence level) of the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, an immune-score expression level of PD-L1, CXCL9, and IFNG in the sample that is in about the top 25^{th} percentile (i.e., equal to, or higher than, the 75% prevalence level) of the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, an immune-score expression level of PD-L1, CXCL9, and IFNG in the sample that is in about the top 20^{th} percentile (i.e., equal to, or higher than, the 80% prevalence level) of the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level that is higher than a reference immune-score expression level refers to an overall increase of about 10%, 20%, 30%, 40%, 50%, 60%, 70%, 80%, 90%, 95%, 96%, 97%, 98%, or 99% or greater in the immune-score expression level of PD-L1, CXCL9, and IFNG, detected by standard art-known methods such as those described herein, as compared to the immune-score expression level of PD-L1, CXCL9, and IFNG in a reference sample, reference cell, reference tissue, control sample, control cell, or control tissue. In certain instances, an immune-score expression level that is higher than a reference immune-score expression level refers to an increase in the immune-score expression level of PD-L1, CXCL9, and IFNG in the sample, wherein the increase is at least about 1.5×, 1.75×, 2×, 3×, 4×, 5×, 6×, 7×, 8×, 9×, 10×, 25×, 50×, 75×, or 100× the immune-score expression level of PD-L1, CXCL9, and IFNG in a reference sample, reference cell, reference tissue, control sample, control cell, or control tissue. In some instances, an immune-score expression level that is higher than a reference immune-score expression level refers to an overall increase in the immune-score expression level of PD-L1, CXCL9, and IFNG that is greater than about 1.5-fold, about 1.75-fold, about 2-fold, about 2.25-fold, about 2.5-fold, about 2.75-fold, about 3.0-fold, or about 3.25-fold as compared to the immune-score expression level of PD-L1, CXCL9, and IFNG in a reference sample, reference cell, reference tissue, control sample, control cell, or control tissue.

In some instances, an immune-score expression level for PD-L1, CXCL9, and IFNG that is higher than a reference immune-score expression level refers to an overall increase of about 10%, 20%, 30%, 40%, 50%, 60%, 70%, 80%, 90%, 95%, 96%, 97%, 98%, or 99% or greater in the immune-score expression level of PD-L1, CXCL9, and IFNG, detected by standard art-known methods such as those described herein, as compared to a pre-assigned immune-score expression level of PD-L1, CXCL9, and IFNG. In certain instances, an immune-score expression level for PD-L1, CXCL9, and IFNG that is higher than a reference immune-score expression level refers to an increase in the immune-score expression level of PD-L1, CXCL9, and IFNG in the sample, wherein the increase is at least about 1.5×, 1.75×, 2×, 3×, 4×, 5×, 6×, 7×, 8×, 9×, 10×, 25×, 50×, 75×, or 100× a pre-assigned immune-score expression level of PD-L1, CXCL9, and IFNG. In some instances, an immune-score expression level for PD-L1, CXCL9, and IFNG that is higher than a reference immune-score expression level refers to an overall increase in the immune-score expression level of PD-L1, CXCL9, and IFNG that is greater than about 1.5-fold, about 1.75-fold, about 2-fold, about 2.25-fold, about 2.5-fold, about 2.75-fold, about 3.0-fold, or about 3.25-fold as compared to a pre-assigned immune-score expression level of PD-L1, CXCL9, and IFNG.

(iii) Decreased Immune-Score Expression Level of PD-L1, CXCL9, and IFNG

An immune-score expression level of PD-L1, CXCL9, and IFNG in a sample from the individual having cancer that is below or lower than a reference immune-score expression level of PD-L1, CXCL9, and IFNG may indicate that the individual is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein the reference immune-score expression level is an immune-score expression level of PD-L1, CXCL9, and IFNG in a reference population.

In some instances, an immune-score expression level of PD-L1, CXCL9, and IFNG in the sample that is in about the bottom 99^{th} percentile (equal to, or lower than, about the 99% prevalence level), about the bottom 95^{th} percentile (equal to, or lower than, about the 95% prevalence level), about the bottom 90^{th} percentile (equal to, or lower than, about the 90% prevalence level), about the bottom 85^{th} percentile (equal to, or lower than, about the 85% prevalence level), about the bottom 80^{th} percentile (equal to, or lower than, about the 80% prevalence level), about the bottom 75^{th} percentile (equal to, or lower than, about the 75% prevalence level), about the bottom 70^{th} percentile (equal to, or lower than, about the 70% prevalence level), about the bottom 65^{th} percentile (equal to, or lower than, about the 65% prevalence level), about the bottom 60^{th} percentile (equal to, or lower than, about the 60% prevalence level), about the bottom 55^{th} percentile (equal to, or lower than, about the 55% prevalence level), about the bottom 50^{th} percentile (equal to, or lower than, about the 50% prevalence level), about the bottom 45^{th} percentile (equal to, or lower than, about the 45% prevalence level), about the bottom 40^{th} percentile (equal to, or lower than, about the 40% prevalence level), about the bottom 35^{th} percentile (equal to, or lower than, about the 35% prevalence level), about the bottom 30^{th} percentile (equal to, or lower than, about the 30% prevalence level), about the bottom 25^{th} percentile (equal to, or lower than, about the 25% prevalence level), about the bottom 20^{th} percentile (equal to, or lower than, about the 20% prevalence level), about the bottom 15^{th} percentile (equal to, or lower than, about the 15% prevalence level), about the bottom 10^{th} percentile (equal to, or lower than, about the 10% prevalence level), about the bottom 5^{th} percentile (equal to, or lower than, about the 5% prevalence level), or about the bottom 1^{st} percentile (equal to, or lower than, about the 1% prevalence level) of the immune-score expression level of PD-L1, CXCL9, and IFNG in the reference population identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level of PD-L1, CXCL9, and IFNG in the sample that is in about the bottom 10^{th} to about the bottom 90^{th} percentile, about the bottom 20^{th} to about the bottom 80^{th} percentile, about the bottom 30^{th} to about the bottom 70^{th} percentile, about the bottom 40^{th} to about the bottom 60^{th} percentile, about the bottom 45^{th} to about the bottom 55^{th} percentile, about the bottom 48^{th} to about the bottom 52^{th} percentile, about the bottom 49.5^{th} to about the bottom 50.5^{th} percentile, about the bottom 49.9^{th} to about the bottom 50.1^{th} percentile, or about the bottom 50^{th} percentile of the immune-score expression level of PD-L1, CXCL9, and IFNG in the reference population identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). For example, in some instances, an immune-score expression level of PD-L1, CXCL9, and IFNG in the sample that is between about 10% to about 90% prevalence, about 15 to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level that is lower than a reference immune-score expression level refers to a decrease of about 10%, 20%, 30%, 40%, 50%, 60%, 70%, 80%, 90%, 95%, 96%, 97%, 98%, or 99% or greater in the immune-score expression level of PD-L1, CXCL9, and IFNG, detected by standard art-known methods such as those described herein, as compared to the immune-score expression level of PD-L1, CXCL9, and IFNG in a reference sample, reference cell, reference tissue, control sample, control cell, or control tissue. In certain instances, an immune-score expression level that is lower than a reference immune-score expression level refers to a decrease in the immune-score expression level of PD-L1, CXCL9, and IFNG in the sample, wherein the decrease is at least about 1.5×, 1.75×, 2×, 3×, 4×, 5×, 6×, 7×, 8×, 9×, 10×, 25×, 50×, 75×, or 100× the immune-score expression level of PD-L1, CXCL9, and IFNG in a reference sample, reference cell, reference tissue, control sample, control cell, or control tissue. In some instances, an immune-score expression level that is lower than a reference immune-score expression level refers to a decrease in the immune-score expression level of PD-L1, CXCL9, and IFNG that is greater than about 1.5-fold, about 1.75-fold, about 2-fold, about 2.25-fold, about 2.5-fold, about 2.75-fold, about 3.0-fold, or about 3.25-fold as compared to the immune-score expression level of PD-L1, CXCL9, and IFNG in a reference sample, reference cell, reference tissue, control sample, control cell, or control tissue.

In some instances, an immune-score expression level that is lower than a reference immune-score expression level refers to an overall decrease of about 10%, 20%, 30%, 40%, 50%, 60%, 70%, 80%, 90%, 95%, 96%, 97%, 98%, or 99% or greater in the immune-score expression level of PD-L1, CXCL9, and IFNG, detected by standard art-known methods such as those described herein, as compared to a pre-assigned immune-score expression level of PD-L1, CXCL9, and IFNG. In certain instances, an immune-score expression level that is lower than a reference immune-score expression level refers to a decrease in the immune-score expression level of PD-L1, CXCL9, and IFNG in the sample, wherein the decrease is at least about 1.5×, 1.75×, 2×, 3×, 4×, 5×, 6×, 7×, 8×, 9×, 10×, 25×, 50×, 75×, or 100× a pre-assigned immune-score expression level of PD-L1, CXCL9, and IFNG. In some instances, an immune-score expression level that is lower than a reference immune-score expression level refers to an overall decrease in the immune-score expression level of PD-L1, CXCL9, and IFNG that is greater than about 1.5-fold, about 1.75-fold, about 2-fold, about 2.25-fold, about 2.5-fold, about 2.75-fold, about 3.0-fold, or about 3.25-fold as compared to a pre-assigned immune-score expression level of PD-L1, CXCL9, and IFNG.

(iv) Reference Immune-Score Expression Level of PD-L1, CXCL9, and IFNG

The reference immune-score expression level described herein may be based on the immune-score expression level of PD-L1, CXCL9, and IFNG in a reference population. In some instances, the reference immune-score expression level described herein is an immune-score expression level of PD-L1, CXCL9, and IFNG in a reference population that includes two or more (e.g., two or more, three or more, four or more, or five or more) subsets of individuals.

In some instances, the reference immune-score expression level is an immune-score expression level of PD-L1, CXCL9, and IFNG in a reference population, wherein the reference population includes at least one subset of individuals having a cancer (e.g., lung cancer (e.g., NSCLC), bladder cancer (e.g., UBC), kidney cancer (e.g., RCC), or breast cancer (e.g., TNBC)).

In some instances, the reference immune-score expression level is an immune-score expression level of PD-L1, CXCL9, and IFNG in a reference population, wherein the reference population includes at least one subset of individuals having a cancer (e.g., lung cancer (e.g., NSCLC), bladder cancer (e.g., UBC), kidney cancer (e.g., RCC), or breast cancer (e.g., TNBC)) who have been administered one or more doses (e.g., at least one, two, three, four, five, six, seven, eight, nine, or ten or more doses) of a PD-L1 axis binding antagonist (e.g., as part of a PD-L1 axis binding antagonist monotherapy or combination therapy including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody))).

In some instances, the reference immune-score expression level is an immune-score expression level of PD-L1, CXCL9, and IFNG in a reference population, wherein the reference population includes at least one subset of individuals having a cancer (e.g., lung cancer (e.g., NSCLC), bladder cancer (e.g., UBC), kidney cancer (e.g., RCC), or breast cancer (e.g., TNBC)) who have received treatment with a PD-L1 axis binding antagonist therapy, wherein the PD-L1 axis binding antagonist therapy is a monotherapy (e.g., a PD-L1 axis binding antagonist monotherapy including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody))).

In some instances, the reference immune-score expression level is an immune-score expression level of PD-L1, CXCL9, and IFNG in a reference population, wherein the reference population includes at least one subset of individuals having a cancer (e.g., lung cancer (e.g., NSCLC), bladder cancer (e.g., UBC), kidney cancer (e.g., RCC), or breast cancer (e.g., TNBC)) who have received treatment with a PD-L1 axis binding antagonist therapy, wherein the PD-L1 axis binding antagonist therapy is a combination therapy (e.g., a combination therapy including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) and an additional therapeutic agent (e.g., anti-cancer therapy (e.g., a cytotoxic agent, a growth-inhibitory agent, a radiation therapy, an anti-angiogenic agent, or a combination thereof))).

In some instances, the reference immune-score expression level is an immune-score expression level of PD-L1, CXCL9, and IFNG in a reference population, wherein the reference population includes at least one subset of individuals having a cancer (e.g., lung cancer (e.g., NSCLC), bladder cancer (e.g., UBC), kidney cancer (e.g., RCC), or breast cancer (e.g., TNBC)) who have received treatment with a non-PD-L1 axis binding antagonist therapy, wherein the non-PD-L1 axis binding antagonist therapy does not include a PD-L1 axis binding antagonist and includes an anti-cancer therapy (e.g., a cytotoxic agent, a growth-inhibitory agent, a radiation therapy, an anti-angiogenic agent, or a combination thereof))).

For example, in some instances, the reference population includes a first subset of individuals who have been treated with a PD-L1 axis binding antagonist therapy (e.g., PD-L1 binding antagonist (e.g., anti-PD-L1 antibody, e.g., atezolizumab (MPDL3280A)) or PD-1 binding antagonist (e.g., anti-PD-1 antibody)) and a second subset of individuals who have been treated with a non-PD-L1 axis binding antagonist therapy, wherein the non-PD-L1 axis binding antagonist therapy does not include a PD-L1 axis binding antagonist.

In some instances, the reference immune-score expression level of PD-L1, CXCL9, and IFNG significantly separates each of the first and second subsets of individuals based on a significant difference between an individual's responsiveness (e.g., ORR, PFS, or OS) to treatment with the PD-L1 axis binding antagonist therapy and an individual's responsiveness to treatment with the non-PD-L1 axis binding antagonist therapy above the reference immune-score expression level, wherein the individual's responsiveness to treatment with the PD-L1 axis binding antagonist therapy is significantly improved relative to the individual's responsiveness to treatment with the non-PD-L1 axis binding antagonist therapy. For example, in some instances, the reference immune-score expression level of PD-L1, CXCL9, and IFNG optimally separates each of the first and second subsets of individuals based on a maximum difference between an individual's responsiveness (e.g., ORR, PFS, or OS) to treatment with the PD-L1 axis binding antagonist therapy and an individual's responsiveness to treatment with the non-PD-L1 axis binding antagonist therapy above the reference immune-score expression level, wherein the individual's responsiveness to treatment with the PD-L1 axis binding antagonist therapy is significantly improved relative to the individual's responsiveness to treatment with the non-PD-L1 axis binding antagonist therapy.

In some instances, the reference immune-score expression level of PD-L1, CXCL9, and IFNG significantly separates each of the first and second subsets of individuals based on a significant difference between an individual's responsiveness (e.g., ORR, PFS, or OS) to treatment with the PD-L1 axis binding antagonist therapy and an individual's responsiveness to treatment with the non-PD-L1 axis binding antagonist therapy below the reference immune-score expression level, wherein the individual's responsiveness to treatment with the non-PD-L1 axis binding antagonist therapy is significantly improved relative to the individual's responsiveness to treatment with the PD-L1 axis binding antagonist therapy. For example, in some instances, the reference immune-score expression level of PD-L1, CXCL9, and IFNG optimally separates each of the first and second subsets of individuals based on a maximum difference between an individual's responsiveness (e.g., ORR, PFS, or OS) to treatment with the PD-L1 axis binding antagonist therapy and an individual's responsiveness to treatment with the non-PD-L1 axis binding antagonist therapy below the reference immune-score expression level, wherein the individual's responsiveness to treatment with the non-PD-L1 axis binding antagonist therapy is significantly improved relative to the individual's responsiveness to treatment with the PD-L1 axis binding antagonist therapy.

In some instances, an optimal separation or significant separation may be based on a hazard ratio (HR) determined from an analysis of the immune-score expression level of PD-L1, CXCL9, and IFNG in the first and second subsets of individuals, wherein the HR is less than 1, e.g., an HR of about 0.95, about 0.9, about 0.8, about 0.7, about 0.6, about 0.5, about 0.4, about 0.3, about 0.2, about 0.1 or lower. For example, in particular instances, an optimal separation or significant separation may be based on a hazard ratio (HR) determined from an analysis of the immune-score expression level of PD-L1, CXCL9, and IFNG in the first and second subsets of individuals, wherein the upper bound of the 95% confidence interval of the HR is less than 1, e.g., an upper bound of the 95% confidence interval of the HR of about 0.95, about 0.9, about 0.8, about 0.7, about 0.6, about 0.5, about 0.4, about 0.3, about 0.2, about 0.1 or lower.

Additionally, or alternatively, the reference immune-score expression level may be an immune-score expression level of PD-L1, CXCL9, and IFNG in a reference population, wherein the reference population includes at least one subset of individuals who do not have a cancer (e.g., individuals not having NSCLC, UBC, RCC, or TNBC) or have cancer but are treatment naïve.

(v) Indications

The methods described herein are useful for predicting the therapeutic response of an individual having a cancer to treatment with a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, the cancer may be a lung cancer, a kidney cancer, a bladder cancer, a breast cancer, a colorectal cancer, an ovarian cancer, a pancreatic cancer, a gastric carcinoma, an esophageal cancer, mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a merkel cell cancer, or a hematologic malignancy.

In certain instances, the cancer may be a lung cancer. For example, the lung cancer may be a non-small cell lung cancer (NSCLC), including but not limited to a locally advanced or metastatic (e.g., stage IIIB, stage IV, or recurrent) NSCLC. In some instances, the lung cancer (e.g., NSCLC) is unresectable/inoperable lung cancer (e.g., NSCLC). For example, the methods described herein may be used for identifying an individual having a lung cancer (e.g., NSCLC) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining an immune-score expression level of PD-L1, CXCL9, and IFNG in a sample from the individual (e.g., a tumor tissue sample), wherein the immune-score expression level of at least one, at least two, or all three of PD-L1, CXCL9, and IFNG in the sample that is above a reference immune-score expression level (e.g., an immune-score expression level of PD-L1, CXCL9, and IFNG in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In certain instances, the cancer may be a bladder cancer. For example, the bladder cancer may be a urothelial bladder cancer, including but not limited to a non-muscle invasive urothelial bladder cancer, a muscle-invasive urothelial bladder cancer, or a metastatic urothelial bladder cancer. In some instances, the urothelial bladder cancer is a metastatic urothelial bladder cancer. For example, the methods described herein may be used for identifying an individual having a bladder cancer (e.g., UBC) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining an immune-score expression level of PD-L1, CXCL9, and IFNG in a sample from the individual (e.g., a tumor tissue sample), wherein the immune-score expression level of at least one, at least two, or all three of PD-L1, CXCL9, and IFNG in the sample that is above a reference immune-score expression level (e.g., an immune-score expression level of PD-L1, CXCL9, and IFNG in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In certain instances, the cancer may be a kidney cancer. In some instances, the kidney cancer may be a renal cell carcinoma (RCC), including stage I RCC, stage II RCC, stage III RCC, stage IV RCC, or recurrent RCC. For example, the methods described herein may be used for identifying an individual having a kidney cancer (e.g., RCC) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining an immune-score expression level of PD-L1, CXCL9, and IFNG in a sample from the individual (e.g., a tumor tissue sample), wherein the immune-score expression level of at least one, at least two, or all three of PD-L1, CXCL9, and IFNG in the sample that is above a reference immune-score expression level (e.g., an immune-score expression level of PD-L1, CXCL9, and IFNG in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In certain instances, the cancer may be a breast cancer. For example, the breast cancer may be TNBC, estrogen receptor-positive breast cancer, estrogen receptor-positive/HER2-negative breast cancer, HER2-negative breast cancer, HER2-positive breast cancer, estrogen receptor-negative breast cancer, progesterone receptor-positive breast cancer, or progesterone receptor-negative breast cancer. In some instances, the breast cancer may be a TNBC. For example, the methods described herein may be used for identifying an individual having a breast cancer (e.g., TNBC) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining an immune-score expression level of PD-L1, CXCL9, and IFNG in a sample from the individual (e.g., a tumor tissue sample), wherein the immune-score expression level of at least one, at least two, or all three of PD-L1, CXCL9, and IFNG in the sample that is above a reference immune-score expression level (e.g., an immune-score expression level of PD-L1, CXCL9, and IFNG in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, the individual having a cancer, e.g., cancers described herein, has not been previously treated for the cancer (treatment naïve). For example, in some instances, the individual having a cancer has not previously received a PD-L1 axis binding antagonist therapy (e.g., PD-L1 binding antagonist (e.g., anti-PD-L1 antibody, e.g., atezolizumab (MPDL3280A)) or PD-1 binding antagonist (e.g., anti-PD-1 antibody)). For example, in some instances, an immune-score expression level of at least one, at least two, or all three of PD-L1, CXCL9, and IFNG that is above a reference immune-score expression level (e.g., an immune-score expression level of PD-L1, CXCL9, and IFNG in a reference population) identifies the individual having cancer (e.g., lung cancer (e.g., NSCLC), bladder cancer (e.g., UBC), kidney cancer (e.g., RCC), or breast cancer (e.g., TNBC)) as one who may benefit from a first-line treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, the individual having a cancer has previously received treatment for the cancer. In some instances, the individual having a cancer has previously received treatment including a non-PD-L1 axis binding antagonist therapy (e.g., an anti-cancer therapy (e.g., a cytotoxic agent, a growth-inhibitory agent, a radiation therapy, an anti-angiogenic agent, or a combination thereof)). For example, in some instances, an immune-score expression level of at least one, at least two, or all three of PD-L1, CXCL9, and IFNG that is above a reference immune-score expression level (e.g., an immune-score expression level of PD-L1, CXCL9, and IFNG in a reference population) identifies the individual having cancer (e.g., lung cancer (e.g., NSCLC), bladder cancer (e.g., UBC), kidney cancer (e.g., RCC), or breast cancer (e.g., TNBC)) as one who may benefit from a second-line treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

(vi) Treatment Benefits

An individual who benefits from receiving treatment with a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may experience, for example, a delay or prevention in the occurrence or recurrence of a cancer (e.g., a lung cancer (e.g., NSCLC), a bladder cancer (e.g., UBC), a kidney cancer (e.g., RCC), or a breast cancer (e.g., TNBC)), alleviation of symptoms, diminishment of any direct or indirect pathological consequences of the cancer, prevention of metastasis, decrease in the rate of disease progression, amelioration or palliation of the disease state, or remission or improved prognosis. In some instances, the treatments described herein are used to delay development of a cancer or to slow the progression of a cancer (e.g., a lung cancer (e.g., NSCLC), a bladder cancer (e.g., UBC), a kidney cancer (e.g., RCC), or a breast cancer (e.g., TNBC)). In some instances, the benefit may be an increase in overall survival (OS), progression-free survival (PFS), complete response (CR), partial response (PR), or a combination thereof.

In some instances, an immune-score expression level of at least one, at least two, or all three of PD-L1, CXCL9, and IFNG that is above a reference immune-score expression level (e.g., an immune-score expression level of PD-L1, CXCL9, and IFNG in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein the benefit is an increase in OS, PFS, CR, PR, or a combination thereof, relative to a treatment that does not include a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level of at least one, at least two, or all three of PD-L1, CXCL9, and IFNG that is above a reference immune-score expression level (e.g., an immune-score expression level of PD-L1, CXCL9, and IFNG in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein the benefit is an increase in OS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level of at least one, at least two, or all three of PD-L1, CXCL9, and IFNG that is above a reference immune-score expression level (e.g., an immune-score expression level of PD-L1, CXCL9, and IFNG in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein the benefit is an increase in PFS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

### C. Four-Gene Immune-Score Combinations

In particular instances, the methods and assays provided herein may be used to determine an immune-score expression level of four genes selected from PD-L1, CXCL9, IFNG, GZMB, CD8A, and PD-1. For example, the determination step may include determining the expression levels of any one of the combination of four genes listed in Table 3.

In some instances, the determination step includes determining the expression levels of a particular combination of the four genes listed in Table 3 and one or more additional genes associated with T-effector cells, e.g., determining the expression level of (i) four genes selected from the group consisting of PD-L1, CXCL9, IFNG, GZMB, CD8A, and PD-1 (e.g., any one of the combinations of genes listed in Table 3) and (ii) one or more genes associated with T-effector cells (e.g., at least one, at least two, at least three, at least four, at least five, at least six, at least seven, at least eight, at least nine, at least ten, at least eleven, at least twelve, at least thirteen, at least fourteen, at least fifteen, or sixteen of CD8A, GZMA, GZMB, IFNG, EOMES, PRF1, PD-L1, PD-1, CXCL9, CD27, FOXP3, CTLA4, TIGIT, IDO1, CXCL10, CXCL11, PSMB8, PSMB9, TAP1, and/or TAP2), wherein the one or more genes associated with T-effector cells are different from the four genes selected from the group consisting of PD-L1, CXCL9, IFNG, GZMB, CD8A, and PD-1.

TABLE 3
Exemplary four gene immune-score combinations

PD-L1; CXCL9; IFNG; and GZMB
PD-L1; CXCL9; IFNG; and CD8A
PD-L1; CXCL9; IFNG; and PD-1
PD-L1; CXCL9; GZMB; and CD8A
PD-L1; CXCL9; GZMB; and PD-1
PD-L1; CXCL9; CD8A; and PD-1
PD-L1; IFNG; GZMB; and CD8A
PD-L1; IFNG; GZMB; and PD-1
PD-L1; IFNG; CD8A; and PD-1
PD-L1; GZMB; CD8A; and PD-1
CXCL9; IFNG; GZMB; and CD8A
CXCL9; IFNG; GZMB; and PD-1
CXCL9; IFNG; CD8A; and PD-1
CXCL9; GZMB; CD8A; and PD-1
IFNG; GZMB; CD8A; and PD-1

Provided herein are methods for identifying an individual having a cancer (e.g., lung cancer (e.g., NSCLC), bladder cancer (e.g., UBC), kidney cancer (e.g., RCC), or breast cancer (e.g., TNBC)) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of any one of the combinations of four genes listed in Table 3 in a sample from the individual (e.g., a tumor tissue sample), wherein an immune-score expression level of the combination of four genes listed in Table 3 in the sample that is above a reference immune-score expression level (e.g., an immune-score expression level of the same combination of four genes listed in Table 3 in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of a combination of four genes listed in Table 3 in the sample that is below a reference immune-score expression level (e.g., an immune-score expression level of the same combination of four genes listed in Table 3 in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

Also provided herein are methods for selecting a therapy for an individual having a cancer (e.g., lung cancer (e.g., NSCLC), bladder cancer (e.g., UBC), kidney cancer (e.g., RCC), or breast cancer (e.g., TNBC)), the methods including determining the expression level of a combination of four genes listed in Table 3 in a sample from the individual, wherein an immune-score expression level of a combination of four genes listed in Table 3 in the sample that is above a reference immune-score expression level (e.g., an immune-score expression level of the same combination of four genes listed in Table 3 in a reference population) identities an individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of a combination of four genes listed in Table 3 in the sample that is below a reference immune-score expression level (e.g., an immune-score expression level of the same combination of four genes listed in Table 3 in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

The examples and instances outlined below for the combination of the genes PD-L1, IFNG, GZMB, and CD8A may also apply to any of the four-gene combinations listed in Table 3.

(i) Expression of PD-L1, IFNG, GZMB, and CD8A

The methods and assays provided herein may be used to determine the immune-score expression level of PD-L1, IFNG, GZMB, and CD8A. Various diagnostic methods based on a determination of the immune-score expression level of PD-L1, IFNG, GZMB, and CD8A are further described below.

Provided herein are methods for identifying an individual having a cancer (e.g., lung cancer (e.g., NSCLC), bladder cancer (e.g., UBC), kidney cancer (e.g., RCC), or breast cancer (e.g., TNBC)) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of PD-L1, IFNG, GZMB, and CD8A in a sample from the individual (e.g., a tumor tissue sample), wherein an immune-score expression level of at least one, at least two, at least three, or all four of PD-L1, IFNG, GZMB, and CD8A in the sample that is above a reference immune-score expression level (e.g., an immune-score expression level of PD-L1, IFNG, GZMB, and CD8A in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of at least one, at least two, at least three, or all four of PD-L1, IFNG, GZMB, and CD8A in the sample that is below the reference immune-score expression level (e.g., an immune-score expression level of PD-L1, IFNG, GZMB, and CD8A in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

Also provided herein are methods for selecting a therapy for an individual having a cancer (e.g., lung cancer (e.g., NSCLC), bladder cancer (e.g., UBC), kidney cancer (e.g., RCC), or breast cancer (e.g., TNBC)), the methods including determining the expression level of PD-L1, IFNG, GZMB, and CD8A in a sample from the individual, wherein an immune-score expression level of at least one, at least two, at least three, or all four of PD-L1, IFNG, GZMB, and CD8A in the sample relative to a reference immune-score expression level (e.g., an immune-score expression level of PD-L1, IFNG, GZMB, and CD8A in a reference population) identities an individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of at least one, at least two, at least three, or all four of PD-L1, IFNG, GZMB, and CD8A in the sample that is below the reference immune-score expression level (e.g., an immune-score expression level of PD-L1, IFNG, GZMB, and CD8A in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

Further provided herein are methods for determining whether an individual with a cancer (e.g., lung cancer (e.g., NSCLC), bladder cancer (e.g., UBC), kidney cancer (e.g., RCC), or breast cancer (e.g., TNBC)) is likely to respond to treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of PD-L1, IFNG, GZMB, and CD8A in a sample from the individual (e.g., a tumor tissue sample), wherein an immune-score expression level of at least one, at least two, at least three, or all four of PD-L1, IFNG, GZMB, and CD8A relative to a reference immune-score expression level (e.g., an immune-score expression level of PD-L1, IFNG, GZMB, and CD8A in a reference population) indicates that the individual is likely to respond to treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level (e.g., an immune-score expression level of PD-L1, IFNG, GZMB, and CD8A in a reference population) of at least one, at least two, at least three, or all four of PD-L1, IFNG, GZMB, and CD8A in the sample that is below the reference immune-score expression level indicates that the individual is not likely to respond to a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

Further provided herein are methods for predicting the responsiveness of an individual with a cancer (e.g., lung cancer (e.g., NSCLC), bladder cancer (e.g., UBC), kidney cancer (e.g., RCC), or breast cancer (e.g., TNBC)) to treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of PD-L1, IFNG, GZMB, and CD8A in a sample from the individual (e.g., tumor tissue), wherein an immune-score expression level of at least one, at least two, at least three, or all four of PD-L1, IFNG, GZMB, and CD8A relative to a reference immune-score expression level (e.g., an immune-score expression level of PD-L1, IFNG, GZMB, and CD8A in a reference population) indicates that the individual is more likely to be responsive to treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of at least one, at least two, at least three, or all four of PD-L1, IFNG, GZMB, and CD8A in the sample that is below the reference immune-score expression level (e.g., an immune-score expression level of PD-L1, IFNG, GZMB, and CD8A in a reference population) indicates that the individual is more likely to be responsive to a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

Further provided herein are methods for determining the likelihood that an individual with a cancer (e.g., lung cancer (e.g., NSCLC), bladder cancer (e.g., UBC), kidney cancer (e.g., RCC), or breast cancer (e.g., TNBC)) will exhibit benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of PD-L1, IFNG, GZMB, and CD8A in a sample from the individual (e.g., tumor tissue), wherein an immune-score expression level of at least one, at least two, at least three, or all four of PD-L1, IFNG, GZMB, and CD8A relative to a reference immune-score expression level (e.g., an immune-score expression level of PD-L1, IFNG, GZMB, and CD8A in a reference population) indicates that the individual will have an increased likelihood of benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of at least one, at least two, at least three, or all four of PD-L1, IFNG, GZMB, and CD8A in the sample that is below the reference immune-score expression level (e.g., an immune-score expression level of PD-L1, IFNG, GZMB, and CD8A in a reference population) indicates that the individual will have a decreased likelihood of benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, the individual having a cancer (e.g., lung cancer (e.g., NSCLC), bladder cancer (e.g., UBC), kidney cancer (e.g., RCC), or breast cancer (e.g., TNBC)) may be provided a recommendation prior to administration of the PD-L1 binding antagonist, based on the immune-score expression level of PD-L1, IFNG, GZMB, and CD8A determined in accordance with any of the above methods. In some instances, the methods further include providing a recommendation that the individual will be likely to respond to or benefit from treatment with a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, the methods include providing a recommendation that the therapy selected for the individual includes treatment with a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, the methods may further include administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) to the individual. In some instances, the methods further include administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein the immune-score expression level of at least one, at least two, at least three, or all four of PD-L1, IFNG, GZMB, and CD8A in the sample from the individual is above a reference immune-score expression level (e.g., an immune-score expression level of PD-L1, IFNG, GZMB, and CD8A in a reference population). The PD-L1 axis binding antagonist may be any PD-L1 axis binding antagonist known in the art or described herein, for example, in Section III.F, below. For example, in some instances, the PD-L1 axis binding antagonist is a PD-L1 binding antagonist. In some instances, the PD-L1 binding antagonist is an antibody. In some instances, the antibody is selected from the group consisting of: YW243.55.S70, MPDL3280A (atezolizumab), MDX-1105, MEDI4736 (durvalumab), and MSB0010718C (avelumab). In some instances, the antibody comprises a heavy chain comprising HVR-H1 sequence of SEQ ID NO: 9, HVR-H2 sequence of SEQ ID NO: 10, and HVR-H3 sequence of SEQ ID NO: 11; and a light chain comprising HVR-L1 sequence of SEQ ID NO: 12, HVR-L2 sequence of SEQ ID NO: 13, and HVR-L3 sequence of SEQ ID NO: 14. In some instances, the antibody comprises a heavy chain variable region comprising the amino acid sequence of SEQ ID NO: 15 and a light chain variable region comprising the amino acid sequence of SEQ ID NO: 16.

In some instances, the methods further include administering to the individual an effective amount of an additional therapeutic agent. In some instances, the additional therapeutic agent is selected from the group consisting of a cytotoxic agent, a growth-inhibitory agent, a radiation therapy, an anti-angiogenic agent, as described herein, or a combination thereof.

Alternatively, in cases for which an individual is determined to have a decreased immune-score expression level of at least one, at least two, at least three, or all four of PD-L1, IFNG, GZMB, and CD8A relative to a reference immune-score expression level (e.g., an immune-score expression level of PD-L1, IFNG, GZMB, and CD8A in a reference population), the methods may further include administering to the individual an effective amount of an anti-cancer therapy other than, or in addition to, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). For example, the anti-cancer therapy other than, or in addition to, a PD-L1 axis binding antagonist may include a cytotoxic agent, a growth-inhibitory agent, a radiation therapy, an anti-angiogenic agent, as described herein, or a combination thereof, alone, or in addition to a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) and/or any additional therapeutic agent described herein.

(ii) Increased Immune-Score Expression Level of PD-L1, IFNG, GZMB, and CD8A

An immune-score expression level of PD-L1, IFNG, GZMB, and CD8A in a sample from the individual having cancer that is above or higher than a reference immune-score expression level of PD-L1, CXCL9, and/or IFNG (e.g., in a reference population or a pre-assigned score) may indicate that the individual is more likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

For example, in some instances, an immune-score expression level of PD-L1, IFNG, GZMB, and CD8A in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, IFNG, GZMB, and CD8A in the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level of PD-L1, IFNG, GZMB, and CD8A in the sample that is in about the top 10^{th} to about the top 90^{th} percentile, about the top 20^{th} to about the top 80^{th} percentile, about the top 30^{th} to about the top 70^{th} percentile, about the top 40^{th} to about the top 60^{th} percentile, about the top 45^{th} to about the top 55^{th} percentile, about the top 48^{th} to about the top 52^{th} percentile, about the top 49.5^{th} to about the top 50.5^{th} percentile, about the top 49.9^{th} to about the top 50.1^{th}percentile, or about the top 50^{th} percentile of the immune-score expression level of PD-L1, IFNG, GZMB, and CD8A in the reference population of the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). For example, in some instances, an immune-score expression level of PD-L1, IFNG, GZMB, and CD8A in the sample that is between about 10% to about 90% prevalence, about 15 to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level of PD-L1, IFNG, GZMB, and CD8A in the sample that is in about the top 80^{th} percentile (i.e., equal to, or higher than, the 20% prevalence level) of the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, an immune-score expression level of PD-L1, IFNG, GZMB, and CD8A in the sample that is in about the top 75^{th} percentile (i.e., equal to, or higher than, the 25% prevalence level) of the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, an immune-score expression level of PD-L1, IFNG, GZMB, and CD8A in the sample that is in about the top 50^{th} percentile (i.e., equal to, or higher than, the 50% prevalence level) of the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, an immune-score expression level of PD-L1, IFNG, GZMB, and CD8A in the sample that is in about the top 25^{th} percentile (e.g., equal to, or higher, than the 25% prevalence level) of the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, an immune-score expression level of PD-L1, IFNG, GZMB, and CD8A in the sample that is in about the top 20^{th} percentile (i.e., equal to, or higher than, the 80% prevalence) of the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level that is higher than a reference immune-score expression level refers to an overall increase of about 10%, 20%, 30%, 40%, 50%, 60%, 70%, 80%, 90%, 95%, 96%, 97%, 98%, or 99% or greater in the expression level of PD-L1, IFNG, GZMB, and CD8A, detected by standard art-known methods such as those described herein, as compared to the immune-score expression level of PD-L1, IFNG, GZMB, and CD8A in a reference sample, reference cell, reference tissue, control sample, control cell, or control tissue. In certain instances, an immune-score expression level that is higher than a reference immune-score expression level refers to an increase in the expression level of PD-L1, IFNG, GZMB, and CD8A in the sample, wherein the increase is at least about 1.5×, 1.75×, 2×, 3×, 4×, 5×, 6×, 7×, 8×, 9×, 10×, 25×, 50×, 75×, or 100× the immune-score expression level of PD-L1, IFNG, GZMB, and CD8A in a reference sample, reference cell, reference tissue, control sample, control cell, or control tissue. In some instances, an immune-score expression level that is higher than a reference immune-score expression level refers to an overall increase in the immune-score expression level of PD-L1, IFNG, GZMB, and CD8A that is greater than about 1.5-fold, about 1.75-fold, about 2-fold, about 2.25-fold, about 2.5-fold, about 2.75-fold, about 3.0-fold, or about 3.25-fold as compared to the immune-score expression level of PD-L1, IFNG, GZMB, and CD8A in a reference sample, reference cell, reference tissue, control sample, control cell, or control tissue.

In some instances, an immune-score expression level for PD-L1, IFNG, GZMB, and CD8A that is higher than a reference immune-score expression level refers to an overall increase of about 10%, 20%, 30%, 40%, 50%, 60%, 70%, 80%, 90%, 95%, 96%, 97%, 98%, or 99% or greater in the expression level of PD-L1, IFNG, GZMB, and CD8A, detected by standard art-known methods such as those described herein, as compared to a pre-assigned immune-score expression level of PD-L1, IFNG, GZMB, and CD8A. In certain instances, an immune-score expression level for PD-L1, IFNG, GZMB, and CD8A that is higher than a reference immune-score expression level refers to an increase in the expression level of PD-L1, IFNG, GZMB, and CD8A in the sample, wherein the increase is at least about 1.5×, 1.75×, 2×, 3×, 4×, 5×, 6×, 7×, 8×, 9×, 10×, 25×, 50×, 75×, or 100× a pre-assigned immune-score expression level of PD-L1, IFNG, GZMB, and CD8A. In some instances, an immune-score expression level for PD-L1, IFNG, GZMB, and CD8A that is higher than a reference immune-score expression level refers to an overall increase in the immune-score expression level of PD-L1, IFNG, GZMB, and CD8A that is greater than about 1.5-fold, about 1.75-fold, about 2-fold, about 2.25-fold, about 2.5-fold, about 2.75-fold, about 3.0-fold, or about 3.25-fold as compared to a pre-assigned immune-score expression level of PD-L1, IFNG, GZMB, and CD8A.

(iii) Decreased Immune-Score Expression Level of PD-L1, IFNG, GZMB, and CD8A

An immune-score expression level of PD-L1, IFNG, GZMB, and CD8A in a sample from the individual having cancer that is below or lower than a reference immune-score expression level of PD-L1, IFNG, GZMB, and CD8A (e.g., in a reference population or pre-assigned score) may indicate that the individual is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein the reference immune-score expression level is an immune-score expression level of PD-L1, IFNG, GZMB, and CD8A in a reference population.

In some instances, an immune-score expression level of PD-L1, IFNG, GZMB, and CD8A in the sample that is in about the bottom 99^{th} percentile (equal to, or lower than, about the 99% prevalence level), about the bottom 95^{th} percentile (equal to, or lower than, about the 95% prevalence level), about the bottom 90^{th} percentile (equal to, or lower than, about the 90% prevalence level), about the bottom 85^{th} percentile (equal to, or lower than, about the 85% prevalence level), about the bottom 80^{th} percentile (equal to, or lower than, about the 80% prevalence level), about the bottom 75^{th} percentile (equal to, or lower than, about the 75% prevalence level), about the bottom 70^{th} percentile (equal to, or lower than, about the 70% prevalence level), about the bottom 65^{th} percentile (equal to, or lower than, about the 65% prevalence level), about the bottom 60^{th} percentile (equal to, or lower than, about the 60% prevalence level), about the bottom 55^{th} percentile (equal to, or lower than, about the 55% prevalence level), about the bottom 50^{th} percentile (equal to, or lower than, about the 50% prevalence level), about the bottom 45^{th} percentile (equal to, or lower than, about the 45% prevalence level), about the bottom 40^{th} percentile (equal to, or lower than, about the 40% prevalence level), about the bottom 35^{th} percentile (equal to, or lower than, about the 35% prevalence level), about the bottom 30^{th} percentile (equal to, or lower than, about the 30% prevalence level), about the bottom 25^{th} percentile (equal to, or lower than, about the 25% prevalence level), about the bottom 20^{th} percentile (equal to, or lower than, about the 20% prevalence level), about the bottom 15^{th} percentile (equal to, or lower than, about the 15% prevalence level), about the bottom 10^{th} percentile (equal to, or lower than, about the 10% prevalence level), about the bottom 5^{th} percentile (equal to, or lower than, about the 5% prevalence level), or about the bottom 1^{st} percentile (equal to, or lower than, about the 1% prevalence level) of the immune-score expression level of PD-L1, IFNG, GZMB, and CD8A in the reference population identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level of PD-L1, IFNG, GZMB, and CD8A in the sample that is in about the bottom 10^{th} to about the bottom 90^{th} percentile, about the bottom 20^{th} to about the bottom 80^{th} percentile, about the bottom 30^{th} to about the bottom 70^{th} percentile, about the bottom 40^{th} to about the bottom 60^{th} percentile, about the bottom 45^{th} to about the bottom 55^{th} percentile, about the bottom 48^{th} to about the bottom 52^{th} percentile, about the bottom 49.5^{th} to about the bottom 50.5^{th} percentile, about the bottom 49.9^{th} to about the bottom 50.1^{th} percentile, or about the bottom 50^{th} percentile of the immune-score expression level of PD-L1, IFNG, GZMB, and CD8A in the reference population identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). For example, in some instances, an immune-score expression level of PD-L1, IFNG, GZMB, and CD8A in the sample that is between about 10% to about 90% prevalence, about 15 to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level for PD-L1, IFNG, GZMB, and CD8A that is lower than a reference immune-score expression level refers to a decrease of about 10%, 20%, 30%, 40%, 50%, 60%, 70%, 80%, 90%, 95%, 96%, 97%, 98%, or 99% or greater in the expression level of PD-L1, IFNG, GZMB, and CD8A, detected by standard art-known methods such as those described herein, as compared to the immune-score expression level of PD-L1, IFNG, GZMB, and CD8A in a reference sample, reference cell, reference tissue, control sample, control cell, or control tissue. In certain instances, an immune-score expression level for PD-L1, IFNG, GZMB, and CD8A that is lower than a reference immune-score expression level refers to a decrease in the expression level of PD-L1, IFNG, GZMB, and CD8A in the sample, wherein the decrease is at least about 1.5×, 1.75×, 2×, 3×, 4×, 5×, 6×, 7×, 8×, 9×, 10×, 25×, 50×, 75×, or 100× the immune-score expression level of PD-L1, IFNG, GZMB, and CD8A in a reference sample, reference cell, reference tissue, control sample, control cell, or control tissue. In some instances, an immune-score expression level for PD-L1, IFNG, GZMB, and CD8A that is lower than a reference immune-score expression level refers to a decrease in the immune-score expression level of PD-L1, IFNG, GZMB, and CD8A that is greater than about 1.5-fold, about 1.75-fold, about 2-fold, about 2.25-fold, about 2.5-fold, about 2.75-fold, about 3.0-fold, or about 3.25-fold as compared to the immune-score expression level of PD-L1, IFNG, GZMB, and CD8A in a reference sample, reference cell, reference tissue, control sample, control cell, or control tissue.

In some instances, an immune-score expression level that is lower than a reference immune-score expression level refers to an overall decrease of about 10%, 20%, 30%, 40%, 50%, 60%, 70%, 80%, 90%, 95%, 96%, 97%, 98%, or 99% or greater in the expression level of PD-L1, IFNG, GZMB, and CD8A, detected by standard art-known methods such as those described herein, as compared to a pre-assigned immune-score expression level of PD-L1, IFNG, GZMB, and CD8A. In certain instances, an immune-score expression level that is lower than a reference immune-score expression level refers to a decrease in the expression level of PD-L1, IFNG, GZMB, and CD8A in the sample, wherein the decrease is at least about 1.5×, 1.75×, 2×, 3×, 4×, 5×, 6×, 7×, 8×, 9×, 10×, 25×, 50×, 75×, or 100× a pre-assigned immune-score expression level of PD-L1, IFNG, GZMB, and CD8A. In some instances, an immune-score expression level that is lower than a reference immune-score expression level refers to an overall decrease in the immune-score expression level of PD-L1, IFNG, GZMB, and CD8A that is greater than about 1.5-fold, about 1.75-fold, about 2-fold, about 2.25-fold, about 2.5-fold, about 2.75-fold, about 3.0-fold, or about 3.25-fold as compared to a pre-assigned immune-score expression level of PD-L1, IFNG, GZMB, and CD8A.

(iv) Reference Immune-Score Expression Level of PD-L1, IFNG, GZMB, and CD8A

The reference immune-score expression level described herein may be based on the immune-score expression level of PD-L1, IFNG, GZMB, and CD8A in a reference population. In some instances, the reference immune-score expression level described herein is an immune-score expression level of PD-L1, IFNG, GZMB, and CD8A in a reference population that includes two or more (e.g., two or more, three or more, four or more, or five or more) subsets of individuals.

In some instances, the reference immune-score expression level is an immune-score expression level of PD-L1, IFNG, GZMB, and CD8A in a reference population, wherein the reference population includes at least one subset of individuals having a cancer (e.g., lung cancer (e.g., NSCLC), bladder cancer (e.g., UBC), kidney cancer (e.g., RCC), or breast cancer (e.g., TNBC)).

In some instances, the reference immune-score expression level is an immune-score expression level of PD-L1, IFNG, GZMB, and CD8A in a reference population, wherein the reference population includes at least one subset of individuals having a cancer (e.g., lung cancer (e.g., NSCLC), bladder cancer (e.g., UBC), kidney cancer (e.g., RCC), or breast cancer (e.g., TNBC)) who have been administered one or more doses (e.g., at least one, two, three, four, five, six, seven, eight, nine, or ten or more doses) of a PD-L1 axis binding antagonist (e.g., as part of a PD-L1 axis binding antagonist monotherapy or combination therapy including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody))).

In some instances, the reference immune-score expression level is an immune-score expression level of PD-L1, IFNG, GZMB, and CD8A in a reference population, wherein the reference population includes at least one subset of individuals having a cancer (e.g., lung cancer (e.g., NSCLC), bladder cancer (e.g., UBC), kidney cancer (e.g., RCC), or breast cancer (e.g., TNBC)) who have received treatment with a PD-L1 axis binding antagonist therapy, wherein the PD-L1 axis binding antagonist therapy is a monotherapy (e.g., a PD-L1 axis binding antagonist monotherapy including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody))).

In some instances, the reference immune-score expression level is an immune-score expression level of PD-L1, IFNG, GZMB, and CD8A in a reference population, wherein the reference population includes at least one subset of individuals having a cancer (e.g., lung cancer (e.g., NSCLC), bladder cancer (e.g., UBC), kidney cancer (e.g., RCC), or breast cancer (e.g., TNBC)) who have received treatment with a PD-L1 axis binding antagonist therapy, wherein the PD-L1 axis binding antagonist therapy is a combination therapy (e.g., a combination therapy including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) and an additional therapeutic agent (e.g., anti-cancer therapy (e.g., a cytotoxic agent, a growth-inhibitory agent, a radiation therapy, an anti-angiogenic agent, or a combination thereof))).

For example, in some instances, the reference population includes a first subset of individuals who have been treated with a PD-L1 axis binding antagonist therapy (e.g., PD-L1 binding antagonist (e.g., anti-PD-L1 antibody, e.g., atezolizumab (MPDL3280A)) or PD-1 binding antagonist (e.g., anti-PD-1 antibody)) and a second subset of individuals who have been treated with a non-PD-L1 axis binding antagonist therapy, wherein the non-PD-L1 axis binding antagonist therapy does not include a PD-L1 axis binding antagonist.

In some instances, the reference immune-score expression level of PD-L1, IFNG, GZMB, and CD8A significantly separates each of the first and second subsets of individuals based on a significant difference between an individual's responsiveness (e.g., ORR, PFS, or OS) to treatment with the PD-L1 axis binding antagonist therapy and an individual's responsiveness to treatment with the non-PD-L1 axis binding antagonist therapy above the reference immune-score expression level, wherein the individual's responsiveness to treatment with the PD-L1 axis binding antagonist therapy is significantly improved relative to the individual's responsiveness to treatment with the non-PD-L1 axis binding antagonist therapy. For example, in some instances, the reference immune-score expression level of PD-L1, IFNG, GZMB, and CD8A optimally separates each of the first and second subsets of individuals based on a maximum difference between an individual's responsiveness (e.g., ORR, PFS, or OS) to treatment with the PD-L1 axis binding antagonist therapy and an individual's responsiveness to treatment with the non-PD-L1 axis binding antagonist therapy above the reference immune-score expression level, wherein the individual's responsiveness to treatment with the PD-L1 axis binding antagonist therapy is significantly improved relative to the individual's responsiveness to treatment with the non-PD-L1 axis binding antagonist therapy.

In some instances, the reference immune-score expression level of PD-L1, IFNG, GZMB, and CD8A significantly separates each of the first and second subsets of individuals based on a significant difference between an individual's responsiveness (e.g., ORR, PFS, or OS) to treatment with the PD-L1 axis binding antagonist therapy and an individual's responsiveness to treatment with the non-PD-L1 axis binding antagonist therapy below the reference immune-score expression level, wherein the individual's responsiveness to treatment with the non-PD-L1 axis binding antagonist therapy is significantly improved relative to the individual's responsiveness to treatment with the PD-L1 axis binding antagonist therapy. For example, in some instances, the reference immune-score expression level of PD-L1, IFNG, GZMB, and

CD8A optimally separates each of the first and second subsets of individuals based on a maximum difference between an individual's responsiveness (e.g., ORR, PFS, or OS) to treatment with the PD-L1 axis binding antagonist therapy and an individual's responsiveness to treatment with the non-PD-L1 axis binding antagonist therapy below the reference immune-score expression level, wherein the individual's responsiveness to treatment with the non-PD-L1 axis binding antagonist therapy is significantly improved relative to the individual's responsiveness to treatment with the PD-L1 axis binding antagonist therapy.

In some instances, an optimal separation or significant separation may be based on a hazard ratio (HR) determined from an analysis of the immune-score expression level of PD-L1, IFNG, GZMB, and CD8A in the first and second subsets of individuals, wherein the HR is less than 1, e.g., an HR of about 0.95, about 0.9, about 0.8, about 0.7, about 0.6, about 0.5, about 0.4, about 0.3, about 0.2, about 0.1 or lower. For example, in particular instances, an optimal separation or significant separation may be based on a hazard ratio (HR) determined from an analysis of the immune-score expression level of PD-L1, CXCL9, and IFNG in the first and second subsets of individuals, wherein the upper bound of the 95% confidence interval of the HR is less than 1, e.g., an upper bound of the 95% confidence interval of the HR of about 0.95, about 0.9, about 0.8, about 0.7, about 0.6, about 0.5, about 0.4, about 0.3, about 0.2, about 0.1 or lower.

Additionally, or alternatively, the reference immune-score expression level may be an immune-score expression level of PD-L1, IFNG, GZMB, and CD8A in a reference population, wherein the reference population includes at least one subset of individuals who do not have a cancer (e.g., individuals not having NSCLC, UBC, RCC, or TNBC) or have cancer but are treatment naïve.

(v) Indications

The methods described herein are useful for predicting the therapeutic response of an individual having a cancer to treatment with a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, the cancer may be a lung cancer, a kidney cancer, a bladder cancer, a breast cancer, a colorectal cancer, an ovarian cancer, a pancreatic cancer, a gastric carcinoma, an esophageal cancer, mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a merkel cell cancer, or a hematologic malignancy.

In certain instances, the cancer may be a lung cancer. For example, the lung cancer may be a non-small cell lung cancer (NSCLC), including but not limited to a locally advanced or metastatic (e.g., stage IIIB, stage IV, or recurrent) NSCLC. In some instances, the lung cancer (e.g., NSCLC) is unresectable/inoperable lung cancer (e.g., NSCLC). For example, the methods described herein may be used for identifying an individual having a lung cancer (e.g., NSCLC) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining an immune-score expression level of PD-L1, IFNG, GZMB, and CD8A in a sample from the individual (e.g., a tumor tissue sample), wherein the immune-score expression level of at least one, at least two, at least three, or all four of PD-L1, IFNG, GZMB, and CD8A in the sample that is above a reference immune-score expression level (e.g., an immune-score expression level of PD-L1, IFNG, GZMB, and CD8A in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In certain instances, the cancer may be a bladder cancer. For example, the bladder cancer may be a urothelial bladder cancer, including but not limited to a non-muscle invasive urothelial bladder cancer, a muscle-invasive urothelial bladder cancer, or a metastatic urothelial bladder cancer. In some instances, the urothelial bladder cancer is a metastatic urothelial bladder cancer. For example, the methods described herein may be used for identifying an individual having a bladder cancer (e.g., UBC) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining an immune-score expression level of PD-L1, IFNG, GZMB, and CD8A in a sample from the individual (e.g., a tumor tissue sample), wherein the immune-score expression level of at least one, at least two, at least three, or all four of PD-L1, IFNG, GZMB, and CD8A in the sample that is above a reference immune-score expression level (e.g., an immune-score expression level of PD-L1, IFNG, GZMB, and CD8A in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In certain instances, the cancer may be a kidney cancer. In some instances, the kidney cancer may be a renal cell carcinoma (RCC), including stage I RCC, stage II RCC, stage III RCC, stage IV RCC, or recurrent RCC. For example, the methods described herein may be used for identifying an individual having a kidney cancer (e.g., RCC) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining an immune-score expression level of PD-L1, IFNG, GZMB, and CD8A in a sample from the individual (e.g., a tumor tissue sample), wherein the immune-score expression level of at least one, at least two, at least three, or all four of PD-L1, IFNG, GZMB, and CD8A in the sample that is above a reference immune-score expression level (e.g., an immune-score expression level of PD-L1, IFNG, GZMB, and CD8A in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In certain instances, the cancer may be a breast cancer. For example, the breast cancer may be TNBC, estrogen receptor-positive breast cancer, estrogen receptor-positive/HER2-negative breast cancer, HER2-negative breast cancer, HER2-positive breast cancer, estrogen receptor-negative breast cancer, progesterone receptor-positive breast cancer, or progesterone receptor-negative breast cancer. In some instances, the breast cancer may be a TNBC. For example, the methods described herein may be used for identifying an individual having a breast cancer (e.g., TNBC) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining an immune-score expression level of PD-L1, IFNG, GZMB, and CD8A in a sample from the individual (e.g., a tumor tissue sample), wherein the immune-score expression level of at least one, at least two, at least three, or all four of PD-L1, IFNG, GZMB, and CD8A in the sample that is above a reference immune-score expression level (e.g., an immune-score expression level of PD-L1, IFNG, GZMB, and CD8A in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, the individual having a cancer, e.g., cancers described herein, has not been previously treated for the cancer (treatment naïve). For example, in some instances, the individual having a cancer has not previously received a PD-L1 axis binding antagonist therapy (e.g., PD-L1 binding antagonist (e.g., anti-PD-L1 antibody, e.g., atezolizumab (MPDL3280A)) or PD-1 binding antagonist (e.g., anti-PD-1 antibody)). For example, in some instances, an immune-score expression level of at least one, at least two, at least three, or all four of PD-L1, IFNG, GZMB, and CD8A that is above a reference immune-score expression level (e.g., an immune-score expression level of PD-L1, IFNG, GZMB, and CD8A in a reference population) identifies the individual having cancer (e.g., lung cancer (e.g., NSCLC), bladder cancer (e.g., UBC), kidney cancer (e.g., RCC), or breast cancer (e.g., TNBC)) as one who may benefit from a first-line treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, the individual having a cancer has previously received treatment for the cancer. In some instances, the individual having a cancer has previously received treatment including a non-PD-L1 axis binding antagonist therapy (e.g., an anti-cancer therapy (e.g., a cytotoxic agent, a growth-inhibitory agent, a radiation therapy, an anti-angiogenic agent, or a combination thereof)). For example, in some instances, an immune-score expression level of at least one, at least two, at least three, or all four of PD-L1, IFNG, GZMB, and CD8A that is above a reference immune-score expression level (e.g., an immune-score expression level of PD-L1, IFNG, GZMB, and CD8A in a reference population) identifies the individual having cancer (e.g., lung cancer (e.g., NSCLC), bladder cancer (e.g., UBC), kidney cancer (e.g., RCC), or breast cancer (e.g., TNBC)) as one who may benefit from a second-line treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

(vi) Treatment Benefits

An individual who benefits from receiving treatment with a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may experience, for example, a delay or prevention in the occurrence or recurrence of a cancer (e.g., a lung cancer (e.g., NSCLC), a bladder cancer (e.g., UBC), a kidney cancer (e.g., RCC), or a breast cancer (e.g., TNBC)), alleviation of symptoms, diminishment of any direct or indirect pathological consequences of the cancer, prevention of metastasis, decrease in the rate of disease progression, amelioration or palliation of the disease state, or remission or improved prognosis. In some instances, the treatments described herein are used to delay development of a cancer or to slow the progression of a cancer (e.g., a lung cancer (e.g., NSCLC), a bladder cancer (e.g., UBC), a kidney cancer (e.g., RCC), or a breast cancer (e.g., TNBC)). In some instances, the benefit may be an increase in overall survival (OS), progression-free survival (PFS), complete response (CR), partial response (PR), or a combination thereof.

In some instances, an immune-score expression level of at least one, at least two, at least three, or all four of PD-L1, IFNG, GZMB, and CD8A that is above a reference immune-score expression level (e.g., an immune-score expression level of PD-L1, IFNG, GZMB, and CD8A in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein the benefit is an increase in OS, PFS, CR, PR, or a combination thereof, relative to a treatment that does not include a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level of at least one, at least two, at least three, or all four of PD-L1, IFNG, GZMB, and CD8A that is above a reference immune-score expression level (e.g., an immune-score expression level of PD-L1, IFNG, GZMB, and CD8A in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein the benefit is an increase in OS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level of at least one, at least two, at least three, or all four of PD-L1, IFNG, GZMB, and CD8A that is above a reference immune-score expression level (e.g., an immune-score expression level of PD-L1, IFNG, GZMB, and CD8A in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein the benefit is an increase in PFS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

### D. Five-Gene Immune-Score Combinations

In particular instances, the methods and assays provided herein may be used to determine an immune-score expression level of five genes selected from PD-L1, CXCL9, IFNG, GZMB, CD8A, and PD-1. For example, the determination step may include determining the expression levels of any one of the combination of five genes listed in Table 4.

In some instances, the determination step includes determining the expression levels of a particular combination of the five genes listed in Table 4 and one or more additional genes associated with T-effector cells, e.g., determining the expression level of (i) five genes selected from the group consisting of PD-L1, CXCL9, IFNG, GZMB, CD8A, and PD-1 (e.g., any one of the combinations of genes listed in Table 4) and (ii) one or more genes associated with T-effector cells (e.g., at least one, at least two, at least three, at least four, at least five, at least six, at least seven, at least eight, at least nine, at least ten, at least eleven, at least twelve, at least thirteen, at least fourteen, or fifteen of CD8A, GZMA, GZMB, IFNG, EOMES, PRF1, PD-L1, PD-1, CXCL9, CD27, FOXP3, CTLA4, TIGIT, IDO1, CXCL10, CXCL11, PSMB8, PSMB9, TAP1, and/or TAP2), wherein the one or more genes associated with T-effector cells are different from the five genes selected from the group consisting of PD-L1, CXCL9, IFNG, GZMB, CD8A, and PD-1.

TABLE 4
Exemplary five gene immune-score combinations

PD-L1; CXCL9; IFNG; GZMB; and CD8A
PD-L1; CXCL9; IFNG; GZMB; and PD-1
PD-L1; CXCL9; IFNG; CD8A; and PD-1
PD-L1; CXCL9; GZMB; CD8A; and PD-1
PD-L1; IFNG; GZMB; CD8A; and PD-1
CXCL9; IFNG; GZMB; CD8A; and PD-1

Provided herein are methods for identifying an individual having a cancer (e.g., lung cancer (e.g., NSCLC), bladder cancer (e.g., UBC), kidney cancer (e.g., RCC), or breast cancer (e.g., TNBC)) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of a combination of five genes listed in Table 4 in a sample from the individual (e.g., a tumor tissue sample), wherein an immune-score expression level of a combination of five genes listed in Table 4 in the sample that is above a reference immune-score expression level (e.g., an immune-score expression level of the same combination of five genes listed in Table 4 in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of at a combination of five genes listed in Table 4 in the sample that is below a reference immune-score expression level (e.g., an immune-score expression level of the same combination of five genes listed in Table 4 in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Also provided herein are methods for selecting a therapy for an individual having a cancer (e.g., lung cancer (e.g., NSCLC), bladder cancer (e.g., UBC), kidney cancer (e.g., RCC), or breast cancer (e.g., TNBC)), the methods including determining the expression level of a combination of five genes listed in Table 4 in a sample from the individual, wherein an immune-score expression level of a combination of five genes listed in Table 4 in the sample that is above a reference immune-score expression level (e.g., an immune-score expression level of the same combination of five genes listed in Table 4 in a reference population) identities an individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of a combination of five genes listed in Table 4 in the sample that is below a reference immune-score expression level (e.g., an immune-score expression level of the same combination of five genes listed in Table 4 in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

The examples and instances outlined below for the combination of the genes PD-L1, IFNG, GZMB, CD8A, and PD-1 may also apply to any one of the five-gene combinations listed in Table 4.

(i) Expression of PD-L1, IFNG, GZMB, CD8A, and PD-1

The methods and assays provided herein may be used to determine the immune-score expression level of PD-L1, IFNG, GZMB, CD8A, and PD-1. Various diagnostic methods based on a determination of the immune-score expression level of PD-L1, IFNG, GZMB, CD8A, and PD-1 are further described below.

Provided herein are methods for identifying an individual having a cancer (e.g., lung cancer (e.g., NSCLC), bladder cancer (e.g., UBC), kidney cancer (e.g., RCC), or breast cancer (e.g., TNBC)) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of PD-L1, IFNG, GZMB, CD8A, and PD-1 in a sample from the individual (e.g., a tumor tissue sample), wherein an immune-score expression level of at least one, at least two, at least three, at least four, or all five of PD-L1, IFNG, GZMB, CD8A, and PD-1 in the sample that is above a reference immune-score expression level (e.g., an immune-score expression level of PD-L1, IFNG, GZMB, CD8A, and PD-1 in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of at least one, at least two, at least three, at least four, or all five of PD-L1, IFNG, GZMB, CD8A, and PD-1 in the sample that is below the reference immune-score expression level (e.g., an immune-score expression level of PD-L1, IFNG, GZMB, CD8A, and PD-1 in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

Also provided herein are methods for selecting a therapy for an individual having a cancer (e.g., lung cancer (e.g., NSCLC), bladder cancer (e.g., UBC), kidney cancer (e.g., RCC), or breast cancer (e.g., TNBC)), the methods including determining the expression level of PD-L1, IFNG, GZMB, CD8A, and PD-1 in a sample from the individual, wherein an immune-score expression level of at least one, at least two, at least three, at least four, or all five of PD-L1, IFNG, GZMB, CD8A, and PD-1 in the sample relative to a reference immune-score expression level (e.g., an immune-score expression level of PD-L1, IFNG, GZMB, CD8A, and PD-1 in a reference population) identities an individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of at least one, at least two, at least three, at least four, or all five of PD-L1, IFNG, GZMB, CD8A, and PD-1 in the sample that is below the reference immune-score expression level (e.g., an immune-score expression level of PD-L1, IFNG, GZMB, CD8A, and PD-1 in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

Further provided herein are methods for determining whether an individual with a cancer (e.g., lung cancer (e.g., NSCLC), bladder cancer (e.g., UBC), kidney cancer (e.g., RCC), or breast cancer (e.g., TNBC)) is likely to respond to treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of PD-L1, IFNG, GZMB, CD8A, and PD-1 in a sample from the individual (e.g., a tumor tissue sample), wherein an immune-score expression level of at least one, at least two, at least three, at least four, or all five of PD-L1, IFNG, GZMB, CD8A, and PD-1 relative to a reference immune-score expression level (e.g., an immune-score expression level of PD-L1, IFNG, GZMB, CD8A, and PD-1 in a reference population) indicates that the individual is likely to respond to treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of at least one, at least two, at least three, at least four, or all five of PD-L1, IFNG, GZMB, CD8A, and PD-1 in the sample that is below the reference immune-score expression level (e.g., an immune-score expression level of PD-L1, IFNG, GZMB, CD8A, and PD-1 in a reference population) indicates that the individual is not likely to respond to a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

Further provided herein are methods for predicting the responsiveness of an individual with a cancer (e.g., lung cancer (e.g., NSCLC), bladder cancer (e.g., UBC), kidney cancer (e.g., RCC), or breast cancer (e.g., TNBC)) to treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of PD-L1, IFNG, GZMB, CD8A, and PD-1 in a sample from the individual (e.g., tumor tissue), wherein an immune-score expression level of at least one, at least two, at least three, at least four, or all five of PD-L1, IFNG, GZMB, CD8A, and PD-1 relative to a reference immune-score expression level (e.g., an immune-score expression level of PD-L1, IFNG, GZMB, CD8A, and PD-1 in a reference population) indicates that the individual is more likely to be responsive to treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of a at least one, at least two, at least three, at least four, or all five of PD-L1, IFNG, GZMB, CD8A, and PD-1 in the sample that is below the reference immune-score expression level (e.g., an immune-score expression level of PD-L1, IFNG, GZMB, CD8A, and PD-1 in a reference population) indicates that the individual is more likely to be responsive to a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

Further provided herein are methods for determining the likelihood that an individual with a cancer (e.g., lung cancer (e.g., NSCLC), bladder cancer (e.g., UBC), kidney cancer (e.g., RCC), or breast cancer (e.g., TNBC)) will exhibit benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of PD-L1, IFNG, GZMB, CD8A, and PD-1 in a sample from the individual (e.g., tumor tissue), wherein an immune-score expression level of at least one, at least two, at least three, at least four, or all five of PD-L1, IFNG, GZMB, CD8A, and PD-1 relative to a reference immune-score expression level (e.g., an immune-score expression level of PD-L1, IFNG, GZMB, CD8A, and PD-1 in a reference population) indicates that the individual will have an increased likelihood of benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of at least one, at least two, at least three, at least four, or all five of PD-L1, IFNG, GZMB, CD8A, and PD-1 in the sample that is below the reference immune-score expression level (e.g., an immune-score expression level of PD-L1, IFNG, GZMB, CD8A, and PD-1 in a reference population) indicates that the individual will have a decreased likelihood of benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, the individual having a cancer (e.g., lung cancer (e.g., NSCLC), bladder cancer (e.g., UBC), kidney cancer (e.g., RCC), or breast cancer (e.g., TNBC)) may be provided a recommendation prior to administration of the PD-L1 binding antagonist, based on the immune-score expression level of PD-L1, IFNG, GZMB, CD8A, and PD-1 determined in accordance with any of the above methods. In some instances, the methods further include providing a recommendation that the individual will be likely to respond to or benefit from treatment with a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, the methods include providing a recommendation that the therapy selected for the individual includes treatment with a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, the methods may further include administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) to the individual. In some instances, the methods further include administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein the immune-score expression level of at least one, at least two, at least three, at least four, or all five of PD-L1, IFNG, GZMB, CD8A, and PD-1 in the sample from the individual is above a reference immune-score expression level (e.g., an immune-score expression level of PD-L1, IFNG, GZMB, CD8A, and PD-1 in a reference population). The PD-L1 axis binding antagonist may be any PD-L1 axis binding antagonist known in the art or described herein, for example, in Section III.F, below. For example, in some instances, the PD-L1 axis binding antagonist is a PD-L1 binding antagonist. In some instances, the PD-L1 binding antagonist is an antibody. In some instances, the antibody is selected from the group consisting of: YW243.55.S70, MPDL3280A (atezolizumab), MDX-1105, MEDI4736 (durvalumab), and MSB0010718C (avelumab). In some instances, the antibody comprises a heavy chain comprising HVR-H1 sequence of SEQ ID NO: 9, HVR-H2 sequence of SEQ ID NO: 10, and HVR-H3 sequence of SEQ ID NO: 11; and a light chain comprising HVR-L1 sequence of SEQ ID NO: 12, HVR-L2 sequence of SEQ ID NO: 13, and HVR-L3 sequence of SEQ ID NO: 14. In some instances, the antibody comprises a heavy chain variable region comprising the amino acid sequence of SEQ ID NO: 15 and a light chain variable region comprising the amino acid sequence of SEQ ID NO: 16.

In some instances, the methods further include administering to the individual an effective amount of an additional therapeutic agent. In some instances, the additional therapeutic agent is selected from the group consisting of a cytotoxic agent, a growth-inhibitory agent, a radiation therapy, an anti-angiogenic agent, as described herein, or a combination thereof.

Alternatively, in cases for which an individual is determined to have a decreased immune-score expression level of at least one, at least two, at least three, at least four, or all five of PD-L1, IFNG, GZMB, CD8A, and PD-1 relative to a reference immune-score expression level (e.g., an immune-score expression level of PD-L1, IFNG, GZMB, CD8A, and PD-1 in a reference population), the methods may further include administering to the individual an effective amount of an anti-cancer therapy other than, or in addition to, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). For example, the anti-cancer therapy other than, or in addition to, a PD-L1 axis binding antagonist may include a cytotoxic agent, a growth-inhibitory agent, a radiation therapy, an anti-angiogenic agent, as described herein, or a combination thereof, alone, or in addition to a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) and/or any additional therapeutic agent described herein.

(ii) Increased Immune-Score Expression Level of PD-L1, IFNG, GZMB, CD8A, and PD-1

An immune-score expression level of PD-L1, IFNG, GZMB, CD8A, and PD-1 in a sample from the individual having cancer that is above or higher than a reference immune-score expression level of PD-L1, CXCL9, and/or IFNG (e.g., in a reference population or a pre-assigned score) may indicate that the individual is more likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

For example, in some instances, an immune-score expression level of PD-L1, IFNG, GZMB, CD8A, and PD-1 in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, IFNG, GZMB, CD8A, and PD-1 in the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level of PD-L1, IFNG, GZMB, CD8A, and PD-1 in the sample that is in about the top 10^{th} to about the top 90^{th} percentile, about the top 20^{th} to about the top 80^{th} percentile, about the top 30^{th} to about the top 70^{th} percentile, about the top 40^{th} to about the top 60^{th} percentile, about the top 45^{th} to about the top 55^{th} percentile, about the top 48^{th} to about the top 52^{th} percentile, about the top 49.5^{th} to about the top 50.5^{th} percentile, about the top 49.9^{th} to about the top 50.1^{th} percentile, or about the top 50^{th} percentile of the immune-score expression level of PD-L1, IFNG, GZMB, CD8A, and PD-1 in the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). For example, in some instances, an immune-score expression level of PD-L1, IFNG, GZMB, CD8A, and PD-1 in the sample that is between about 10% to about 90% prevalence, about 15% to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level of PD-L1, IFNG, GZMB, CD8A, and PD-1 in the sample that is in about the top 80^{th} percentile (i.e., equal to, or higher than, the 20% prevalence level) of the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, an immune-score expression level of PD-L1, IFNG, GZMB, CD8A, and PD-1 in the sample that is in about the top 75^{th} percentile (i.e., equal to, or higher than, the 25% prevalence level) of the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, an immune-score expression level of PD-L1, IFNG, GZMB, CD8A, and PD-1 in the sample that is in about the top 50^{th} percentile (i.e., equal to, or higher than, the 50% prevalence level) of the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, an immune-score expression level of PD-L1, IFNG, GZMB, CD8A, and PD-1 in the sample that is in about the top 25^{th} percentile (e.g., equal to, or higher, than the 25% prevalence level) of the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, an immune-score expression level of PD-L1, IFNG, GZMB, CD8A, and PD-1 is in about the top 20^{th} percentile (i.e., equal to, or higher than, the 80% prevalence level) of the reference population identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level that is higher than a reference immune-score expression level refers to an overall increase of about 10%, 20%, 30%, 40%, 50%, 60%, 70%, 80%, 90%, 95%, 96%, 97%, 98%, or 99% or greater in the immune-score expression level of PD-L1, IFNG, GZMB, CD8A, and PD-1, detected by standard art-known methods such as those described herein, as compared to the immune-score expression level of PD-L1, IFNG, GZMB, CD8A, and PD-1 in a reference sample, reference cell, reference tissue, control sample, control cell, or control tissue. In certain instances, an immune-score expression level that is higher than a reference immune-score expression level refers to an increase in the immune-score expression level of PD-L1, IFNG, GZMB, CD8A, and PD-1 in the sample, wherein the increase is at least about 1.5×, 1.75×, 2×, 3×, 4×, 5×, 6×, 7×, 8×, 9×, 10×, 25×, 50×, 75×, or 100× the immune-score expression level of PD-L1, IFNG, GZMB, CD8A, and PD-1 in a reference sample, reference cell, reference tissue, control sample, control cell, or control tissue. In some instances, an immune-score expression level that is higher than a reference immune-score expression level refers to an overall increase in the immune-score expression level of PD-L1, IFNG, GZMB, CD8A, and PD-1 that is greater than about 1.5-fold, about 1.75-fold, about 2-fold, about 2.25-fold, about 2.5-fold, about 2.75-fold, about 3.0-fold, or about 3.25-fold as compared to the immune-score expression level of PD-L1, IFNG, GZMB, CD8A, and PD-1 in a reference sample, reference cell, reference tissue, control sample, control cell, or control tissue.

In some instances, an immune-score expression level for PD-L1, IFNG, GZMB, CD8A, and PD-1 that is higher than a reference immune-score expression level refers to an overall increase of about 10%, 20%, 30%, 40%, 50%, 60%, 70%, 80%, 90%, 95%, 96%, 97%, 98%, or 99% or greater in the immune-score expression level of PD-L1, IFNG, GZMB, CD8A, and PD-1, detected by standard art-known methods such as those described herein, as compared to a pre-assigned immune-score expression level of PD-L1, IFNG, GZMB, CD8A, and PD-1. In certain instances, an immune-score expression level for PD-L1, IFNG, GZMB, CD8A, and PD-1 that is higher than a reference immune-score expression level refers to an increase in the immune-score expression level of PD-L1, IFNG, GZMB, CD8A, and PD-1 in the sample, wherein the increase is at least about 1.5×, 1.75×, 2×, 3×, 4×, 5×, 6×, 7×, 8×, 9×, 10×, 25×, 50×, 75×, or 100× a pre-assigned immune-score expression level of PD-L1, IFNG, GZMB, CD8A, and PD-1. In some instances, an immune-score expression level for PD-L1, IFNG, GZMB, CD8A, and PD-1 that is higher than a reference immune-score expression level refers to an overall increase in the immune-score expression level of PD-L1, IFNG, GZMB, CD8A, and PD-1 that is greater than about 1.5-fold, about 1.75-fold, about 2-fold, about 2.25-fold, about 2.5-fold, about 2.75-fold, about 3.0-fold, or about 3.25-fold as compared to a pre-assigned immune-score expression level of PD-L1, IFNG, GZMB, CD8A, and PD-1.

(iii) Decreased Immune-Score Expression Level of PD-L1, IFNG, GZMB, CD8A, and PD-1

An immune-score expression level of PD-L1, IFNG, GZMB, CD8A, and PD-1 in a sample from the individual having cancer that is below or lower than a reference immune-score expression level of PD-L1, IFNG, GZMB, CD8A, and PD-1 (e.g., in a reference population or pre-assigned score) may indicate that the individual is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein the reference immune-score expression level is an immune-score expression level of PD-L1, IFNG, GZMB, CD8A, and PD-1 in a reference population.

For example, in some instances, an immune-score expression level of PD-L1, IFNG, GZMB, CD8A, and PD-1 in the sample that is in about the bottom 99^{th} percentile (equal to, or lower than, about the 99% prevalence level), about the bottom 95^{th} percentile (equal to, or lower than, about the 95% prevalence level), about the bottom 90^{th} percentile (equal to, or lower than, about the 90% prevalence level), about the bottom 85^{th} percentile (equal to, or lower than, about the 85% prevalence level), about the bottom 80^{th} percentile (equal to, or lower than, about the 80% prevalence level), about the bottom 75^{th} percentile (equal to, or lower than, about the 75% prevalence level), about the bottom 70^{th} percentile (equal to, or lower than, about the 70% prevalence level), about the bottom 65^{th} percentile (equal to, or lower than, about the 65% prevalence level), about the bottom 60^{th} percentile (equal to, or lower than, about the 60% prevalence level), about the bottom 55^{th} percentile (equal to, or lower than, about the 55% prevalence level), about the bottom 50^{th} percentile (equal to, or lower than, about the 50% prevalence level), about the bottom 45^{th} percentile (equal to, or lower than, about the 45% prevalence level), about the bottom 40^{th} percentile (equal to, or lower than, about the 40% prevalence level), about the bottom 35^{th} percentile (equal to, or lower than, about the 35% prevalence level), about the bottom 30^{th} percentile (equal to, or lower than, about the 30% prevalence level), about the bottom 25^{th} percentile (equal to, or lower than, about the 25% prevalence level), about the bottom 20^{th} percentile (equal to, or lower than, about the 20% prevalence level), about the bottom 15^{th} percentile (equal to, or lower than, about the 15% prevalence level), about the bottom 10^{th} percentile (equal to, or lower than, about the 10% prevalence level), about the bottom 5^{th} percentile (equal to, or lower than, about the 5% prevalence level), or about the bottom 1^{st} percentile (equal to, or lower than, about the 1% prevalence level) of the immune-score expression level of PD-L1, IFNG, GZMB, CD8A, and PD-1 in the reference population identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level of PD-L1, IFNG, GZMB, CD8A, and PD-1 in the sample that is in about the bottom 10^{th} to about the bottom 90^{th} percentile, about the bottom 20^{th} to about the bottom 80^{th} percentile, about the bottom 30^{th} to about the bottom 70^{th} percentile, about the bottom 40^{th} to about the bottom 60^{th} percentile, about the bottom 45^{th} to about the bottom 55^{th} percentile, about the bottom 48^{th} to about the bottom 52^{th} percentile, about the bottom 49.5^{th} to about the bottom 50.5^{th} percentile, about the bottom 49.9^{th} to about the bottom 50.1^{th} percentile, or about the bottom 50^{th} percentile of the immune-score expression level of PD-L1, IFNG, GZMB, CD8A, and PD-1 in the reference population identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). For example, in some instances, an immune-score expression level of PD-L1, IFNG, GZMB, CD8A, and PD-1 in the sample that is between about 10% to about 90% prevalence, about 15% to about 85% prevalence, about 20% to about 80% prevalence, about 25% to about 75% prevalence, about 30% to about 70% prevalence, about 35% to about 65% prevalence, about 40% to about 60% prevalence, about 45% to about 55% prevalence, about 48% to about 52% prevalence, about 49.5% to about 50.5% prevalence, about 49.9% to about 50.1% prevalence, or about 50% prevalence in the reference population identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level for PD-L1, IFNG, GZMB, CD8A, and PD-1 that is lower than a reference immune-score expression level refers to a decrease of about 10%, 20%, 30%, 40%, 50%, 60%, 70%, 80%, 90%, 95%, 96%, 97%, 98%, or 99% or greater in the immune-score expression level of PD-L1, IFNG, GZMB, CD8A, and PD-1, detected by standard art-known methods such as those described herein, as compared to the immune-score expression level of PD-L1, IFNG, GZMB, CD8A, and PD-1 in a reference sample, reference cell, reference tissue, control sample, control cell, or control tissue. In certain instances, an immune-score expression level for PD-L1, IFNG, GZMB, CD8A, and PD-1 that is lower than a reference immune-score expression level refers to a decrease in the immune-score expression level of PD-L1, IFNG, GZMB, CD8A, and PD-1 in the sample, wherein the decrease is at least about 1.5×, 1.75×, 2×, 3×, 4×, 5×, 6×, 7×, 8×, 9×, 10×, 25×, 50×, 75×, or 100× the immune-score expression level of PD-L1, IFNG, GZMB, CD8A, and PD-1 in a reference sample, reference cell, reference tissue, control sample, control cell, or control tissue. In some instances, an immune-score expression level for PD-L1, IFNG, GZMB, CD8A, and PD-1 that is lower than a reference immune-score expression level refers to a decrease in the immune-score expression level of PD-L1, IFNG, GZMB, CD8A, and PD-1 that is greater than about 1.5-fold, about 1.75-fold, about 2-fold, about 2.25-fold, about 2.5-fold, about 2.75-fold, about 3.0-fold, or about 3.25-fold as compared to the immune-score expression level of PD-L1, IFNG, GZMB, CD8A, and PD-1 in a reference sample, reference cell, reference tissue, control sample, control cell, or control tissue.

In some instances, an immune-score expression level that is lower than a reference immune-score expression level refers to an overall decrease of about 10%, 20%, 30%, 40%, 50%, 60%, 70%, 80%, 90%, 95%, 96%, 97%, 98%, or 99% or greater in the immune-score expression level of PD-L1, IFNG, GZMB, CD8A, and PD-1, detected by standard art-known methods such as those described herein, as compared to a pre-assigned immune-score expression level of PD-L1, IFNG, GZMB, CD8A, and PD-1. In certain instances, an immune-score expression level that is lower than a reference immune-score expression level refers to a decrease in the immune-score expression level of PD-L1, IFNG, GZMB, CD8A, and PD-1 in the sample, wherein the decrease is at least about 1.5×, 1.75×, 2×, 3×, 4×, 5×, 6×, 7×, 8×, 9×, 10×, 25×, 50×, 75×, or 100× a pre-assigned immune-score expression level of PD-L1, IFNG, GZMB, CD8A, and PD-1. In some instances, an immune-score expression level that is lower than a reference immune-score expression level refers to an overall decrease in the immune-score expression level of PD-L1, IFNG, GZMB, CD8A, and PD-1 that is greater than about 1.5-fold, about 1.75-fold, about 2-fold, about 2.25-fold, about 2.5-fold, about 2.75-fold, about 3.0-fold, or about 3.25-fold as compared to a pre-assigned immune-score expression level of PD-L1, IFNG, GZMB, CD8A, and PD-1.

(iv) Reference Immune-Score Expression Level of PD-L1, IFNG, GZMB, CD8A, and/or PD-1

The reference immune-score expression level described herein may be based on an immune-score expression level of PD-L1, IFNG, GZMB, CD8A, and PD-1 in a reference population. In some instances, the reference immune-score expression level described herein is an immune-score expression level of PD-L1, IFNG, GZMB, CD8A, and PD-1 in a reference population that includes two or more (e.g., two or more, three or more, four or more, or five or more) subsets of individuals.

In some instances, the reference immune-score expression level is the immune-score expression level of PD-L1, IFNG, GZMB, CD8A, and PD-1 in a reference population, wherein the reference population includes at least one subset of individuals having a cancer (e.g., lung cancer (e.g., NSCLC), bladder cancer (e.g., UBC), kidney cancer (e.g., RCC), or breast cancer (e.g., TNBC)).

In some instances, the reference immune-score expression level of PD-L1, IFNG, GZMB, CD8A, and PD-1 in a reference population, wherein the reference population includes at least one subset of individuals having a cancer (e.g., lung cancer (e.g., NSCLC), bladder cancer (e.g., UBC), kidney cancer (e.g., RCC), or breast cancer (e.g., TNBC)) who have been administered one or more doses (e.g., at least one, two, three, four, five, six, seven, eight, nine, or ten or more doses) of a PD-L1 axis binding antagonist (e.g., as part of a PD-L1 axis binding antagonist monotherapy or combination therapy including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)))).

In some instances, the reference immune-score expression level is an immune-score expression level of PD-L1, IFNG, GZMB, CD8A, and PD-1 in a reference population, wherein the reference population includes at least one subset of individuals having a cancer (e.g., lung cancer (e.g., NSCLC), bladder cancer (e.g., UBC), kidney cancer (e.g., RCC), or breast cancer (e.g., TNBC)) who have received treatment with a PD-L1 axis binding antagonist therapy, wherein the PD-L1 axis binding antagonist therapy is a monotherapy (e.g., a PD-L1 axis binding antagonist monotherapy including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody))).

In some instances, the reference immune-score expression level is an immune-score expression level of PD-L1, IFNG, GZMB, CD8A, and PD-1 in a reference population, wherein the reference population includes at least one subset of individuals having a cancer (e.g., lung cancer (e.g., NSCLC), bladder cancer (e.g., UBC), kidney cancer (e.g., RCC), or breast cancer (e.g., TNBC)) who have received treatment with a PD-L1 axis binding antagonist therapy, wherein the PD-L1 axis binding antagonist therapy is a combination therapy (e.g., a combination therapy including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) and an additional therapeutic agent (e.g., anti-cancer therapy (e.g., a cytotoxic agent, a growth-inhibitory agent, a radiation therapy, an anti-angiogenic agent, or a combination thereof))).

In some instances, the reference immune-score expression level is an immune-score expression level of PD-L1, IFNG, GZMB, CD8A, and PD-1 in a reference population, wherein the reference population includes at least one subset of individuals having a cancer (e.g., lung cancer (e.g., NSCLC), bladder cancer (e.g., UBC), kidney cancer (e.g., RCC), or breast cancer (e.g., TNBC)) who have received treatment with a non-PD-L1 axis binding antagonist therapy, wherein the non-PD-L1 axis binding antagonist therapy does not include a PD-L1 axis binding antagonist and includes an anti-cancer therapy (e.g., a cytotoxic agent, a growth-inhibitory agent, a radiation therapy, an anti-angiogenic agent, or a combination thereof))).

For example, in some instances, the reference population includes a first subset of individuals who have been treated with a PD-L1 axis binding antagonist therapy (e.g., PD-L1 binding antagonist (e.g., anti-PD-L1 antibody, e.g., atezolizumab (MPDL3280A)) or PD-1 binding antagonist (e.g., anti-PD-1 antibody)) and a second subset of individuals who have been treated with a non-PD-L1 axis binding antagonist therapy, wherein the non-PD-L1 axis binding antagonist therapy does not include a PD-L1 axis binding antagonist.

In some instances, the reference immune-score expression level of PD-L1, IFNG, GZMB, CD8A, and PD-1 significantly separates each of the first and second subsets of individuals based on a significant difference between an individual's responsiveness (e.g., ORR, PFS, or OS) to treatment with the PD-L1 axis binding antagonist therapy and an individual's responsiveness to treatment with the non-PD-L1 axis binding antagonist therapy above the reference immune-score expression level, wherein the individual's responsiveness to treatment with the PD-L1 axis binding antagonist therapy is significantly improved relative to the individual's responsiveness to treatment with the non-PD-L1 axis binding antagonist therapy. For example, in some instances, the reference immune-score expression level of PD-L1, IFNG, GZMB, CD8A, and PD-1 optimally separates each of the first and second subsets of individuals based on a maximum difference between an individual's responsiveness (e.g., ORR, PFS, or OS) to treatment with the PD-L1 axis binding antagonist therapy and an individual's responsiveness to treatment with the non-PD-L1 axis binding antagonist therapy above the reference immune-score expression level, wherein the individual's responsiveness to treatment with the PD-L1 axis binding antagonist therapy is significantly improved relative to the individual's responsiveness to treatment with the non-PD-L1 axis binding antagonist therapy.

In some instances, the reference immune-score expression level of PD-L1, IFNG, GZMB, CD8A, and PD-1 significantly separates each of the first and second subsets of individuals based on a significant difference between an individual's responsiveness (e.g., ORR, PFS, or OS) to treatment with the PD-L1 axis binding antagonist therapy and an individual's responsiveness to treatment with the non-PD-L1 axis binding antagonist therapy below the reference immune-score expression level, wherein the individual's responsiveness to treatment with the non-PD-L1 axis binding antagonist therapy is significantly improved relative to the individual's responsiveness to treatment with the PD-L1 axis binding antagonist therapy. For example, in some instances, the reference immune-score expression level of PD-L1, IFNG, GZMB, CD8A, and PD-1 optimally separates each of the first and second subsets of individuals based on a maximum difference between an individual's responsiveness (e.g., ORR, PFS, or OS) to treatment with the PD-L1 axis binding antagonist therapy and an individual's responsiveness to treatment with the non-PD-L1 axis binding antagonist therapy below the reference immune-score expression level, wherein the individual's responsiveness to treatment with the non-PD-L1 axis binding antagonist therapy is significantly improved relative to the individual's responsiveness to treatment with the PD-L1 axis binding antagonist therapy.

In some instances, an optimal separation or significant separation may be based on a hazard ratio (HR) determined from an analysis of the immune-score expression level of PD-L1, IFNG, GZMB, CD8A, and PD-1 in the first and second subsets of individuals, wherein the HR is less than 1, e.g., an HR of about 0.95, about 0.9, about 0.8, about 0.7, about 0.6, about 0.5, about 0.4, about 0.3, about 0.2, about 0.1 or lower. For example, in particular instances, an optimal separation or significant separation may be based on a hazard ratio (HR) determined from an analysis of the immune-score expression level of PD-L1, CXCL9, and IFNG in the first and second subsets of individuals, wherein the upper bound of the 95% confidence interval of the HR is less than 1, e.g., an upper bound of the 95% confidence interval of the HR of about 0.95, about 0.9, about 0.8, about 0.7, about 0.6, about 0.5, about 0.4, about 0.3, about 0.2, about 0.1 or lower.

Additionally, or alternatively, the reference immune-score expression level may be an immune-score expression level of PD-L1, IFNG, GZMB, CD8A, and PD-1 in a reference population, wherein the reference population includes at least one subset of individuals who do not have a cancer (e.g., individuals not having NSCLC, UBC, RCC, or TNBC) or have cancer but are treatment naïve.

(v) Indications

The methods described herein are useful for predicting the therapeutic response of an individual having a cancer to treatment with a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, the cancer may be a lung cancer, a kidney cancer, a bladder cancer, a breast cancer, a colorectal cancer, an ovarian cancer, a pancreatic cancer, a gastric carcinoma, an esophageal cancer, mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a merkel cell cancer, or a hematologic malignancy.

In certain instances, the cancer may be a lung cancer. For example, the lung cancer may be a non-small cell lung cancer (NSCLC), including but not limited to a locally advanced or metastatic (e.g., stage IIIB, stage IV, or recurrent) NSCLC. In some instances, the lung cancer (e.g., NSCLC) is unresectable/inoperable lung cancer (e.g., NSCLC). For example, the methods described herein may be used for identifying an individual having a lung cancer (e.g., NSCLC) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining an immune-score expression level of PD-L1, IFNG, GZMB, CD8A, and PD-1 in a sample from the individual (e.g., a tumor tissue sample), wherein the immune-score expression level of at least one, at least two, at least three, at least four, or all five of PD-L1, IFNG, GZMB, CD8A, and PD-1 in the sample that is above a reference immune-score expression level (e.g., an immune-score expression level of PD-L1, IFNG, GZMB, CD8A, and PD-1 in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In certain instances, the cancer may be a bladder cancer. For example, the bladder cancer may be a urothelial bladder cancer, including but not limited to a non-muscle invasive urothelial bladder cancer, a muscle-invasive urothelial bladder cancer, or a metastatic urothelial bladder cancer. In some instances, the urothelial bladder cancer is a metastatic urothelial bladder cancer. For example, the methods described herein may be used for identifying an individual having a bladder cancer (e.g., UBC) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining an immune-score expression level of PD-L1, IFNG, GZMB, CD8A, and PD-1 in a sample from the individual (e.g., a tumor tissue sample), wherein the immune-score expression level of at least one, at least two, at least three, at least four, or all five of PD-L1, IFNG, GZMB, CD8A, and PD-1 in the sample that is above a reference immune-score expression level (e.g., an immune-score expression level of PD-L1, IFNG, GZMB, CD8A, and PD-1 in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In certain instances, the cancer may be a kidney cancer. In some instances, the kidney cancer may be a renal cell carcinoma (RCC), including stage I RCC, stage II RCC, stage III RCC, stage IV RCC, or recurrent RCC. For example, the methods described herein may be used for identifying an individual having a kidney cancer (e.g., RCC) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining an immune-score expression level of PD-L1, IFNG, GZMB, CD8A, and PD-1 in a sample from the individual (e.g., a tumor tissue sample), wherein the immune-score expression level of at least one, at least two, at least three, at least four, or all five of PD-L1, IFNG, GZMB, CD8A, and PD-1 in the sample that is above a reference immune-score expression level (e.g., an immune-score expression level of PD-L1, IFNG, GZMB, CD8A, and PD-1 in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In certain instances, the cancer may be a breast cancer. For example, the breast cancer may be TNBC, estrogen receptor-positive breast cancer, estrogen receptor-positive/HER2-negative breast cancer, HER2-negative breast cancer, HER2-positive breast cancer, estrogen receptor-negative breast cancer, progesterone receptor-positive breast cancer, or progesterone receptor-negative breast cancer. In some instances, the breast cancer may be a TNBC. For example, the methods described herein may be used for identifying an individual having a breast cancer (e.g., TNBC) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining an immune-score expression level of PD-L1, IFNG, GZMB, CD8A, and PD-1 in a sample from the individual (e.g., a tumor tissue sample), wherein the immune-score expression level of at least one, at least two, at least three, at least four, or all five of PD-L1, IFNG, GZMB, CD8A, and PD-1 in the sample that is above a reference immune-score expression level (e.g., an immune-score expression level of PD-L1, IFNG, GZMB, CD8A, and PD-1 in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, the individual having a cancer, e.g., cancers described herein, has not been previously treated for the cancer (treatment naïve). For example, in some instances, the individual having a cancer has not previously received a PD-L1 axis binding antagonist therapy (e.g., PD-L1 binding antagonist (e.g., anti-PD-L1 antibody, e.g., atezolizumab (MPDL3280A)) or PD-1 binding antagonist (e.g., anti-PD-1 antibody)). For example, in some instances, an immune-score expression level of at least one, at least two, at least three, at least four, or all five of PD-L1, IFNG, GZMB, CD8A, and PD-1 that is above a reference immune-score expression level (e.g., an immune-score expression level of PD-L1, IFNG, GZMB, CD8A, and PD-1 in a reference population) identifies the individual having cancer (e.g., lung cancer (e.g., NSCLC), bladder cancer (e.g., UBC), kidney cancer (e.g., RCC), or breast cancer (e.g., TNBC)) as one who may benefit from a first-line treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, the individual having a cancer has previously received treatment for the cancer. In some instances, the individual having a cancer has previously received treatment including a non-PD-L1 axis binding antagonist therapy (e.g., an anti-cancer therapy (e.g., a cytotoxic agent, a growth-inhibitory agent, a radiation therapy, an anti-angiogenic agent, or a combination thereof)). For example, in some instances, an immune-score expression level of at least one, at least two, at least three, at least four, or all five of PD-L1, IFNG, GZMB, CD8A, and PD-1 that is above a reference immune-score expression level (e.g., an immune-score expression level of PD-L1, IFNG, GZMB, CD8A, and PD-1 in a reference population) identifies the individual having cancer (e.g., lung cancer (e.g., NSCLC), bladder cancer (e.g., UBC), kidney cancer (e.g., RCC), or breast cancer (e.g., TNBC)) as one who may benefit from a second-line treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

(vi) Treatment Benefits

An individual who benefits from receiving treatment with a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may experience, for example, a delay or prevention in the occurrence or recurrence of a cancer (e.g., a lung cancer (e.g., NSCLC), a bladder cancer (e.g., UBC), a kidney cancer (e.g., RCC), or a breast cancer (e.g., TNBC)), alleviation of symptoms, diminishment of any direct or indirect pathological consequences of the cancer, prevention of metastasis, decrease in the rate of disease progression, amelioration or palliation of the disease state, or remission or improved prognosis. In some instances, the treatments described herein are used to delay development of a cancer or to slow the progression of a cancer (e.g., a lung cancer (e.g., NSCLC), a bladder cancer (e.g., UBC), a kidney cancer (e.g., RCC), or a breast cancer (e.g., TNBC)). In some instances, the benefit may be an increase in overall survival (OS), progression-free survival (PFS), complete response (CR), partial response (PR), or a combination thereof.

In some instances, an immune-score expression level of at least one, at least two, at least three, at least four, or all five of PD-L1, IFNG, GZMB, CD8A, and PD-1 that is above a reference immune-score expression level (e.g., an immune-score expression level of PD-L1, IFNG, GZMB, CD8A, and PD-1 in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein the benefit is an increase in OS, PFS, CR, PR, or a combination thereof, relative to a treatment that does not include a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level of at least one, at least two, at least three, at least four, or all five of PD-L1, IFNG, GZMB, CD8A, and PD-1 that is above a reference immune-score expression level (e.g., an immune-score expression level of PD-L1, IFNG, GZMB, CD8A, and PD-1 in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein the benefit is an increase in OS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, an immune-score expression level of at least one, at least two, at least three, at least four, or all five of PD-L1, IFNG, GZMB, CD8A, and PD-1 that is above a reference immune-score expression level (e.g., an immune-score expression level of PD-L1, IFNG, GZMB, CD8A, and PD-1 in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein the benefit is an increase in PFS (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) relative to a treatment that does not include a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

### E. Six-Gene Immune-Score Combination

In particular instances, the methods and assays provided herein may be used to determine an immune-score expression level of all six of PD-L1, CXCL9, IFNG, GZMB, CD8A, and PD-1.

In some instances, the determination step includes determining the expression levels of all six genes and one or more additional genes associated with T-effector cells, e.g., determining the expression level of (i) all six of PD-L1, CXCL9, IFNG, GZMB, CD8A, and PD-1 and (ii) one or more genes associated with T-effector cells (e.g., at least one, at least two, at least three, at least four, at least five, at least six, at least seven, at least eight, at least nine, at least ten, at least eleven, at least twelve, at least thirteen, or fourteen of CD8A, GZMA, GZMB, IFNG, EOMES, PRF1, PD-L1, PD-1, CXCL9, CD27, FOXP3, CTLA4, TIGIT, IDO1, CXCL10, CXCL11, PSMB8, PSMB9, TAP1, and/or TAP2), wherein the one or more genes associated with T-effector cells are different from PD-L1, CXCL9, IFNG, GZMB, CD8A, and PD-1.

Provided herein are methods for identifying an individual having a cancer (e.g., lung cancer (e.g., NSCLC), bladder cancer (e.g., UBC), kidney cancer (e.g., RCC), or breast cancer (e.g., TNBC)) who may benefit from treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), the methods including determining the expression level of all six of PD-L1, CXCL9, IFNG, GZMB, CD8A, and PD-1 in a sample from the individual (e.g., a tumor tissue sample), wherein an immune-score expression level of at least one, at least two, at least three, at least four, at least five, or all six of PD-L1, CXCL9, IFNG, GZMB, CD8A, and PD-1 in the sample that is above a reference immune-score expression level (e.g., an immune-score expression level of PD-L1, CXCL9, IFNG, GZMB, CD8A, and PD-1 in a reference population) identifies the individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of at least one, at least two, at least three, at least four, at least five, or all six of PD-L1, CXCL9, IFNG, GZMB, CD8A, and PD-1 in the sample that is below a reference immune-score expression level (e.g., an immune-score expression level of PD-L1, CXCL9, IFNG, GZMB, CD8A, and PD-1 in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

Also provided herein are methods for selecting a therapy for an individual having a cancer (e.g., lung cancer (e.g., NSCLC), bladder cancer (e.g., UBC), kidney cancer (e.g., RCC), or breast cancer (e.g., TNBC)), the methods including determining the expression level of PD-L1, CXCL9, IFNG, GZMB, CD8A, and PD-1 in a sample from the individual, wherein an immune-score expression level of at least one, at least two, at least three, at least four, at least five, or all six of PD-L1, CXCL9, IFNG, GZMB, CD8A, and PD-1 in the sample that is above a reference immune-score expression level (e.g., an immune-score expression level of PD-L1, CXCL9, IFNG, GZMB, CD8A, and PD-1 in a reference population) identities an individual as one who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Alternatively, an immune-score expression level of at least one, at least two, at least three, at least four, at least five, or all six of PD-L1, CXCL9, IFNG, GZMB, CD8A, and PD-1 in the sample that is below a reference immune-score expression level (e.g., an immune-score expression level of PD-L1, CXCL9, IFNG, GZMB, CD8A, and PD-1 in a reference population) identifies the individual as one who is less likely to benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). The examples and embodiments described in Sections II.B (i-vi), II.C (i-vi), II.D (i-vi), and II.E (i-vi), below, are also specifically contemplated to apply to the immune-score expression level of all six of PD-L1, CXCL9, IFNG, GZMB, CD8A, and PD-1.

### F. Determination of Expression Levels

(i) Detection Methods

The immune-score expression level of the genes described herein (e.g., at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) may be based on a nucleic acid expression level, and preferably, an mRNA expression level. Presence and/or expression levels/amount of the genes described herein can be determined qualitatively and/or quantitatively based on any suitable criterion known in the art, including but not limited to DNA, mRNA, cDNA, proteins, protein fragments, and/or gene copy number.

In some instances, nucleic acid expression levels of the genes described herein (e.g., at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) may be measured by polymerase chain reaction (PCR)-based assays, e.g., quantitative PCR, real-time PCR, quantitative real-time PCR (qRT-PCR), reverse transcriptase PCR (RT-PCR), and reverse transcriptase quantitative PCR (RT-qPCR). Platforms for performing quantitative PCR assays include Fluidigm (e.g., BIOMARK™ HD System). Other amplification-based methods include, for example, transcript-mediated amplification (TMA), strand displacement amplification (SDA), nucleic acid sequence based amplification (NASBA), and signal amplification methods such as bDNA.

In some instances, nucleic acid expression levels of the genes described herein (e.g., at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) also may be measured by sequencing-based techniques, such as, for example, RNA-seq, serial analysis of gene expression (SAGE), high-throughput sequencing technologies (e.g., massively parallel sequencing), and Sequenom MassARRAY® technology. Nucleic acid expression levels (e.g., expression levels of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4))) also may be measured by, for example, NanoString nCounter, and high-coverage expression profiling (HiCEP). Additional protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

Other methods for detecting nucleic acid levels of the genes described herein (e.g., at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. Hybridization of a labeled probe with a particular array member indicates that the sample from which the probe was derived expresses that gene.

Primers and probes may be labeled with a detectable marker, such as, for example, a radioisotope, fluorescent compound, bioluminescent compound, a chemiluminescent compound, metal chelator, or enzyme. Such probes and primers can be used to detect the presence of expressed genes, such as at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4, in a sample. As will be understood by the skilled artisan, many different primers and probes may be prepared based on the sequences provided herein (or, in the case of genomic DNA, their adjacent sequences) and used effectively to amplify, clone, and/or determine the presence and/or expression levels of the genes described herein.

Other methods to detect nucleic acid expression levels of the genes described herein (e.g., at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) include electrophoresis, Northern and Southern blot analyses, in situ hybridization (e.g., single or multiplex nucleic acid in situ hybridization), RNAse protection assays, and microarrays (e.g., Illumina BEADARRAY™ technology; Beads Array for Detection of Gene Expression (BADGE)).

In some instances, the immune-score expression level of the genes described herein (e.g., the immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) can be analyzed by a number of methodologies, including, but not limited to, RNA-seq, PCR, RT-qPCR, qPCR, multiplex qPCR, multiplex RT-qPCR, NANOSTRING® nCOUNTER® Gene Expression Assay, microarray analysis, serial analysis of gene expression (SAGE), Northern blot analysis, MassARRAY, ISH, and whole genome sequencing, or combinations thereof.

In further instances, the immune-score expression level of the gene described herein (e.g., the immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) may be detected in the sample using a method selected from the group consisting of RNA-seq, RT-qPCR, qPCR, multiplex qPCR, multiplex RT-qPCR, microarray analysis, SAGE, MassARRAY technique, FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometery, HPLC, and ISH, or combinations thereof.

In some instances, the immune-score expression level of the genes described herein (e.g., the immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) is detected using RT-qPCR. For example, in some instances, the immune-score expression level of at least one, at least two, at least three, at least four, at least five, or all six genes selected from the group consisting of PD-L1, CXCL9, IFNG, GZMB, CD8A, and PD-1, or combinations thereof is detected based on mRNA expression level(s) using RT-qPCR. In some instances, the immune-score expression level based on mRNA expression levels of any one of the combinations of two genes listed in Table 1 is detected using RT-qPCR. In some instances, the immune-score expression level based on mRNA expression levels of any one of the combinations of three genes listed in Table 2 (e.g., PD-L1, IFNG, and CXCL9) is detected using RT-qPCR. In some instances, the immune-score expression level based on mRNA expression levels of any one of the combinations of four genes listed in Table 3 (e.g., PD-L1, IFNG, GZMB, and CD8A) is detected using RT-qPCR. In some instances, the immune-score expression level based on mRNA expression levels of any one of the five genes listed in Table 3 (e.g., PD-L1, IFNG, GZMB, CD8A, and PD-1) is detected using RT-qPCR. In some instances, the immune-score expression level based on mRNA expression levels of all six of PD-L1, CXCL9, IFNG, GZMB, and CD8A is detected using RT-qPCR.

In some instances, the immune-score expression level for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) is detected using RNA-seq. For example, in some instances, the immune-score expression level based on mRNA expression level of any one of the combinations of one of PD-L1, CXCL9, IFNG, GZMB, or CD8A is detected using RNA-seq. In some instances, the immune-score expression level based on mRNA expression levels of any of the combinations of two genes listed in Table 1 is detected using RNA-seq. In some instances, the immune-score expression level based on mRNA expression levels of any one of the combinations of three genes listed in Table 2 (e.g., PD-L1, IFNG, and CXCL9) is detected using RNA-seq. In some instances, the immune-score expression level based on mRNA expression levels of any one of the combinations of four genes listed in Table 3 (e.g., PD-L1, IFNG, GZMB, and CD8A) is detected using RNA-seq. In some instances, the immune-score expression level based on mRNA expression levels of any one of the five genes listed in Table 4 (e.g., PD-L1, IFNG, GZMB, CD8A, and PD-1) is detected using RNA-seq. In some instances, the immune-score expression level based on mRNA expression levels of all six of PD-L1, CXCL9, IFNG, GZMB, and CD8A is detected using RNA-seq.

(ii) RT-qPCR

In some instances, nucleic acid expression levels of the genes described herein (e.g., at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) can be detected using reverse transcription quantitative polymerase chain reaction (RT-qPCR). The technique of RT-qPCR is a form of PCR wherein the nucleic acid to be amplified is RNA that is first reverse transcribed into cDNA and the amount of PCR product is measured at each step in a PCR reaction. As RNA cannot serve as a template for PCR, the first step in gene expression profiling by PCR is the reverse transcription of the RNA template into cDNA, followed by its amplification in a PCR reaction. For example, reverse transcriptases may include avilo myeloblastosis virus reverse transcriptase (AMY-RT) or Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GENEAMP™ RNA PCR kit (Perkin Elmer, Calif., USA), following the manufacturer's instructions. The derived cDNA can then be used as a template in the subsequent PCR reaction.

A variation of the PCR technique is quantitative real time PCR (qRT-PCR), which measures PCR product accumulation through a dual-labeled fluorigenic probe (i.e., TAQMAN® probe). The technique of quantitative real time polymerase chain reaction refers to a form of PCR wherein the amount of PCR product is measured at each step in a PCR reaction. This technique has been described in various publications including Cronin et al., Am. J. Pathol. 164(I):35-42 (2004); and Ma et al., Cancer Cell 5:607-616 (2004). Real time PCR is compatible both with quantitative competitive PCR, where an internal competitor for each target sequence is used for normalization, and/or with quantitative comparative PCR using a normalization gene contained within the sample, or a housekeeping gene for PCR. For further details see, e.g. Held et al., Genome Research 6:986-994 (1996).

The steps of a representative protocol for profiling gene expression using fixed, paraffin-embedded tissues as the RNA source, including mRNA isolation, purification, primer extension and amplification are given in various published journal articles (for example: Godfrey et al., Malec. Diagnostics 2: 84-91 (2000); Specht et al., Am. J. Pathol. 158: 419-29 (2001)). Briefly, a representative process starts with cutting a section (e.g., a 10 microgram section) of a paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by PCR.

The nucleic acid expression level determined by an amplification-based method (e.g., RT-qPCR) may be expressed as a cycle threshold value (Ct). From this value, a normalized expression level for each gene can be determined, e.g., using the delta Ct (dCt) method as follows: Ct(Control/Reference Gene)−Ct(Gene of Interest/Target Gene)=dCt (Gene of Interest/Target Gene). One of skill in the art will appreciate that the dCt value obtained may be a negative dCt value or a positive dCt value. As defined herein, a higher dCt value indicates a higher expression level of the gene of interest relative to the control gene. Conversely, a lower dCt value indicates a lower expression level of the gene of interest relative to the control gene. In cases where the expression levels of a plurality of genes has been determined, the expression level for each gene, e.g., expressed as a dCt value, may then be used to determine a single value that represents an aggregate or composite expression level for the plurality of genes (e.g., an immune-score expression level). The immune-score expression level may be the mean or median of dCt values determined for each target gene/gene of interest. Thus, in some instances, the immune-score expression level described herein may be the mean or median of dCt values determined for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4). As defined herein, a higher averaged dCt or median dCt value indicates a higher aggregative expression level of the plurality of target genes relative to the control gene (or plurality of control genes). A lower averaged dCt or median dCt value indicates a lower aggregative expression level of the plurality of target genes relative to the control gene (or plurality of control genes). As described herein, an immune-score expression level may in turn be compared to a reference immune-score expression level as further defined herein.

In one particular instance, the nucleic acid expression levels described herein may be determined using a method including:

(a) obtaining or providing a sample from the individual, wherein the sample includes a tumor tissue sample (e.g., a paraffin-embedded, formalin-fixed NSCLC, UBC, RCC, or TNBC tumor tissue sample);

(b) isolating mRNA from said sample;

(c) performing reverse transcription of the mRNA into cDNA (e.g., for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4));

(d) amplifying the cDNA (e.g., for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) using PCR; and

(e) quantifying the nucleic acid expression levels (e.g., for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)).

One or more genes (e.g., one, two, three, four, five, or six of genes selected from PD-L1, IFNG, GZMB, CD8A, CXCL9, or PD-1) may be detected in a single assay depending on the primers or probes used. Further, the assay may be performed across one or more tubes (e.g., one, two, three, four, five, or six or more tubes).

In some instances, the method further comprises (f) normalizing the nucleic acid expression level of the gene(s) (e.g., at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in said sample to the expression level of one or more reference genes (e.g., one, two, three, four, five, six, seven, eight, nine, or more reference genes, e.g., a housekeeping gene (e.g., TMEM55B)). For example, RT-qPCR may be used to analyze the immune-score expression level of the genes described herein ((e.g., at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) to generate an immune-score expression level that reflects a normalized, averaged dCT value for the analyzed genes. Exemplary immune-score expression levels generated by such a method can be found in Examples 1-4, provided herein.

(iii) RNA-Seq

In some instances, nucleic acid expression levels of the genes described herein (e.g., at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) can be detected using RNA-seq. RNA-seq, also called Whole Transcriptome Shotgun Sequencing (WTSS), refers to the use of high-throughput sequencing technologies to sequence and/or quantify cDNA in order to obtain information about a sample's RNA content. Publications describing RNA-Seq include: Wang et al. “RNA-Seq: a revolutionary tool for transcriptomics” Nature Reviews Genetics 10 (1): 57-63 (January 2009); Ryan et al. BioTechniques 45 (1): 81-94 (2008); and Maher et al. “Transcriptome sequencing to detect gene fusions in cancer”. Nature 458 (7234): 97-101 (January 2009).

(iv) Samples

The sample may be taken from an individual who is suspected of having, or is diagnosed as having a cancer, and hence is likely in need of treatment, or from a healthy individual who is not suspected of having a cancer or who does not have cancer but has a family history of a cancer. For assessment of gene expression, samples, such as those containing cells, or proteins or nucleic acids produced by these cells, may be used in the methods of the present invention. The expression level of a gene can be determined by assessing the amount (e.g., the absolute amount or concentration) of the markers in a sample (e.g., a tissue sample, e.g., a tumor tissue sample, such as a biopsy). In addition, the level of a gene can be assessed in bodily fluids or excretions containing detectable levels of genes. Bodily fluids or secretions useful as samples in the present invention include, e.g., blood, urine, saliva, stool, pleural fluid, lymphatic fluid, sputum, ascites, prostatic fluid, cerebrospinal fluid (CSF), or any other bodily secretion or derivative thereof. The word blood is meant to include whole blood, plasma, serum, or any derivative of blood. Assessment of a gene in such bodily fluids or excretions can sometimes be preferred in circumstances where an invasive sampling method is inappropriate or inconvenient. In other embodiments, a tumor tissue sample is preferred.

The sample may be frozen, fresh, fixed (e.g., formalin fixed), centrifuged, and/or embedded (e.g., paraffin embedded), etc. The cell sample can be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, biopsies may also be subjected to post-collection preparative and storage techniques, e.g., fixation, such as formalin fixation.

In one particular instance, the sample is a clinical sample. In another instance, the sample is used in a diagnostic assay, such as a diagnostic assay or diagnostic method of the invention. In some instances, the sample is obtained from a primary or metastatic tumor. Tissue biopsy is often used to obtain a representative piece of tumor tissue. Alternatively, tumor cells can be obtained indirectly in the form of tissues or fluids that are known or thought to contain the tumor cells of interest. For example, samples of lung cancer lesions may be obtained by resection, bronchoscopy, fine needle aspiration, bronchial brushings, or from sputum, pleural fluid or blood. Genes or gene products can be detected from cancer or tumor tissue or from other body samples such as urine, sputum, serum or plasma. The same techniques discussed above for detection of target genes or gene products in cancerous samples can be applied to other body samples. Cancer cells may be sloughed off from cancer lesions and appear in such body samples. By screening such body samples, a simple early diagnosis can be achieved for these cancers. In addition, the progress of therapy can be monitored more easily by testing such body samples for target genes or gene products.

In some instances, the sample from the individual is a tissue sample, a whole blood sample, a plasma sample, a serum sample, or a combination thereof. In some instances, the sample is a tissue sample. In some instances, the sample is a tumor tissue sample. In some instances, the sample is obtained prior to treatment with a PD-L1 axis binding antagonist (e.g., PD-L1 binding antagonist, e.g., anti-PD-L1 antibody, e.g., atezolizumab (MPDL3280A)). In some instances, the tissue sample is formalin-fixed and paraffin-embedded (FFPE) sample, an archival sample, a fresh sample, or a frozen sample.

In some instances, the sample from the individual is a tissue sample. In some instances, the tissue sample is a tumor tissue sample (e.g., biopsy tissue). In some instances, the tumor tissue sample includes tumor cells, tumor infiltrating immune cells, stromal cells, or a combination thereof. In some instances, the tissue sample is lung tissue. In some instances, the tissue sample is bladder tissue. In some instances, the tissue sample is renal tissue. In some instances, the tissue sample is breast tissue. In some instances, the tissue sample is skin tissue. In some instances, the tissue sample is pancreatic tissue. In some instances, the tissue sample is gastric tissue. In some instances, the tissue sample is esophageal tissue. In some instances, the tissue sample is mesothelial tissue. In some instances, the tissue sample is thyroid tissue. In some instances, the tissue sample is colorectal tissue. In some instances, the tissue sample is head or neck tissue. In some instances, the tissue sample is osteosarcoma tissue. In some instances, the tissue sample is prostate tissue. In some instances, the tissue sample is ovarian tissue, HCC (liver), blood cells, lymph nodes, or bone/bone marrow.

In some instances, the tumor tissue sample is extracted from a malignant cancerous tumor (i.e., cancer). In some instances, the cancer is a solid tumor, or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, polymphocytic leukemia, or hairy cell leukemia) or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further divided into those of epithelial cell origin and those of non-epithelial cell origin. Examples of epithelial cell solid tumors include tumors of the gastrointestinal tract, colon, colorectal (e.g., basaloid colorectal carcinoma), breast, prostate, lung, kidney, liver, pancreas, ovary (e.g., endometrioid ovarian carcinoma), head and neck, oral cavity, stomach, duodenum, small intestine, large intestine, anus, gall bladder, labium, nasopharynx, skin, uterus, male genital organ, urinary organs (e.g., urothelium carcinoma, dysplastic urothelium carcinoma, transitional cell carcinoma), bladder, and skin. Solid tumors of non-epithelial origin include sarcomas, brain tumors, and bone tumors. In some instances, the cancer is non-small cell lung cancer (NSCLC). In some instances, the cancer is second-line or third-line locally advanced or metastatic non-small cell lung cancer. In some instances, the cancer is adenocarcinoma. In some instances, the cancer is squamous cell carcinoma.

(v) RNA Extraction

Prior to detecting the level of a nucleic acid, mRNA may be isolated from a target sample. In some instances, the mRNA is total RNA isolated from tumors or tumor cell lines or, alternatively, normal tissues or cell lines. RNA can be isolated from a variety of tumor tissues, including breast, lung, colon, prostate, brain, liver, kidney, pancreas, stomach, gall bladder, spleen, thymus, testis, ovary, uterus, etc., the corresponding normal tissues, or tumor cell lines. If the source of mRNA is a primary tumor, mRNA can be extracted, for example, from frozen or archived paraffin-embedded and fixed (e.g. formalin-fixed) tissue samples. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., Bio Techniques 18:42044 (1995). In particular, RNA isolation can be performed using a purification kit, buffer set, and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE® Complete DNA and RNA Purification Kit (EPICENTRE®, Madison, Wis.), and Paraffin Block RNA Isolation Kit (Ambion, Inc.). Total RNA from tissue samples can be isolated, for example, by using RNA Stat-60 (TelTest). RNA prepared from tumor tissue samples can also be isolated, for example, by cesium chloride density gradient centrifugation.

(vi) Immune-Score Expression Level

The immune-score expression level may reflect the expression levels of one or more genes described herein (e.g., at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)). In certain instances, to determine an immune-score expression level, the detected expression level of each gene is normalized using any one of the standard normalization methods known in the art. One of skill in the art will appreciate that the normalization method used may depend on the gene expression methodology used (e.g., one or more housekeeping genes may be used for normalization in the context of an RT-qPCR methodology, but a whole genome or substantially whole genome may be used as a normalization baseline in the context of an RNA-seq methodology). For example, the detected expression level of each gene assayed can be normalized for both differences in the amount of the gene(s) assayed, variability in the quality of the samples used, and/or variability between assay runs.

In some instances, normalization may be accomplished by detecting expression of certain one or more normalizing gene(s), including reference gene(s) (e.g., a housekeeping gene (e.g., TMEM55B)). For example, in some instances, the nucleic acid expression levels detected using the methods described herein (e.g., for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) may be normalized to the expression level of one or more reference genes (e.g., one, two, three, four, five, six, seven, eight, nine, or more reference genes, e.g., a housekeeping gene (e.g., TMEM55B)). Alternatively, normalization can be based on the average signal or median signal of all of the assayed genes. On a gene-by-gene basis, a measured normalized amount of a subject tumor mRNA can be compared to the amount found in a reference immune-score expression level. The presence and/or expression level/amount measured in a particular subject sample to be analyzed will fall at some percentile within this range, which can be determined by methods well known in the art.

In other instances, to determine an immune-score expression level, the detected expression level of each assayed gene is not normalized.

The immune-score expression level may reflect the aggregate or composite expression level of a single gene or a plurality of genes described herein (e.g., for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)). Any statistical approaches known in the art may be used to determine the immune-score expression level.

For example, the immune-score expression level may reflect the median expression level, mean expression level, or a numerical value that reflects the aggregated Z-score expression level for the combination of genes assayed (e.g., for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)).

In some instances, the immune-score expression level reflects the median normalized expression level, mean normalized expression level, or a numerical value that reflects the aggregated Z-score normalized expression level for the combinations of genes assayed (e.g., for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)).

For example, the immune-score expression level may reflect an average (mean) of the expression levels of each gene in a combination of two genes listed in Table 1. In some instances, the immune-score expression level reflects an average (mean) of the normalized expression levels of each gene in a combination of two genes listed in Table 1 (e.g., normalized to a reference gene, e.g., a housekeeping gene, e.g., TMEM55B). In some instances, the immune-score expression level reflects a median of the expression levels of each gene in a combination of two genes listed in Table 1. In some instances, the immune-score expression level reflects a median of the normalized expression levels of each gene in a combination of two genes listed in Table 1 (e.g., normalized to a reference gene, e.g., a housekeeping gene, e.g., TMEM55B). In some instances, the immune-score expression level reflects the Z-score for each gene in a combination of two genes listed in Table 1. In some instances, the immune-score expression level is a numerical value that reflects the aggregated Z-score expression level of a combination of two genes listed in Table 1.

For example, the immune-score expression level may reflect an average (mean) of the expression levels of each gene in a combination of three genes listed in Table 2 (e.g., each of PD-L1, CXCL9, and IFNG). In some instances, the immune-score expression level reflects an average (mean) of the normalized expression levels of each gene in a combination of three genes listed in Table 2 (e.g., each of PD-L1, CXCL9, and IFNG) (e.g., normalized to a reference gene, e.g., a house-keeping gene, e.g., TMEM55B). In some instances, the immune-score expression level reflects a median of the expression levels of each gene in a combination of three genes listed in Table 2 (e.g., each of PD-L1, CXCL9, and IFNG). In some instances, the immune-score expression level reflects a median of the normalized expression levels of each gene in a combination of three genes listed in Table 2 (e.g., each of PD-L1, CXCL9, and IFNG) (e.g., normalized to a reference gene, e.g., a house-keeping gene, e.g., TMEM55B). In some instances, the immune-score expression level reflects the Z-score for each gene in a combination of three genes listed in Table 2 (e.g., each of PD-L1, CXCL9, and IFNG). In some instances, the immune-score expression level is a numerical value that reflects the aggregated Z-score expression level of a combination of three genes listed in Table 2 (e.g., each of PD-L1, CXCL9, and IFNG).

In another particular instance, the immune-score expression level may reflect an average (mean) of the expression levels of each gene in a combination of four genes listed in Table 3 (e.g., each of PD-L1, IFNG, GZMB, and CD8A). In some instances, the immune-score expression level reflects an average (mean) of the normalized expression levels of each gene in a combination of four genes listed in Table 3 (e.g., each of PD-L1, IFNG, GZMB, and CD8A) (e.g., normalized to a reference gene, e.g., a house-keeping gene, e.g., TMEM55B). In some instances, the immune-score expression level reflects a median of the expression levels of each gene in a combination of four genes listed in Table 3 (e.g., each of PD-L1, IFNG, GZMB, and CD8A). In some instances, the immune-score expression level reflects a median of the normalized expression levels of each gene in a combination of four genes listed in Table 3 (e.g., each of PD-L1, IFNG, GZMB, and CD8A) (e.g., normalized to a reference gene, e.g., a house-keeping gene, e.g., TMEM55B). In some instances, the immune-score expression level reflects the Z-score for each gene in a combination of four genes listed in Table 3 (e.g., each of PD-L1, IFNG, GZMB, and CD8A). In some instances, the immune-score expression level is a numerical value that reflects the aggregated Z-score expression level of a combination of four genes listed in Table 3 (e.g., each of PD-L1, IFNG, GZMB, and CD8A).

In yet another instance, the immune-score expression level reflects an average (mean) of the expression levels each gene in a combination of five genes listed in Table 4 (e.g., each of PD-L1, IFNG, GZMB, CD8A, and PD-1). In some instances, the immune-score expression level reflects an average (mean) of the normalized expression levels of each gene in a combination of five genes listed in Table 4 (e.g., each of PD-L1, IFNG, GZMB, CD8A, and PD-1) (e.g., normalized to a reference gene, e.g., a house-keeping gene, e.g., TMEM55B). In some instances, the immune-score expression level reflects a median of the expression levels of each gene in a combination of five genes listed in Table 4 (e.g., each of PD-L1, IFNG, GZMB, CD8A, and PD-1). In some instances, the immune-score expression level reflects a median of the normalized expression levels of each gene in a combination of five genes listed in Table 4 (e.g., each of PD-L1, IFNG, GZMB, CD8A, and PD-1) (e.g., normalized to a reference gene, e.g., a house-keeping gene, e.g., TMEM55B). In some instances, the immune-score expression level reflects the Z-score for each gene in a combination of five genes listed in Table 4 (e.g., each of PD-L1, IFNG, GZMB, CD8A, and PD-1). In some instances, the immune-score expression level is a numerical value that reflects the aggregated Z-score expression level of a combination of five genes listed in Table 4 (e.g., each of PD-L1, IFNG, GZMB, CD8A, and PD-1).

In yet another instance, the immune-score expression level reflects an average (mean) of the expression levels each of PD-L1, IFNG, GZMB, CD8A, and PD-1. In some instances, the immune-score expression level reflects an average (mean) of the normalized expression levels of each of PD-L1, IFNG, GZMB, CD8A, and PD-1 (e.g., normalized to a reference gene, e.g., a house-keeping gene, e.g., TMEM55B). In some instances, the immune-score expression level reflects a median of the expression levels of PD-L1, IFNG, GZMB, CD8A, and PD-1. In some instances, the immune-score expression level reflects a median of the normalized expression levels of PD-L1, IFNG, GZMB, CD8A, and PD-1 (e.g., normalized to a reference gene, e.g., a house-keeping gene, e.g., TMEM55B). In some instances, the immune-score expression level reflects the Z-score for PD-L1, IFNG, GZMB, CD8A, and PD-1. In some instances, the immune-score expression level is a numerical value that reflects the aggregated Z-score expression level for PD-L1, IFNG, GZMB, CD8A, and PD-1.

(vii) Reference Immune-Score Expression Level

The reference immune-score expression level may be a value derived from analysis of any of the reference populations described herein. In some instances, the reference immune-score expression level may be a “cut-off” value selected based on a reference immune-score expression level that divides a reference population into subsets, e.g., subsets that exhibit significant differences (e.g., statistically significant differences) in treatment response to a PD-L1 axis binding antagonist therapy and a non-PD-L1 axis binding antagonist therapy. In such instances, relative treatment response may be evaluated based on progression-free survival (PFS) or overall survival (OS), expressed for example as a hazard ratio (HR) (e.g., progression-free survival HR (PFS HR) or overall survival HR (OS HR)).

In certain instances, the reference immune-score expression level is an immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that significantly (e.g., statistically significantly) separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a significant difference between an individual's responsiveness to treatment with the PD-L1 axis binding antagonist therapy and an individual's responsiveness to treatment with the non-PD-L1 axis binding antagonist therapy above the reference immune-score expression level (i.e., above the cut-off), wherein the individual's responsiveness to treatment with the PD-L1 axis binding antagonist therapy is significantly improved relative to the individual's responsiveness to treatment with the non-PD-L1 axis binding antagonist therapy.

In some instances, the reference immune-score expression level is an immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that substantially optimally separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a substantially maximal difference between an individual's responsiveness to treatment with the PD-L1 axis binding antagonist therapy and an individual's responsiveness to treatment with the non-PD-L1 axis binding antagonist therapy above the reference immune-score expression level (i.e., above the cut-off), wherein the individual's responsiveness to treatment with the PD-L1 axis binding antagonist therapy is significantly (e.g., statistically significantly) improved relative to the individual's responsiveness to treatment with the non-PD-L1 axis binding antagonist therapy.

In certain particular instances, the reference immune-score expression level is an immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that optimally separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a maximal difference between an individual's responsiveness to treatment with the PD-L1 axis binding antagonist therapy and an individual's responsiveness to treatment with the non-PD-L1 axis binding antagonist therapy above the reference immune-score expression level (i.e., above the cut-off), wherein the individual's responsiveness to treatment with the PD-L1 axis binding antagonist therapy is significantly (e.g., statistically significantly) improved relative to the individual's responsiveness to treatment with the non-PD-L1 axis binding antagonist therapy.

In certain instances, the reference immune-score expression level is an immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that significantly (e.g., statistically significantly) separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a significant difference between an individual's responsiveness to treatment with the PD-L1 axis binding antagonist therapy and an individual's responsiveness to treatment with the non-PD-L1 axis binding antagonist therapy below the reference immune-score expression level (i.e., below the cut-off), wherein the individual's responsiveness to treatment with the non-PD-L1 axis binding antagonist therapy is significantly (e.g., statistically significantly) improved relative to the individual's responsiveness to treatment with the PD-L1 axis binding antagonist therapy.

In some instances, the reference immune-score expression level is an immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that substantially optimally separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a substantially maximal difference between an individual's responsiveness to treatment with the PD-L1 axis binding antagonist therapy and an individual's responsiveness to treatment with the non-PD-L1 axis binding antagonist therapy below the reference immune-score expression level (i.e., below the cut-off), wherein the individual's responsiveness to treatment with the non-PD-L1 axis binding antagonist therapy is significantly (e.g., statistically significantly) improved relative to the individual's responsiveness to treatment with the PD-L1 axis binding antagonist therapy.

In certain particular instances, the reference immune-score expression level is an immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that optimally separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a maximal difference between an individual's responsiveness to treatment with the PD-L1 axis binding antagonist therapy and an individual's responsiveness to treatment with the non-PD-L1 axis binding antagonist therapy below the reference immune-score expression level (i.e., below the cut-off), wherein the individual's responsiveness to treatment with the non-PD-L1 axis binding antagonist therapy is significantly (e.g., statistically significantly) improved relative to the individual's responsiveness to treatment with the PD-L1 axis binding antagonist therapy.

In some instances, the reference immune-score expression level is defined by an immune-score expression level with a certain prevalence in a reference population. For example, in certain instances, the reference immune-score expression level is an immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that significantly (e.g., statistically significantly) separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a significant difference between an individual's responsiveness to treatment with the PD-L1 axis binding antagonist therapy and an individual's responsiveness to treatment with the non-PD-L1 axis binding antagonist therapy in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 45% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in the reference population, wherein the individual's responsiveness to treatment with the PD-L1 axis binding antagonist therapy is significantly improved relative to the individual's responsiveness to treatment with the non-PD-L1 axis binding antagonist therapy.

In some instances, the reference immune-score expression level is an immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that substantially optimally separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a substantially maximal difference between an individual's responsiveness to treatment with the PD-L1 axis binding antagonist therapy and an individual's responsiveness to treatment with the non-PD-L1 axis binding antagonist therapy in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 45% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th}percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in the reference population, wherein the individual's responsiveness to treatment with the PD-L1 axis binding antagonist therapy is significantly improved relative to the individual's responsiveness to treatment with the non-PD-L1 axis binding antagonist therapy.

In certain particular instances, the reference immune-score expression level is an immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that optimally separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a maximal difference between an individual's responsiveness to treatment with the PD-L1 axis binding antagonist therapy and an individual's responsiveness to treatment with the non-PD-L1 axis binding antagonist therapy in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 45% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in the reference population, wherein the individual's responsiveness to treatment with the PD-L1 axis binding antagonist therapy is significantly (e.g., statistically significantly) improved relative to the individual's responsiveness to treatment with the non-PD-L1 axis binding antagonist therapy.

In certain instances, the reference immune-score expression level is an immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that significantly (e.g., statistically significantly) separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a significant difference between an individual's responsiveness to treatment with the PD-L1 axis binding antagonist therapy and an individual's responsiveness to treatment with the non-PD-L1 axis binding antagonist therapy in about the bottom 99^{th} percentile (equal to, or lower than, about the 99% prevalence level), about the bottom 95^{th} percentile (equal to, or lower than, about the 95% prevalence level), about the bottom 90^{th} percentile (equal to, or lower than, about the 90% prevalence level), about the bottom 85^{th} percentile (equal to, or lower than, about the 85% prevalence level), about the bottom 80^{th} percentile (equal to, or lower than, about the 80% prevalence level), about the bottom 75^{th} percentile (equal to, or lower than, about the 75% prevalence level), about the bottom 70^{th} percentile (equal to, or lower than, about the 70% prevalence level), about the bottom 65^{th} percentile (equal to, or lower than, about the 65% prevalence level), about the bottom 60^{th} percentile (equal to, or lower than, about the 60% prevalence level), about the bottom 55^{th} percentile (equal to, or lower than, about the 55% prevalence level), about the bottom 50^{th} percentile (equal to, or lower than, about the 50% prevalence level), about the bottom 45^{th} percentile (equal to, or lower than, about the 45% prevalence level), about the bottom 40^{th} percentile (equal to, or lower than, about the 40% prevalence level), about the bottom 35^{th} percentile (equal to, or lower than, about the 35% prevalence level), about the bottom 30^{th} percentile (equal to, or lower than, about the 30% prevalence level), about the bottom 25^{th} percentile (equal to, or lower than, about the 25% prevalence level), about the bottom 20^{th} percentile (equal to, or lower than, about the 20% prevalence level), about the bottom 15^{th} percentile (equal to, or lower than, about the 15% prevalence level), about the bottom 10^{th} percentile (equal to, or lower than, about the 10% prevalence level), about the bottom 5^{th} percentile (equal to, or lower than, about the 5% prevalence level), or about the bottom 1^{st} percentile (equal to, or lower than, about the 1% prevalence level) of the immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in the reference population, wherein the individual's responsiveness to treatment with the non-PD-L1 axis binding antagonist therapy is significantly (e.g., statistically significantly) improved relative to the individual's responsiveness to treatment with the PD-L1 axis binding antagonist therapy.

In some instances, the reference immune-score expression level is an immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that substantially optimally separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a substantially maximal difference between an individual's responsiveness to treatment with the PD-L1 axis binding antagonist therapy and an individual's responsiveness to treatment with the non-PD-L1 axis binding antagonist therapy in about the bottom 99^{th} percentile (equal to, or lower than, about the 99% prevalence level), about the bottom 95^{th} percentile (equal to, or lower than, about the 95% prevalence level), about the bottom 90^{th} percentile (equal to, or lower than, about the 90% prevalence level), about the bottom 85^{th} percentile (equal to, or lower than, about the 85% prevalence level), about the bottom 80^{th} percentile (equal to, or lower than, about the 80% prevalence level), about the bottom 75^{th} percentile (equal to, or lower than, about the 75% prevalence level), about the bottom 70^{th} percentile (equal to, or lower than, about the 70% prevalence level), about the bottom 65^{th} percentile (equal to, or lower than, about the 65% prevalence level), about the bottom 60^{th} percentile (equal to, or lower than, about the 60% prevalence level), about the bottom 55^{th} percentile (equal to, or lower than, about the 55% prevalence level), about the bottom 50^{th} percentile (equal to, or lower than, about the 50% prevalence level), about the bottom 45^{th} percentile (equal to, or lower than, about the 45% prevalence level), about the bottom 40^{th} percentile (equal to, or lower than, about the 40% prevalence level), about the bottom 35^{th} percentile (equal to, or lower than, about the 35% prevalence level), about the bottom 30^{th} percentile (equal to, or lower than, about the 30% prevalence level), about the bottom 25^{th} percentile (equal to, or lower than, about the 25% prevalence level), about the bottom 20^{th} percentile (equal to, or lower than, about the 20% prevalence level), about the bottom 15^{th} percentile (equal to, or lower than, about the 15% prevalence level), about the bottom 10^{th} percentile (equal to, or lower than, about the 10% prevalence level), about the bottom 5^{th} percentile (equal to, or lower than, about the 5% prevalence level), or about the bottom 1^{st} percentile (equal to, or lower than, about the 1% prevalence level) of the immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in the reference population, wherein the individual's responsiveness to treatment with the non-PD-L1 axis binding antagonist therapy is significantly (e.g., statistically significantly) improved relative to the individual's responsiveness to treatment with the PD-L1 axis binding antagonist therapy.

In certain particular instances, the reference immune-score expression level is an immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that optimally separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a maximal difference between an individual's responsiveness to treatment with the PD-L1 axis binding antagonist therapy and an individual's responsiveness to treatment with the non-PD-L1 axis binding antagonist therapy in about the bottom 99^{th} percentile (equal to, or lower than, about the 99% prevalence level), about the bottom 95^{th} percentile (equal to, or lower than, about the 95% prevalence level), about the bottom 90^{th} percentile (equal to, or lower than, about the 90% prevalence level), about the bottom 85^{th} percentile (equal to, or lower than, about the 85% prevalence level), about the bottom 80^{th} percentile (equal to, or lower than, about the 80% prevalence level), about the bottom 75^{th} percentile (equal to, or lower than, about the 75% prevalence level), about the bottom 70^{th} percentile (equal to, or lower than, about the 70% prevalence level), about the bottom 65^{th} percentile (equal to, or lower than, about the 65% prevalence level), about the bottom 60^{th} percentile (equal to, or lower than, about the 60% prevalence level), about the bottom 55^{th} percentile (equal to, or lower than, about the 55% prevalence level), about the bottom 50^{th} percentile (equal to, or lower than, about the 50% prevalence level), about the bottom 45^{th} percentile (equal to, or lower than, about the 45% prevalence level), about the bottom 40^{th} percentile (equal to, or lower than, about the 40% prevalence level), about the bottom 35^{th} percentile (equal to, or lower than, about the 35% prevalence level), about the bottom 30^{th} percentile (equal to, or lower than, about the 30% prevalence level), about the bottom 25^{th} percentile (equal to, or lower than, about the 25% prevalence level), about the bottom 20^{th} percentile (equal to, or lower than, about the 20% prevalence level), about the bottom 15^{th} percentile (equal to, or lower than, about the 15% prevalence level), about the bottom 10^{th} percentile (equal to, or lower than, about the 10% prevalence level), about the bottom 5^{th} percentile (equal to, or lower than, about the 5% prevalence level), or about the bottom 1^{st} percentile (equal to, or lower than, about the 1% prevalence level) of the immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in the reference population, wherein the individual's responsiveness to treatment with the non-PD-L1 axis binding antagonist therapy is significantly (e.g., statistically significantly) improved relative to the individual's responsiveness to treatment with the PD-L1 axis binding antagonist therapy. In some instances, the reference immune-score expression level is a median immune-score expression level (e.g., a median of a normalized immune-score expression level) for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that significantly (e.g., statistically significantly) separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a significant difference between an individual's responsiveness to treatment with the PD-L1 axis binding antagonist therapy and an individual's responsiveness to treatment with the non-PD-L1 axis binding antagonist therapy above the reference immune-score expression level (i.e., above the median cut-off), wherein the individual's responsiveness to treatment with the PD-L1 axis binding antagonist therapy is significantly improved relative to the individual's responsiveness to treatment with the non-PD-L1 axis binding antagonist therapy.

In some instances, the reference immune-score expression level is a median immune-score expression level (e.g., a median of a normalized immune-score expression level) for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that substantially optimally separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a substantially maximal difference between an individual's responsiveness to treatment with the PD-L1 axis binding antagonist therapy and an individual's responsiveness to treatment with the non-PD-L1 axis binding antagonist therapy above the reference immune-score expression level (i.e., above the median cut-off), wherein the individual's responsiveness to treatment with the PD-L1 axis binding antagonist therapy is significantly improved relative to the individual's responsiveness to treatment with the non-PD-L1 axis binding antagonist therapy.

In some instances, the reference immune-score expression level is a median immune-score expression level (e.g., a median of a normalized immune-score expression level) for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that optimally separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a maximal difference between an individual's responsiveness to treatment with the PD-L1 axis binding antagonist therapy and an individual's responsiveness to treatment with the non-PD-L1 axis binding antagonist therapy above the reference immune-score expression level (i.e., above the median cut-off), wherein the individual's responsiveness to treatment with the PD-L1 axis binding antagonist therapy is significantly improved relative to the individual's responsiveness to treatment with the non-PD-L1 axis binding antagonist therapy.

In some instances, the reference immune-score expression level is a median immune-score expression level (e.g., a median of a normalized immune-score expression level) for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that significantly (e.g., statistically significantly) separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a significant difference between an individual's responsiveness to treatment with the PD-L1 axis binding antagonist therapy and an individual's responsiveness to treatment with the non-PD-L1 axis binding antagonist therapy below the reference immune-score expression level (i.e., below the median cut-off), wherein the individual's responsiveness to treatment with the non-PD-L1 axis binding antagonist therapy is significantly (e.g., statistically significantly) improved relative to the individual's responsiveness to treatment with the PD-L1 axis binding antagonist therapy.

In some instances, the reference immune-score expression level is a median immune-score expression level (e.g., a median of a normalized immune-score expression level) for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that substantially optimally separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a substantially maximal difference between an individual's responsiveness to treatment with the PD-L1 axis binding antagonist therapy and an individual's responsiveness to treatment with the non-PD-L1 axis binding antagonist therapy below the reference immune-score expression level (i.e., below the median cut-off), wherein the individual's responsiveness to treatment with the non-PD-L1 axis binding antagonist therapy is significantly (e.g., statistically significantly) improved relative to the individual's responsiveness to treatment with the PD-L1 axis binding antagonist therapy.

In some instances, the reference immune-score expression level is a median immune-score expression level (e.g., a median of a normalized immune-score expression level) for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that optimally separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a maximal difference between an individual's responsiveness to treatment with the PD-L1 axis binding antagonist therapy and an individual's responsiveness to treatment with the non-PD-L1 axis binding antagonist therapy below the reference immune-score expression level (i.e., below the median cut-off), wherein the individual's responsiveness to treatment with the non-PD-L1 axis binding antagonist therapy is significantly (e.g., statistically significantly) improved relative to the individual's responsiveness to treatment with the PD-L1 axis binding antagonist therapy.

In some instances, the reference immune-score expression level is the average (e.g., an average (mean) of a normalized immune-score expression level) expression level for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that significantly (e.g., statistically significantly) separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a significant difference between an individual's responsiveness to treatment with the PD-L1 axis binding antagonist therapy and an individual's responsiveness to treatment with the non-PD-L1 axis binding antagonist therapy above the reference immune-score expression level (i.e., above the mean cut-off), wherein the individual's responsiveness to treatment with the PD-L1 axis binding antagonist therapy is significantly improved relative to the individual's responsiveness to treatment with the non-PD-L1 axis binding antagonist therapy.

In some instances, the reference immune-score expression level is the average (e.g., an average (mean) of a normalized immune-score expression level) expression level for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that substantially optimally separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a substantially maximal difference between an individual's responsiveness to treatment with the PD-L1 axis binding antagonist therapy and an individual's responsiveness to treatment with the non-PD-L1 axis binding antagonist therapy above the reference immune-score expression level (i.e., above the mean cut-off), wherein the individual's responsiveness to treatment with the PD-L1 axis binding antagonist therapy is significantly improved relative to the individual's responsiveness to treatment with the non-PD-L1 axis binding antagonist therapy.

In some instances, the reference immune-score expression level is the average (e.g., an average (mean) of a normalized immune-score expression level) expression level for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that optimally separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a maximal difference between an individual's responsiveness to treatment with the PD-L1 axis binding antagonist therapy and an individual's responsiveness to treatment with the non-PD-L1 axis binding antagonist therapy above the reference immune-score expression level (i.e., above the mean cut-off), wherein the individual's responsiveness to treatment with the PD-L1 axis binding antagonist therapy is significantly (e.g., statistically significantly) improved relative to the individual's responsiveness to treatment with the non-PD-L1 axis binding antagonist therapy.

In some instances, the reference immune-score expression level is the average (e.g., an average (mean) of a normalized immune-score expression level) expression level for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that significantly (e.g., statistically significantly) separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a significant difference between an individual's responsiveness to treatment with the PD-L1 axis binding antagonist therapy and an individual's responsiveness to treatment with the non-PD-L1 axis binding antagonist therapy below the reference immune-score expression level (i.e., below the mean cut-off), wherein the individual's responsiveness to treatment with the non-PD-L1 axis binding antagonist therapy is significantly (e.g., statistically significantly) improved relative to the individual's responsiveness to treatment with the PD-L1 axis binding antagonist therapy.

In some instances, the reference immune-score expression level is the average (e.g., an average (mean) of a normalized immune-score expression level) expression level for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that substantially optimally separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a substantially maximal difference between an individual's responsiveness to treatment with the PD-L1 axis binding antagonist therapy and an individual's responsiveness to treatment with the non-PD-L1 axis binding antagonist therapy below the reference immune-score expression level (i.e., below the mean cut-off), wherein the individual's responsiveness to treatment with the non-PD-L1 axis binding antagonist therapy is significantly (e.g., statistically significantly) improved relative to the individual's responsiveness to treatment with the PD-L1 axis binding antagonist therapy.

In some instances, the reference immune-score expression level is the average (e.g., an average (mean) of a normalized immune-score expression level) expression level for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population that optimally separates a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a maximal difference between an individual's responsiveness to treatment with the PD-L1 axis binding antagonist therapy and an individual's responsiveness to treatment with the non-PD-L1 axis binding antagonist therapy below the reference immune-score expression level (i.e., below the mean cut-off), wherein the individual's responsiveness to treatment with the non-PD-L1 axis binding antagonist therapy is significantly (e.g., statistically significantly) improved relative to the individual's responsiveness to treatment with the PD-L1 axis binding antagonist therapy.

In some instances, the reference immune-score expression level is defined by an immune-score expression level with a certain prevalence in a reference population, as further discussed herein. In some instances, the reference-immune score expression level is a pre-assigned value (e.g., a cut-off value previously determined to significantly (e.g., statistically significantly) separate a first subset of individuals who have been treated with a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)) therapy in a reference population and a second subset of individuals who have been treated with a non-PD-L1 axis binding antagonist therapy that does not comprise a PD-L1 axis binding antagonist in the same reference population based on a significant difference between an individual's responsiveness to treatment with the PD-L1 axis binding antagonist therapy and an individual's responsiveness to treatment with the non-PD-L1 axis binding antagonist therapy above and/or below the cut-off value, wherein the individual's responsiveness to treatment with the PD-L1 axis binding antagonist therapy is significantly (e.g., statistically significantly) improved relative to the individual's responsiveness to treatment with the non-PD-L1 axis binding antagonist therapy above the cut-off value and/or the individual's responsiveness to treatment with the non-PD-L1 axis binding antagonist therapy is significantly (e.g., statistically significantly) improved relative to the individual's responsiveness to treatment with the PD-L1 axis binding antagonist therapy below the cut-off value).

In some instances, the reference immune-score expression level may also be determined at one or more time points from a sample or samples obtained from the individual undergoing testing and/or treatment using the methods and/or assays described herein. In some instances, the reference immune-score expression level is the expression level for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a sample previously obtained from the individual at a time point prior to administration of a PD-L1 axis binding antagonist (e.g., PD-L1 binding antagonist, e.g., anti-PD-L1 antibody, e.g., atezolizumab (MPDL3280A)).

In some instances, the reference immune-score expression level is the expression level for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a sample obtained from the individual at a time point following administration of a PD-L1 axis binding antagonist (e.g., PD-L1 binding antagonist, e.g., anti-PD-L1 antibody, e.g., atezolizumab (MPDL3280A)). Such reference immune-score expression levels obtained from the individual may be useful for monitoring the response of the individual to treatment with a PD-L1 axis binding antagonist (e.g., PD-L1 binding antagonist, e.g., anti-PD-L1 antibody, e.g., atezolizumab (MPDL3280A)) over time.

The reference immune-score expression level may be determined from any number of individuals in a reference population and/or any number of reference samples (e.g., reference cell, reference tissue, control sample, control cell, or control tissue). The reference sample may be a single sample or a combination of multiple samples. A reference immune-score expression level based on a reference sample may be based on any number of reference samples (e.g., 2 or more, 5 or more, 10 or more, 50 or more, 100 or more, 500 or more, or 1000 or more reference samples). In certain instances, a reference sample includes pooled mRNA samples derived from samples obtained from multiple individuals. Further, a reference immune-score expression level based on a reference population, or samples therefrom, may be based on any number of individuals in the reference population (e.g., 2 or more, 5 or more, 10 or more, 50 or more, 100 or more, 500 or more, or 1000 or more individuals in a reference population). Any statistical methods known in the art may be used to determine a reference immune-score expression level from measurements based on multiple samples or multiple individuals in a reference population. See e.g., Sokal R. R. and Rholf, F. J. (1995) “Biometry: the principles and practice of statistics in biological research,” W.H. Freeman and Co. New York, N.Y.

(viii) Reference Population

The reference immune-score expression level may reflect the expression level(s) of one or more genes described herein (e.g., at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in one or more reference populations (or reference samples), or as a pre-assigned reference value.

In some instances, the reference immune-score expression level is an immune-score expression level for at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a reference population.

In some instances, the reference population is a population of individuals having a cancer. In some instances, the reference population is a population of individuals having lung cancer (e.g., NSCLC). In some instances, the reference population is a population of individuals having kidney cancer (e.g., RCC). In some instances, the reference population is a population of individuals having bladder cancer (e.g., UBC). In some instances, the reference population is a population of individuals having breast cancer (e.g., TNBC). In some instances, the reference population is a population of individuals who do not have a cancer.

Further, the reference population may include one or more subsets of individuals (e.g., one or more, two or more, three or more, four or more, five or more, six or more, seven or more, eight or more, nine or more, or ten or more subsets).

In some instances, the reference population is a population of individuals having the cancer, wherein the population of individuals includes a subset of individuals who have been treated with at least one dose (e.g., at least one, at least two, at least three, at least four, at least five, at least six, at least seven, at least eight, at least nine, at least ten, or more than ten doses) of a therapy including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, the therapy including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) is a monotherapy. In other instances, the therapy including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) is a combination treatment that includes, in addition to the PD-L1 axis binding antagonist, at least one additional therapeutic agent (e.g., an anti-cancer therapy (e.g., an anti-neoplastic agent, a chemotherapeutic agent, a growth inhibitory agent, a cytotoxic agent, a radiotherapy, or combinations thereof)).

In some instances, the reference population is a population of individuals having the cancer, wherein the population of individuals includes a subset of individuals who have been treated with a non-PD-L1 axis binding antagonist therapy (e.g., an anti-cancer therapy, (e.g., an anti-neoplastic agent, a chemotherapeutic agent, a growth inhibitory agent, a cytotoxic agent, a radiotherapy, or combinations thereof)) that does not include a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, the reference population includes a combination of individuals from different subsets. For example, in some instances, the reference population may be a population of individuals having the cancer, the population of individuals consisting of (i) a first subset of individuals who have been treated with a PD-L1 axis binding antagonist therapy (e.g., a PD-L1 binding antagonist therapy) that includes a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) and (ii) a second subset of individuals who have been treated with a non-PD-L1 axis binding antagonist therapy (e.g., a non-PD-L1 binding antagonist therapy) that does not include a PD-L1 axis binding antagonist (e.g., an anti-cancer therapy (e.g., an anti-neoplastic agent, a chemotherapeutic agent, a growth inhibitory agent, a cytotoxic agent, a radiotherapy, or combinations thereof). The PD-L1 axis binding antagonist therapy (e.g., a PD-L1 binding antagonist therapy) in the first subset may have been administered as either a monotherapy or a combination therapy.

### III. METHODS OF TREATMENT

Provided herein are methods, medicaments, and uses thereof, for treating an individual having a cancer (e.g., lung cancer (e.g., NSCLC), bladder cancer (e.g., UBC), kidney cancer (e.g., RCC), or breast cancer (e.g., TNBC)), the methods including administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) based on expression levels of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)), that have been determined in a sample from the individual.

In one aspect, provided herein are methods for treating an individual having a cancer (e.g., lung cancer (e.g., NSCLC), bladder cancer (e.g., UBC), kidney cancer (e.g., RCC), or breast cancer (e.g., TNBC)), the methods including (a) determining the expression level of at least one, at least two, at least three genes at least four, at least five, or all six genes selected from PD-L1, CXCL9, IFNG, GZMB, CD8A, and PD-1, in a sample from the individual, wherein an immune-score expression level of at least one, at least two, at least three, at least four, at least five, or all six of PD-L1, CXCL9, IFNG, GZMB, CD8A, or PD-1 in the sample has been determined to be above a reference immune-score expression (e.g., an immune-score expression level of at least one, at least two, at least three genes at least four, at least five, or all six genes selected from PD-L1, CXCL9, IFNG, GZMB, CD8A, and PD-1 in a reference population), and (b) administering an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) to the individual based on the immune-score expression level of at least one, at least two, at least three genes at least four, at least five, or all six genes selected from PD-L1, CXCL9, IFNG, GZMB, CD8A, and PD-1 determined in step (a).

In another aspect, provided herein are methods for treating an individual having a cancer, the methods including administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein prior to treatment, the expression level of at least one, at least two, at least three genes at least four, at least five, or all six genes selected from PD-L1, CXCL9, IFNG, GZMB, CD8A, and PD-1, in a sample from the individual has been determined and an immune-score expression level of at least one, at least two, at least three genes at least four, at least five, or all six genes selected from PD-L1, CXCL9, IFNG, GZMB, CD8A, and PD-1 in the sample that is above a reference immune-score expression level (e.g., an immune-score expression level of at least one, at least two, at least three genes at least four, at least five, or all six genes selected from PD-L1, CXCL9, IFNG, GZMB, CD8A, and PD-1 in a reference population) has been determined.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered as a first-line therapy. Alternatively, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered as a second-line therapy.

### A. Single-Gene and Two-Gene Immune-Scores

In particular instances, the methods and medicaments provided herein may be used to treat an individual having a cancer (e.g., lung cancer (e.g., NSCLC), bladder cancer (e.g., UBC), kidney cancer (e.g., RCC), or breast cancer (e.g., TNBC)) based on a determination of the immune-score expression levels of any one gene selected from PD-L1, CXCL9, IFNG, GZMB, CD8A, and PD-1. In some instances, the determination step includes determining the expression levels of a particular combination of any one gene selected from PD-L1, CXCL9, IFNG, GZMB, CD8A, and PD-1 and one or more additional genes associated with T-effector cells, e.g., determining the expression level of (i) any one gene selected from PD-L1, CXCL9, IFNG, GZMB, CD8A, and PD-1 and (ii) one or more genes associated with T-effector cells (e.g., at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, or nineteen of CD8A, GZMA, GZMB, IFNG, EOMES, PRF1, PD-L1, PD-1, CXCL9, CD27, FOXP3, CTLA4, TIGIT, IDO1, CXCL10, CXCL11, PSMB8, PSMB9, TAP1, and/or TAP2), wherein the one or more genes associated with T-effector cells are different from the one gene selected from the group consisting of PD-L1, CXCL9, IFNG, GZMB, CD8A, and PD-1.

The examples and embodiments of methods of treatment, medicaments, and uses thereof, described in Sections III.B (i-iii), III.C (i-iii), III.D (i-iii), and III.E (i-iii) may also apply to the immune-score expression level for any one gene selected from PD-L1, CXCL9, IFNG, GZMB, CD8A, and PD-1.

In particular instances, the methods and medicaments provided herein may be used to treat an individual having a cancer (e.g., lung cancer (e.g., NSCLC), bladder cancer (e.g., UBC), kidney cancer (e.g., RCC), or breast cancer (e.g., TNBC)) based on a determination of the immune-score expression levels of two genes selected from PD-L1, CXCL9, IFNG, GZMB, CD8A, and PD-1. For example, the determination step may include determining the expression levels of any of the two-gene combinations listed in Table 1. In some instances, the determination step includes determining the expression levels of a particular combination of the three genes listed in Table 1 and one or more additional genes associated with T-effector cells, e.g., determining the expression level of (i) two genes selected from the group consisting of PD-L1, CXCL9, IFNG, GZMB, CD8A, and PD-1 (e.g., any one of the gene combinations listed in Table 1) and (ii) one or more genes associated with T-effector cells (e.g., at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, or eighteen of CD8A, GZMA, GZMB, IFNG, EOMES, PRF1, PD-L1, PD-1, CXCL9, CD27, FOXP3, CTLA4, TIGIT, IDO1, CXCL10, CXCL11, PSMB8, PSMB9, TAP1, and/or TAP2), wherein the one or more genes associated with T-effector cells are different from the two genes selected from the group consisting of PD-L1, CXCL9, IFNG, GZMB, CD8A, and PD-1.

The examples and embodiments of methods of treatment, medicaments, and uses thereof, described in Sections III.B (i-iii), III.C (i-iii), III.D (i-iii), and III.E (i-iii) may also apply to the immune-score expression levels for any two gene combinations listed in Table 1.

### B. Three-Gene Immune-Score Combinations

In particular instances, the methods and medicaments provided herein may be used to treat an individual having a cancer (e.g., lung cancer (e.g., NSCLC), bladder cancer (e.g., UBC), kidney cancer (e.g., RCC), or breast cancer (e.g., TNBC)) based on a determination of the immune-score expression levels of three genes selected from PD-L1, CXCL9, IFNG, GZMB, CD8A, and PD-1. For example, the determination step may include determining the expression levels of any of the three-gene combinations listed in Table 2. In some instances, the determination step includes determining the expression levels of a particular combination of the three genes listed in Table 2 and one or more additional genes associated with T-effector cells, e.g., determining the expression level of (i) three genes selected from the group consisting of PD-L1, CXCL9, IFNG, GZMB, CD8A, and PD-1 (e.g., any one of the gene combinations listed in Table 2) and (ii) one or more genes associated with T-effector cells (e.g., at least one, at least two, at least three, at least four, at least five, at least six, at least seven, at least eight, at least nine, at least ten, at least eleven, at least twelve, at least thirteen, at least fourteen, at least fifteen, at least sixteen, or seventeen of CD8A, GZMA, GZMB, IFNG, EOMES, PRF1, PD-L1, PD-1, CXCL9, CD27, FOXP3, CTLA4, TIGIT, IDO1, CXCL10, CXCL11, PSMB8, PSMB9, TAP1, and/or TAP2), wherein the one or more genes associated with T-effector cells are different from the three genes selected from the group consisting of PD-L1, CXCL9, IFNG, GZMB, CD8A, and PD-1.

The examples and instances outlined below for the PD-L1, CXCL9, and IFNG gene set may also apply to any one of the three-gene combinations listed in Table 2.

(i) Expression of PD-L1, CXCL9, and IFNG

In some instances, the methods may be used for treating an individual having a cancer (e.g., lung cancer (e.g., NSCLC), bladder cancer (e.g., UBC), kidney cancer (e.g., RCC), or breast cancer (e.g., TNBC)), the methods including (a) determining the expression level of PD-L1, CXCL9, and IFNG, in a sample from the individual, wherein an immune-score expression level of at least one, at least two, or all three of PD-L1, CXCL9, and IFNG in the sample has been determined to be above a reference immune-score expression level (e.g., an immune-score expression level of PD-L1, CXCL9, and IFNG in a reference population), and (b) administering an effective amount of a PD-L1 axis binding antagonist (e.g., PD-L1 binding antagonist, e.g., an anti-PD-L1 antibody, e.g., atezolizumab (MPDL3280A)) to the individual based on the immune-score expression level of at least one, at least two, or all three of PD-L1, CXCL9, and IFNG determined in step (a) (e.g., an immune-score expression level of PD-L1, CXCL9, and IFNG in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, CXCL9, and IFNG in a reference population (e.g., a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

In some instances, the methods provided herein may be used to treat an individual having a cancer, the methods including administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein prior to treatment, the expression level of PD-L1, CXCL9, and IFNG, in a sample from the individual has been determined and an immune-score expression level of at least one, at least two, or all three of PD-L1, CXCL9, and IFNG in the sample that is above a reference immune-score expression level has been determined (e.g., an immune-score expression level of PD-L1, CXCL9, and IFNG in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, CXCL9, and IFNG in a reference population (e.g., a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

(ii) Medicaments and Uses Thereof

In a further aspect, the invention provides for the use of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) in the manufacture or preparation of a medicament for treating an individual having a cancer (e.g., lung cancer (e.g., NSCLC), bladder cancer (e.g., UBC), kidney cancer (e.g., RCC), or breast cancer (e.g., TNBC)).

In some instances, the medicament is for use in a method of treating an individual having a cancer (e.g., lung cancer (e.g., NSCLC), bladder cancer (e.g., UBC), kidney cancer (e.g., RCC), or breast cancer (e.g., TNBC)), the methods including (a) determining the expression level of PD-L1, CXCL9, and IFNG, in a sample from the individual, wherein an immune-score expression level of at least one, at least two, or all three of PD-L1, CXCL9, and IFNG in the sample has been determined to be above a reference immune-score expression level (e.g., an immune-score expression level of at least one, at least two, or all three of PD-L1, CXCL9, and IFNG in a reference population), and (b) administering an effective amount of a PD-L1 axis binding antagonist (e.g., PD-L1 binding antagonist, e.g., an anti-PD-L1 antibody, e.g., atezolizumab (MPDL3280A)) to the individual based on the immune-score expression level of at least one, at least two, or all three of PD-L1, CXCL9, and IFNG determined in step (a) (e.g., an immune-score expression level of PD-L1, CXCL9, and IFNG in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, CXCL9, and IFNG in a reference population (e.g., a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

In some instances, the medicament is for use in a method of treating an individual having a cancer, the methods including administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein prior to treatment, the expression level of PD-L1, CXCL9, and IFNG, in a sample from the individual has been determined and an immune-score expression level of at least one, at least two, or all three of PD-L1, CXCL9, and IFNG in the sample that is above a reference immune-score expression level has been determined (e.g., an immune-score expression level of at least one, at least two, at least three of PD-L1, CXCL9, and IFNG in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, CXCL9, and IFNG in a reference population (e.g., a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

(iii) Uses of a PD-L1 Axis Binding Antagonist

In a further aspect, the invention provides for the use of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) in treating an individual having a cancer (e.g., lung cancer (e.g., NSCLC), bladder cancer (e.g., UBC), kidney cancer (e.g., RCC), or breast cancer (e.g., TNBC)).

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) is for use in a method of treating an individual having a cancer (e.g., lung cancer (e.g., NSCLC), bladder cancer (e.g., UBC), kidney cancer (e.g., RCC), or breast cancer (e.g., TNBC)), the methods including (a) determining the expression level of PD-L1, CXCL9, and IFNG, in a sample from the individual, wherein an immune-score expression level of at least one, at least two, or all three of PD-L1, CXCL9, and IFNG in the sample has been determined to be above a reference immune-score expression level (e.g., an immune-score expression level of at least one, at least two, or all three of PD-L1, CXCL9, and IFNG in a reference population), and (b) administering an effective amount of a PD-L1 axis binding antagonist (e.g., PD-L1 binding antagonist, e.g., an anti-PD-L1 antibody, e.g., atezolizumab (MPDL3280A)) to the individual based on the immune-score expression level of at least one, at least two, or all three of PD-L1, CXCL9, and IFNG determined in step (a) (e.g., an immune-score expression level of at least one, at least two, at least three of PD-L1, CXCL9, and IFNG in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, CXCL9, and IFNG in a reference population (e.g., a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) is for use in a method of treating an individual having a cancer (e.g., lung cancer (e.g., NSCLC), bladder cancer (e.g., UBC), kidney cancer (e.g., RCC), or breast cancer (e.g., TNBC)), the methods including administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein prior to treatment, the expression level of PD-L1, CXCL9, and IFNG, in a sample from the individual has been determined and an immune-score expression level of at least one, at least two, or all three of PD-L1, CXCL9, and IFNG in the sample that is above a reference immune-score expression level has been determined (e.g., an immune-score expression level of at least one, at least two, at least three of PD-L1, CXCL9, and IFNG in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, CXCL9, and IFNG in a reference population (e.g., a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

### C. Four-Gene Immune-Score Combinations

In particular instances, the methods and medicaments provided herein may be used to treat an individual having a cancer (e.g., lung cancer (e.g., NSCLC), bladder cancer (e.g., UBC), kidney cancer (e.g., RCC), or breast cancer (e.g., TNBC)) based on a determination of the immune-score expression levels of four genes selected from PD-L1, CXCL9, IFNG, GZMB, CD8A, and PD-1. For example, the determination step may include determining the expression levels of any of the combinations of four genes listed in Table 3. In some instances, the determination step includes determining the expression levels of a particular combination of the four genes listed in Table 3 and one or more additional genes associated with T-effector cells, e.g., determining the expression level of (i) four genes selected from the group consisting of PD-L1, CXCL9, IFNG, GZMB, CD8A, and PD-1 (e.g., any one of the four gene combinations listed in Table 3) and (ii) one or more genes associated with T-effector cells (e.g., at least one, at least two, at least three, at least four, at least five, at least six, at least seven, at least eight, at least nine, at least ten, at least eleven, at least twelve, at least thirteen, at least fourteen, at least fifteen, or sixteen of CD8A, GZMA, GZMB, IFNG, EOMES, PRF1, PD-L1, PD-1, CXCL9, CD27, FOXP3, CTLA4, TIGIT, IDO1, CXCL10, CXCL11, PSMB8, PSMB9, TAP1, and/or TAP2), wherein the one or more genes associated with T-effector cells are different from the four genes selected from the group consisting of PD-L1, CXCL9, IFNG, GZMB, CD8A, and PD-1.

The examples and instances outlined below for the PD-L1, IFNG, GZMB, and CD8A gene set may also apply to any of the four-gene combinations listed in Table 3.

(i) Expression of PD-L1, IFNG, GZMB, and CD8A

In some instances, the methods may be used for treating an individual having a cancer (e.g., lung cancer (e.g., NSCLC), bladder cancer (e.g., UBC), kidney cancer (e.g., RCC), or breast cancer (e.g., TNBC)), the methods including (a) determining the expression level of PD-L1, IFNG, GZMB, and CD8A, in a sample from the individual, wherein an immune-score expression level of at least one, at least two, at least three, or all four of PD-L1, IFNG, GZMB, and CD8A in the sample has been determined to be above a reference immune-score expression level (e.g., an immune-score expression level of at least one, at least two, at least three, or all four of PD-L1, IFNG, GZMB, and CD8A in a reference population), and (b) administering an effective amount of a PD-L1 axis binding antagonist (e.g., PD-L1 binding antagonist, e.g., an anti-PD-L1 antibody, e.g., atezolizumab (MPDL3280A)) to the individual based on the immune-score expression level of at least one, at least two, at least three, or all four of PD-L1, IFNG, GZMB, and CD8A determined in step (a) (e.g., an immune-score expression level of at least one, at least two, at least three, or all four of PD-L1, IFNG, GZMB, and CD8A in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level PD-L1, IFNG, GZMB, and CD8A in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

In some instances, the methods provided herein may be used to treat an individual having a cancer, the methods including administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein prior to treatment, the expression level of PD-L1, IFNG, GZMB, and CD8A, in a sample from the individual has been determined and an immune-score expression level of at least one, at least two, at least three, or all four of PD-L1, IFNG, GZMB, and CD8A in the sample that is above a reference immune-score expression level has been determined (e.g., an immune-score expression level of at least one, at least two, at least three, or all four of PD-L1, IFNG, GZMB, and CD8A in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, IFNG, GZMB, and CD8A in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

(ii) Medicaments and Uses Thereof

In a further aspect, the invention provides for the use of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) in the manufacture or preparation of a medicament for treating an individual having a cancer (e.g., lung cancer (e.g., NSCLC), bladder cancer (e.g., UBC), kidney cancer (e.g., RCC), or breast cancer (e.g., TNBC)).

In some instances, the medicament is for use in a method of treating an individual having a cancer (e.g., lung cancer (e.g., NSCLC), bladder cancer (e.g., UBC), kidney cancer (e.g., RCC), or breast cancer (e.g., TNBC)), the methods including (a) determining the expression level of PD-L1, IFNG, GZMB, and CD8A, in a sample from the individual, wherein an immune-score expression level of at least one, at least two, at least three, or all four of PD-L1, IFNG, GZMB, and CD8A in the sample has been determined to be above a reference immune-score expression level (e.g., an immune-score expression level of at least one, at least two, at least three, or all four of PD-L1, IFNG, GZMB, and CD8A in a reference population), and (b) administering an effective amount of a PD-L1 axis binding antagonist (e.g., PD-L1 binding antagonist, e.g., an anti-PD-L1 antibody, e.g., atezolizumab (MPDL3280A)) to the individual based on the immune-score expression level of at least one, at least two, at least three, or all four of PD-L1, IFNG, GZMB, and CD8A determined in step (a) (e.g., an immune-score expression level of at least one, at least two, at least three, or all four of PD-L1, IFNG, GZMB, and CD8A in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, IFNG, GZMB, and CD8A in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

In some instances, the medicament is for use in a method of treating an individual having a cancer, the methods including administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein prior to treatment, the expression level of PD-L1, IFNG, GZMB, and CD8A, in a sample from the individual has been determined and an immune-score expression level of at least one, at least two, at least three, or all four of PD-L1, IFNG, GZMB, and CD8A in the sample that is above a reference immune-score expression level has been determined (e.g., an immune-score expression level of at least one, at least two, at least three, or all four of PD-L1, IFNG, GZMB, and CD8A in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, IFNG, GZMB, and CD8A in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

(iii) Uses of a PD-L1 Axis Binding Antagonist

In a further aspect, the invention provides for the use of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) in treating an individual having a cancer (e.g., lung cancer (e.g., NSCLC), bladder cancer (e.g., UBC), kidney cancer (e.g., RCC), or breast cancer (e.g., TNBC)).

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) is for use in a method of treating an individual having a cancer (e.g., lung cancer (e.g., NSCLC), bladder cancer (e.g., UBC), kidney cancer (e.g., RCC), or breast cancer (e.g., TNBC)), the methods including (a) determining the expression level of PD-L1, IFNG, GZMB, and CD8A, in a sample from the individual, wherein an immune-score expression level of at least one, at least two, at least three, or all four of PD-L1, IFNG, GZMB, and CD8A in the sample has been determined to be above a reference immune-score expression level (e.g., an immune-score expression level of at least one, at least two, at least three, or all four of PD-L1, IFNG, GZMB, and CD8A in a reference population), and (b) administering an effective amount of a PD-L1 axis binding antagonist (e.g., PD-L1 binding antagonist, e.g., an anti-PD-L1 antibody, e.g., atezolizumab (MPDL3280A)) to the individual based on the immune-score expression level of at least one, at least two, at least three, or all four of PD-L1, IFNG, GZMB, and CD8A determined in step (a) (e.g., an immune-score expression level of at least one, at least two, at least three, or all four of PD-L1, IFNG, GZMB, and CD8A in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, IFNG, GZMB, and CD8A in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) is for use in a method of treating an individual having a cancer (e.g., lung cancer (e.g., NSCLC), bladder cancer (e.g., UBC), kidney cancer (e.g., RCC), or breast cancer (e.g., TNBC)), the methods including administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein prior to treatment, the expression level of PD-L1, IFNG, GZMB, and CD8A, in a sample from the individual has been determined and an immune-score expression level of at least one, at least two, at least three, or all four of PD-L1, IFNG, GZMB, and CD8A in the sample that is above a reference immune-score expression level has been determined (e.g., an immune-score expression level of at least one, at least two, at least three, or all four of PD-L1, IFNG, GZMB, and CD8A in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, IFNG, GZMB, and CD8A in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

### D. Five-Gene Immune-Score Combinations

In particular instances, the methods of treatment and medicaments provided herein may be used to treat an individual having a cancer (e.g., lung cancer (e.g., NSCLC), bladder cancer (e.g., UBC), kidney cancer (e.g., RCC), or breast cancer (e.g., TNBC)) based on a determination of the immune-score expression levels of five genes selected from PD-L1, CXCL9, IFNG, GZMB, CD8A, and PD-1. For example, the determination step may include determining the expression levels of any one of the combinations of five genes listed in Table 4. In some instances, the determination step includes determining the expression levels of a particular combination of the fives genes listed in Table 4 and one or more additional genes associated with T-effector cells, e.g., determining the expression level of (i) five genes selected from the group consisting of PD-L1, CXCL9, IFNG, GZMB, CD8A, and PD-1 (e.g., any one of the combinations of genes listed in Table 4) and (ii) one or more genes associated with T-effector cells (e.g., at least one, at least two, at least three, at least four, at least five, at least six, at least seven, at least eight, at least nine, at least ten, at least eleven, at least twelve, at least thirteen, at least fourteen, or fifteen of CD8A, GZMA, GZMB, IFNG, EOMES, PRF1, PD-L1, PD-1, CXCL9, CD27, FOXP3, CTLA4, TIGIT, IDO1, CXCL10, CXCL11, PSMB8, PSMB9, TAP1, and/or TAP2), wherein the one or more genes associated with T-effector cells are different from the five genes selected from the group consisting of PD-L1, CXCL9, IFNG, GZMB, CD8A, and PD-1. The examples and embodiments described below for the PD-L1, IFNG, GZMB, CD8A, and PD-1 gene set may also apply to any of the five-gene combinations listed in Table 4.

(i) Expression of PD-L1, IFNG, GZMB, CD8A, and PD-1

In some instances, the methods may be used for treating an individual having a cancer (e.g., lung cancer (e.g., NSCLC), bladder cancer (e.g., UBC), kidney cancer (e.g., RCC), or breast cancer (e.g., TNBC)), the methods including (a) determining the expression level of PD-L1, IFNG, GZMB, CD8A, and PD-1, in a sample from the individual, wherein an immune-score expression level of at least one, at least two, at least three, at least four, or all five of PD-L1, IFNG, GZMB, CD8A, and PD-1 in the sample has been determined to be above a reference immune-score expression level (e.g., an immune-score expression level of PD-L1, IFNG, GZMB, CD8A, and PD-1 in a reference population), and (b) administering an effective amount of a PD-L1 axis binding antagonist (e.g., PD-L1 binding antagonist, e.g., an anti-PD-L1 antibody, e.g., atezolizumab (MPDL3280A)) to the individual based on the immune-score expression level of at least one, at least two, at least three, at least four, or all five of PD-L1, IFNG, GZMB, CD8A, and PD-1 determined in step (a) (e.g., an immune-score expression level of at least one, at least two, at least three, at least four, or all five of PD-L1, IFNG, GZMB, CD8A, and PD-1 in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, IFNG, GZMB, CD8A, and PD-1 in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

In some instances, the methods provided herein may be used to treat an individual having a cancer, the methods including administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein prior to treatment, the expression level of PD-L1, IFNG, GZMB, CD8A, and PD-1, in a sample from the individual has been determined and an immune-score expression level of at least one, at least two, at least three, at least four, or all five of PD-L1, IFNG, GZMB, CD8A, and PD-1 in the sample that is above a reference immune-score expression level has been determined (e.g., an immune-score expression level of at least one, at least two, at least three, at least four, or all five of PD-L1, IFNG, GZMB, CD8A, and PD-1 in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, IFNG, GZMB, CD8A, and PD-1 in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

(ii) Medicaments and Uses Thereof

In a further aspect, the invention provides for the use of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) in the manufacture or preparation of a medicament for treating an individual having a cancer (e.g., lung cancer (e.g., NSCLC), bladder cancer (e.g., UBC), kidney cancer (e.g., RCC), or breast cancer (e.g., TNBC)).

In some instances, the medicament is for use in a method of treating an individual having a cancer (e.g., lung cancer (e.g., NSCLC), bladder cancer (e.g., UBC), kidney cancer (e.g., RCC), or breast cancer (e.g., TNBC)), the methods including (a) determining the expression level of PD-L1, IFNG, GZMB, CD8A, and PD-1, in a sample from the individual, wherein an immune-score expression level of at least one, at least two, at least three, at least four, or all five of PD-L1, IFNG, GZMB, CD8A, and PD-1 in the sample has been determined to be above a reference immune-score expression level (e.g., an immune-score expression level of PD-L1, IFNG, GZMB, CD8A, and PD-1 in a reference population), and (b) administering an effective amount of a PD-L1 axis binding antagonist (e.g., PD-L1 binding antagonist, e.g., an anti-PD-L1 antibody, e.g., atezolizumab (MPDL3280A)) to the individual based on the immune-score expression level of at least one, at least two, at least three, at least four, or all five of PD-L1, IFNG, GZMB, CD8A, and PD-1 determined in step (a) (e.g., an immune-score expression level of at least one, at least two, at least three, at least four, or all five of PD-L1, IFNG, GZMB, CD8A, and PD-1 in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, IFNG, GZMB, CD8A, and PD-1 in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

In some instances, the medicament is for use in a method of treating an individual having a cancer, the methods including administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein prior to treatment, the expression level of PD-L1, IFNG, GZMB, CD8A, and PD-1, in a sample from the individual has been determined and an immune-score expression level of at least one, at least two, at least three, at least four, or all five of PD-L1, IFNG, GZMB, CD8A, and PD-1 in the sample that is above a reference immune-score expression level has been determined (e.g., an immune-score expression level of at least one, at least two, at least three, at least four, or all five of PD-L1, IFNG, GZMB, CD8A, and PD-1 in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, IFNG, GZMB, CD8A, and PD-1 in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

(iii) Uses of a PD-L1 Axis Binding Antagonist

In a further aspect, the invention provides for the use of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) in treating an individual having a cancer (e.g., lung cancer (e.g., NSCLC), bladder cancer (e.g., UBC), kidney cancer (e.g., RCC), or breast cancer (e.g., TNBC)).

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) is for use in a method of treating an individual having a cancer (e.g., lung cancer (e.g., NSCLC), bladder cancer (e.g., UBC), kidney cancer (e.g., RCC), or breast cancer (e.g., TNBC)), the methods including (a) determining the expression level of PD-L1, IFNG, GZMB, CD8A, and PD-1, in a sample from the individual, wherein an immune-score expression level of at least one, at least two, at least three, at least four, or all five of PD-L1, IFNG, GZMB, CD8A, and PD-1 in the sample has been determined to be above a reference immune-score expression level (e.g., an immune-score expression level of PD-L1, IFNG, GZMB, CD8A, and PD-1 in a reference population), and (b) administering an effective amount of a PD-L1 axis binding antagonist (e.g., PD-L1 binding antagonist, e.g., an anti-PD-L1 antibody, e.g., atezolizumab (MPDL3280A)) to the individual based on the immune-score expression level of at least one, at least two, at least three, at least four, or all five of PD-L1, IFNG, GZMB, CD8A, and PD-1 determined in step (a) (e.g., an immune-score expression level of at least one, at least two, at least three, at least four, or all five of PD-L1, IFNG, GZMB, CD8A, and PD-1 in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, IFNG, GZMB, CD8A, and PD-1 in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) is for use in a method of treating an individual having a cancer (e.g., lung cancer (e.g., NSCLC), bladder cancer (e.g., UBC), kidney cancer (e.g., RCC), or breast cancer (e.g., TNBC)), the methods including administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein prior to treatment, the expression level of PD-L1, IFNG, GZMB, CD8A, and PD-1, in a sample from the individual has been determined and an immune-score expression level of at least one, at least two, at least three, at least four, or all five of PD-L1, IFNG, GZMB, CD8A, and PD-1 in the sample that is above a reference immune-score expression level has been determined (e.g., an immune-score expression level of at least one, at least two, at least three, at least four, or all five of PD-L1, IFNG, GZMB, CD8A, and PD-1 in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, IFNG, GZMB, CD8A, and PD-1 in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

### E. Six-Gene Immune-Score Combination

In particular instances, the methods of treatment and medicaments provided herein may be used to treat an individual having a cancer (e.g., lung cancer (e.g., NSCLC), bladder cancer (e.g., UBC), kidney cancer (e.g., RCC), or breast cancer (e.g., TNBC)) based on a determination of the immune-score expression levels of all six genes selected from PD-L1, CXCL9, IFNG, GZMB, CD8A, and PD-1. In some instances, the determination step includes determining the expression levels of all six genes selected from PD-L1, CXCL9, IFNG, GZMB, CD8A, and PD-1 and one or more additional genes associated with T-effector cells, e.g., determining the expression level of (i) all six genes selected from the group consisting of PD-L1, CXCL9, IFNG, GZMB, CD8A, and PD-1 (e.g., any one of the combinations of genes listed in Table 4) and (ii) one or more genes associated with T-effector cells (e.g., at least one, at least two, at least three, at least four, at least five, at least six, at least seven, at least eight, at least nine, at least ten, at least eleven, at least twelve, at least thirteen, or fourteen of CD8A, GZMA, GZMB, IFNG, EOMES, PRF1, PD-L1, PD-1, CXCL9, CD27, FOXP3, CTLA4, TIGIT, IDO1, CXCL10, CXCL11, PSMB8, PSMB9, TAP1, and/or TAP2), wherein the one or more genes associated with T-effector cells are different from PD-L1, CXCL9, IFNG, GZMB, CD8A, and PD-1.

(i) Expression of PD-L1, CXCL9, IFNG, GZMB, CD8A, and PD-1

In some instances, the methods may be used for treating an individual having a cancer (e.g., lung cancer (e.g., NSCLC), bladder cancer (e.g., UBC), kidney cancer (e.g., RCC), or breast cancer (e.g., TNBC)), the methods including (a) determining the expression level of PD-L1, CXCL9, IFNG, GZMB, CD8A, and PD-1, in a sample from the individual, wherein an immune-score expression level of PD-L1, CXCL9, IFNG, GZMB, CD8A, and PD-1 in the sample has been determined to be above a reference immune-score expression level (e.g., an immune-score expression level of PD-L1, CXCL9, IFNG, GZMB, CD8A, and PD-1 in a reference population), and (b) administering an effective amount of a PD-L1 axis binding antagonist (e.g., PD-L1 binding antagonist, e.g., an anti-PD-L1 antibody, e.g., atezolizumab (MPDL3280A)) to the individual based on the immune-score expression level of PD-L1, CXCL9, IFNG, GZMB, CD8A, and PD-1 determined in step (a) (e.g., an immune-score expression level of PD-L1, CXCL9, IFNG, GZMB, CD8A, and PD-1 in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, CXCL9, IFNG, GZMB, CD8A, and PD-1 in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

In some instances, the methods provided herein may be used to treat an individual having a cancer, the methods including administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein prior to treatment, the expression level of PD-L1, CXCL9, IFNG, GZMB, CD8A, and PD-1, in a sample from the individual has been determined and an immune-score expression level of PD-L1, CXCL9, IFNG, GZMB, CD8A, and PD-1 in the sample that is above a reference immune-score expression level has been determined (e.g., an immune-score expression level of PD-L1, CXCL9, IFNG, GZMB, CD8A, and PD-1 in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, CXCL9, IFNG, GZMB, CD8A, and PD-1 in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

(ii) Medicaments and Uses Thereof

In a further aspect, the invention provides for the use of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) in the manufacture or preparation of a medicament for treating an individual having a cancer (e.g., lung cancer (e.g., NSCLC), bladder cancer (e.g., UBC), kidney cancer (e.g., RCC), or breast cancer (e.g., TNBC)).

In some instances, the medicament is for use in a method of treating an individual having a cancer (e.g., lung cancer (e.g., NSCLC), bladder cancer (e.g., UBC), kidney cancer (e.g., RCC), or breast cancer (e.g., TNBC)), the methods including (a) determining the expression level of PD-L1, CXCL9, IFNG, GZMB, CD8A, and PD-1, in a sample from the individual, wherein an immune-score expression level of PD-L1, CXCL9, IFNG, GZMB, CD8A, and PD-1 in the sample has been determined to be above a reference immune-score expression level (e.g., an immune-score expression level of PD-L1, CXCL9, IFNG, GZMB, CD8A, and PD-1 in a reference population), and (b) administering an effective amount of a PD-L1 axis binding antagonist (e.g., PD-L1 binding antagonist, e.g., an anti-PD-L1 antibody, e.g., atezolizumab (MPDL3280A)) to the individual based on the immune-score expression level of PD-L1, CXCL9, IFNG, GZMB, CD8A, and PD-1 determined in step (a) (e.g., an immune-score expression level of PD-L1, CXCL9, IFNG, GZMB, CD8A, and PD-1 in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, CXCL9, IFNG, GZMB, CD8A, and PD-1 in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

In some instances, the medicament is for use in a method of treating an individual having a cancer, the methods including administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein prior to treatment, the expression level of PD-L1, CXCL9, IFNG, GZMB, CD8A, and PD-1, in a sample from the individual has been determined and an immune-score expression level of PD-L1, CXCL9, IFNG, GZMB, CD8A, and PD-1 in the sample that is above a reference immune-score expression level has been determined (e.g., an immune-score expression level of PD-L1, CXCL9, IFNG, GZMB, CD8A, and PD-1 in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, CXCL9, IFNG, GZMB, CD8A, and PD-1 in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

(iii) Uses of a PD-L1 Axis Binding Antagonist

In a further aspect, the invention provides for the use of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) in treating an individual having a cancer (e.g., lung cancer (e.g., NSCLC), bladder cancer (e.g., UBC), kidney cancer (e.g., RCC), or breast cancer (e.g., TNBC)).

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) is for use in a method of treating an individual having a cancer (e.g., lung cancer (e.g., NSCLC), bladder cancer (e.g., UBC), kidney cancer (e.g., RCC), or breast cancer (e.g., TNBC)), the methods including (a) determining the expression level of PD-L1, CXCL9, IFNG, GZMB, CD8A, and PD-1, in a sample from the individual, wherein an immune-score expression level of PD-L1, CXCL9, IFNG, GZMB, CD8A, and PD-1 in the sample has been determined to be above a reference immune-score expression level (e.g., an immune-score expression level of PD-L1, CXCL9, IFNG, GZMB, CD8A, and PD-1 in a reference population), and (b) administering an effective amount of a PD-L1 axis binding antagonist (e.g., PD-L1 binding antagonist, e.g., an anti-PD-L1 antibody, e.g., atezolizumab (MPDL3280A)) to the individual based on the immune-score expression level of PD-L1, CXCL9, IFNG, GZMB, CD8A, and PD-1 determined in step (a) (e.g., an immune-score expression level of PD-L1, CXCL9, IFNG, GZMB, CD8A, and PD-1 in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, CXCL9, IFNG, GZMB, CD8A, and PD-1 in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) is for use in a method of treating an individual having a cancer (e.g., lung cancer (e.g., NSCLC), bladder cancer (e.g., UBC), kidney cancer (e.g., RCC), or breast cancer (e.g., TNBC)), the methods including administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), wherein prior to treatment, the expression level of PD-L1, CXCL9, IFNG, GZMB, CD8A, and PD-1, in a sample from the individual has been determined and an immune-score expression level of PD-L1, CXCL9, IFNG, GZMB, CD8A, and PD-1 in the sample that is above a reference immune-score expression level has been determined (e.g., an immune-score expression level of PD-L1, CXCL9, IFNG, GZMB, CD8A, and PD-1 in the sample that is in about the top 99^{th} percentile (equal to, or higher than, about the 1% prevalence level), about the top 95^{th} percentile (equal to, or higher than, about the 5% prevalence level), about the top 90^{th} percentile (equal to, or higher than, about the 10% prevalence level), about the top 85^{th} percentile (equal to, or higher than, about the 15% prevalence level), about the top 80^{th} percentile (equal to, or higher than, about the 20% prevalence level), about the top 75^{th} percentile (equal to, or higher than, about the 25% prevalence level), about the top 70^{th} percentile (equal to, or higher than, about the 30% prevalence level), about the top 65^{th} percentile (equal to, or higher than, about the 35% prevalence level), about the top 60^{th} percentile (equal to, or higher than, about the 40% prevalence level), about the top 55^{th} percentile (equal to, or higher than, about the 10% prevalence level), about the top 50^{th} percentile (equal to, or higher than, about the 50% prevalence level), about the top 45^{th} percentile (equal to, or higher than, about the 55% prevalence level), about the top 40^{th} percentile (equal to, or higher than, about the 60% prevalence level), about the top 35^{th} percentile (equal to, or higher than, about the 65% prevalence level), about the top 30^{th} percentile (equal to, or higher than, about the 70% prevalence level), about the top 25^{th} percentile (equal to, or higher than, about the 75% prevalence level), about the top 20^{th} percentile (equal to, or higher than, about the 80% prevalence level), about the top 15^{th} percentile (equal to, or higher than, about the 85% prevalence level), about the top 10^{th} percentile (equal to, or higher than, about the 90% prevalence level), about the top 5^{th} percentile (equal to, or higher than, about the 95% prevalence level), or about the top 1^{st} percentile (equal to, or higher than, about the 99% prevalence level) of the immune-score expression level of PD-L1, CXCL9, IFNG, GZMB, CD8A, and PD-1 in a reference population (e.g., a population of individuals who do not have cancer or a population of individuals having cancer (e.g., patients having a cancer (e.g., lung cancer (e.g., NSCLC), bladder cancer (e.g., UBC), kidney cancer (e.g., RCC), or breast cancer (e.g., TNBC)) who have undergone one or more treatments with a PD-L1 axis binding antagonist therapy or a non-PD-L1 axis binding antagonist therapy).

### F. PD-L1 Axis Binding Antagonist

PD-L1 axis binding antagonists include PD-1 binding antagonists, PD-L1 binding antagonists, and PD-L2 binding antagonists. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1LG1,” “CD274,” “B7-H,” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some embodiments, PD-1, PD-L1, and PD-L2 are human PD-1, PD-L1, and PD-L2. The PD-1 axis binding antagonist may, in some instances, be a PD-1 binding antagonist, a PD-L1 binding antagonist, or a PD-L2 binding antagonist.

(i) PD-L1 Binding Antagonist

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. In some instances, the PD-L1 binding antagonist is an antibody. In some instances, the antibody is selected from the group consisting of: YW243.55.S70, MPDL3280A (atezolizumab), MDX-1105, MEDI4736 (durvalumab), and MSB0010718C (avelumab).

In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)₂ fragments. In some instances, the anti-PD-L1 antibody is a humanized antibody. In some instances, the anti-PD-L1 antibody is a human antibody. In some instances, the anti-PD-L1 antibody described herein binds to human PD-L1. In some particular instances, the anti-PD-L1 antibody is atezolizumab (CAS Registry Number: 1422185-06-5). Atezolizumab (Genentech) is also known as MPDL3280A.

In some instances, the anti-PD-L1 antibody comprises a heavy chain variable region (HVR-H) comprising an HVR-H1, HVR-H2, and HVR-H3 sequence, wherein:

(a) the HVR-H1 sequence is GFTFSDSWIH (SEQ ID NO: 9);

(b) the HVR-H2 sequence is AWISPYGGSTYYADSVKG (SEQ ID NO: 10); and

(c) the HVR-H3 sequence is RHWPGGFDY (SEQ ID NO: 11).

In some instances, the anti-PD-L1 antibody further comprises a light chain variable region (HVR-L) comprising an HVR-L1, HVR-L2, and HVR-L3 sequence, wherein:

(a) the HVR-L1 sequence is RASQDVSTAVA (SEQ ID NO: 12);

(b) the HVR-L2 sequence is SASFLYS (SEQ ID NO: 13); and

(c) the HVR-L3 sequence is QQYLYHPAT (SEQ ID NO: 14).

In some instances, the anti-PD-L1 antibody comprises a heavy chain and a light chain sequence, wherein:

(a) the heavy chain variable (VH) region sequence comprises the amino acid sequence:

(SEQ ID NO: 15)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS;

and

(b) the light chain variable (VL) region sequence comprises the amino acid sequence:

(SEQ ID NO: 16)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In some instances, the anti-PD-L1 antibody comprises a heavy chain and a light chain sequence, wherein:

(a) the heavy chain comprises the amino acid sequence:

(SEQ ID NO: 17)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG;

and

(b) the light chain comprises the amino acid sequence:

(SEQ ID NO: 18)
DIQMTQSPSSLSASVGDRVTITCRASQDVSTAVAWYQQKPGKAPKWYSAS
FLYSGVPSRFSGSGSGTDFTLTISSLQPEDFATYYCQQYLYHPATFGQGT
KVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLS
SPVTKSFNRGEC.

In some instances, the anti-PD-L1 antibody comprises (a) a VH domain comprising an amino acid sequence comprising having at least 95% sequence identity (e.g., at least 95%, 96%, 97%, 98%, or 99% sequence identity) to, or the sequence of (SEQ ID NO: 15); (b) a VL domain comprising an amino acid sequence comprising having at least 95% sequence identity (e.g., at least 95%, 96%, 97%, 98%, or 99% sequence identity) to, or the sequence of (SEQ ID NO: 16); or (c) a VH domain as in (a) and a VL domain as in (b). In other instances, the anti-PD-L1 antibody is selected from the group consisting of YW243.55.S70, MDX-1105, MEDI4736 (durvalumab), and MSB0010718C (avelumab). Antibody YW243.55.S70 is an anti-PD-L1 described in PCT Pub. No. WO 2010/077634. MDX-1105, also known as BMS-936559, is an anti-PD-L1 antibody described in PCT Pub. No. WO 2007/005874. MEDI4736 (durvalumab) is an anti-PD-L1 monoclonal antibody described in PCT Pub. No. WO 2011/066389 and U.S. Pub. No. 2013/034559. Examples of anti-PD-L1 antibodies useful for the methods of this invention, and methods for making thereof are described in PCT Pub. Nos. WO 2010/077634, WO 2007/005874, and WO 2011/066389, and also in U.S. Pat. No. 8,217,149, and U.S. Pub. No. 2013/034559, which are incorporated herein by reference.

(ii) PD-1 Binding Antagonist

In some instances, the PD-L1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. In some instances, the PD-1 binding antagonist is an antibody. In some instances, the antibody is selected from the group consisting of: MDX 1106 (nivolumab), MK-3475 (pembrolizumab), CT-011 (pidilizumab), MEDI-0680 (AMP-514), PDR001, REGN2810, and BGB-108. In some instances, the PD-1 binding antagonist is an Fc-fusion protein. For example, in some instances, the Fc-fusion protein is AMP-224.

In a further aspect, the invention provides for the use of a PD-L1 axis binding antagonist in the manufacture or preparation of a medicament. In one embodiment, the medicament is for treatment of a cancer. In a further embodiment, the medicament is for use in a method of treating a cancer comprising administering to a patient suffering from kidney cancer (e.g., a renal cell carcinoma (RCC), e.g., advanced RCC or metastatic RCC (mRCC), e.g., previously untreated advanced RCC or mRCC) an effective amount of the medicament. In one such embodiment, the method further comprises administering to the individual an effective amount of at least one additional therapeutic agent, e.g., as described below.

In some embodiments, the PD-1 binding antagonist is a molecule that inhibits the binding of PD-1 to its ligand binding partners. In a specific aspect the PD-1 ligand binding partners are PD-L1 and/or PD-L2. In another embodiment, a PD-L1 binding antagonist is a molecule that inhibits the binding of PD-L1 to its binding ligands. In a specific aspect, PD-L1 binding partners are PD-1 and/or B7-1. In another embodiment, the PD-L2 binding antagonist is a molecule that inhibits the binding of PD-L2 to its ligand binding partners. In a specific aspect, the PD-L2 binding ligand partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody), for example, as described below. In some embodiments, the anti-PD-1 antibody is selected from the group consisting of MDX-1106 (nivolumab), MK-3475 (pembrolizumab), CT-011 (pidilizumab), MEDI-0680 (AMP-514), PDR001, REGN2810, and BGB-108. MDX-1106, also known as MDX-1106-04, ONO-4538, BMS-936558, or nivolumab, is an anti-PD-1 antibody described in WO2006/121168. MK-3475, also known as pembrolizumab or lambrolizumab, is an anti-PD-1 antibody described in WO 2009/114335. CT-011, also known as hBAT, hBAT-1 or pidilizumab, is an anti-PD-1 antibody described in WO 2009/101611. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 binding antagonist is AMP-224. AMP-224, also known as B7-DClg, is a PD-L2-Fc fusion soluble receptor described in WO 2010/027827 and WO 2011/066342.

In some embodiments, the anti-PD-1 antibody is MDX-1106. Alternative names for “MDX-1106” include MDX-1106-04, ONO-4538, BMS-936558, and nivolumab. In some embodiments, the anti-PD-1 antibody is nivolumab (CAS Registry Number: 946414-94-4). In a still further embodiment, provided is an isolated anti-PD-1 antibody comprising a heavy chain variable region comprising the heavy chain variable region amino acid sequence from SEQ ID NO: 19 and/or a light chain variable region comprising the light chain variable region amino acid sequence from SEQ ID NO: 20.

In a still further embodiment, provided is an isolated anti-PD-1 antibody comprising a heavy chain and/or a light chain sequence, wherein:

(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 19)
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK,

and

(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 20)
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

(iii) Substitution, Insertion, and Deletion Variants

In certain instances, the anti-PD-L1 antibody (e.g., atezolizumab (MPDL3280A) variants having one or more amino acid substitutions are provided for use in the methods, compositions, and/or kits of the invention. Sites of interest for substitutional mutagenesis include the HVRs and FRs. Conservative substitutions are shown in Table 5 under the heading of “preferred substitutions.” More substantial changes are provided in Table 5 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 5
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

(iv) Glycosylation Variants

In some instances, the anti-PD-L1 antibody (e.g., atezolizumab (MPDL3280A)) variant has been modified to increase or decrease the extent to which the bispecific antibody is glycosylated. Addition or deletion of glycosylation sites to an anti-PD-L1 antibody *e.g., atezolizumab (MPDL3280A)) may be conveniently accomplished by altering the amino acid sequence such that one or more glycosylation sites is created or removed.

Where the bispecific antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In some instances, the anti-PD-L1 antibody (e.g., atezolizumab (MPDL3280A)) variant has a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO 2003/085107).

In view of the above, in some instances, the methods of the invention involve administering to the subject in the context of a fractionated, dose-escalation dosing regimen an anti-PD-L1 antibody (e.g., atezolizumab (MPDL3280A)) variant that comprises an aglycosylation site mutation. In some instances, the aglycosylation site mutation reduces effector function of the bispecific antibody. In some instances, the aglycosylation site mutation is a substitution mutation. In some instances, the bispecific antibody comprises a substitution mutation in the Fc region that reduces effector function. In some instances, the substitution mutation is at amino acid residue N297, L234, L235, and/or D265 (EU numbering). In some instances, the substitution mutation is selected from the group consisting of N297G, N297A, L234A, L235A, D265A, and P329G. In some instances, the substitution mutation is at amino acid residue N297. In a preferred embodiment, the substitution mutation is N297A.

In other instances, bispecific antibody variants with bisected oligosaccharides are used in accordance with the methods of the invention, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

(v) Fc Region Variants

In some instances, an anti-PD-L1 antibody, e.g., atezolizumab (MPDL3280A) variant that has one or more amino acid modifications introduced into the Fc region (i.e., an Fc region variant (see e.g., US 2012/0251531)) of the bispecific antibody may be administered to a subject having cancer (e.g., prostate cancer, e.g., CRPC, e.g., mCRPC or locally confined, inoperable CRPC) in accordance with the methods of the invention. The Fc region variant may comprise a human Fc region sequence (e.g., a human IgG1, IgG2, IgG3 or IgG4 Fc region) comprising an amino acid modification (e.g., a substitution) at one or more amino acid positions.

In some instances, the bispecific Fc region antibody variant possesses some but not all effector functions, which makes it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CYTOTOX96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Intl. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain instances, the proline at position 329 of a wild-type human Fc region in the antibody is substituted with glycine or arginine or an amino acid residue large enough to destroy the proline sandwich within the Fc/Fcγ receptor interface that is formed between the proline 329 of the Fc and tryptophan residues Trp 87 and Trp 110 of FcgRIII (Sondermann et al. Nature. 406, 267-273 (2000)). In certain embodiments, the bispecific antibody comprises at least one further amino acid substitution. In one embodiment, the further amino acid substitution is S228P, E233P, L234A, L235A, L235E, N297A, N297D, or P331S, and still in another embodiment the at least one further amino acid substitution is L234A and L235A of the human IgG1 Fc region or S228P and L235E of the human IgG4 Fc region (see e.g., US 2012/0251531), and still in another embodiment the at least one further amino acid substitution is L234A and L235A and P329G of the human IgG1 Fc region.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain instances, the anti-PD-L1 antibody (e.g., atezolizumab (MPDL3280A)) comprises an Fc region with one or more amino acid substitutions which improve ADCC, e.g., substitutions at positions 298, 333, and/or 334 of the Fc region (EU numbering of residues).

In some instances, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

(vi) Cysteine Engineered Antibody Variants

In certain embodiments, it may be desirable to create cysteine engineered anti-PD-L1 antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541

(vii) Other Antibody Derivatives

In some instances, the anti-PD-L1 antibody (e.g., atezolizumab (MPDL3280A)) may be modified to contain additional non-proteinaceous moieties that are known in the art and readily available and administered to the subject in accordance with the methods described herein. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### G. Administration

The PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), or compositions thereof, utilized in the methods, uses, assays, and kits described herein can be formulated for administration or administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some instances, the PD-L1 axis binding antagonist (e.g., PD-L1 binding antagonist, e.g., an anti-PD-L1 antibody, e.g., atezolizumab (MPDL3280A)) is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The PD-L1 axis binding antagonist (e.g., PD-L1 binding antagonist, e.g., an anti-PD-L1 antibody, e.g., atezolizumab (MPDL3280A)) and any additional therapeutic agent may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The PD-L1 axis binding antagonist (e.g., PD-L1 binding antagonist, e.g., an anti-PD-L1 antibody, e.g., atezolizumab (MPDL3280A)) need not be, but is optionally formulated with and/or administered concurrently with, one or more agents currently used to prevent or treat the disorder in question. The effective amount of such other agents depends on the amount of the PD-L1 axis binding antagonist (e.g., PD-L1 binding antagonist, e.g., an anti-PD-L1 antibody, e.g., atezolizumab (MPDL3280A)) present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of a cancer (e.g., a lung cancer (NSCLC), a bladder cancer, (UBC), a kidney cancer (RCC), or a breast cancer (TNBC)), the appropriate dosage of a PD-L1 axis binding antagonist (e.g., PD-L1 binding antagonist, e.g., an anti-PD-L1 antibody, e.g., atezolizumab (MPDL3280A)) described herein (when used alone or in combination with one or more other additional therapeutic agents) will depend on the type of disease to be treated, the severity and course of the disease, whether the PD-L1 axis binding antagonist (e.g., PD-L1 binding antagonist, e.g., an anti-PD-L1 antibody, e.g., atezolizumab (MPDL3280A)) is administered for preventive or therapeutic purposes, previous therapy, the patient's clinical history and response to the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)), and the discretion of the attending physician. The PD-L1 axis binding antagonist (e.g., PD-L1 binding antagonist, e.g., an anti-PD-L1 antibody, e.g., atezolizumab (MPDL3280A)) is suitably administered to the patient at one time or over a series of treatments. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g., every week or every three weeks (e.g., such that the patient receives, for example, from about two to about twenty, or e.g., about six doses of the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody))). An initial higher loading dose, followed by one or more lower doses may be administered. However, other dosage regimens may be useful. The progress of this therapy is easily monitored by conventional techniques and assays.

In some instances, an effective amount of the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be between about 60 mg to about 5000 mg (e.g., between about 60 mg to about 4500 mg, between about 60 mg to about 4000 mg, between about 60 mg to about 3500 mg, between about 60 mg to about 3000 mg, between about 60 mg to about 2500 mg, between about 650 mg to about 2000 mg, between about 60 mg to about 1500 mg, between about 100 mg to about 1500 mg, between about 300 mg to about 1500 mg, between about 500 mg to about 1500 mg, between about 700 mg to about 1500 mg, between about 1000 mg to about 1500 mg, between about 1000 mg to about 1400 mg, between about 1100 mg to about 1300 mg, between about 1150 mg to about 1250 mg, between about 1175 mg to about 1225 mg, or between about 1190 mg to about 1210 mg, e.g., about 1200 mg±5 mg, about 1200±2.5 mg, about 1200±1.0 mg, about 1200±0.5 mg, about 1200±0.2 mg, or about 1200±0.1 mg). In some instances, the methods include administering to the individual the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) at about 1200 mg (e.g., a fixed dose of about 1200 mg or about 15 mg/kg).

In some instances, the amount of the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) administered to individual (e.g., human) may be in the range of about 0.01 to about 50 mg/kg of the individual's body weight (e.g., between about 0.01 to about 45 mg/kg, between about 0.01 mg/kg to about 40 mg/kg, between about 0.01 mg/kg to about 35 mg/kg, between about 0.01 mg/kg to about 30 mg/kg, between about 0.1 mg/kg to about 30 mg/kg, between about 1 mg/kg to about 30 mg/kg, between about 2 mg/kg to about 30 mg/kg, between about 5 mg/kg to about 30 mg/kg, between about 5 mg/kg to about 25 mg/kg, between about 5 mg/kg to about 20 mg/kg, between about 10 mg/kg to about 20 mg/kg, or between about 12 mg/kg to about 18 mg/kg, e.g., about 15±2 mg/kg, about 15±1 mg/kg, about 15±0.5 mg/kg, about 15±0.2 mg/kg, or about 15±0.1 mg/kg). In some instances, the methods include administering to the individual the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) at about 15 mg/kg.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) is administered to the individual (e.g., a human) at 1200 mg intravenously every three weeks (q3w). The dose may be administered as a single dose or as multiple doses (e.g., 2, 3, 4, 5, 6, 7, or more than 7 doses), such as infusions. In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) administered to the individual (e.g., a human) may be administered alone or in combination with an additional therapeutic agent described herein (e.g., a VEGF antagonist (e.g., bevacizumab) and/or a chemotherapeutic (e.g., carboplatin and paclitaxel)), in four to six doses (e.g., every three weeks). The dose of the antibody administered in a combination treatment may be reduced as compared to a single treatment. The progress of this therapy is easily monitored by conventional techniques. In one instance, the PD-L1 axis binding antagonist (e.g., PD-L1 binding antagonist, e.g., anti-PD-L1 antibody, e.g., atezolizumab (e.g., MPDL3280A)) is administered as a monotherapy to the individual to treat a cancer. In other instances, the PD-L1 axis binding antagonist (e.g., PD-L1 binding antagonist, e.g., anti-PD-L1 antibody, e.g., atezolizumab (e.g., MPDL3280A)) is administered as a combination therapy, as described herein, to the individual to treat a cancer.

### H. Indications

The methods and medicaments described herein are useful for treating a patient having a cancer (e.g., lung cancer (e.g., NSCLC), bladder cancer (e.g., UBC), kidney cancer (e.g., RCC), or breast cancer (e.g., TNBC)) by administering to the individual an effective amount of a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). For example, the cancer may be a lung cancer, a kidney cancer, a bladder cancer, a breast cancer, a colorectal cancer, an ovarian cancer, a pancreatic cancer, a gastric carcinoma, an esophageal cancer, mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a merkel cell cancer, or a hematologic malignancy.

In some instances, the cancer is a lung cancer. For example, the lung cancer may be a non-small cell lung cancer (NSCLC), including but not limited to a locally advanced or metastatic (e.g., stage IIIB, stage IV, or recurrent) NSCLC. In some instances, the lung cancer (e.g., NSCLC) is unresectable/inoperable lung cancer (e.g., NSCLC). In some instances, the lung cancer is a chemotherapy-naïve lung cancer (e.g., a chemotherapy-naïve metastatic NSCLC (mNSCLC)). In some instances, the lung cancer is a non-squamous lung cancer (e.g., a non-squamous mNSCLC). In some instances, the lung cancer is a stage IV lung cancer (e.g., a stage IV mNSCLC). In some instances, the lung cancer is a recurrent lung cancer (e.g., a recurrent mNSCLC). In some instances, the patient having the lung cancer (e.g., NSCLC) has an EGFR or ALK genomic alteration. In some instances, the patient having lung cancer with a EGFR or ALK genomic alteration has disease progression/treatment intolerance with one or more approved tyrosine kinase inhibitors (TKI).

In some instances, the cancer may be a bladder cancer. For example, the bladder cancer may be a urothelial bladder cancer (UBC), including but not limited to a non-muscle invasive urothelial bladder cancer, a muscle-invasive urothelial bladder cancer, or a metastatic urothelial bladder cancer. In some instances, the urothelial bladder cancer is a metastatic urothelial bladder cancer.

In some instances, the cancer may be a kidney cancer. For example, the kidney cancer may be a renal cell carcinoma (RCC), including stage I RCC, stage II RCC, stage III RCC, stage IV RCC, or recurrent RCC.

In some instances, the cancer may be a breast cancer. In some instances, the breast cancer may be a triple-negative breast cancer. For example, the breast cancer may be triple-negative breast cancer, estrogen receptor-positive breast cancer, estrogen receptor-positive/HER2-negative breast cancer, HER2-negative breast cancer, HER2-positive breast cancer, estrogen receptor-negative breast cancer, progesterone receptor-positive breast cancer, or progesterone receptor-negative breast cancer.

In some instances, the individual having a cancer, e.g., cancers described herein, has not been previously treated for the cancer. For example, the individual having a cancer has not previously received a PD-L1 axis binding antagonist therapy (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, the individual having a cancer has previously received treatment for the cancer. In some instances, the individual having a cancer has previously received treatment including a non-PD-L1 axis binding antagonist therapy (e.g., an anti-cancer therapy (e.g., a cytotoxic agent, a growth-inhibitory agent, a radiation therapy, an anti-angiogenic agent, or a combination thereof)).

### I. Combination Therapies

In any of the methods herein, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in combination with an effective amount of one or more additional therapeutic agents. Suitable additional therapeutic agents include, for example, an anti-neoplastic agent, a chemotherapeutic agent, a growth inhibitory agent, a cytotoxic agent, a radiotherapy, or combinations thereof.

In some instances, the methods further involve administering to the patient an effective amount of one or more additional therapeutic agents. In some instances, the additional therapeutic agent is selected from the group consisting of a cytotoxic agent, a chemotherapeutic agent, a growth-inhibitory agent, a radiation therapy agent, an anti-angiogenic agent, and combinations thereof. In some instances, a PD-L1 axis binding antagonist (e.g., PD-L1 binding antagonist, e.g., anti-PD-L1 antibody, e.g., atezolizumab (MPDL3280A)) may be administered in conjunction with a chemotherapy or chemotherapeutic agent. In some instances, a PD-L1 axis binding antagonist (e.g., PD-L1 binding antagonist, e.g., an anti-PD-L1 antibody, e.g., atezolizumab (MPDL3280A)) may be administered in conjunction with a radiation therapy agent. In some instances, a PD-L1 axis binding antagonist (e.g., PD-L1 binding antagonist, e.g., an anti-PD-L1 antibody, e.g., atezolizumab (MPDL3280A)) may be administered in conjunction with a targeted therapy or targeted therapeutic agent. In some instances, a PD-L1 axis binding antagonist (e.g., PD-L1 binding antagonist, e.g., an anti-PD-L1 antibody, e.g., atezolizumab (MPDL3280A)) may be administered in conjunction with an immunotherapy or immunotherapeutic agent, for example a monoclonal antibody. In some instances, the additional therapeutic agent is an agonist directed against an activating co-stimulatory molecule. In some instances, the additional therapeutic agent is an antagonist directed against an inhibitory co-stimulatory molecule.

Such combination therapies noted above encompass combined administration (where two or more therapeutic agents are included in the same or separate formulations), and separate administration, in which case, administration of a PD-L1 axis binding antagonist (e.g., PD-L1 binding antagonist, e.g., an anti-PD-L1 antibody, e.g., atezolizumab (MPDL3280A)) can occur prior to, simultaneously, and/or following, administration of the additional therapeutic agent or agents. In one instance, administration of PD-L1 axis binding antagonist (e.g., PD-L1 binding antagonist, e.g., an anti-PD-L1 antibody, e.g., atezolizumab (MPDL3280A)) and administration of an additional therapeutic agent occur within about one month, or within about one, two or three weeks, or within about one, two, three, four, five, or six days, of each other.

Without wishing to be bound to theory, it is thought that enhancing T-cell stimulation, by promoting an activating co-stimulatory molecule or by inhibiting a negative co-stimulatory molecule, may promote tumor cell death thereby treating or delaying progression of cancer. In some instances, a PD-L1 axis binding antagonist (e.g., PD-L1 binding antagonist, e.g., an anti-PD-L1 antibody, e.g., atezolizumab (MPDL3280A)) may be administered in conjunction with an agonist directed against an activating co-stimulatory molecule. In some instances, an activating co-stimulatory molecule may include CD40, CD226, CD28, OX40, GITR, CD137, CD27, HVEM, or CD127. In some instances, the agonist directed against an activating co-stimulatory molecule is an agonist antibody that binds to CD40, CD226, CD28, OX40, GITR, CD137, CD27, HVEM, or CD127. In some instances, a PD-L1 axis binding antagonist (e.g., PD-L1 binding antagonist, e.g., an anti-PD-L1 antibody, e.g., atezolizumab (MPDL3280A)) may be administered in conjunction with an antagonist directed against an inhibitory co-stimulatory molecule. In some instances, an inhibitory co-stimulatory molecule may include CTLA-4 (also known as CD152), TIM-3, BTLA, VISTA, LAG-3, B7-H3, B7-H4, IDO, TIGIT, MICA/B, or arginase. In some instances, the antagonist directed against an inhibitory co-stimulatory molecule is an antagonist antibody that binds to CTLA-4, TIM-3, BTLA, VISTA, LAG-3, B7-H3, B7-H4, IDO, TIGIT, MICA/B, or arginase.

In some instances, a PD-L1 axis binding antagonist (e.g., PD-L1 binding antagonist, e.g., an anti-PD-L1 antibody, e.g., atezolizumab (MPDL3280A)) may be administered in conjunction with an antagonist directed against CTLA-4 (also known as CD152), e.g., a blocking antibody. In some instances, a PD-L1 axis binding antagonist (e.g., PD-L1 binding antagonist, e.g., an anti-PD-L1 antibody, e.g., atezolizumab (MPDL3280A)) may be administered in conjunction with ipilimumab (also known as MDX-010, MDX-101, or YERVOY®). In some instances, a PD-L1 axis binding antagonist (e.g., PD-L1 binding antagonist, e.g., an anti-PD-L1 antibody, e.g., atezolizumab (MPDL3280A)) may be administered in conjunction with tremelimumab (also known as ticilimumab or CP-675,206). In some instances, a PD-L1 axis binding antagonist (e.g., PD-L1 binding antagonist, e.g., an anti-PD-L1 antibody, e.g., atezolizumab (MPDL3280A)) may be administered in conjunction with an antagonist directed against B7-H3 (also known as CD276), e.g., a blocking antibody. In some instances, a PD-L1 axis binding antagonist (e.g., PD-L1 binding antagonist, e.g., an anti-PD-L1 antibody, e.g., atezolizumab (MPDL3280A)) may be administered in conjunction with MGA271. In some instances, a PD-L1 axis binding antagonist (e.g., PD-L1 binding antagonist, e.g., an anti-PD-L1 antibody, e.g., atezolizumab (MPDL3280A)) may be administered in conjunction with an antagonist directed against a TGF-beta, e.g., metelimumab (also known as CAT-192), fresolimumab (also known as GC1008), or LY2157299.

In some instances, a PD-L1 axis binding antagonist (e.g., PD-L1 binding antagonist, e.g., an anti-PD-L1 antibody, e.g., atezolizumab (MPDL3280A)) may be administered in conjunction with a treatment comprising adoptive transfer of a T-cell (e.g., a cytotoxic T-cell or CTL) expressing a chimeric antigen receptor (CAR). In some instances, a PD-L1 axis binding antagonist (e.g., PD-L1 binding antagonist, e.g., an anti-PD-L1 antibody, e.g., atezolizumab (MPDL3280A)) may be administered in conjunction with a treatment comprising adoptive transfer of a T-cell comprising a dominant-negative TGF beta receptor, e.g., a dominant-negative TGF beta type II receptor. In some instances, a PD-L1 axis binding antagonist (e.g., PD-L1 binding antagonist, e.g., an anti-PD-L1 antibody, e.g., atezolizumab (MPDL3280A)) may be administered in conjunction with a treatment comprising a HERCREEM protocol (see, e.g., ClinicalTrials.gov Identifier NCT00889954).

In some instances, a PD-L1 axis binding antagonist (e.g., PD-L1 binding antagonist, e.g., an anti-PD-L1 antibody, e.g., atezolizumab (MPDL3280A)) may be administered in conjunction with an agonist directed against CD137 (also known as TNFRSF9, 4-1BB, or ILA), e.g., an activating antibody. In some instances, a PD-L1 axis binding antagonist (e.g., PD-L1 binding antagonist, e.g., an anti-PD-L1 antibody, e.g., atezolizumab (MPDL3280A)) may be administered in conjunction with urelumab (also known as BMS-663513). In some instances, a PD-L1 axis binding antagonist (e.g., PD-L1 binding antagonist, e.g., an anti-PD-L1 antibody, e.g., atezolizumab (MPDL3280A)) may be administered in conjunction with an agonist directed against CD40, e.g., an activating antibody. In some instances, a PD-L1 axis binding antagonist (e.g., PD-L1 binding antagonist, e.g., an anti-PD-L1 antibody, e.g., atezolizumab (MPDL3280A)) may be administered in conjunction with CP-870893. In some instances, a PD-L1 axis binding antagonist (e.g., PD-L1 binding antagonist, e.g., an anti-PD-L1 antibody, e.g., atezolizumab (MPDL3280A)) may be administered in conjunction with an agonist directed against OX40 (also known as CD134), e.g., an activating antibody. In some instances, a PD-L1 axis binding antagonist (e.g., PD-L1 binding antagonist, e.g., an anti-PD-L1 antibody, e.g., atezolizumab (MPDL3280A)) may be administered in conjunction with an anti-OX40 antibody (e.g., AgonOX). In some instances, a PD-L1 axis binding antagonist (e.g., PD-L1 binding antagonist, e.g., an anti-PD-L1 antibody, e.g., atezolizumab (MPDL3280A)) may be administered in conjunction with an agonist directed against CD27, e.g., an activating antibody. In some instances, a PD-L1 axis binding antagonist (e.g., PD-L1 binding antagonist, e.g., an anti-PD-L1 antibody, e.g., atezolizumab (MPDL3280A)) may be administered in conjunction with CDX-1127. In some instances, a PD-L1 axis binding antagonist (e.g., PD-L1 binding antagonist, e.g., an anti-PD-L1 antibody, e.g., atezolizumab (MPDL3280A)) may be administered in conjunction with an antagonist directed against indoleamine-2,3-dioxygenase (IDO). In some instances, with the IDO antagonist is 1-methyl-D-tryptophan (also known as 1-D-MT).

In some instances, a PD-L1 axis binding antagonist (e.g., PD-L1 binding antagonist, e.g., an anti-PD-L1 antibody, e.g., atezolizumab (MPDL3280A)) may be administered in conjunction with an antibody-drug conjugate. In some instances, the antibody-drug conjugate comprises mertansine or monomethyl auristatin E (MMAE). In some instances, a PD-L1 axis binding antagonist (e.g., PD-L1 binding antagonist, e.g., an anti-PD-L1 antibody, e.g., atezolizumab (MPDL3280A)) may be administered in conjunction with an anti-NaPi2b antibody-MMAE conjugate (also known as DNIB0600A or RG7599). In some instances, a PD-L1 axis binding antagonist (e.g., PD-L1 binding antagonist, e.g., an anti-PD-L1 antibody, e.g., atezolizumab (MPDL3280A)) may be administered in conjunction with trastuzumab emtansine (also known as T-DM1, ado-trastuzumab emtansine, or KADCYLA®, Genentech). In some instances, a PD-L1 axis binding antagonist (e.g., PD-L1 binding antagonist, e.g., an anti-PD-L1 antibody, e.g., atezolizumab (MPDL3280A)) may be administered in conjunction with DMUC5754A. In some instances, a PD-L1 axis binding antagonist (e.g., PD-L1 binding antagonist, e.g., an anti-PD-L1 antibody, e.g., atezolizumab (MPDL3280A)) may be administered in conjunction with an antibody-drug conjugate targeting the endothelin B receptor (EDNBR), e.g., an antibody directed against EDNBR conjugated with MMAE.

In some instances, a PD-L1 axis binding antagonist (e.g., PD-L1 binding antagonist, e.g., an anti-PD-L1 antibody, e.g., atezolizumab (MPDL3280A)) may be administered in conjunction with an anti-angiogenesis agent. In some instances, a PD-L1 axis binding antagonist (e.g., PD-L1 binding antagonist, e.g., an anti-PD-L1 antibody, e.g., atezolizumab (MPDL3280A)) may be administered in conjunction with an antibody directed against a VEGF, e.g., VEGF-A. In some instances, a PD-L1 axis binding antagonist (e.g., PD-L1 binding antagonist, e.g., an anti-PD-L1 antibody, e.g., atezolizumab (MPDL3280A)) may be administered in conjunction with bevacizumab (also known as AVASTIN®, Genentech). For example, atezolizumab (MPDL3280A) may be administered in combination with bevacizumab. In further instances, atezolizumab (MPDL3280A)) may be administered in combination with bevacizumab and one or more chemotherapeutic agents (e.g., carboplatin and/or paclitaxel). In certain instances, atezolizumab (MPDL3280A)) may be administered in combination with bevacizumab, carboplatin, and paclitaxel. In some instances, a PD-L1 axis binding antagonist (e.g., PD-L1 binding antagonist, e.g., an anti-PD-L1 antibody, e.g., atezolizumab (MPDL3280A)) may be administered in conjunction with an antibody directed against angiopoietin 2 (also known as Ang2). In some instances, a PD-L1 axis binding antagonist (e.g., PD-L1 binding antagonist, e.g., an anti-PD-L1 antibody, e.g., atezolizumab (MPDL3280A)) may be administered in conjunction with MEDI3617.

The VEGF antagonist (e.g., bevacizumab) administered to the individual (e.g., human) in conjunction with a PD-L1 axis binding antagonist (e.g., PD-L1 binding antagonist, e.g., an anti-PD-L1 antibody, e.g., atezolizumab (MPDL3280A)) may be in the range of about 0.01 to about 50 mg/kg of the individual's body weight (e.g., between about 0.01 to about 45 mg/kg, between about 0.01 mg/kg to about 40 mg/kg, between about 0.01 mg/kg to about 35 mg/kg, between about 0.01 mg/kg to about 30 mg/kg, between about 0.1 mg/kg to about 30 mg/kg, between about 1 mg/kg to about 30 mg/kg, between about 2 mg/kg to about 30 mg/kg, between about 5 mg/kg to about 30 mg/kg, between about 5 mg/kg to about 25 mg/kg, between about 5 mg/kg to about 20 mg/kg, between about 10 mg/kg to about 20 mg/kg, or between about 12 mg/kg to about 18 mg/kg, e.g., about 15±2 mg/kg, about 15±1 mg/kg, about 15±0.5 mg/kg, about 15±0.2 mg/kg, or about 15±0.1 mg/kg). For example, in some instances, the methods include administering to the individual a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) at about 1200 mg in conjunction with a VEGF antagonist (e.g., bevacizumab) at about 15 mg/kg of the individual's body weight. The method may further include administration of one or more chemotherapeutic agents, such as carboplatin and/or paclitaxel.

In some instances, a PD-L1 axis binding antagonist (e.g., PD-L1 binding antagonist, e.g., an anti-PD-L1 antibody, e.g., atezolizumab (MPDL3280A)) may be administered in conjunction with an antineoplastic agent. In some instances, a PD-L1 axis binding antagonist (e.g., PD-L1 binding antagonist, e.g., an anti-PD-L1 antibody, e.g., atezolizumab (MPDL3280A)) may be administered in conjunction with an agent targeting CSF-1R (also known as M-CSFR or CD115). In some instances, a PD-L1 axis binding antagonist (e.g., PD-L1 binding antagonist, e.g., an anti-PD-L1 antibody, e.g., atezolizumab (MPDL3280A)) may be administered in conjunction with anti-CSF-1R (also known as IMC-CS4). In some instances, a PD-L1 axis binding antagonist (e.g., PD-L1 binding antagonist, e.g., an anti-PD-L1 antibody, e.g., atezolizumab (MPDL3280A)) may be administered in conjunction with an interferon, for example interferon alpha or interferon gamma. In some instances, a PD-L1 axis binding antagonist (e.g., PD-L1 binding antagonist, e.g., an anti-PD-L1 antibody, e.g., atezolizumab (MPDL3280A)) may be administered in conjunction with Roferon-A (also known as recombinant Interferon alpha-2a). In some instances, a PD-L1 axis binding antagonist (e.g., PD-L1 binding antagonist, e.g., an anti-PD-L1 antibody, e.g., atezolizumab (MPDL3280A)) may be administered in conjunction with GM-CSF (also known as recombinant human granulocyte macrophage colony stimulating factor, rhu GM-CSF, sargramostim, or LEUKINE®). In some instances, a PD-L1 axis binding antagonist (e.g., PD-L1 binding antagonist, e.g., an anti-PD-L1 antibody, e.g., atezolizumab (MPDL3280A)) may be administered in conjunction with IL-2 (also known as aldesleukin or PROLEUKIN®). In some instances, a PD-L1 axis binding antagonist (e.g., PD-L1 binding antagonist, e.g., an anti-PD-L1 antibody, e.g., atezolizumab (MPDL3280A)) may be administered in conjunction with IL-12. In some instances, a PD-L1 axis binding antagonist (e.g., PD-L1 binding antagonist, e.g., an anti-PD-L1 antibody, e.g., atezolizumab (MPDL3280A)) may be administered in conjunction with an antibody targeting CD20. In some instances, the antibody targeting CD20 is obinutuzumab (also known as GA101 or GAZYVA®) or rituximab. In some instances, a PD-L1 axis binding antagonist (e.g., PD-L1 binding antagonist, e.g., an anti-PD-L1 antibody, e.g., atezolizumab (MPDL3280A)) may be administered in conjunction with an antibody targeting GITR. In some instances, the antibody targeting GITR is TRX518.

In some instances, a PD-L1 axis binding antagonist (e.g., PD-L1 binding antagonist, e.g., an anti-PD-L1 antibody, e.g., atezolizumab (MPDL3280A)) may be administered in conjunction with a cancer vaccine. In some instances, the cancer vaccine is a peptide cancer vaccine, which in some instances is a personalized peptide vaccine. In some instances the peptide cancer vaccine is a multivalent long peptide, a multi-peptide, a peptide cocktail, a hybrid peptide, or a peptide-pulsed dendritic cell vaccine (see, e.g., Yamada et al., Cancer Sci. 104:14-21, 2013). In some instances, a PD-L1 axis binding antagonist (e.g., PD-L1 binding antagonist, e.g., an anti-PD-L1 antibody, e.g., atezolizumab (MPDL3280A)) may be administered in conjunction with an adjuvant. In some instances, a PD-L1 axis binding antagonist (e.g., PD-L1 binding antagonist, e.g., an anti-PD-L1 antibody, e.g., atezolizumab (MPDL3280A)) may be administered in conjunction with a treatment comprising a TLR agonist, e.g., Poly-ICLC (also known as HILTONOL®), LPS, MPL, or CpG ODN. In some instances, a PD-L1 axis binding antagonist (e.g., PD-L1 binding antagonist, e.g., an anti-PD-L1 antibody, e.g., atezolizumab (MPDL3280A)) may be administered in conjunction with tumor necrosis factor (TNF) alpha (TNF-α). In some instances, a PD-L1 axis binding antagonist (e.g., PD-L1 binding antagonist, e.g., an anti-PD-L1 antibody, e.g., atezolizumab (MPDL3280A)) may be administered in conjunction with IL-1. In some instances, a PD-L1 axis binding antagonist (e.g., PD-L1 binding antagonist, e.g., an anti-PD-L1 antibody, e.g., atezolizumab (MPDL3280A)) may be administered in conjunction with HMGB1. In some instances, a PD-L1 axis binding antagonist (e.g., PD-L1 binding antagonist, e.g., an anti-PD-L1 antibody, e.g., atezolizumab (MPDL3280A)) may be administered in conjunction with an IL-10 antagonist. In some instances, a PD-L1 axis binding antagonist (e.g., PD-L1 binding antagonist, e.g., an anti-PD-L1 antibody, e.g., atezolizumab (MPDL3280A)) may be administered in conjunction with an IL-4 antagonist. In some instances, a PD-L1 axis binding antagonist (e.g., PD-L1 binding antagonist, e.g., an anti-PD-L1 antibody, e.g., atezolizumab (MPDL3280A)) may be administered in conjunction with an IL-13 antagonist. In some instances, a PD-L1 axis binding antagonist (e.g., PD-L1 binding antagonist, e.g., an anti-PD-L1 antibody, e.g., atezolizumab (MPDL3280A)) may be administered in conjunction with an HVEM antagonist. In some instances, a PD-L1 axis binding antagonist (e.g., PD-L1 binding antagonist, e.g., an anti-PD-L1 antibody, e.g., atezolizumab (MPDL3280A)) may be administered in conjunction with an ICOS agonist, e.g., by administration of ICOS-L, or an agonistic antibody directed against ICOS. In some instances, a PD-L1 axis binding antagonist (e.g., PD-L1 binding antagonist, e.g., an anti-PD-L1 antibody, e.g., atezolizumab (MPDL3280A)) may be administered in conjunction with a treatment targeting CX3CL1. In some instances, a PD-L1 axis binding antagonist (e.g., PD-L1 binding antagonist, e.g., an anti-PD-L1 antibody, e.g., atezolizumab (MPDL3280A)) may be administered in conjunction with a treatment targeting CXCL9. In some instances, a PD-L1 axis binding antagonist (e.g., PD-L1 binding antagonist, e.g., an anti-PD-L1 antibody, e.g., atezolizumab (MPDL3280A)) may be administered in conjunction with a treatment targeting CXCL10. In some instances, a PD-L1 axis binding antagonist (e.g., PD-L1 binding antagonist, e.g., an anti-PD-L1 antibody, e.g., atezolizumab (MPDL3280A)) may be administered in conjunction with a treatment targeting CCL5. In some instances, a PD-L1 axis binding antagonist (e.g., PD-L1 binding antagonist, e.g., an anti-PD-L1 antibody, e.g., atezolizumab (MPDL3280A)) may be administered in conjunction with an LFA-1 or ICAM1 agonist. In some instances, a PD-L1 axis binding antagonist (e.g., PD-L1 binding antagonist, e.g., an anti-PD-L1 antibody, e.g., atezolizumab (MPDL3280A)) may be administered in conjunction with a Selectin agonist.

In some instances, a PD-L1 axis binding antagonist (e.g., PD-L1 binding antagonist, e.g., an anti-PD-L1 antibody, e.g., atezolizumab (MPDL3280A)) may be administered in conjunction with a targeted therapy. In some instances, a PD-L1 axis binding antagonist (e.g., PD-L1 binding antagonist, e.g., an anti-PD-L1 antibody, e.g., atezolizumab (MPDL3280A)) may be administered in conjunction with an inhibitor of B-Raf. In some instances, a PD-L1 axis binding antagonist (e.g., PD-L1 binding antagonist, e.g., an anti-PD-L1 antibody, e.g., atezolizumab (MPDL3280A)) may be administered in conjunction with vemurafenib (also known as ZELBORAF®). In some instances, a PD-L1 axis binding antagonist (e.g., PD-L1 binding antagonist, e.g., an anti-PD-L1 antibody, e.g., atezolizumab (MPDL3280A)) may be administered in conjunction with dabrafenib (also known as TAFINLAR®). In some instances, a PD-L1 axis binding antagonist (e.g., PD-L1 binding antagonist, e.g., an anti-PD-L1 antibody, e.g., atezolizumab (MPDL3280A)) may be administered in conjunction with erlotinib (also known as TARCEVA®). In some instances, a PD-L1 axis binding antagonist (e.g., PD-L1 binding antagonist, e.g., an anti-PD-L1 antibody, e.g., atezolizumab (MPDL3280A)) may be administered in conjunction with an inhibitor of a MEK, such as MEK1 (also known as MAP2K1) or MEK2 (also known as MAP2K2). In some instances, a PD-L1 axis binding antagonist (e.g., PD-L1 binding antagonist, e.g., an anti-PD-L1 antibody, e.g., atezolizumab (MPDL3280A)) may be administered in conjunction with cobimetinib (also known as GDC-0973 or XL-518). In some instances, a PD-L1 axis binding antagonist (e.g., PD-L1 binding antagonist, e.g., an anti-PD-L1 antibody, e.g., atezolizumab (MPDL3280A)) may be administered in conjunction with trametinib (also known as MEKINIST®). In some instances, a PD-L1 axis binding antagonist (e.g., PD-L1 binding antagonist, e.g., an anti-PD-L1 antibody, e.g., atezolizumab (MPDL3280A)) may be administered in conjunction with an inhibitor of K-Ras. In some instances, a PD-L1 axis binding antagonist (e.g., PD-L1 binding antagonist, e.g., an anti-PD-L1 antibody, e.g., atezolizumab (MPDL3280A)) may be administered in conjunction with an inhibitor of c-Met. In some instances, a PD-L1 axis binding antagonist (e.g., PD-L1 binding antagonist, e.g., an anti-PD-L1 antibody, e.g., atezolizumab (MPDL3280A)) may be administered in conjunction with onartuzumab (also known as MetMAb). In some instances, a PD-L1 axis binding antagonist (e.g., PD-L1 binding antagonist, e.g., an anti-PD-L1 antibody, e.g., atezolizumab (MPDL3280A)) may be administered in conjunction with an inhibitor of Alk. In some instances, a PD-L1 axis binding antagonist (e.g., PD-L1 binding antagonist, e.g., an anti-PD-L1 antibody, e.g., atezolizumab (MPDL3280A)) may be administered in conjunction with AF802 (also known as CH5424802 or alectinib). In some instances, a PD-L1 axis binding antagonist (e.g., PD-L1 binding antagonist, e.g., an anti-PD-L1 antibody, e.g., atezolizumab (MPDL3280A)) may be administered in conjunction with an inhibitor of a phosphatidylinositol 3-kinase (P13K). In some instances, a PD-L1 axis binding antagonist (e.g., PD-L1 binding antagonist, e.g., an anti-PD-L1 antibody, e.g., atezolizumab (MPDL3280A)) may be administered in conjunction with BKM120. In some instances, a PD-L1 axis binding antagonist (e.g., PD-L1 binding antagonist, e.g., an anti-PD-L1 antibody, e.g., atezolizumab (MPDL3280A)) may be administered in conjunction with idelalisib (also known as GS-1101 or CAL-101). In some embodiments, a PD-L1 axis binding antagonist (e.g., PD-L1 binding antagonist, e.g., an anti-PD-L1 antibody, e.g., atezolizumab (MPDL3280A)) may be administered in conjunction with perifosine (also known as KRX-0401). In some embodiments, a PD-L1 axis binding antagonist (e.g., PD-L1 binding antagonist, e.g., an anti-PD-L1 antibody, e.g., atezolizumab (MPDL3280A)) may be administered in conjunction with an inhibitor of an Akt. In some embodiments, a PD-L1 axis binding antagonist (e.g., PD-L1 binding antagonist, e.g., an anti-PD-L1 antibody, e.g., atezolizumab (MPDL3280A)) may be administered in conjunction with MK2206. In some instances, a PD-L1 axis binding antagonist (e.g., PD-L1 binding antagonist, e.g., an anti-PD-L1 antibody, e.g., atezolizumab (MPDL3280A)) may be administered in conjunction with GSK690693. In some instances, a PD-L1 axis binding antagonist (e.g., PD-L1 binding antagonist, e.g., an anti-PD-L1 antibody, e.g., atezolizumab (MPDL3280A)) may be administered in conjunction with GDC-0941. In some instances, a PD-L1 axis binding antagonist (e.g., PD-L1 binding antagonist, e.g., an anti-PD-L1 antibody, e.g., atezolizumab (MPDL3280A)) may be administered in conjunction with an inhibitor of mTOR. In some instances, a PD-L1 axis binding antagonist (e.g., PD-L1 binding antagonist, e.g., an anti-PD-L1 antibody, e.g., atezolizumab (MPDL3280A)) may be administered in conjunction with sirolimus (also known as rapamycin). In some instances, a PD-L1 axis binding antagonist (e.g., PD-L1 binding antagonist, e.g., an anti-PD-L1 antibody, e.g., atezolizumab (MPDL3280A)) may be administered in conjunction with temsirolimus (also known as CCI-779 or TORISEL®). In some instances, a PD-L1 axis binding antagonist (e.g., PD-L1 binding antagonist, e.g., an anti-PD-L1 antibody, e.g., atezolizumab (MPDL3280A)) may be administered in conjunction with everolimus (also known as RAD001). In some instances, a PD-L1 axis binding antagonist (e.g., PD-L1 binding antagonist, e.g., an anti-PD-L1 antibody, e.g., atezolizumab (MPDL3280A)) may be administered in conjunction with ridaforolimus (also known as AP-23573, MK-8669, or deforolimus). In some instances, a PD-L1 axis binding antagonist (e.g., PD-L1 binding antagonist, e.g., an anti-PD-L1 antibody, e.g., atezolizumab (MPDL3280A)) may be administered in conjunction with OSI-027. In some instances, a PD-L1 axis binding antagonist (e.g., PD-L1 binding antagonist, e.g., an anti-PD-L1 antibody, e.g., atezolizumab (MPDL3280A)) may be administered in conjunction with AZD8055. In some instances, a PD-L1 axis binding antagonist (e.g., PD-L1 binding antagonist, e.g., an anti-PD-L1 antibody, e.g., atezolizumab (MPDL3280A)) may be administered in conjunction with INK128. In some instances, a PD-L1 axis binding antagonist (e.g., PD-L1 binding antagonist, e.g., an anti-PD-L1 antibody, e.g., atezolizumab (MPDL3280A)) may be administered in conjunction with a dual P13K/mTOR inhibitor. In some instances, a PD-L1 axis binding antagonist (e.g., PD-L1 binding antagonist, e.g., an anti-PD-L1 antibody, e.g., atezolizumab (MPDL3280A)) may be administered in conjunction with XL765. In some instances, a PD-L1 axis binding antagonist (e.g., PD-L1 binding antagonist, e.g., an anti-PD-L1 antibody, e.g., atezolizumab (MPDL3280A)) may be administered in conjunction with GDC-0980. In some instances, a PD-L1 axis binding antagonist (e.g., PD-L1 binding antagonist, e.g., an anti-PD-L1 antibody, e.g., atezolizumab (MPDL3280A)) may be administered in conjunction with BEZ235 (also known as NVP-BEZ235). In some instances, a PD-L1 axis binding antagonist (e.g., PD-L1 binding antagonist, e.g., an anti-PD-L1 antibody, e.g., atezolizumab (MPDL3280A)) may be administered in conjunction with BGT226. In some instances, a PD-L1 axis binding antagonist (e.g., PD-L1 binding antagonist, e.g., an anti-PD-L1 antibody, e.g., atezolizumab (MPDL3280A)) may be administered in conjunction with GSK2126458. In some instances, a PD-L1 axis binding antagonist (e.g., PD-L1 binding antagonist, e.g., an anti-PD-L1 antibody, e.g., atezolizumab (MPDL3280A)) may be administered in conjunction with PF-04691502. In some instances, a PD-L1 axis binding antagonist (e.g., PD-L1 binding antagonist, e.g., an anti-PD-L1 antibody, e.g., atezolizumab (MPDL3280A)) may be administered in conjunction with PF-05212384 (also known as PKI-587).

(i) Combination Therapies in Clinical Trial

PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) can be administered to an individual in conjunction with one or more additional therapeutic agents, wherein, prior or subsequent to treatment, the individual has undergone diagnostic testing according to any one of the diagnostic methods described herein and has been identified as one who is likely to benefit from treatment with a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). As described further below, the additional therapeutic agents may be one that has been tested or is undergoing testing in a clinical trial for cancer therapies that include atezolizumab.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with obinutuzumab and polatuzumab vedotin (e.g., in the treatment of lymphoma (e.g., relapsed or refractory follicular lymphoma or diffuse large B-cell lymphoma)), as in the clinical trial NCT02729896.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with paclitaxel (e.g., albumin-bound paclitaxel (nab-paclitaxel (ABRAXANE®), e.g., in the treatment of breast cancer (e.g., TNBC)), as in the clinical trial NCT02530489.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with bevacizumab (also known as AVASTIN®) (e.g., in the treatment of locally advanced or metastatic tumors (e.g., in breast cancer, cervical cancer, kidney cancer, gastric cancer, ovarian cancer, or bladder cancer), as in the clinical trial NCT01633970.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with bevacizumab (also known as AVASTIN®) and leucovorin/oxaliplatin/5-fluorouracil (FOLFOX) (e.g., in the treatment of locally advanced or metastatic tumors, e.g., in breast cancer, cervical cancer, kidney cancer, gastric cancer, ovarian cancer, or bladder cancer), as in the clinical trial NCT01633970.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with paclitaxel (e.g., albumin-bound paclitaxel (nab-paclitaxel (ABRAXANE®)) and carboplatin (e.g., PARAPLATIN®) (e.g., in the treatment of locally advanced or metastatic tumors, e.g., in the treatment of lung cancer (NSCLC), breast cancer, cervical cancer, kidney cancer, gastric cancer, ovarian cancer, or bladder cancer), as in the clinical trial NCT01633970.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with paclitaxel (e.g., albumin-bound paclitaxel (nab-paclitaxel (ABRAXANE®)), e.g., in the treatment of locally advanced or metastatic tumors (e.g., in the treatment of lung cancer (NSCLC), breast cancer, cervical cancer, kidney cancer, gastric cancer, ovarian cancer, or bladder cancer), as in the clinical trial NCT01633970.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with pemetrexed (e.g., ALIMTA®) and carboplatin (e.g., PARAPLATIN®) (e.g., in the treatment of locally advanced or metastatic tumors, e.g., in the treatment of breast cancer, cervical cancer, kidney cancer, gastric cancer, ovarian cancer, or bladder cancer), as in the clinical trial NCT01633970.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with etoposide (e.g., ETOPOPHOS®, TOPOSAR®) and carboplatin (e.g., PARAPLATIN®) (e.g., in the treatment of lung cancer (e.g., small cell lung cancer (SCLC))), as in the clinical trial NCT02748889.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with paclitaxel (e.g., albumin-bound paclitaxel (nab-paclitaxel (ABRAXANE®)) and carboplatin (e.g., PARAPLATIN®) (e.g., in the treatment of locally advanced or metastatic tumors, e.g., in the treatment of lung cancer (NSCLC)), as in the clinical trial NCT02716038.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with epacadostat (e.g., INCB024360) (e.g., in the treatment of lung cancer (e.g., NSCLC) or bladder cancer (e.g., urothelial carcinoma)), as in the clinical trial NCT02298153.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with radiation therapy and a chemotherapy (e.g., carboplatin and/or paclitaxel), e.g., in the treatment of lung cancer (e.g., NSCLC), as in the clinical trial NCT02525757.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with veliparib (e.g., in the treatment of breast cancer, e.g., TNBC, BRCA1 gene mutation, BRCA2 gene mutation, estrogen receptor negative breast cancer, Her2/Neu negative breast cancer, stage IIIA breast cancer, stage IIIB breast cancer, stage IIIC breast cancer, or stage IV breast cancer), as in the clinical trial NCT02849496.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with alectinib (also known as ALECENSA®) (e.g., in the treatment of lung cancer (e.g., NSCLC), as in the clinical trial NCT02013219.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with erlotinib (also known as TARCEVA®) (e.g., in the treatment of lung cancer (e.g., NSCLC), as in the clinical trial NCT02013219.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with MTIG7192A (e.g., in the treatment of advanced metastatic tumors), as in the clinical trial NCT02794571.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with vemurafenib (also known as ZELBORAF®) (e.g., in the treatment of skin cancer (e.g., a malignant melanoma), as in the clinical trial NCT01656642.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with vemurafenib (also known as ZELBORAF®) and cobimetinib (also known as (COTELLIC®) (e.g., in the treatment of skin cancer (e.g., a malignant melanoma)), as in the clinical trial NCT01656642.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with bevacizumab (also known as AVASTIN®, Genentech) (e.g., in the treatment of ovarian, fallopian tube, or peritoneal cancer), as in the clinical trial NCT02839707.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with obinutuzumab (e.g., in the treatment of lymphoma (e.g., lymphocytic lymphoma or relapsed refractory or chronic lymphocytic leukemia (CLL))), as in the clinical trial NCT02846623.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with carboplatin and pemetrexed (e.g., in the treatment of lung cancer (e.g., NSCLC)), as in clinical trial NCT02657434.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with cisplatin and pemetrexed (e.g., in the treatment of lung cancer (e.g., NSCLC)), as in the clinical trial NCT02657434.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with tazemetostat (e.g., in the treatment of lymphoma (e.g., follicular lymphoma or diffuse large b-cell lymphoma)), as in the clinical trial NCT02220842.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with obinutuzumab (e.g., in the treatment of lymphoma (e.g., follicular lymphoma or diffuse large b-cell lymphoma)), as in the clinical trial NCT02220842.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with lenalidomide (e.g., in the treatment of multiple myeloma), as in the clinical trial NCT02431208.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with daratumumab (e.g., in the treatment of multiple myeloma), as in the clinical trial NCT02431208.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with daratumumab and lenalidomide (e.g., in the treatment of multiple myeloma), as in the clinical trial NCT02431208.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with daratumumab and pomalidomide (e.g., in the treatment of multiple myeloma), as in the clinical trial NCT02431208.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with bevacizumab (also known as AVASTIN®, Genentech) (e.g., in the treatment of kidney cancer (e.g., renal cell carcinoma)), as in the clinical trial NCT02420821.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with stereotactic body radiation (e.g., in the treatment of lung cancer (e.g., NSCLC)), as in the clinical trial NCT02400814.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with rociletinib (e.g., in the treatment of lung cancer (e.g., NSCLC)), as in the clinical trial NCT02630186.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with GDC-0919 (e.g., in the treatment of a solid tumor (e.g., renal cell cancer (RCC), urothelial bladder cancer (UBC), triple-negative breast cancer (TNBC), non-small cell lung cancer (NSCLC), melanoma, head and neck squamous cell carcinoma (HNSCC), gastric cancer, ovarian cancer, cervical cancer, endometrial cancer, or Merkel cell cancer)), as in the clinical trial NCT02471846.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with radium-223 dichloride (e.g., in the treatment of lung prostate cancer (e.g., castrate-resistant prostate cancer)), as in the clinical trial NCT02814669.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with MOXR0916 (e.g., in the treatment of a solid tumor (e.g., locally advanced or metastatic solid tumors)), as in the clinical trial NCT02410512.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with bevacizumab (also known as AVASTIN®, Genentech) and MOXR0916 (e.g., in the treatment of a solid tumor (e.g., locally advanced or metastatic solid tumors)), as in the clinical trial NCT02410512.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with azacitidine (e.g., in the treatment of a solid tumor (e.g., myelodysplastic syndromes)), as in the clinical trial NCT02508870.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with paclitaxel (e.g., albumin-bound paclitaxel (nab-paclitaxel (ABRAXANE®)) (e.g., in the treatment of breast cancer (e.g., TNBC))) as in the clinical trial NCT02425891.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with lenalidomide and obinutuzumab (e.g., in the treatment of lymphoma), as in the clinical trial NCT02631577.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with etoposide (e.g., ETOPOPHOS®, TOPOSAR®) and carboplatin (e.g., PARAPLATIN®) (e.g., in the treatment of lung cancer (e.g., small cell lung cancer (SCLC))), as in the clinical trial NCT02763579.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with ipilimumab (e.g., in the treatment of locally advanced or metastatic solid tumors), as in the clinical trial NCT02174172.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with interferon alfa-2b (e.g., in the treatment of locally advanced or metastatic solid tumors (e.g., NSCLC, melanoma, or RCC)), as in the clinical trial NCT02174172.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with hypofractionated image-guided radiotherapy (e.g., in the treatment of lung cancer (e.g., NSCLC)), as in the clinical trial NCT02463994.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with CDX-1401 (e.g., in the treatment of lung cancer (e.g., NSCLC)), as in the clinical trial NCT02495636.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with CDX-1401 (e.g., in the treatment of lung cancer (e.g., NSCLC)), as in the clinical trial NCT02495636.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with trastuzumab and pertuzumab (e.g., in the treatment of breast cancer (e.g., Her2-positive breast cancer)), as in the clinical trial NCT02605915.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with trastuzumab emtansine (e.g., in the treatment of breast cancer (e.g., Her2-positive breast cancer)), as in the clinical trial NCT02605915.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with doxorubicin and cyclophosphamide (e.g., in the treatment of breast cancer (e.g., Her2-positive breast cancer)), as in the clinical trial NCT02605915.

In some instances, the PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with trastuzumab, pertuzumab, and docetaxel (e.g., in the treatment of breast cancer (e.g., Her2-positive breast cancer)), as in the clinical trial NCT02605915.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with bevacizumab (also known as AVASTIN®) (e.g., in the treatment of kidney cancer (e.g., advanced non-clear cell kidney cancer)), as in the clinical trial NCT02724878.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with CMB305 (e.g., in the treatment of sarcoma (e.g., myxoid/round cell liposarcoma, synovial sarcoma, metastatic sarcoma, recurrent adult soft tissue sarcoma, locally advanced sarcoma, or liposarcoma)), as in the clinical trial NCT02609984.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with R07009789 (e.g., in the treatment of solid cancers (e.g., locally advanced and metastatic solid tumors)), as in the clinical trial NCT02304393.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with Bacille Calmette-Guérin (also known as ONCOTICE®) (e.g., in the treatment of bladder cancer (e.g., non-muscle invasive bladder cancer)), as in the clinical trial NCT02792192.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with stereotactic body radiation therapy (e.g., in the treatment of lung cancer (e.g., NSCLC)), as in the clinical trial NCT02599454.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with carboplatin, and nab-paclitaxel (also known as ABRAXANE®) (e.g., in the treatment of breast cancer (e.g., invasive ductal breast carcinoma)), as in the clinical trial NCT02620280.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with carboplatin, nab-paclitaxel (also known as ABRAXANE®), and an adjuvant chemotherapy including AC or EC (adriamycin or epirubicin and cyclophosphamide) or FEC (fluorouracil, epirubicin, and cyclophosphamide) (e.g., in the treatment of breast cancer (e.g., invasive ductal breast carcinoma)), as in the clinical trial NCT02620280.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with gemcitabine and carboplatin or cisplatin (e.g., in the treatment of urothelial carcinoma), as in the clinical trial NCT02807636.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with paclitaxel and carboplatin (e.g., in the treatment of lung cancer (e.g., NSCLC, e.g., non-squamous NSCLC)), as in the clinical trial NCT02366143.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with bevacizumab, paclitaxel, and carboplatin (e.g., in the treatment of lung cancer (e.g., NSCLC, e.g., non-squamous NSCLC)), as in the clinical trial NCT02366143.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with cergutuzumab (also known as R06895882) (e.g., in the treatment of locally advanced and/or metastatic solid tumors), as in the clinical trial NCT02350673.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with bendamustine and obinutuzumab (e.g., in the treatment of lymphoma (e.g., diffuse large B-cell lymphoma or follicular lymphoma)), as in the clinical trial NCT02596971.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with bendamustine, cyclophosphamide, obinutuzumab, prednisone, and vincristine (e.g., in the treatment of lymphoma (e.g., diffuse large B-cell lymphoma or follicular lymphoma)), as in the clinical trial NCT02596971.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with cyclophosphamide, doxorubicin, obinutuzumab, prednisone, and vincristine (e.g., in the treatment of lymphoma (e.g., diffuse large B-cell lymphoma or follicular lymphoma)), as in the clinical trial NCT02596971.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with cyclophosphamide, doxorubicin, prednisone, vincristine, and rituximab (e.g., in the treatment of lymphoma (e.g., diffuse large B-cell lymphoma or follicular lymphoma)), as in the clinical trial NCT02596971.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with R06958688 (e.g., in the treatment of locally advanced and/or metastatic solid tumors (e.g., carcinoembryonic antigen (CEA)-positive solid tumors)), as in the clinical trial NCT02650713.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with acetylsalicylic acid (e.g., in the treatment of ovarian cancer (e.g., ovarian neoplasms)), as in the clinical trial NCT02659384.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with bevacizumab (e.g., in the treatment of ovarian cancer (e.g., ovarian neoplasms)), as in the clinical trial NCT02659384.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with vanucizumab (also known as R05520985) (e.g., in the treatment of locally advanced and/or metastatic solid tumors), as in the clinical trial NCT01688206.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with carboplatin and nab-paclitaxel (e.g., in the treatment of lung cancer (e.g., non-squamous NSCLC)), as in the clinical trial NCT02367781.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with bevacizumab (also known as AVASTIN®) (e.g., in the treatment of kidney cancer (e.g., renal cell carcinoma)), as in the clinical trial NCT01984242.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with cobimetinib (also known as GDC-0973) (e.g., in the treatment of locally advanced or metastatic solid tumors), as in the clinical trial NCT01988896.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with R05509554 (e.g., in the treatment of locally advanced solid tumors (e.g., locally advanced and/or metastatic triple negative breast cancer, ovarian cancer, bladder cancer, gastric cancer, or soft tissue sarcoma)), as in the clinical trial NCT02323191.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with varlilumab (e.g., in the treatment of advanced cancer (e.g., melanoma, RCC, triple negative breast cancer, bladder cancer, head and neck cancer, or non-small cell lung cancer)), as in the clinical trial NCT02543645.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with cobimetinib (e.g., in the treatment of colorectal cancer), as in the clinical trial NCT02788279.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with cobimetinib (e.g., in the treatment of colorectal cancer), as in the clinical trial NCT02788279.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with bevacizumab (also known as AVASTIN®) (e.g., in the treatment of solid tumors), as in the clinical trial NCT02715531.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with bevacizumab (also known as AVASTIN®), leucovorin, oxaliplatin, and optionally, capecitabine (e.g., in the treatment of solid tumors), as in the clinical trial NCT02715531.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with nab-paclitaxel and gemcitabine (e.g., in the treatment of solid tumors), as in the clinical trial NCT02715531.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with oxaliplatin, leucovorin, 5-fluorouracil (5-FU), oxaliplatin, and cisplatin (e.g., in the treatment of solid tumors), as in the clinical trial NCT02715531.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with nab-paclitaxel and carboplatin (e.g., in the treatment of lung cancer (e.g., squamous NSCLC)), as in the clinical trial NCT02367794.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with paclitaxel and carboplatin (e.g., in the treatment of lung cancer (e.g., squamous NSCLC)), as in the clinical trial NCT02367794.

In some instances, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)) may be administered in conjunction with CPI-444 (e.g., in the treatment of advanced cancers (e.g., non-small cell lung cancer, malignant melanoma, renal cell cancer, triple negative breast cancer, colorectal cancer with microsatellite instability (MSI), and bladder cancer)), as in the clinical trial NCT02655822.

### IV. PHARMACEUTICAL COMPOSITIONS AND FORMULATIONS

Pharmaceutical compositions and formulations as described herein can be prepared by mixing the active ingredient(s) (e.g., an anti-PD-L1 antibody (MPDL3280A) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®; Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases. It is understood that any of the above pharmaceutical compositions or formulations may include an immunoconjugate described herein in place of, or in addition to, a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The compositions and formulations herein may also contain more than one active ingredients as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g., films, or microcapsules. The formulations to be used for in vivo administration are generally sterile. Sterility may be readily accomplished, for example, by filtration through sterile filtration membranes.

### V. ARTICLES OF MANUFACTURE AND KITS

In another aspect of the invention, an article of manufacture or kit containing materials useful for the treatment, prevention, and/or diagnosis of individuals is provided.

In some instances, such articles of manufacture or kits can be used to identify an individual having a cancer (e.g., lung cancer (e.g., NSCLC), bladder cancer (e.g., UBC), kidney cancer (e.g., RCC), or breast cancer (e.g., TNBC)) who may benefit from a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). Such articles of manufacture or kits may include (a) reagents for determining the immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4) in a sample from the individual and (b) instructions for using the reagents to identify an individual having a cancer (e.g., lung cancer (e.g., NSCLC), bladder cancer (e.g., UBC), kidney cancer (e.g., RCC), or breast cancer (e.g., TNBC)) who may benefit from a treatment including a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

For example, in some instances, the article of manufacture or kit includes (a) reagents for determining the immune-score expression level of PD-L1, CXCL9, and IFNG in a sample from the individual and (b) instructions for using the reagents to identify an individual having a cancer (e.g., lung cancer (e.g., NSCLC), bladder cancer (e.g., UBC), kidney cancer (e.g., RCC), or breast cancer (e.g., TNBC)) who may benefit from a treatment comprising a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, the article of manufacture or kit includes (a) reagents for determining the immune-score expression level of PD-L1, IFNG, GZMB, and CD8A in a sample from the individual and (b) instructions for using the reagents to identify an individual having a cancer (e.g., lung cancer (e.g., NSCLC), bladder cancer (e.g., UBC), kidney cancer (e.g., RCC), or breast cancer (e.g., TNBC)) who may benefit from a treatment comprising a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)). In some instances, the article of manufacture or kit includes (a) reagents for determining the immune-score expression level of PD-L1, IFNG, GZMB, CD8A, and PD-1 in a sample from the individual and (b) instructions for using the reagents to identify an individual having a cancer (e.g., lung cancer (e.g., NSCLC), bladder cancer (e.g., UBC), kidney cancer (e.g., RCC), or breast cancer (e.g., TNBC)) who may benefit from a treatment comprising a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)).

In some instances, such articles of manufacture or kits include a PD-L1 axis binding antagonist (e.g., PD-L1 binding antagonist, e.g., an anti-PD-L1 antibody, e.g., atezolizumab (MPDL3280A)) for treating an individual with a cancer (e.g., lung cancer (e.g., NSCLC), bladder cancer (e.g., UBC), kidney cancer (e.g., RCC), or breast cancer (e.g., TNBC)). In some instances, the article of manufacture or kit includes (a) a PD-L1 axis binding antagonist (e.g., PD-L1 binding antagonist, e.g., an anti-PD-L1 antibody, e.g., atezolizumab (MPDL3280A)) and (b) a package insert including instructions for administration of the PD-L1 axis binding antagonist (e.g., PD-L1 binding antagonist, e.g., an anti-PD-L1 antibody, e.g., atezolizumab (MPDL3280A)) to an individual having a cancer (e.g., lung cancer (e.g., NSCLC), bladder cancer (e.g., UBC), kidney cancer (e.g., RCC), or breast cancer (e.g., TNBC)), wherein, prior to treatment, the immune-score expression level of at least one, at least two, at least three, at least four, at least five, or all six genes selected from the group consisting of PD-L1, CXCL9, IFNG, GZMB, CD8A, and PD-1, or combinations thereof (e.g., PD-L1, CXCL9, and IFNG; PD-L1, IFNG, GZMB, and CD8A; PD-L1, IFNG, GZMB, CD8A, and PD-1; or any one of the combinations of genes listed in Tables 1-4)) in a sample from the individual has been determined and at least one, at least two, at least three, at least four, at least five, or all six of PD-L1, CXCL9, IFNG, GZMB, CD8A, or PD-1 in the sample is above a reference immune-score expression level.

For example, in some instances, the article of manufacture or kit includes (a) a PD-L1 axis binding antagonist (e.g., PD-L1 binding antagonist, e.g., an anti-PD-L1 antibody, e.g., atezolizumab (MPDL3280A)) and (b) a package insert including instructions for administration of the PD-L1 axis binding antagonist (e.g., PD-L1 binding antagonist, e.g., an anti-PD-L1 antibody, e.g., atezolizumab (MPDL3280A)) to an individual having a cancer (e.g., lung cancer (e.g., NSCLC), bladder cancer (e.g., UBC), kidney cancer (e.g., RCC), or breast cancer (e.g., TNBC)), wherein, prior to treatment, the immune-score expression level of PD-L1, CXCL9, and IFNG in a sample from the individual has been determined and at least one, at least two, or all three of PD-L1, CXCL9, and IFNG in the sample is above a reference immune-score expression level. In some instances, the article of manufacture or kit includes (a) a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)); and (b) a package insert including instructions for administration of the PD-L1 axis binding antagonist (e.g., PD-L1 binding antagonist, e.g., an anti-PD-L1 antibody, e.g., atezolizumab (MPDL3280A)) to an individual having a cancer (e.g., lung cancer (e.g., NSCLC), bladder cancer (e.g., UBC), kidney cancer (e.g., RCC), or breast cancer (e.g., TNBC)), wherein, prior to treatment, the immune-score expression level of PD-L1, IFNG, GZMB, and CD8A in a sample from the individual has been determined and at least one, at least two, at least three, or all four of PD-L1, IFNG, GZMB, and CD8A in the sample is above a reference immune-score expression level. In some instances, the article of manufacture or kit includes (a) a PD-L1 axis binding antagonist (e.g., PD-L1 binding antagonist (e.g., anti-PD-L1 antibody, e.g., atezolizumab (MPDL3280A)) or PD-1 binding antagonist (e.g., anti-PD-1 antibody)); and (b) a package insert including instructions for administration of the PD-L1 axis binding antagonist (e.g., PD-L1 binding antagonist, e.g., an anti-PD-L1 antibody, e.g., atezolizumab (MPDL3280A)) to an individual having a cancer (e.g., lung cancer (e.g., NSCLC), bladder cancer (e.g., UBC), kidney cancer (e.g., RCC), or breast cancer (e.g., TNBC)), wherein, prior to treatment, the immune-score expression level of PD-L1, IFNG, GZMB, CD8A, and PD-1 in a sample from the individual has been determined and at least one, at least two, at least three, at least four, or all five of PD-L1, IFNG, GZMB, CD8A, and PD-1 in the sample is above a reference immune-score expression level.

Any of the articles of manufacture or kits described may include a carrier means being compartmentalized to receive in close confinement one or more container means such as vials, tubes, and the like, each of the container means comprising one of the separate elements to be used in the method. Where the article of manufacture or kit utilizes nucleic acid hybridization to detect the target nucleic acid, the kit may also have containers containing nucleotide(s) for amplification of the target nucleic acid sequence and/or a container comprising a reporter-means, such as an enzymatic, florescent, or radioisotope label.

In some instances, the article of manufacture or kit includes the container described above and one or more other containers including materials desirable from a commercial and user standpoint, including buffers, diluents, filters, needles, syringes, and package inserts with instructions for use. A label may be present on the container to indicate that the composition is used for a specific application, and may also indicate directions for either in vivo or in vitro use, such as those described above. For example, the article of manufacture or kit may further include a container including a pharmaceutically-acceptable buffer, such as bacteriostatic water for injection (BWFI), phosphate-buffered saline, Ringer's solution, and dextrose solution.

The articles of manufacture or kits described herein may have a number of embodiments. In one instance, the article of manufacture or kit includes a container, a label on said container, and a composition contained within said container, wherein the composition includes one or more polynucleotides that hybridize to a complement of a gene listed herein (e.g., PD-L1, CXCL9, IFNG, GZMB, CD8A, or PD-1) under stringent conditions, and the label on said container indicates that the composition can be used to evaluate the presence of a gene listed herein (e.g., PD-L1, CXCL9, IFNG, GZMB, CD8A, or PD-1) in a sample, and wherein the kit includes instructions for using the polynucleotide(s) for evaluating the presence of the gene RNA or DNA in a particular sample type.

For oligonucleotide-based articles of manufacture or kits, the article of manufacture or kit can include, for example: (1) an oligonucleotide, e.g., a detectably labeled oligonucleotide, which hybridizes to a nucleic acid sequence encoding a protein or (2) a pair of primers useful for amplifying a nucleic acid molecule. The article of manufacture or kit can also include, e.g., a buffering agent, a preservative, or a protein stabilizing agent. The article of manufacture or kit can further include components necessary for detecting the detectable label (e.g., an enzyme or a substrate). The article of manufacture or kit can also contain a control sample or a series of control samples that can be assayed and compared to the test sample. Each component of the article of manufacture or kit can be enclosed within an individual container and all of the various containers can be within a single package, along with instructions for interpreting the results of the assays performed using the kit.

### VI. EXAMPLES

The following is an example of the methods of the invention. It is understood that various other embodiments may be practiced, given the general description provided above.

### Example 1. Association Between Immune-Score Expression Levels of (i) PD-L1, CXCL9, and IFNG or (ii) PD-L1, IFNG, GZMB, and CD8A and Clinical Response of Patients Having Non-Small Cell Lung Cancer (NSCLC) to Treatment with Atezolizumab (MPDL3280A)

An RNA-based molecular assay was used to evaluate the association between clinical response to treatment with atezolizumab (MPDL3280A), an anti-PD-L1-antibody, and immune-score expression levels of (i) PD-L1, CXCL9, and IFNG or (ii) PD-L1, IFNG, GZMB, and CD8A in patients with non-small cell lung cancer (NSCLC) enrolled in a phase III clinical trial in which atezolizumab was administered as a monotherapy.

### Study Design

The OAK (Clinical Trial ID No.: NCT02008227) patient population evaluated for (i) PD-L1, CXCL9, and IFNG and (ii) PD-L1, IFNG, GZMB, and CD8A expression levels consisted of 753 patients. Patients were eligible for enrollment in the OAK Trial if they had locally advanced or metastatic (e.g., stage IIIB, stage IV, or recurrent) NSCLC; disease progression during or following treatment with a prior platinum-containing regimen for locally advanced, unresectable/inoperable, or metastatic NSCLC, or disease recurrence within 6 months of treatment with a platinum-based adjuvant/neoadjuvant regimen; measurable disease, as defined by RECIST v1.1; and an Eastern Cooperative Oncology Group (ECOG) performance status of 0 or 1. Participants were randomized to receive either atezolizumab at a dose of 1200 mg intravenously every three weeks or docetaxel 75 mg per square meter (mg/m²) intravenously every three weeks. Treatment with atezolizumab could be continued as long as participants were experiencing clinical benefit, i.e., in the absence of unacceptable toxicity or symptomatic deterioration attributed to disease progression.

### Analysis of PD-L1, CXCL9, and IFNG Expression and Efficacy of MPDL3280A

To evaluate whether PD-L1, CXCL9, and IFNG gene expression status was associated with patient response to atezolizumab (MPDL3280A) treatment, the immune-score expression level of PD-L1, CXCL9, and IFNG was assessed in pre-treatment, formalin-fixed and paraffin-embedded (FFPE) tumor samples obtained from each patient. RNA was isolated from the FFPE tumor sections and PD-L1, CXCL9, and IFNG gene expression was measured using PCR-based methodology. The expression level, expressed as the cycle threshold (Ct) for each of PD-L1, CXCL9, and IFNG was normalized to the expression level of a housekeeping gene (e.g., TMEM55B). The normalized expression value, dCt, where dCt(target gene)=Ct(control gene)−Ct(target gene) for each of PD-L1, CXCL9, and IFNG, was then averaged to obtain a single numerical averaged dCt value for the immune-score expression level of PD-L1, CXCL9, and IFNG.

Tumor specimens obtained from the patients were categorized into different high or low expression level subgroups based on the immune-score expression level of PD-L1, CXCL9, and IFNG relative to a cut-off value for a given percentile (e.g., 25.5^{th} percentile, 50.2^{th} percentile, 70.3^{th} percentile, and 75.3^{th} percentile) of the population. The 25.5^{th} percentile cut-off value was defined by the immune-score expression levels of PD-L1, CXCL9, and IFNG greater than or equal to 25.5% of all immune-score expression levels of PD-L1, CXCL9, and IFNG in the population analyzed. The 50.2^{th} percentile cut-off was defined by the immune-score expression levels of PD-L1, CXCL9, and IFNG greater than or equal to 50.2% of all immune-score expression levels of PD-L1, CXCL9, and IFNG in the population analyzed. The 70.3^{th} percentile cut-off was defined by the immune-score expression levels of PD-L1, CXCL9, and IFNG greater than or equal to 70.3% of all immune-score expression levels of PD-L1, CXCL9, and IFNG in the population analyzed. The 75.3^{th} percentile cut-off was defined by the immune-score expression levels of PD-L1, CXCL9, and IFNG greater than or equal to 75.3% of all immune-score expression levels of PD-L1, CXCL9, and IFNG in the population analyzed.

The efficacy results for the atezolizumab and the docetaxel arms of the OAK Trial were compared for the high expression level and low expression level subgroups for each percentile cut-off. High expression levels were defined as PD-L1, CXCL9, and IFNG immune-score expression levels at or above each percentile cut-off. Low expression levels were defined as PD-L1, CXCL9, and IFNG immune-score expression levels below each percentile cut-off. The immune-score expression levels of PD-L1, CXCL9, and IFNG across percentile cut-offs are presented in Table 6.

TABLE 6
PD-L1, CXCL9, and IFNG immune-score expression levels
across percentile cut-offs in the OAK Trial

Expression Level Cut-Offs

| Population | 25.5% | 50.2% | 70.3% | 75.3% |
|---|---|---|---|---|
| OAK | −3.2 | −1.9 | −1.03 | −0.8 |

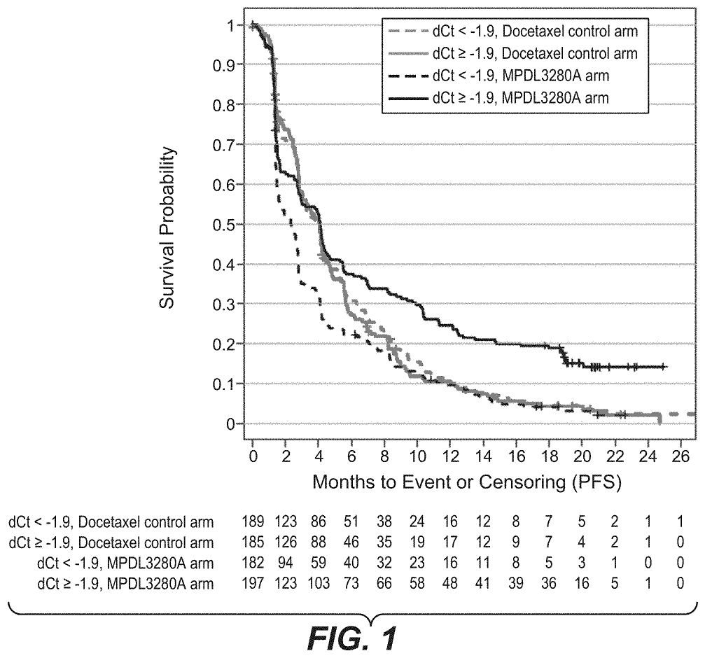

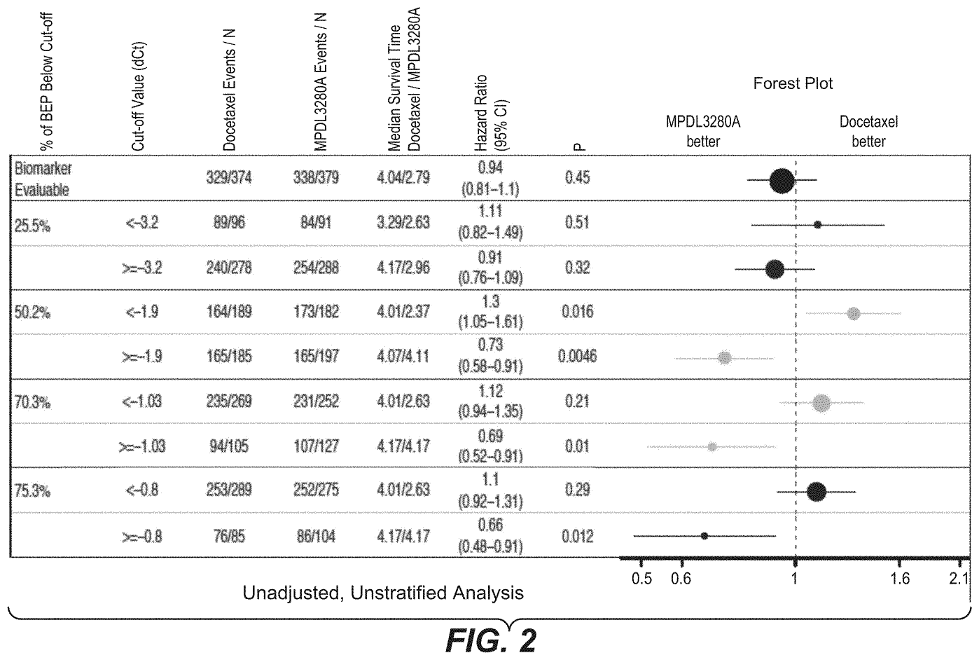

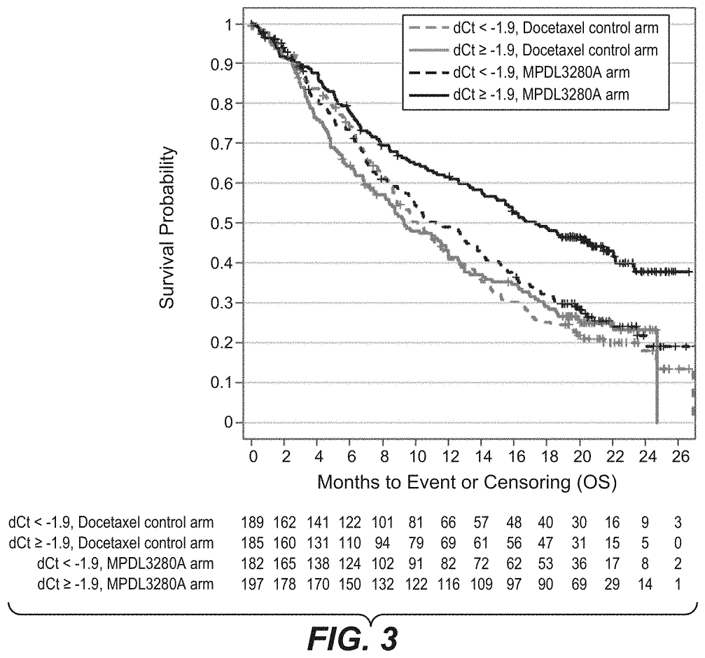

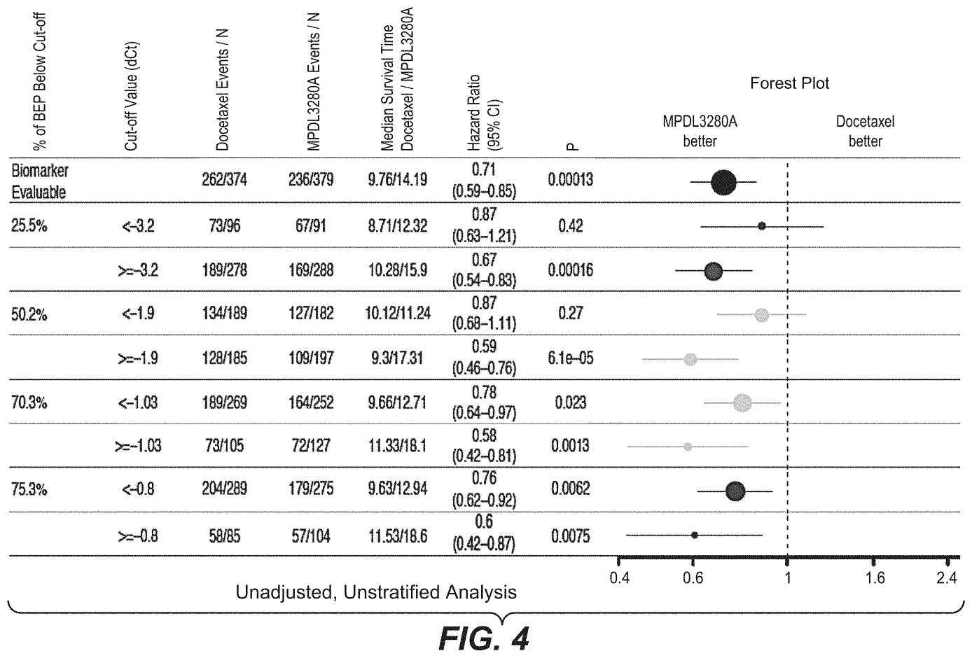

The overall survival (OS) and progression free survival (PFS) endpoints from the OAK Trial were evaluated against the PD-L1, CXCL9, and IFNG expression level cut-offs defined herein (e.g., the 25.5^{th}, 50.2^{th}, 70.3^{th}, or 75.3^{th} expression level percentile cut-offs). The analysis shows a trend towards an association of immune-score expression levels of PD-L1, CXCL9, and IFNG with improved efficacy of treatment with atezolizumab as compared to treatment with docetaxel in patients having NSCLC in the randomized OAK Trial (FIGS. 1-4). A gradient of increasing PFS and OS benefit was observed with increasing PD-L1, CXCL9, and IFNG expression levels (FIGS. 1-4). A summary of the association of expression levels of PD-L1, CXCL9, and IFNG with efficacy endpoints in the OAK patient population is presented in Table 7.

TABLE 7
Summary of the association of PD-L1, CXCL9, and IFNG expression
levels with efficacy endpoints in the OAK Trial

| Expression Level | | | | |
|---|---|---|---|---|
| Percentile | PFS HR (95% CI)^{a} | | OS HR (95% CI)^{a} | |
| ≥25.5% | 0.91 | (0.76, 1.09) | 0.67 | (0.54, 0.83) |
| ≥50.2% | 0.73 | (0.58, 0.91) | 0.59 | (0.46, 0.76) |
| ≥70.3% | 0.69 | (0.52, 0.91) | 0.58 | (0.42, 0.81) |
| ≥75.3% | 0.66 | (0.48, 0.91) | 0.6 | (0.42, 0.87) |
| BEP (n = 379 vs 374)^{b} | 0.94 | (0.81, 1.1) | 0.71 | (0.59, 0.85) |

BEP, biomarker evaluable population.
^{a}Unadjusted and unstratified HRs for atezolizumab vs docetaxel at each expression level percentile cut-off.
^{b}Number of patients in atezolizumab vs docetaxel treatment arms.

Together, these data show that the immune-score expression level of PD-L1, CXCL9, and IFNG can serve as a predictive biomarker that is predictive of therapeutic efficacy of a treatment including a PD-L1 binding antagonist (e.g., an anti-PD-L1 antibody, e.g., atezolizumab (MPDL3280A)). Consequently, evaluation of the expression levels of PD-L1, CXCL9, and IFNG (e.g., the immune-score expression level of PD-L1, CXCL9, and IFNG) can be used, for example, to identify patients having a cancer (e.g., NSCLC) who derive a PFS benefit and an OS benefit from treatment including a PD-L1 binding antagonist (e.g., an anti-PD-L1 antibody, e.g., atezolizumab (MPDL3280A)).

### Analysis of PD-L1, IFNG, GZMB, and CD8A Expression and Efficacy of MPDL3280A

To evaluate whether PD-L1, IFNG, GZMB, and CD8A gene expression status was associated with patient response to atezolizumab (MPDL3280A) treatment, the gene expression level of PD-L1, IFNG, GZMB, and CD8A was assessed in pre-treatment, FFPE tumor sections and PD-L1, IFNG, GZMB, and CD8A gene expression was measured using PCR-based methodology. The expression level, expressed as the cycle threshold (Ct) for each of PD-L1, IFNG, GZMB, and CD8A was normalized to the expression level of a housekeeping gene (e.g., TMEM55B). The normalized expression value, dCt, where dCt(target gene)=Ct(control gene)−Ct(target gene) for each of PD-L1, IFNG, GZMB, and CD8A, was then averaged to obtain a single numerical averaged dCt value for the aggregate expression level of PD-L1, IFNG, GZMB, and CD8A.

Tumor specimens obtained from the patients were categorized into different high or low expression level subgroups based on the immune-score expression level of PD-L1, IFNG, GZMB, and CD8A relative to a cut-off value for a given percentile (e.g., 25.4^{th} percentile, 50.2^{th} percentile, 70.1^{th} percentile, or 75^{th} percentile) of the population. The 25.4^{th} percentile was defined by the immune-score expression levels of PD-L1, IFNG, GZMB, and CD8A greater than or equal to 25.4% of all immune-score expression levels of PD-L1, IFNG, GZMB, and CD8A in the population analyzed. The 50.2^{th} percentile cut-off was defined by the immune-score expression levels of PD-L1, IFNG, GZMB, and CD8A greater than or equal to 50.2% of all immune-score expression levels of PD-L1, IFNG, GZMB, and CD8A in the population analyzed. The 70.1^{th} percentile cut-off was defined by the immune-score expression levels of PD-L1, IFNG, GZMB, and CD8A greater than or equal to 70.1% of all immune-score expression levels of PD-L1, IFNG, GZMB, and CD8A in the population analyzed. The 75^{th} percentile cut-off was defined by the immune-score expression levels of PD-L1, IFNG, GZMB, and CD8A greater than or equal to 75% of all immune-score expression levels of PD-L1, IFNG, GZMB, and CD8A in the population analyzed.

The efficacy results for the atezolizumab and the docetaxel arms of the OAK Trial were compared for the high expression level and low expression level subgroups for each percentile cut-off. High expression levels were defined as PD-L1, IFNG, GZMB, and CD8A immune-score expression levels at or above each percentile cut-off. Low expression levels were defined as PD-L1, IFNG, GZMB, and CD8A immune-score expression levels below each percentile cut-off. The immune-score expression levels of PD-L1, IFNG, GZMB, and CD8A across percentile cut-offs are presented in Table 8.

TABLE 8
PD-L1, IFNG, GZMB, and CD8A immune-score expression
levels across percentile cut-offs in the OAK Trial

Expression Level Cut-Offs

| Population | 25.4% | 50.2% | 70.1% | 75% |
|---|---|---|---|---|
| OAK | −2.48 | −1.32 | −0.7 | −0.48 |

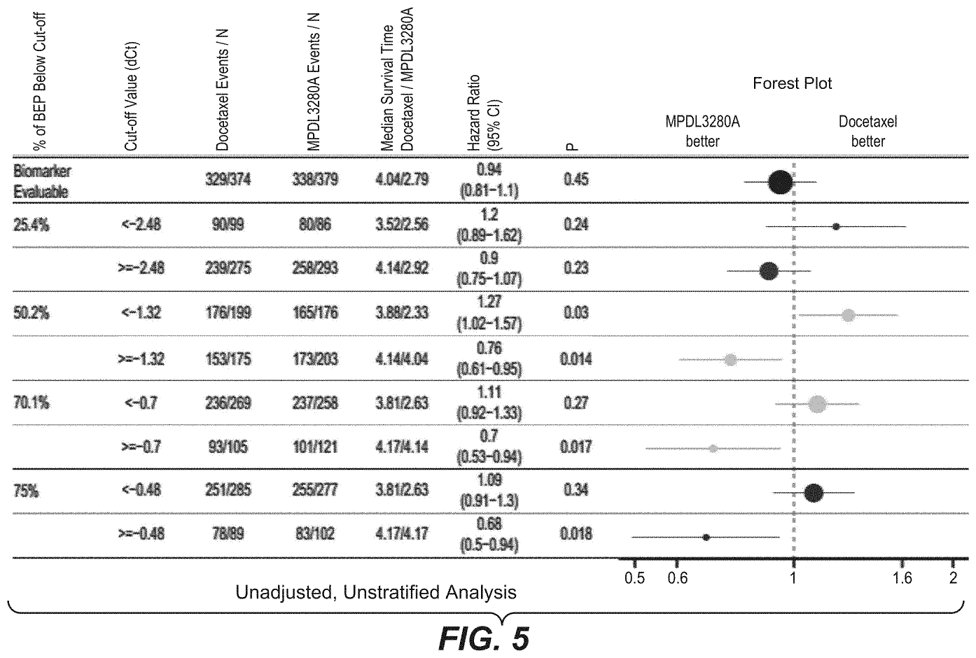

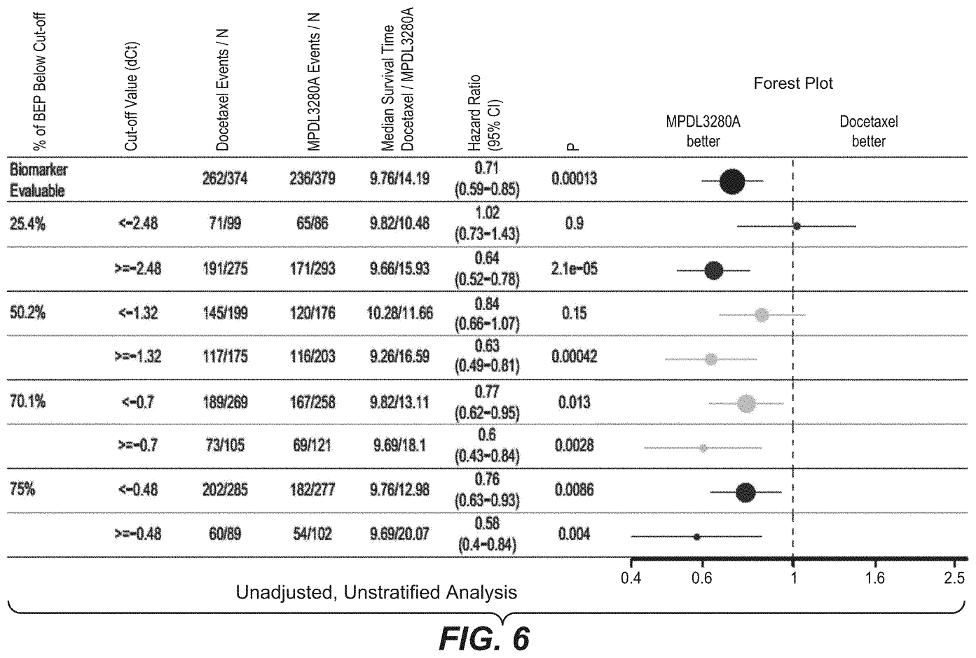

The OS and PFS endpoints from the OAK Trial were evaluated against the PD-L1, IFNG, GZMB, and CD8A expression level cut-offs defined herein (e.g., the 25.4^{th}, 50.2^{th}, 70.1^{th}, and 75^{th} expression level percentile cut-offs). The analysis shows a trend towards an association of immune-score expression levels of PD-L1, IFNG, GZMB, and CD8A with improved efficacy of treatment with atezolizumab as compared to treatment with docetaxel in patients having NSCLC in the randomized OAK Trial (FIGS. 5 and 6). A gradient of increasing PFS and OS benefit was observed with increasing PD-L1, IFNG, GZMB, and CD8A expression levels (FIGS. 5 and 6). A summary of the association of expression levels of PD-L1, IFNG, GZMB, and CD8A with efficacy endpoints in the OAK patient population is presented in Table 9.

TABLE 9
Summary of the association of PD-L1, IFNG, GZMB, and CD8A
expression levels with efficacy endpoints in the OAK Trial

| Expression Level | | | | |
|---|---|---|---|---|
| Percentile | PFS HR (95% CI)^{a} | | OS HR (95% CI)^{a} | |
| ≥25.4% | 0.9 | (0.75, 1.07) | 0.64 | (0.52, 0.78) |
| ≥50.2% | 0.76 | (0.61, 0.95) | 0.63 | (0.49, 0.81) |
| ≥70.3% | 0.7 | (0.53, 0.94) | 0.6 | (0.43, 0.84) |
| ≥75.3% | 0.68 | (0.5, 0.94) | 0.58 | (0.4, 0.84) |
| BEP (n = 379 vs 374)^{b} | 0.94 | (0.81, 1.1) | 0.71 | (0.59, 0.85) |

BEP, biomarker evaluable population.
^{a}Unadjusted and unstratified HRs for atezolizumab vs docetaxel at each expression level percentile cut-off.
^{b}Number of patients in atezolizumab vs docetaxel treatment arms.

Together, these data show that the immune-score expression level of PD-L1, IFNG, GZMB, and CD8A can serve as a predictive biomarker that is predictive of therapeutic efficacy of a treatment including a PD-L1 binding antagonist (e.g., an anti-PD-L1 antibody, e.g., atezolizumab (MPDL3280A)). Consequently, evaluation of the expression levels of PD-L1, IFNG, GZMB, and CD8A (e.g., the immune-score expression level of PD-L1, IFNG, GZMB, and CD8A) can be used, for example, to identify patients having a cancer (e.g., NSCLC) who derive a PFS benefit and an OS benefit from treatment including a PD-L1 binding antagonist (e.g., an anti-PD-L1 antibody, e.g., atezolizumab (MPDL3280A)).

### Analyses of Five-Gene and Six-Gene Immune-Score Expression Levels

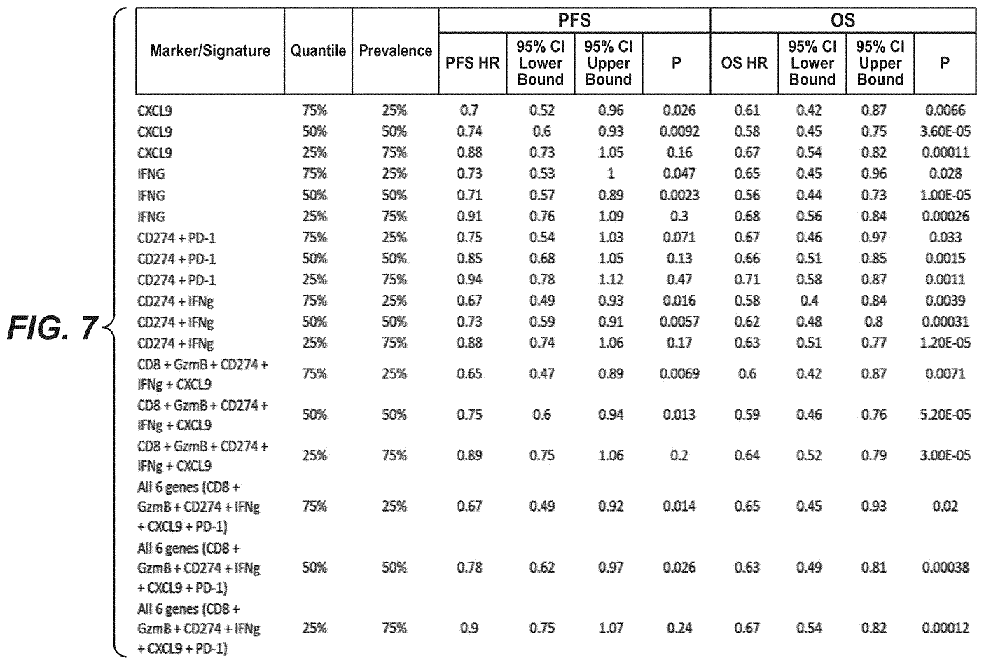

The above methodology was also used to assess the expression levels of five genes (e.g., CD8A, GZMB, PD-L1, IFNG, and CXCL9) or six genes (e.g., CD8A, GZMB, PD-L1, IFNG, CXCL9, and PD-1) in patients in the OAK Trial. Consistent with the analysis of the immune-score expression levels based on three genes (e.g., PD-L1, CXCL9, and IFNG) and four genes (e.g., PD-L1, IFNG, GZMB, and CD8A), the five-gene and six-gene analyses show an association between the immune-score expression levels of (i) CD8A, GZMB, PD-L1, IFNG, and CXCL9 or (ii) CD8A, GZMB, PD-L1, IFNG, CXCL9, and PD-1 with improved efficacy of treatment with atezolizumab as compared to treatment with docetaxel in patients having NSCLC in the OAK Trial (FIG. 7). A gradient of increasing PFS and OS benefit was observed with increasing immune-score expression levels (i.e., scores with decreasing prevalence) for (i) CD8A, GZMB, PD-L1, IFNG, and CXCL9 or (ii) CD8A, GZMB, PD-L1, IFNG, CXCL9, and PD-1 (FIG. 7). Together, these data show that the immune-score expression levels of combinations of the biomarkers comprising five-genes or all six-genes can serve as a predictive biomarker that is predictive of therapeutic efficacy of a treatment including a PD-L1 binding antagonist (e.g., an anti-PD-L1 antibody, e.g., atezolizumab (MPDL3280A)).

### Example 2. Association Between Expression Levels of PD-L1, CXCL9, and IFNG and Clinical Response of Patients Having NSCLC to Treatment with Atezolizumab (MPDL3280A)

An RNA-based molecular assay was used to evaluate the association between clinical response to treatment with atezolizumab (MPDL3280A), an anti-PD-L1 antibody, and expression levels of PD-L1, CXCL9, and IFNG in individuals with NSCLC enrolled in a phase II clinical trial in which atezolizumab was administered as a monotherapy.

### Study Design

The POPLAR (Clinical Trial ID No.: NCT01903993) patient population evaluated for PD-L1, CXCL9, and IFNG expression levels consisted of 215 patients. Patients were eligible for enrollment in the POPLAR study if they had locally advanced or metastatic (e.g., stage IIIB, stage IV, or recurrent) NSCLC; disease progression during or following treatment with a prior platinum-containing regimen for locally advanced, unresectable/inoperable, or metastatic NSCLC, or disease recurrence within 6 months of treatment with a platinum-based adjuvant/neoadjuvant regimen; measurable disease, as defined by RECIST v1.1; and an ECOG performance status of 0 or 1. Participants were randomized to receive either atezolizumab at a dose of 1200 mg intravenously every three weeks or docetaxel 75 mg per square meter (mg/m²) intravenously every three weeks. Treatment with atezolizumab could be continued as long as participants were experiencing clinical benefit, i.e., in the absence of unacceptable toxicity or symptomatic deterioration attributed to disease progression.

### Analysis of PD-L1, CXCL9, and IFNG Expression and Efficacy of MPDL3280A

To evaluate whether PD-L1, CXCL9, and IFNG gene expression status was associated with patient response to atezolizumab (MPDL3280A) treatment, the gene expression level of PD-L1, CXCL9, and IFNG was assessed in pre-treatment, FFPE tumor samples obtained from each patient. RNA was isolated from the FFPE tumor sections and PD-L1, CXCL9, and IFNG gene expression was measured using PCR-based methodology (Fluidigm). The expression level, expressed as the cycle threshold (Ct) for each of PD-L1, CXCL9, and IFNG was normalized to the expression level of a housekeeping gene (e.g., TMEM55B). The normalized expression value, dCt, where dCt(target gene)=Ct(control gene)−Ct(target gene) for each of PD-L1, CXCL9, and IFNG, was then averaged to obtain a single numerical averaged dCt value for the aggregate expression level of PD-L1, CXCL9, and IFNG.

Tumor specimens obtained from the patients were categorized into different high or low expression level subgroups based on the immune-score expression level of PD-L1, CXCL9, and IFNG relative to a cut-off value for a given percentile (25^{th}, 50^{th}, or 75^{th} percentile) of the population. The 25^{th} percentile was defined by the immune-score expression levels of PD-L1, CXCL9, and IFNG greater than or equal to 25% of all immune-score expression levels of PD-L1, CXCL9, and IFNG in the population analyzed. The 50^{th} percentile was defined by the immune-score expression levels of PD-L1, CXCL9, and IFNG greater than or equal to 50% of all immune-score expression levels of PD-L1, CXCL9, and IFNG in the population analyzed. The 75^{th} percentile was defined by the immune-score expression levels of PD-L1, CXCL9, and IFNG greater than or equal to 75% of all immune-score expression levels of PD-L1, CXCL9, and IFNG in the population analyzed.

The efficacy results for the atezolizumab and the docetaxel arms were compared for the high expression level and low expression level subgroups for each percentile cut-off. High expression levels were defined as PD-L1, CXCL9, and IFNG immune-score expression levels at or above each percentile cut-off. Low expression levels were defined as PD-L1, CXCL9, and IFNG immune-score expression levels below each percentile cut-off. The immune-score expression levels of PD-L1, CXCL9, and IFNG across percentile cut-offs are presented in Table 10.

TABLE 10
PD-L1, CXCL9, and IFNG immune-score expression levels
across percentile cut-offs in the POPLAR Trial

| | Expression Level Cut-Offs | | |
|---|---|---|---|
| Population | 25% | 50% | 75% |
| POPLAR | −1.43 | −0.33 | −0.94 |

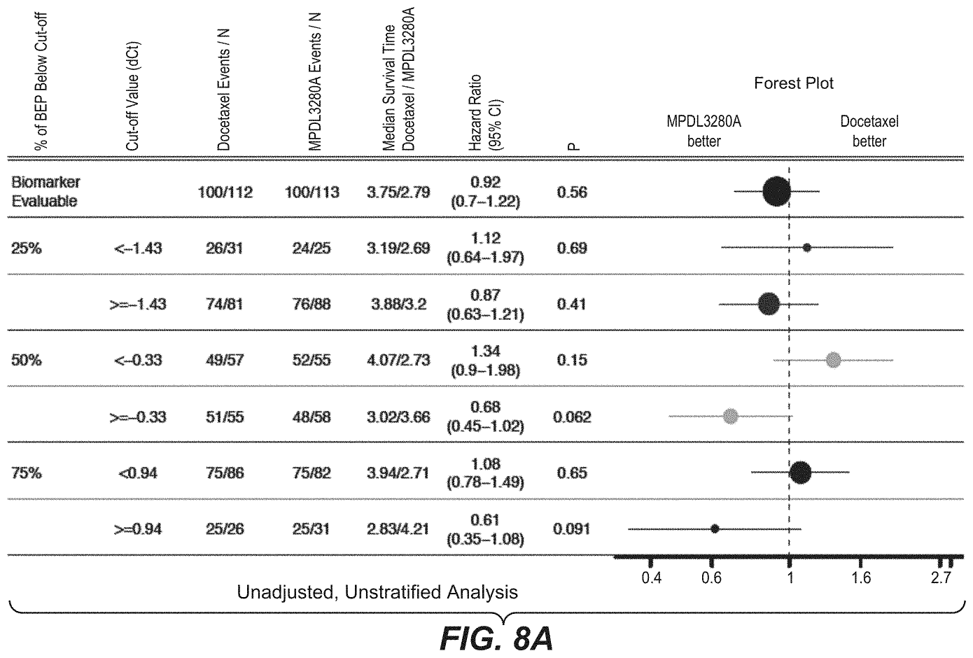

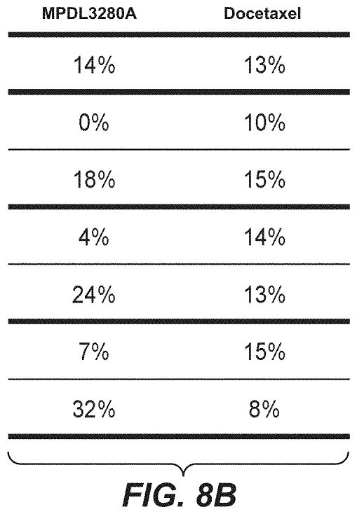

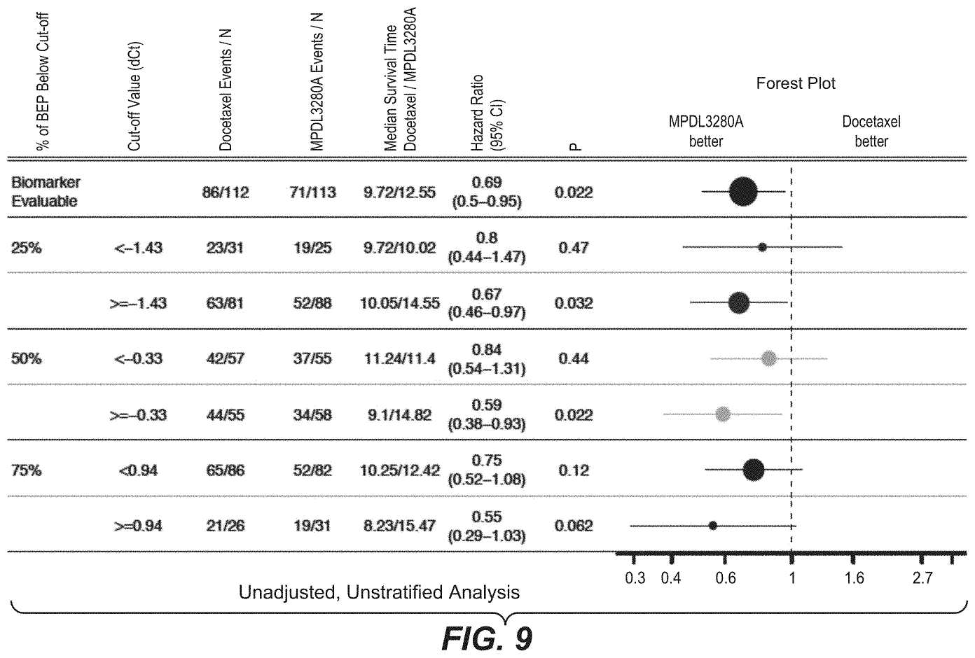

The OS, PFS, and ORR endpoints from the POPLAR clinical trial were evaluated against the PD-L1, CXCL9, and IFNG expression level cut-offs defined herein (e.g., the 25%, 50%, and 75% expression level quartiles). The analysis shows a trend towards an association of immune-score expression levels of PD-L1, CXCL9, and IFNG with improved efficacy of treatment including atezolizumab compared to treatment including docetaxel in patients with NSCLC in the randomized POPLAR study (FIGS. 7A-7B, 8A-8B, and 9). At each percentile cut-off, a higher objective response rate (ORR) was observed in the high expression level subgroup compared to the low expression level subgroup (Table 11). A gradient of increasing PFS and OS benefit was observed with increasing PD-L1, CXCL9, and IFNG expression levels (FIGS. 7A-7B, 8A-8B, and 9). A summary of the association of expression levels of PD-L1, CXCL9, and IFNG with efficacy endpoints in the POPLAR patient population is presented in Table 11. A higher ORR was associated with an increased immune-score expression level of PD-L1, CXCL9, and IFNG in patients treated with atezolizumab, while docetaxel-treated patients did not experience improvement in ORR with increasing immune-score expression level of PD-L1, CXCL9, and IFNG.

TABLE 11
Summary of the association of PD-L1, CXCL9, and IFNG expression
levels with efficacy endpoints in the POPLAR Trial

| | | | | | ORR |
|---|---|---|---|---|---|
| Expression Level | | | | | (atezolizumab |
| Percentile | PFS HR (95% CI)^{a} | | OS HR (95% CI)^{a} | | vs docetaxel) |
| ≥25% | 0.87 | (0.63, 1.21) | 0.67 | (0.46, 0.97) | 14% vs 13% |
| ≥50% (median) | 0.68 | (0.45, 1.02) | 0.59 | (0.38, 0.93) | 18% vs 15% |
| ≥75% | 0.61 | (0.35, 1.08) | 0.55 | (0.29, 1.03) | 24% vs 13% |
| BEP (n = 113 vs 112)^{b} | 0.92 | (0.7, 1.22) | 0.69 | (0.5, 0.95) | 32% vs 8% |
| ITT (n = 144 vs 143)^{b} | 0.92 | (0.71, 1.20) | 0.69 | (0.49, 0.98) | 15% vs 15% |

BEP, biomarker evaluable population.
^{a}Unadjusted and unstratified HRs for atezolizumab vs docetaxel at each expression level percentile cut-off.

Together, these data show that the immune-score expression level of PD-L1, CXCL9, and IFNG can serve as a predictive biomarker that is predictive of therapeutic efficacy of a treatment including a PD-L1 binding antagonist (e.g., an anti-PD-L1 antibody, e.g., atezolizumab (MPDL3280A)). Consequently, evaluation of the expression levels of PD-L1, CXCL9, and IFNG (e.g., the immune-score expression level of PD-L1, CXCL9, and IFNG) can be used, for example, to identify patients having a cancer (e.g., NSCLC) who derive a PFS and an OS benefit from treatment including a PD-L1 binding antagonist (e.g., an anti-PD-L1 antibody, e.g., atezolizumab (MPDL3280A)).

### Example 3. Association Between Expression Level of PD-L1, CXCL9, and IFNG and Clinical Response of Patients Having UBC to Treatment with Atezolizumab (MPDL3280A)

An RNA-based molecular assay was used to evaluate the association between clinical response to treatment with atezolizumab (MPDL3280A), an anti-PD-L1 antibody, and expression levels of PD-L1, CXCL9, and IFNG in individuals with advanced urothelial bladder cancer (UBC) enrolled in a phase II clinical trial (the IMvigor210 Trial) in which atezolizumab was administered as a monotherapy.

### Study Design

Pre-treatment tumor specimens from patients with advanced UBC who were in Cohort 2 of the Phase II IMvigor210 Trial (Clinical Trial ID No.: NCT02108652) were evaluated for the expression level of PD-L1, CXCL9, and IFNG. Patients were eligible for enrollment in Cohort 2 of the IMvigor210 Trial if they had histologically or cytologically documented locally advanced or metastatic transitional cell carcinoma or the urothelium (e.g., renal pelvis, ureters, urinary bladder, or urethra); disease progression during or following a prior platinum-based chemotherapy regimen; ECOG performance status of 0 or 1; life expectancy greater than or equal to 12 weeks; measurable disease, as defined by RECIST v1.1; and adequate hematologic and end-organ function. In this single arm study, all participants received atezolizumab at a dose of 1200 mg intravenously every three weeks on Day 1 of 21-day cycles. Treatment of participants in Cohort 2 of the trial could be continued as long as participants were experiencing clinical benefit, i.e., in the absence of unmanageable toxicity.

### Analysis of PD-L1, CXCL9, and IFNG Expression and Efficacy of MPDL3280A

To evaluate whether PD-L1, CXCL9, and IFNG gene expression status was associated with patient response to treatment with atezolizumab (MPDL3280A), the gene expression levels of PD-L1, CXCL9, and IFNG was assessed in pre-treatment, FFPE tumor samples obtained from each patient. RNA was isolated from FFPE tumor sections and PD-L1, CXCL9, and IFNG gene expression was measured and normalized using RNA-sequencing (RNA-seq).

Tumor specimens obtained from the patients were categorized into different high or low expression level subgroups based on the immune-score expression level of PD-L1, CXCL9, and IFNG relative to a cut-off value for a given percentile (66^{th} percentile) of the population. The 66^{th} percentile cut-off value was defined by the immune-score expression levels of PD-L1, CXCL9, and IFNG greater than or equal to 66% of all immune-score expression levels of PD-L1, CXCL9, and IFNG in the population analyzed.

The efficacy results for the single atezolizumab arm of the IMvigor210 Trial were compared between the high expression level and low expression level subgroups. High expression levels were defined as an immune-score expression level of PD-L1, CXCL9, and IFNG at or above 66^{th} percentile cut-off. Low expression levels were defined as PD-L1, CXCL9, and IFNG immune-score expression levels below the 66^{th} percentile cut-off.

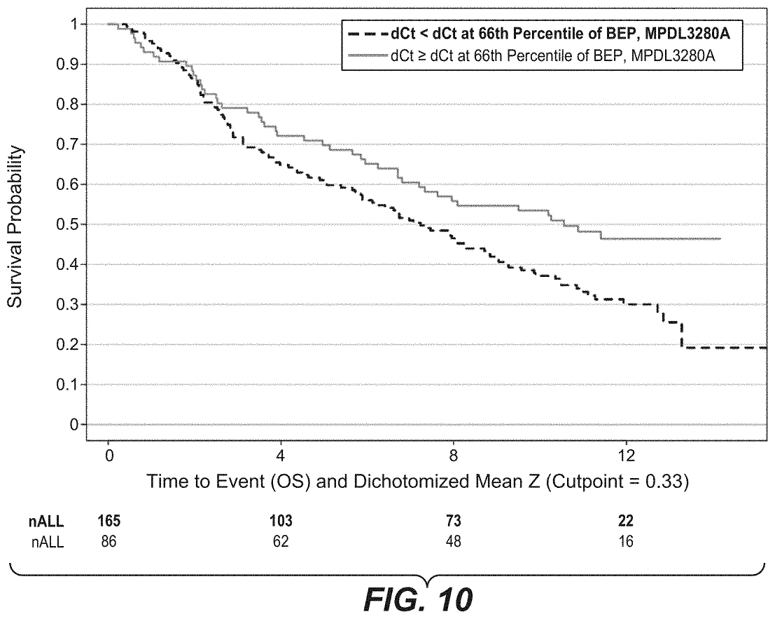

The OS of patients from the IMvigor210 Trial was evaluated against the PD-L1, CXCL9, and IFNG expression level cut-off defined herein (e.g., 66^{th} percentile cut-off). As shown in the Kaplan-Meier Curve of overall survival (OS) shown in FIG. 10, the analysis shows an association of immune-score expression levels of PD-L1, CXCL9, and IFNG with improved treatment benefit with atezolizumab in patients having UBC in Cohort 2 of the IMvigor210 Trial. An increased OS benefit (OS HR (95% CI)=0.66 (0.46-0.93)) was observed in patients with a high immune-score expression level of PD-L1, CXCL9, and IFNG (i.e., at or above the 66% percentile cut-off) as compared to patient with a low normalized expression level of PD-L1, CXCL9, and IFNG (i.e., below the 66% percentile cut-off) (FIG. 10).

Together, this data shows that the immune-score expression level of PD-L1, CXCL9, and IFNG can serve as a predictive biomarker that is predictive of therapeutic efficacy of a treatment including a PD-L1 binding antagonist (e.g., an anti-PD-L1 antibody, e.g., atezolizumab (MPDL3280A)). Consequently, evaluation of the expression levels of PD-L1, CXCL9, and IFNG (e.g., the immune-score expression level of PD-L1, CXCL9, and IFNG) can be used, for example, to identify patients having a cancer (e.g., UBC) who derive an OS benefit from treatment including a PD-L1 binding antagonist (e.g., an anti-PD-L1 antibody, e.g., atezolizumab (MPDL3280A)).

### Example 4. Association Between Expression Levels of PD-L1, IFNG, GZMB, CD8A, and PD-1 and Clinical Response of Patients Having Renal Cell Carcinoma (RCC) to Treatment with MPDL3280A and Bevacizumab

An RNA-based molecular assay was used to evaluate the association between clinical response to treatment with atezolizumab (MPDL3280A), an anti-PD-L1 antibody, in combination with bevacizumab and expression levels of PD-L1, IFNG, GZMB, CD8A, and PD-1 in individuals with advanced renal cell carcinoma (RCC) enrolled in a phase II clinical trial (the IMmotion150 Trial) in which atezolizumab was administered in combination with bevacizumab (AVASTIN®).

### Study Design

Patients were eligible for enrollment in the IMmotion150 Trial (Clinical Trial ID No.: NCT01984242) if they had unresectable advanced or metastatic RCC with component of clear cell histology and/or component of sarcomatoid histology that has not been previously treated with any systemic agents including treatment in the adjuvant stetting; measurable disease, as defined by RECIST v1.1; a Karnofsky performance score greater than or equal to 70; and adequate hematologic and end-organ function. Participants were randomized to receive (i) atezolizumab and bevacizumab at a dose of 15 mg/kg intravenously every three weeks on Day 1 and Day 22 of each 6-week cycle; (ii) atezolizumab at a dose of 1200 mg intravenously every three weeks on Day 1 and Day 22 of each 6-week cycle; or (iii) sunitinib at a dose of 50 mg orally once daily on Days 1 to 28 of each 6-week cycle. Treatment in each arm of the study could be continued as long as participants were experiencing clinical benefit, i.e., in the absence of unacceptable toxicity or symptomatic deterioration attributed to disease progression.

### Analysis of PD-L1, IFNG, GZMB, CD8A, and PD-1 Expression and Efficacy of MPDL3280A

To evaluate whether PD-L1, IFNG, GZMB, CD8A, and PD-1 gene expression status was associated with patient response to treatment with atezolizumab (MPDL3280A) in combination with bevacizumab, the gene expression level of PD-L1, IFNG, GZMB, CD8A, and PD-1 was assessed in pre-treatment, FFPE tumor samples obtained from each patient. RNA was isolated from FFPE tumor sections and PD-L1, IFNG, GZMB, CD8A, and PD-1 gene expression was measured and normalized using RNA-sequencing (RNA-seq).

Tumor specimens obtained from the patients were categorized into different high or low expression level subgroups based on the immune-score expression level of PD-L1, IFNG, GZMB, CD8A, and PD-1 relative to a cut-off value for a given percentile (50^{th} percentile) of the population. The 50^{th} percentile cut-off value was defined by the immune-score expression levels of PD-L1, IFNG, GZMB, CD8A, and PD-1 greater than or equal to 50% of all immune-score expression levels of PD-L1, IFNG, GZMB, CD8A, and PD-1 in the population analyzed.

The efficacy results for the atezolizumab and bevacizumab combination arm and the sunitinib arm of the IMmotion150 Trial were compared for the high expression level and low expression level subgroups. High expression levels were defined as PD-L1, IFNG, GZMB, CD8A, and PD-1 immune-score expression levels at or above the 50^{th} percentile cut-off. Low expression levels were defined as PD-L1, IFNG, GZMB, CD8A, and PD-1 immune-score expression levels below the 50^{th} percentile cut-off.

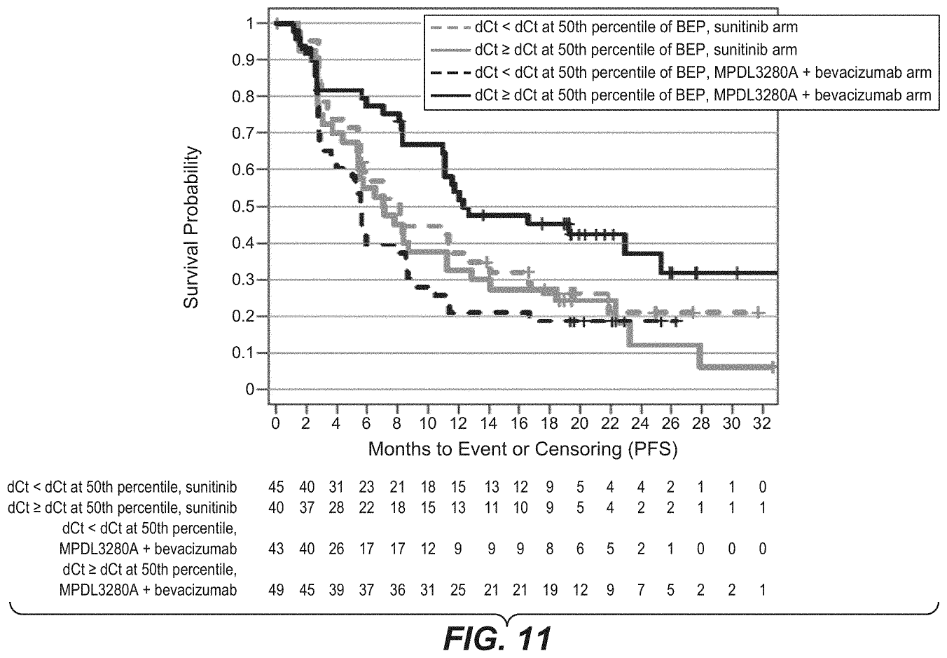

The PFS of patients from the IMMotion150 Trial was evaluated against the PD-L1, IFNG, GZMB, CD8A, and PD-1 expression level cut-off defined herein (i.e., 50^{th} percentile cut-off). The analysis shows a trend towards an association of immune-score expression levels of PD-L1, IFNG, GZMB, CD8A, and PD-1 with improved efficacy of treatment with atezolizumab and bevacizumab as compared to treatment with sunitinib in patients having RCC in the randomized IMmotion150 Trial (FIG. 11). An increased PFS benefit (PFS HR (95% CI)=0.54 (0.33−0.9)) was observed in patients with a high immune-score expression level of PD-L1, IFNG, GZMB, CD8A, and PD-1 relative to the 50% percentile cut-off (FIG. 11).

This data shows that the immune-score expression level of PD-L1, IFNG, GZMB, CD8A, and PD-1 can serve as a predictive biomarker that is predictive of therapeutic efficacy of a treatment including a PD-L1 binding antagonist (e.g., an anti-PD-L1 antibody, e.g., atezolizumab (MPDL3280A)). Consequently, evaluation of the expression levels of PD-L1, IFNG, GZMB, CD8A, and PD-1 (e.g., the immune-score expression level of PD-L1, IFNG, GZMB, CD8A, and PD-1) can be used, for example, to identify patients having a cancer (e.g., RCC) who derive a PFS benefit from treatment including a PD-L1 binding antagonist (e.g., an anti-PD-L1 antibody, e.g., atezolizumab (MPDL3280A)).

### Example 5. Association Between Expression Level of PD-L1, CXCL9, and IFNG and Clinical Response of Patients Having Triple-Negative Breast Cancer (TNBC) to Treatment with Atezolizumab (MPDL3280A)

RNA-sequencing (RNA-seq) was used to evaluate the expression level of PD-L1, CXCL9, and IFNG in patients with triple-negative breast cancer (TNBC) enrolled in a phase I clinical trial, in which atezolizumab (MPDL3280A), an anti-PD-L1 antibody, was administered as a monotherapy

Pre-treatment FFPE tumor specimens from patients with TNBC in the Phase I PCD4989g Trial (Clinical Trial ID No.: NCT01375842) were evaluated for the expression levels of PD-L1, CXCL9, and IFNG. RNA was isolated from the tumor specimens and PD-L1, CXCL9, and IFNG gene expression was measured using RNA-seq.

The tumor specimens obtained from the patients were categorized into high or low expression level subgroups based on their immune-score expression level of PD-L1, CXCL9, and IFNG relative to a cut-off value. The cut-off value was defined by the immune-score expression levels of PD-L1, CXCL9, and IFNG greater than or equal to 50% of all immune-score expression levels of PD-L1, CXCL9, and IFNG in the population analyzed (i.e., the 50^{th} percentile cut-off). The high expression level subgroup was defined by an immune-score expression level of PD-L1, CXCL9, and IFNG at or above the 50^{th} percentile cut-off. The low expression level subgroup was defined by immune-score expression levels of PD-L1, CXCL9, and IFNG below the 50^{th} percentile cut-off.

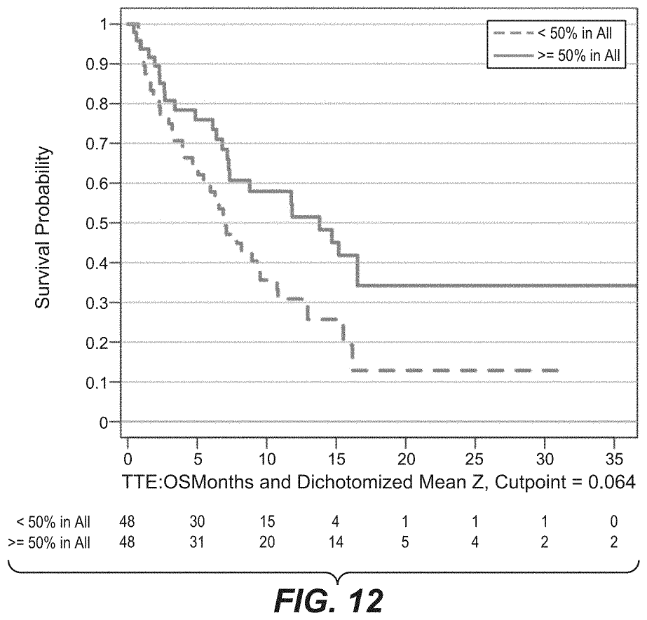

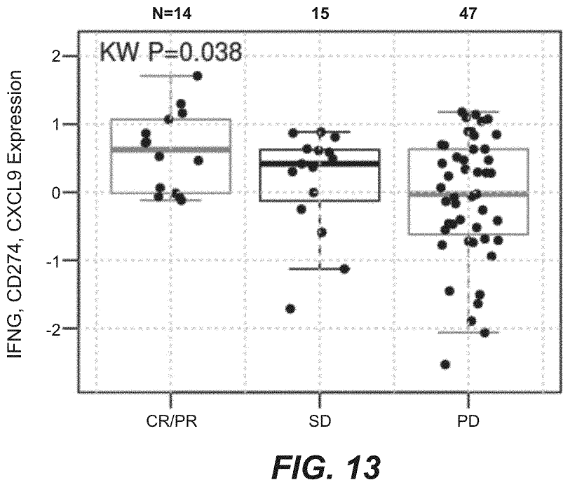

As shown in the Kaplan-Meier Curve of overall survival (OS) in FIG. 12, the analysis shows an association of immune-score expression levels of PD-L1, CXCL9, and IFNG with improved efficacy of treatment with atezolizumab in patients having TNBC in the PCD4989g Trial. An increased OS benefit (OS HR (95% CI)=0.55 (0.33−0.93)) was observed in patients with a high immune-score expression level of PD-L1, CXCL9, and IFNG (i.e., at or above the 50^{th} percentile cut-off) as compared to patient with a low normalized expression level of PD-L1, CXCL9, and IFNG (i.e., below the 50^{th} percentile cut-off) (FIG. 12). Further, as shown in boxplot in FIG. 13, the analysis shows an association of immune-score expression levels of PD-L1, CXCL9, and IFNG with an increased ORR benefit (e.g., complete response or partial response (CR/PR), stable disease (SD), or progressive disease (PD)) was correlated with a higher immune-score expression level of PD-L1, CXCL9, and IFNG.

Together, this data shows that the immune-score expression level of PD-L1, CXCL9, and IFNG can serve as a predictive biomarker that is predictive of the therapeutic efficacy of a treatment including a PD-L1 binding antagonist (e.g., an anti-PD-L1 antibody, e.g., atezolizumab (MPDL3280A)). Consequently, evaluation of the expression levels of PD-L1, CXCL9, and IFNG (e.g., the immune-score expression level of PD-L1, CXCL9, and IFNG) can be used, for example, to identify patients having a cancer (e.g., TNBC) who derive an OS and/or ORR benefit from treatment including a PD-L1 binding antagonist (e.g., an anti-PD-L1 antibody, e.g., atezolizumab (MPDL3280A)).

### Example 6. Association Between Expression Levels of PD-L1, CXCL9, and IFNG and Clinical Response Of Patients Having NSCLC to Treatment with a Combination Therapy Including Atezolizumab (M PDL3280A)

The Phase III IMpower150 Trial (Clinical Trial ID No. NCT02366143) was designed to address whether adding atezolizumab to a bevacizumab and chemotherapy regimen would provide clinical benefit, and also whether atezolizumab could replace bevacizumab in the bevacizumab and chemotherapy regimen.

### Methods

Patients

Patients had chemotherapy-naive, non-squamous, stage IV or recurrent mNSCLC. Patients also had RECIST v1.1-measurable disease, baseline ECOG performance status 0/1, tumor tissue available for biomarker testing, and were bevacizumab-eligible. Patients with EGFR/ALK genomic alterations had disease progression/treatment intolerance with ≥1 approved tyrosine kinase inhibitor (TKI). Patients were excluded if they had untreated central nervous system metastases, autoimmune disease, or received prior immunotherapy/anti-CTLA-4 therapy <6 weeks before randomization or systemic immunosuppressive medications <2 weeks before randomization. Patients who received prior (neo)adjuvant therapy were eligible if their last treatment was ≥6 months before randomization.

Study Design and Treatment

IMpower150 was a global, open-label, Phase III Trial. Patients were randomized 1:1:1 to receive atezolizumab, carboplatin, and paclitaxel (ACP); atezolizumab, bevacizumab, carboplatin, and paclitaxel (ABCP); or bevacizumab, carboplatin, and paclitaxel (BCP). Randomization was stratified by sex, presence of liver metastases at baseline, and PD-L1 expression.

Induction treatment was administered for four or six (at investigator discretion before randomization) 21-day cycles. Doses were 1200 mg atezolizumab, 15 mg/kg bevacizumab, 200 mg/m² paclitaxel (175 mg/m² for Asian race), and area under the concentration-time curve (AUC) 6 mg/mL/min carboplatin, all given on day 1 per cycle. Post-induction, patients continued atezolizumab/bevacizumab until unmanageable toxicity/RECIST v1.1-disease progression. Atezolizumab continuation post-progression was allowed if evidence of clinical benefit existed. No crossover to atezolizumab was allowed.

Endpoints and Assessments

Co-primary endpoints were PFS (RECIST v1.1) in ITT-WT (patients without EGFR/ALK genomic alterations) and in ITT-WT patients with a high immune-score expression level of PD-L1, CXCL9, and IFNG (high immune-score expression level (ISEL^{high})-WT), as well as OS in ITT-WT. The nucleic acid expression level of PD-L1, CXCL9, and IFNG was defined by PDL1, CXCL9, and IFNG mRNA expression using RNA isolated from baseline tumor tissue and measured using quantitative real-time polymerase chain reaction (Roche Molecular Systems). An immune-score expression level that reflects a normalized, averaged dCT value for the analyzed genes was derived from the average expression for each target gene relative to a control gene. In this study, a high immune-score expression level (ISEL^{high}) was defined as an immune-score expression level greater than, or equal to, a pre-defined cut-off value (i.e., an averaged normalized dCt) of −1.91 and a low immune-score expression level (ISEL^{low}) was defined as less than −1.91, based on previous data (Kowanetz et al., J. Thorac. Oncol. 12:S1817-8, 2017).

Key secondary objectives included PFS and OS in ITT, independent review facility (IRF)-assessed PFS in ITT-WT, objective response rate (ORR) and duration of response (DOR; RECIST v1.1), and safety.

Patients underwent tumor assessments during screening, every 6 weeks from cycle 1 day 1 for the first 48 weeks, and every 9 weeks thereafter until RECIST v1.1-disease progression or loss of clinical benefit for patients who continued atezolizumab after initial disease progression. Adverse events (AEs) were assessed using NCI-CTCAE v4.0.

Statistical Analysis

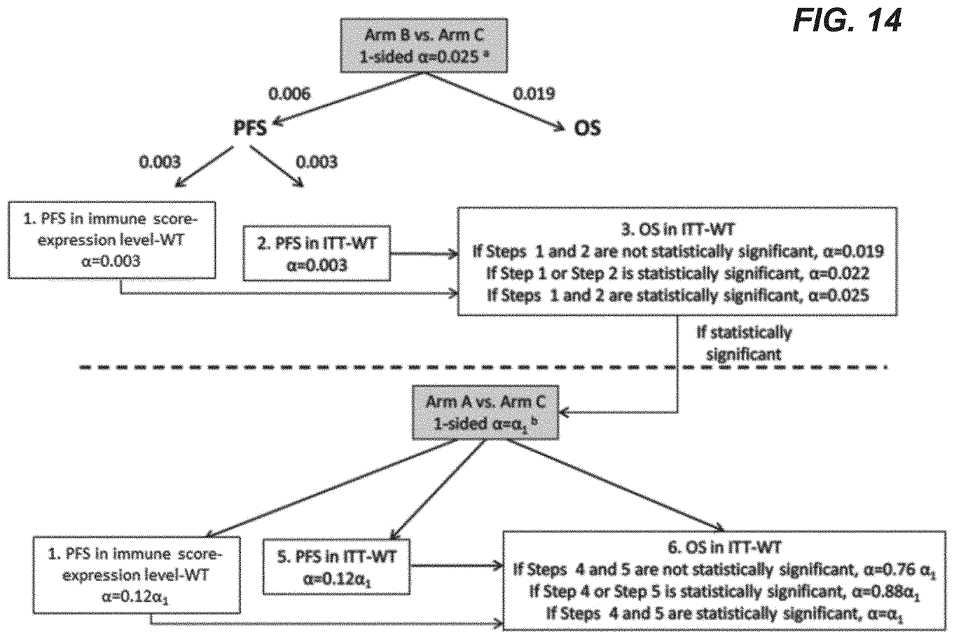

Briefly, co-primary endpoints were tested first between ABCP and BCP due to the improbability of significant PFS or OS benefit with the substitution of bevacizumab for atezolizumab (ACP vs BCP) if the addition of atezolizumab to the BCP regimen did not demonstrate benefit. To strictly control overall type-I error rate at a 1-sided significance level of 0.025, a 1-sided α of 0.006 was allocated to PFS (further split into 0.003 for the 2 primary analysis populations) and of 0.019 to OS (ITT-WT) (FIG. 14) (Dmitrienko et al., Stat. Med. 32:1079-111, 2013 and Dmitrienko et al., Stat. Med. 32:5172-218, 2013). If any PFS comparison was statistically significant, the a would be recycled for the OS comparison (Dmitrienko et al., Stat. Med. 32:1079-111, 2013 and Dmitrienko et al., Stat. Med. 32:5172-218, 2013). If OS was statistically significant with ABCP vs. BCP, the remaining a would be passed down to test PFS and OS between ACP and BCP, followed by testing of PFS and OS in the ITT, including the EGFR/ALK-mutant population, if significant (FIG. 14) (Dmitrienko et al., Stat. Med. 32:1079-111, 2013 and Dmitrienko et al., Stat. Med. 32:5172-218, 2013).

The final PFS and OS analyses were planned when ≈516 PFS and 507 OS events in ITT-WT for ABCP and BCP combined had occurred. An interim OS analysis was planned at the time of the final PFS analysis, and it was expected that ≈370 OS events would have occurred in the ITT-WT population for ABCP and BCP combined. If OS events were significantly <370 at the final PFS analysis, then a nominal 2-sided α of 0.0001 would be spent on the first interim OS analysis. In this case, formal statistical testing of PFS and OS in ACP vs. BCP would be performed later when ABCP vs. BCP OS data matured.

Treatment comparisons for PFS and OS were based on a stratified log-rank test; HRs were estimated using a stratified Cox regression model, and Brookmeyer-Crowley methodology was used to calculate 95% Cls. Kaplan-Meier methodology was used to estimate medians.

Pre-specified subgroup analyses were performed to assess consistency of the treatment effect using unstratified HRs estimated from a Cox proportional hazards model and Kaplan-Meier estimates of medians.

### Results

Patients

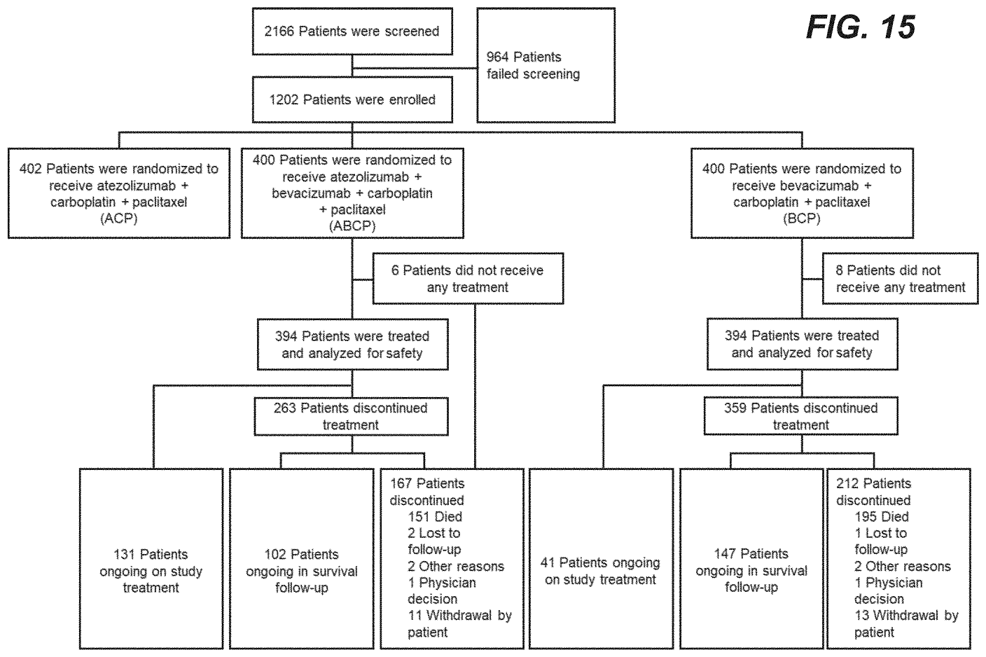

1202 patients were enrolled at 240 sites (26 countries), and 402, 400, and 400 patients were randomized to ACP, ABCP, and BCP, respectively (FIG. 15). The ITT-WT comprised 1040 patients (86.5% of ITT; ACP, 348; ABCP, 356; BCP, 336). An immune-score expression level was evaluable in 95.6% of ITT-WT patients. ISEL^{high}-WT comprised 445 patients (42.8% of ITT-WT; ACP, 161; ABCP, 155; BCP, 129).

Baseline characteristics were generally balanced between ABCP and BCP (Tables 12 and 13). Three patients (12.0%) in ABCP and four patients (12.5%) in BCP with EGFR/ALK genomic alterations did not have prior TKI therapy reported, predominately due to lack of availability of approved TKI therapy in their respective countries.

TABLE 12
Baseline Characteristics of the ITT Population

| | ITT | | |
|---|---|---|---|
| | | ABCP | BCP |
| Characteristic | | (N = 400) | (N = 400) |

Age - yr

| | | |
|---|---|---|
| Median | 63 | 63 |
| Range | 31-89 | 31-90 |

| Age groups - no. (%) | | | | |
|---|---|---|---|---|
| <65 yr | 215 | (53.8) | 226 | (56.5) |
| 65 to 74 yr | 149 | (37.3) | 132 | (33.0) |
| 75 to 84 yr | 33 | (8.3) | 39 | (9.8) |
| ≥85 yr | 3 | (0.8) | 3 | (0.8) |
| Sex-no. (%) | | | | |
| Male | 240 | (60.0) | 239 | (59.8) |
| Liver metastases at enrollment - no. (%) | | | | |
| Absent | 347 | (86.8) | 343 | (85.8) |
| Immune-score expression level - no. (%)* | | | | |
| High | 166 | (41.5) | 148 | (37.0) |
| Low | 217 | (54.3) | 231 | (57.8) |
| Unknown | 17 | (4.3) | 21 | (5.3) |
| Race - no. (%) | | | | |
| White | 322 | (80.5) | 335 | (83.8) |
| Asian | 56 | (14.0) | 46 | (11.5) |
| Black | 3 | (0.8) | 12 | (3. 0) |
| American Indian or Alaska Native | 3 | (0.8) | 1 | (0.3) |
| Multiple | 3 | (0.8) | 0 | |
| Unknown | 13 | (3.3) | 6 | (1.5) |
| ECOG performance status - no. (%) | | | | |
| 0 | 159 | (40.1) | 179 | (45.1) |
| 1 | 238 | (59.9) | 218 | (54.9) |
| Tobacco use history - no. (%) | | | | |
| Never | 82 | (20.5) | 77 | (19.3) |
| Current | 90 | (22.5) | 92 | (23.0) |
| Previous | 228 | (57.0) | 231 | (57.8) |
| Non-squamous histology - no. (%) | | | | |
| Adenocarcinoma | 378 | (94.5) | 377 | (94.3) |
| Other^{†} | 19 | (4.8) | 17 | (4.3) |
| Unknown or not assessed | 3 | (0.8) | 6 | (1.5) |
| EGFR mutation status - no. (%) | | | | |
| Positive | 35 | (8.8) | 45 | (11.3) |
| Negative | 352 | (88.0) | 345 | (86.3) |
| Unknown | 13 | (3.3) | 10 | (2.5) |
| EML4-ALK rearrangement status - no. (%) | | | | |
| Positive | 13 | (3.3) | 21 | (5.3) |
| Negative | 383 | (95.8) | 375 | (93.8) |
| Unknown | 4 | (1.0) | 4 | (1.0) |
| KRAS mutation status - no. (%) | | | | |
| Positive | 47 | (11.8) | 38 | (9.5) |
| Negative | 59 | (14.8) | 77 | (19.3) |
| Unknown | 294 | (73.5) | 285 | (71.3) |

*The ISEL-cut-off of −1.91 was used.
^{†}Other includes adenocarcinoma with neuroendocrine features, adenosquamous, bronchioloalveolar carcinoma, large cell, sarcomatoid, and undifferentiated.
ABCP denotes atezolizumab + bevacizumab + carboplatin + paclitaxel; BCP, bevacizumab + carboplatin + paclitaxel; ECOG, Eastern Cooperative Oncology Group; IC, tumor-infiltrating immune cell; ITT, intention-to-treat; PD-L1, programmed death-ligand 1; TC, tumor cell; WT, wild-type.

TABLE 13
Baseline Characteristics of the Primary Analysis Populations

| | ITT-WT | | | | ISEL^{high}-WT | | | |
|---|---|---|---|---|---|---|---|---|
| | ABCP | | BCP | | ABCP | | BCP | |
| Characteristic | (n = 356) | | (n = 336) | | (n = 155) | | (n = 129) | |
| Age - yr | | | | | | | | |
| Median | 63 | | 63 | | 66 | | 63 | |
| Range | 31-89 | | 41-87 | | 31-89 | | 41-82 | |
| Age groups - no. (%) | | | | | | | | |
| <65 yr | 191 | (53.7) | 184 | (54.8) | 71 | (45.8) | 74 | (57.4) |
| 65 to 74 yr | 130 | (36.5) | 118 | (35.1) | 69 | (44.5) | 44 | (34.1) |
| 75 to 84 yr | 32 | (9.0) | 32 | (9.5) | 12 | (7.7) | 11 | (8.5) |
| ≥85 yr | 3 | (0.8) | 2 | (0.6) | 3 | (1.9) | 0 | |
| Sex - no. (%) | | | | | | | | |
| Male | 217 | (61.0) | 208 | (61.9) | 94 | (60.6) | 74 | (57.4) |
| Liver metastases at enrollment - no. (%) | | | | | | | | |
| No | 309 | (86.8) | 289 | (86.0) | 137 | (88.4) | 115 | (89.1) |
| Immune-score expression level - no. (%)* | | | | | | | | |
| High | 155 | (43.5) | 129 | (38.4) | 155 | (100) | 129 | (100) |
| Low | 186 | (52.2) | 188 | (56.0) | 0 | | 0 | |
| Unknown | 15 | (4.2) | 19 | (5.7) | 0 | | 0 | |
| Race - no. (%) | | | | | | | | |
| White | 294 | (82.6) | 296 | (88.1) | 125 | (80.6) | 106 | (82.2) |
| Asian | 42 | (11.8) | 23 | (6.8) | 22 | (14.2) | 13 | (10.1) |
| Black | 3 | (0.8) | 11 | (3.3) | 2 | (1.3) | 6 | (4.7) |
| American Indian or Alaska Native | 3 | (0.8) | 1 | (0.3) | 0 | | 1 | (0.8) |
| Multiple | 2 | (0.6) | 0 | | 1 | (0.6) | 0 | |
| Unknown | 12 | (3.4) | 5 | (1.5) | 5 | (3.2) | 3 | (2.3) |
| ECOG performance status - no. (%) | | | | | | | | |
| 0 | 139 | (39.0) | 143 | (42.6) | 57 | (36.8) | 64 | (49.6) |
| 1 | 214 | (60.1) | 190 | (56.5) | 98 | (63.2) | 65 | (50.4) |
| Other than 0/1 | 3 | (0.8) | 3 | (0.9) | 0 | | 0 | |
| Tobacco use history - no. (%) | | | | | | | | |
| Never | 58 | (16.3) | 50 | (14.9) | 22 | (14.2) | 19 | (14.7) |
| Current | 83 | (23.3) | 84 | (25.0) | 29 | (18.7) | 29 | (22.5) |
| Previous | 215 | (60.4) | 202 | (60.1) | 104 | (67.1) | 81 | (62.8) |
| Non-squamous histology - no. (%) | | | | | | | | |
| Adenocarcinoma | 335 | (94.1) | 315 | (93.8) | 145 | (93.5) | 118 | (91.5) |
| Other^{†} | 18 | (5.1) | 15 | (4.5) | 10 | (6.5) | 7 | (5.4) |
| Unknown or not assessed | 3 | (0.8) | 6 | (1.8) | 0 | | 4 | (3.1) |
| EGFR mutation status - no. (%) | | | | | | | | |
| Negative | 344 | (96.6) | 328 | (97.6) | 150 | (96.8) | 128 | (99.2) |
| Unknown | 12 | (3.4) | 8 | (2.4) | 5 | (3.2) | 1 | (0.8) |
| EML4-ALK rearrangement status - no. (%) | | | | | | | | |
| Negative | 353 | (99.2) | 334 | (99.4) | 154 | (99.4) | 129 | (100) |
| Unknown | 3 | (0.8) | 2 | (0.6) | 1 | (0.6) | 0 | |
| KRAS mutation status - no. (%) | | | | | | | | |
| Positive | 44 | (12.4) | 36 | (10.7) | 20 | (12.9) | 15 | (11.6) |
| Negative | 55 | (15.4) | 69 | (20.5) | 21 | (13.5) | 27 | (20.9) |
| Unknown | 257 | (72.2) | 231 | (68.8) | 114 | (73.5) | 87 | (67.4) |

*The ISEL cut-off of −1.91 was used.
^{†}Other includes adenocarcinoma with neuroendocrine features, adenosquamous, bronchioloalveolar carcinoma, large cell, sarcomatoid, and undifferentiated.

Primary PFS Analysis—ABCP vs. BCP arms

At data cutoff, minimum survival follow-up was 9.5 months (median ITT-WT follow-up, 15.4 and 15.5 months for ABCP and BCP, respectively).

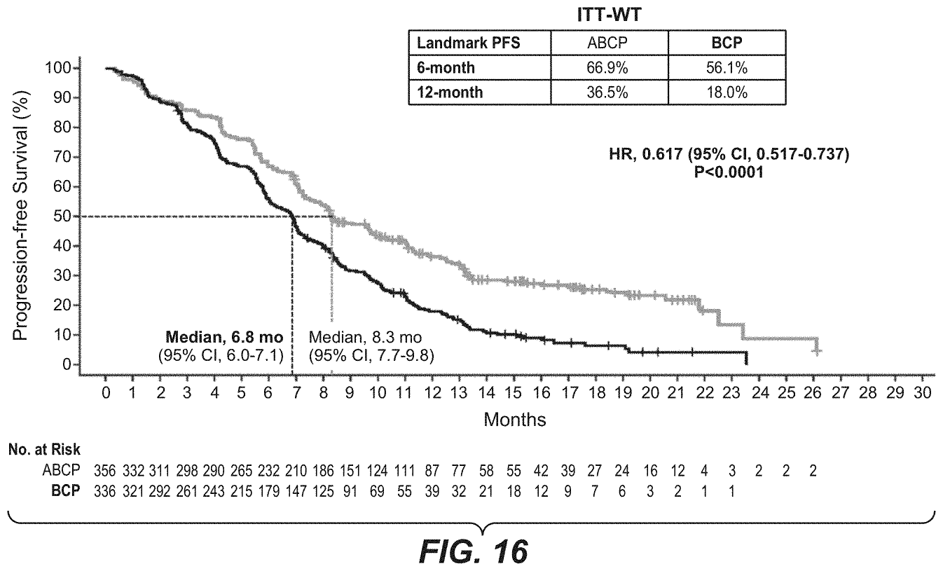

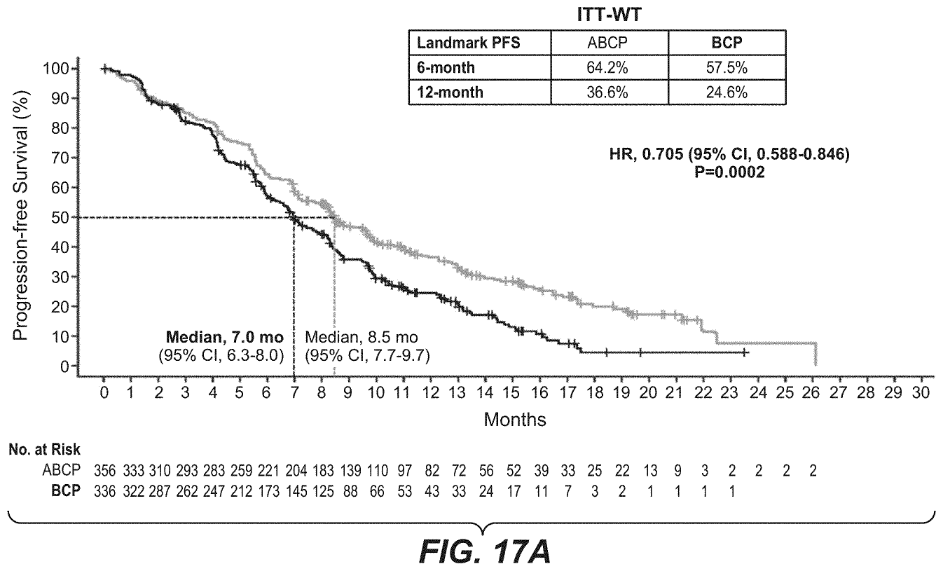

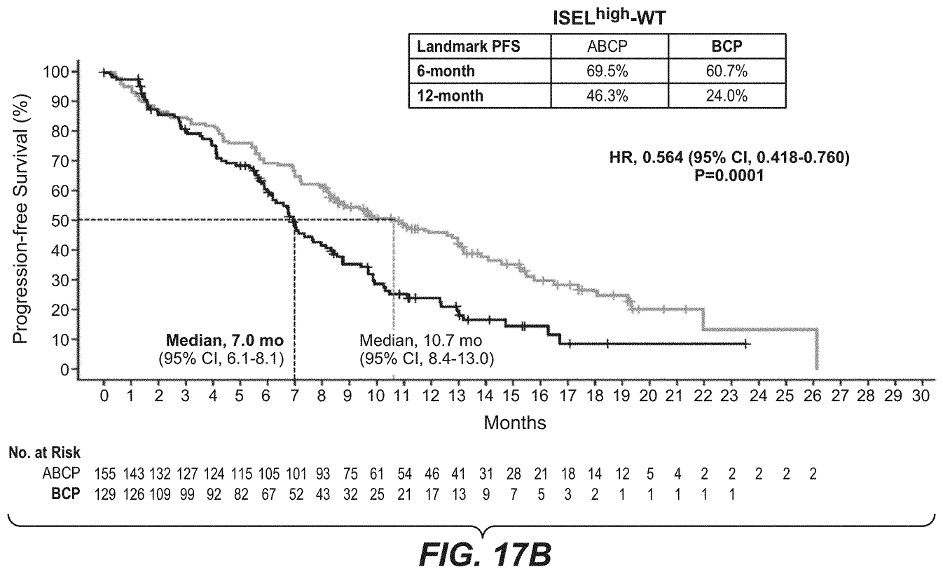

In ITT-WT (ABCP and BCP combined), 517/692 patients (74.7%) had a PFS event. Significant PFS benefit with ABCP vs. BCP was observed; stratified (per randomization factors) HR was 0.617 (95% CI, 0.517-0.737; P<0.0001; ABCP: 241/356 (67.7%) vs. BCP: 276/336 (82.1%) events), with median PFS of 8.3 vs. 6.8 months, respectively (FIG. 16). At 6 months, PFS rates were 66.9% vs. 56.1% with ABCP vs. BCP; at 12 months, 36.5% vs. 18.0%. These results were confirmed by central IRF-assessment (FIGS. 17A and 17B).

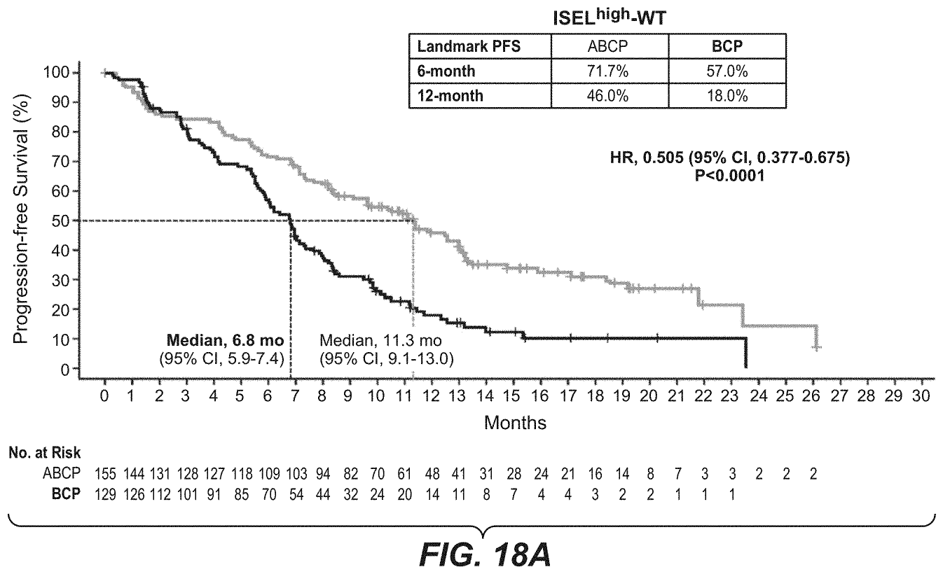

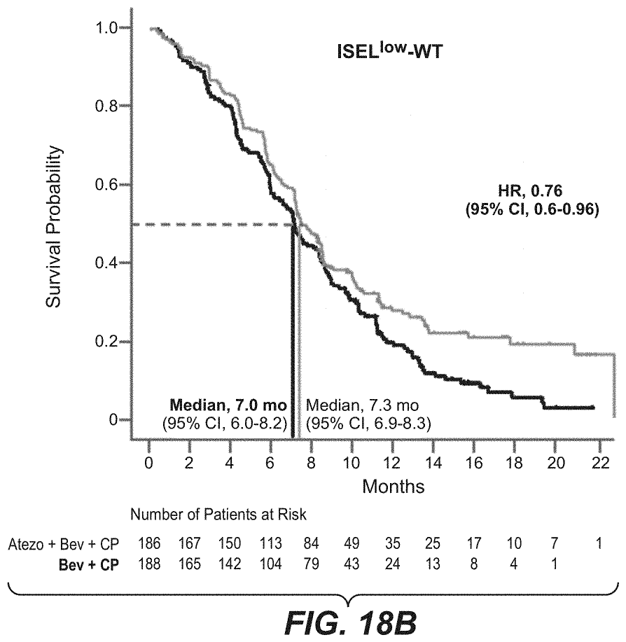

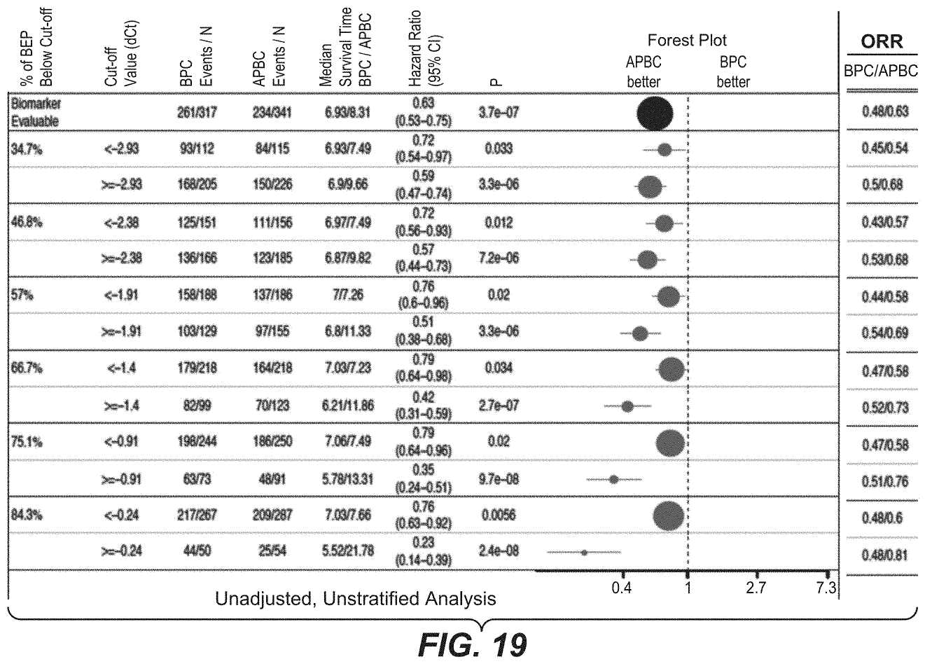

In ISEL^{high}-WT, 200/284 patients (70.4%) had a PFS event. Stratified (by sex and liver metastases) HR was 0.505 (95% CI, 0.377-0.675; P<0.0001; ABCP: 97/155 (62.6%) vs BCP: 103/129 (79.8%) events), with median PFS of 11.3 months vs. 6.8 months with ABCP vs. BCP (FIGS. 18A and 18B). At 6 months, PFS rates were 71.7% vs. 57.0% with ABCP vs. BCP; at 12 months, PFS rates were 46.0% vs. 18.0% with ABCP vs. BCP. A median PFS of up to approximately 21.8 months vs. 5.5 months with ABCP vs. BCP was observed in ISEL^{high} patients using a cut-off value of −0.24, corresponding to a 15.7% prevalence (FIG. 19).

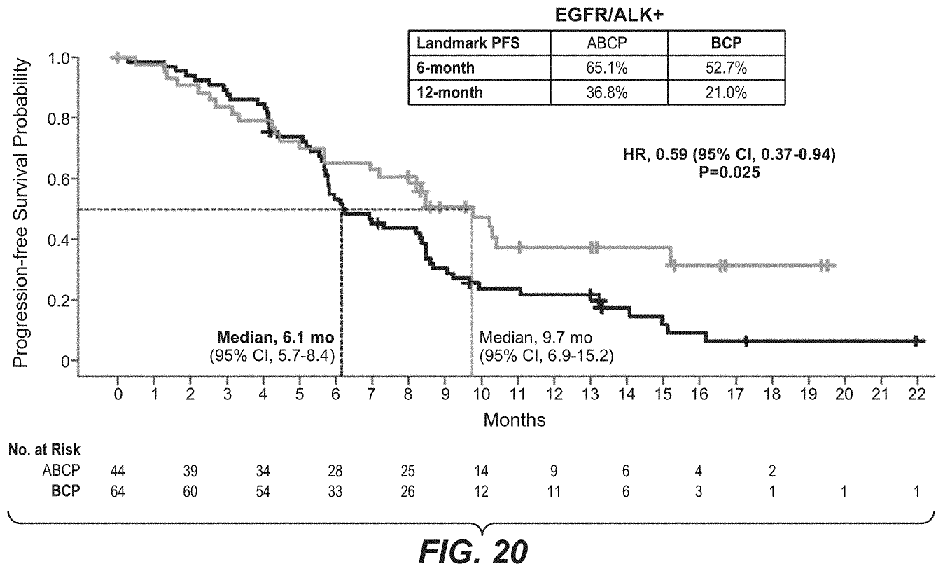

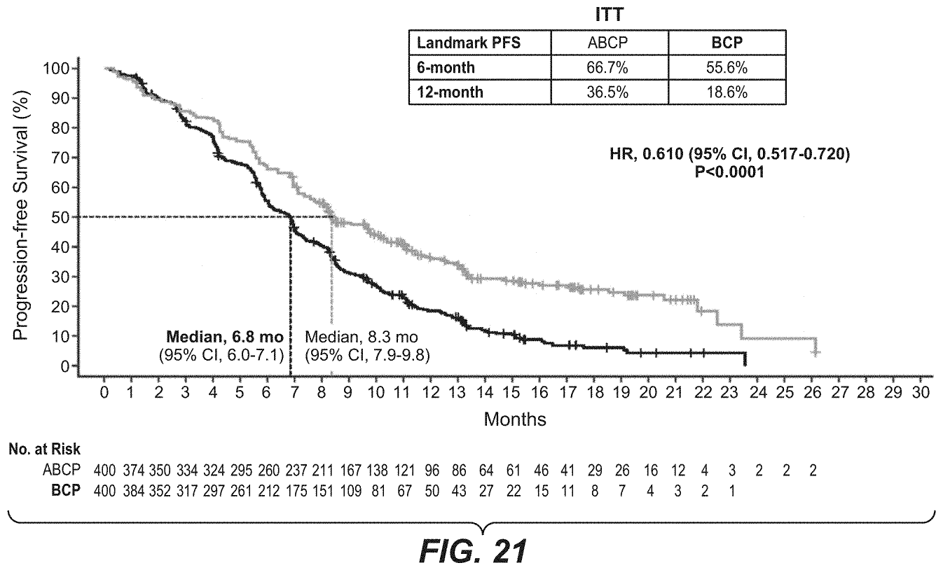

Patients with EGFR mutations or ALK translocations (EGFR/ALK+) also demonstrated PFS benefit with ABCP vs. BCP (FIG. 20). All enrolled patients (ITT), including patients with EGFR/ALK genetic alterations, also benefitted from ABCP therapy as compared to BCP therapy (FIG. 21). The HR was 0.610 (95% CI, 0.517-0.720; P<0.0001), with a median PFS of 8.3 months vs. 6.8 months with ABCP vs. BCP. At 6 months, PFS rates were 66.7% vs. 55.6% with ABCP vs. BCP; at 12 months, PFS rates were 36.5% vs. 18.6% with ABCP vs. BCP.

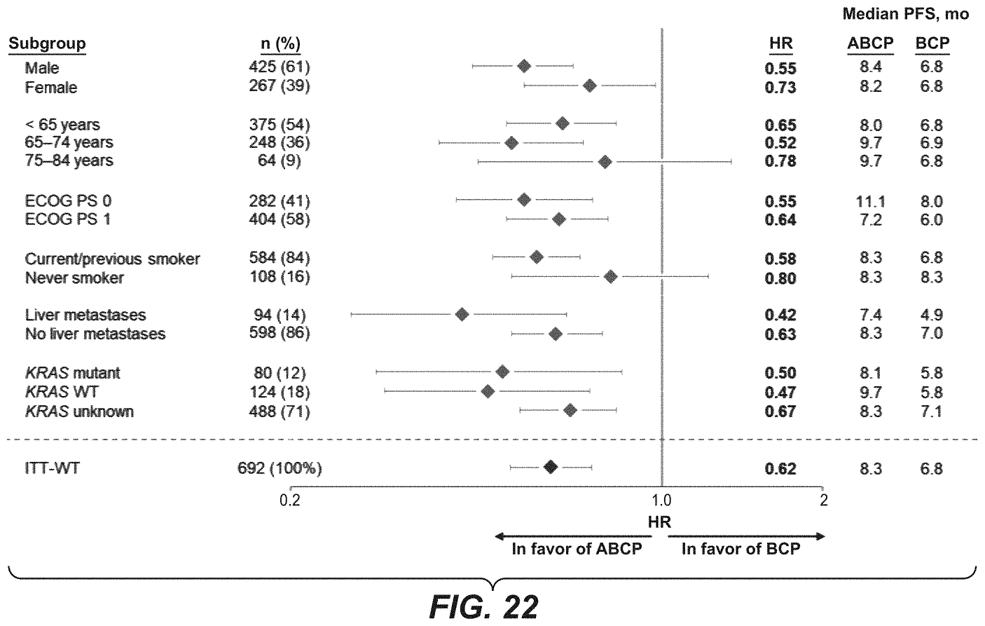

In additional, PFS benefit was observed with ABCP vs. BCP in key clinical and biomarker subgroups, including patients with liver metastases and KRAS mutations (FIG. 22). The benefit observed in patients with EGFR/ALK genetic alterations is notable given that clinical trials investigating the use of PD-L1/PD-1 inhibitors as monotherapy after failure of TKIs have not shown efficacy improvements in comparison to standard chemotherapy in these patients (Rittmeyer et al., Lancet. 389:255-65, 2017, Borghaei et al., N. Engl. J. Med. 373:1627-39, 2015, and Herbst et al., Lancet. 387:1540-50, 2016). Furthermore, such patients have limited proven treatment options, and the effectiveness of platinum-based regimens±PD-L1/PD-1 inhibitors has not been previously investigated in phase 3 trials (Peters et al., J. Clin. Oncol. 35:2781-9, 2017).

Preliminary OS—ABCP vs. BCP Arms

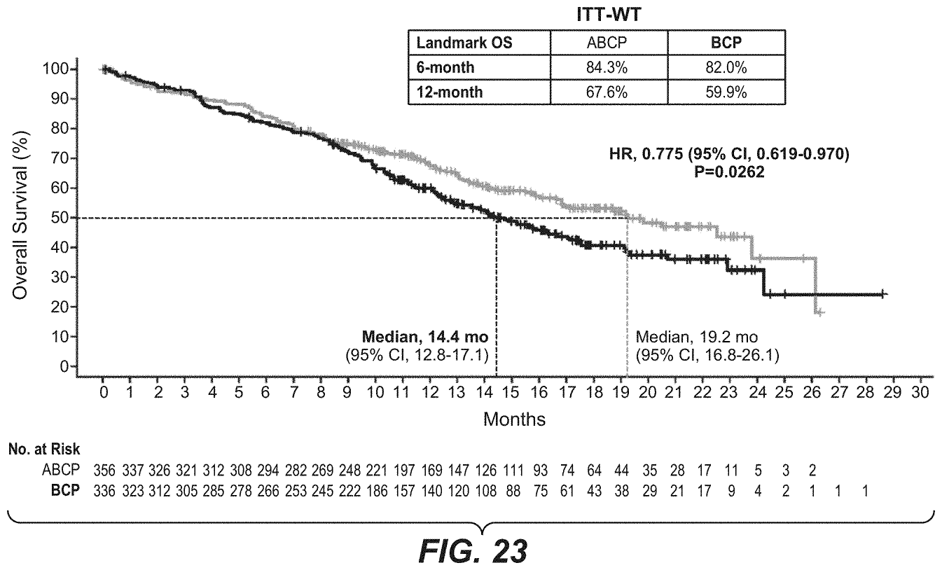

At data cut-off, 310/692 patients (44.8%) in the ITT-WT ABCP and BCP arms had died. Stratified (per randomization factors) HR for OS was 0.775 (95% CI, 0.619-0.970; P=0.0262; ABCP: 144/356 (40.4%) vs BCP: 166/336 (49.4%) events), with median OS of 19.2 vs. 14.4 months with ABCP vs. BCP (FIG. 23). Thus, numerical improvement in the ABCP arm compared to the BCP arm was observed for OS in ITT-WT patients.

ORR and DOR—ABCP vs. BCP Arms

In ITT-WT, unconfirmed ORRs were 63.5% and 48.0% with ABCP and BCP; more complete responses were observed with ABCP. Results were similar in an ISEL^{high}-WT (Table 14). In ITT-WT, median DOR was 9.0 months and 5.7 months with ABCP and BCP. In ISEL^{high}-WT, median DOR was 11.2 months and 5.7 months, respectively (Table 14).

TABLE 14
Objective Response Rate (ORR) and Duration of Response (DOR)

| | |
|---|---|
| ABCP | BCP |

Objective Response Rate

| ITT-WT - no. | n = 353 | | n = 331 | |
|---|---|---|---|---|
| Objective response - no. (%) | 224 | (63.5) (58-68) | 159 | (48.0) (43-54) |
| (95% CI) | | | | |
| Complete response | 13 | (3.7) (2-6) | 4 | (1.2) (0-3) |
| Partial response | 211 | (59.8) (54-65) | 155 | (46.8) (41-52) |
| Stable disease | 77 | (21.8) (18-26) | 115 | (34.7) (30-40) |
| Progressive disease | 18 | (5.1) (3-8) | 27 | (8.2) (5-12) |
| Missing or unevaluable | 34 | (9.6) | 30 | (9.1) |
| ISEL^{high}-WT - no. | n = 153 | | n = 127 | |
| Objective response - no. (%) | 106 | (69.3) (61-76) | 68 | (53.5) (44-62) |
| (95% CI) | | | | |
| Complete response | 6 | (3.9) (1-8) | 3 | (2.4) (0-7) |
| Partial response | 100 | (65.4) (57-73) | 65 | (51.2) (42-60) |
| Stable disease | 23 | (15.0) (10-22) | 39 | (30.7) (23-40) |
| Progressive disease | 6 | (3.9) (1-8) | 10 | (7.9) (4-14) |
| Missing or unevaluable | 18 | (11.8) | 10 | (7.9) |

Duration of Response^{†}

| ITT-WT - no. | n = 224 | | n = 159 | |
|---|---|---|---|---|
| Median (range), mo | 9.0 | (0.4-24.9^{‡}) | 5.7 | (0.0^{‡}-22.1) |
| Patients with ongoing response at cutoff - no. (%) | 91 | (40.6) | 32 | (20.1) |
| ISEL^{high}-WT - no. | n = 106 | | n = 68 | |
| Median (range), mo | 11.2 | (0.5-24.9^{‡}) | 5.7 | (0.0^{‡}-22.1) |
| Patients with ongoing response at cutoff - no. (%) | 49 | (46.2) | 16 | (23.5) |

^{†}Duration of response was assessed in patients who achieved an objective response as determined by the investigator according to RECIST v1.1.
^{‡}Censored value.

The results of this Phase III, randomized trial revealed a clinically and statistically significant improvement in PFS with the addition of atezolizumab to BCP as first-line treatment for non-squamous mNSCLC. ABCP significantly prolonged PFS, resulting in a 38.3% reduction in the risk of disease progression or death, doubling of the 12-month PFS rate from 18.0% to 36.5%, and increased ORR vs. BCP (48.0% vs. 63.5%, respectively). Preliminary OS data, while not yet mature (44.8% event-to-patient ratio), appear promising.

PFS Analysis—ACP vs. BCP Arms

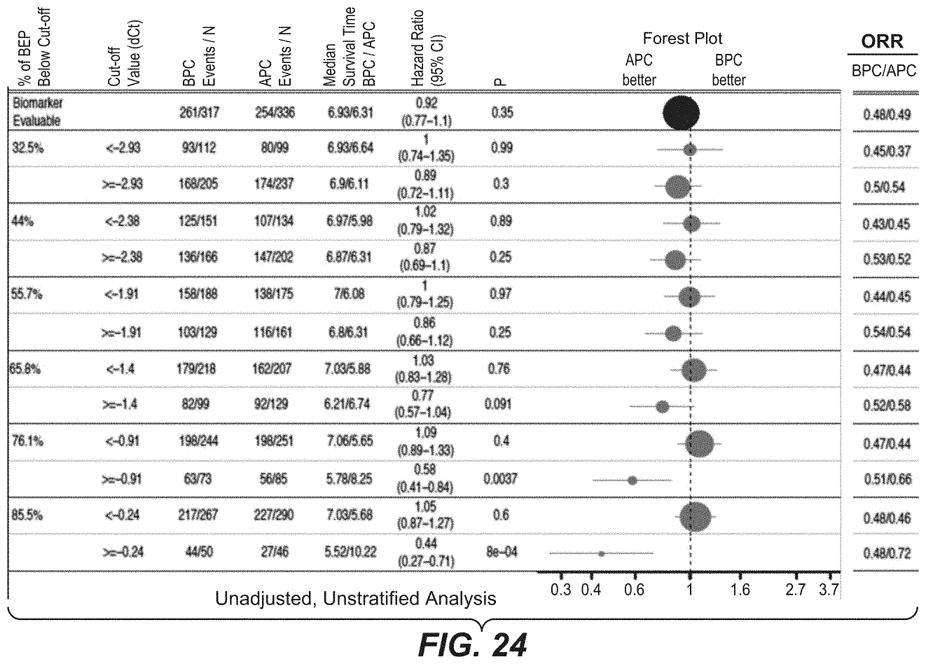

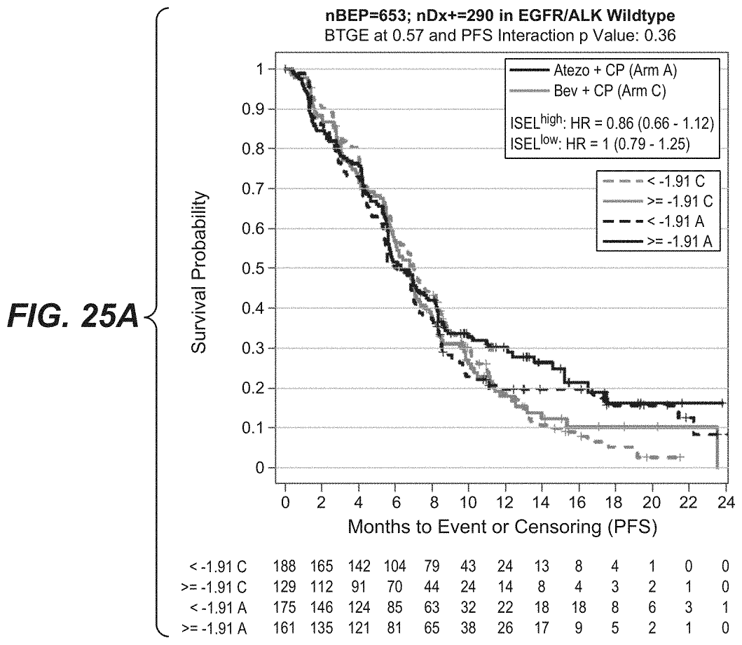

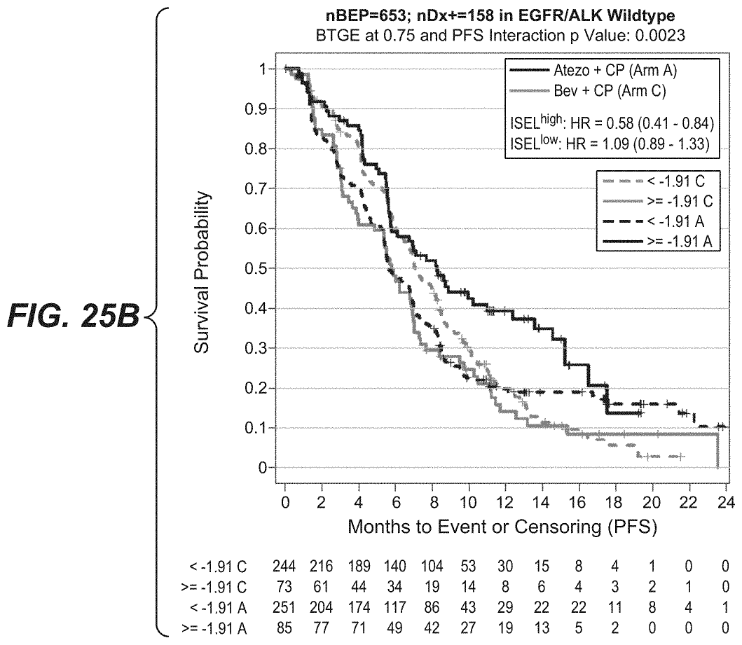

Upon evaluation of PFS enrichment in the ACP arm compared to the BCP arm, a high immune expression-score level did not enrich for PFS at the primary cut-off of −1.91 (FIGS. 24 and 25A). However, PFS enrichment was observed at a higher immune-score expression level cut-off, corresponding to approximately 25% or lower prevalence (dCt=−0.91) (FIGS. 24 and 25B).

### Safety

787 patients received ABCP (393) or BCP (394). For ABCP, median treatment durations were 8.2 months (range, 0-26) with atezolizumab (median doses, 12 [range, 1-38]) and 6.7 months (0-26) with bevacizumab (median doses, 10 [1-38]). For BCP, median treatment duration was 5.1 months (0-22) with bevacizumab (median doses, 8 [1-33]). Median chemotherapy treatment duration across arms was 2.2 months (0-5). 31.7% of patients receiving BCP received subsequent immunotherapy.

Treatment-related AEs occurred in 94.4% and 95.4% of patients receiving ABCP and BCP, respectively (Table 15). Incidences of grade 1-2 treatment-related AEs were 35.9% and 45.4% in ABCP and BCP and were transient; the most common grade 3-4 treatment-related AEs were neutropenia, decreased neutrophil count, febrile neutropenia, and hypertension. There was a <10% increase in the incidence of rash, stomatitis, febrile neutropenia and hemoptysis in patients that received ABCP vs. BCP. Eleven (2.8%) and nine patients (2.3%) experienced treatment-related deaths with ABCP and BCP (Table 15). Five deaths with ABCP were due to pulmonary hemorrhage, four of which occurred in patients with high-risk features (e.g., tumor infiltration of great vessels or cavitation). These events occurred early in the trial and led to a change in eligibility criteria to prevent enrollment of patients with high-risk features. Incidences of treatment-related serious AEs were 25.4% and 19.3% with ABCP and BCP, respectively (Tables 15 and 16).

Most immune-related AEs (irAEs) were grade 1-2, and no grade 5 irAEs were reported with ABCP. The most common irAEs observed were rash, hepatitis, hypothyroidism, hyperthyroidism, pneumonitis, and colitis.

TABLE 15
Treatment-Related Adverse Events

| | ABCP (n = 393) | | | BCP (n = 394) | | |
|---|---|---|---|---|---|---|
| Patients - no. (%) | Grade 1-2 | Grade 3-4 | Grade 5 | Grade 1-2 | Grade 3-4 | Grade 5 |
| Treatment-related AEs | 141 (35.9) | 219 (55.7) | 11 (2.8) | 179 (45.4) | 188 (47.7) | 9 (2.3) |
| Serious treatment- | 15 (3.8) | 74 (18.8) | 11 (2.8) | 9 (2.3) | 58 (14.7) | 9 (2.3) |
| related AEs | | | | | | |

Treatment-related AEs with an incidence of ≥10% in any arm, grade
3-4 severity with incidence of ≥1% in any arm, or grade 5 severity

| | | | | | | |
|---|---|---|---|---|---|---|
| Alopecia | 183 (46.6) | 0 | 0 | 173 (43.9) | 0 | 0 |
| Nausea | 119 (30.3) | 15 (3.8) | 0 | 101 (25.6) | 8 (2.0) | 0 |
| Fatigue | 88 (22.4) | 13 (3.3) | 0 | 79 (20.1) | 10 (2.5) | 0 |
| Anemia | 70 (17.8) | 24 (6.1) | 0 | 71 (18.0) | 23 (5.8) | 0 |
| Peripheral neuropathy | 82 (20.9) | 6 (1.5) | 0 | 63 (16.0) | 3 (0.8) | 0 |
| Decreased appetite | 77 (19.6) | 10 (2.5) | 0 | 56 (14.2) | 3 (0.8) | 0 |
| Diarrhea | 70 (17.8) | 11 (2.8) | 0 | 58 (14.7) | 2 (0.5) | 0 |
| Neutropenia | 18 (4.6) | 54 (13.7) | 0 | 24 (6.1) | 44 (11.2) | 0 |
| Hypertension | 50 (12.7) | 25 (6.4) | 0 | 42 (10.7) | 25 (6.3) | 0 |
| Arthralgia | 63 (16.0) | 3 (0.8) | 0 | 55 (14.0) | 4 (1.0) | 0 |
| Peripheral sensory | 60 (15.3) | 5 (1.3) | 0 | 50 (12.7) | 6 (1.5) | 0 |
| neuropathy | | | | | | |
| Constipation | 65 (16.5) | 0 | 0 | 45 (11.4) | 0 | 0 |
| Asthenia | 52 (13.2) | 5 (1.3) | 0 | 53 (13.5) | 11 (2.8) | 0 |
| Epistaxis | 50 (12.7) | 4 (1.0) | 0 | 68 (17.3) | 0 | 0 |
| Vomiting | 50 (12.7) | 6 (1.5) | 0 | 51 (12.9) | 5 (1.3) | 0 |
| Decreased platelet count | 34 (8.7) | 20 (5.1) | 0 | 35 (8.9) | 9 (2.3) | 0 |
| Myalgia | 51 (13.0) | 2 (0.5) | 0 | 46 (11.7) | 1 (0.3) | 0 |
| Thrombocytopenia | 36 (9.2) | 16 (4.1) | 0 | 28 (7.1) | 17 (4.3) | 0 |
| Proteinuria | 41 (10.4) | 10 (2.5) | 0 | 37 (9.4) | 11 (2.8) | 0 |
| Decreased neutrophil | 14 (3.6) | 34 (8.7) | 0 | 10 (2.5) | 25 (6.3) | 0 |
| count | | | | | | |
| Rash | 47 (12.0) | 5 (1.3) | 0 | 20 (5.1) | 0 | 0 |
| Stomatitis | 43 (10.9) | 4 (1.0) | 0 | 20 (5.1) | 1 (0.3) | 0 |
| Paresthesia | 42 (10.7) | 0 | 0 | 36 (9.1) | 1 (0.3) | 0 |
| Febrile neutropenia | 2 (0.5) | 33 (8.4) | 3 (0.8) | 0 | 23 (5.8) | 0 |
| Decreased white blood | 14 (3.6) | 13 (3.3) | 0 | 10 (2.5) | 11 (2.8) | 0 |
| cell count | | | | | | |
| Decreased weight | 20 (5.1) | 4 (1.0) | 0 | 18 (4.6) | 1 (0.3) | 0 |
| ALT increased | 17 (4.3) | 4 (1.0) | 0 | 12 (3.0) | 1 (0.3) | 0 |
| Dehydration | 9 (2.3) | 8 (2.0) | 0 | 11 (2.8) | 4 (1.0) | 0 |
| AST increased | 16 (4.1) | 4 (1.0) | 0 | 8 (2.0) | 1 (0.3) | 0 |
| Leukopenia | 6 (1.5) | 7(1.8) | 0 | 11 (2.8) | 4 (1.0) | 0 |
| Hemoptysis | 15 (3.8) | 0 | 3 (0.8) | 8 (2.0) | 0 | 0 |
| Hypokalemia | 9 (2.3) | 7 (1.8) | 0 | 5 (1.3) | 2 (0.5) | 0 |
| Pulmonary embolism | 2 (0.5) | 7 (1.8) | 0 | 2 (0.5) | 8 (2.0) | 2 (0.5) |
| Hyponatremia | 4 (1.0) | 8 (2.0) | 0 | 3 (0.8) | 4 (1.0) | 0 |
| Pneumonia | 3 (0.8) | 7 (1.8) | 0 | 2 (0.5) | 2 (0.5) | 1 (0.3) |
| Pneumonitis | 5 (1.3) | 4 (1.0) | 0 | 0 | 0 | 0 |
| Colitis | 3 (0.8) | 5 (1.3) | 0 | 0 | 0 | 0 |
| Transaminases increased | 3 (0.8) | 4 (1.0) | 0 | 1 (0.3) | 0 | 0 |
| Pulmonary hemorrhage | 0 | 0 | 2 (0.5) | 2 (0.5) | 0 | 2 (0.5) |
| Cerebrovascular accident | 1 (0.3) | 1 (0.3) | 1 (0.3) | 0 | 0 | 0 |
| Intestinal perforation | 0 | 0 | 0 | 0 | 0 | 2 (0.5) |
| Posterior reversible | 0 | 0 | 0 | 0 | 1 (0.3) | 1 (0.3) |
| encephalopathy syndrome | | | | | | |
| Sepsis | 0 | 1 (0.3) | 0 | 0 | 0 | 1 (0.3) |
| Aortic dissection | 0 | 0 | 1 (0.3) | 0 | 0 | 0 |
| Intestinal obstruction | 0 | 0 | 1 (0.3) | 0 | 0 | 0 |

TABLE 16
Safety Summary

| | ABCP | | BCP | |
|---|---|---|---|---|
| Patients - no. (%) | (n = 393) | | (n = 394) | |
| Patients with ≥1 AE | 385 | (98.0) | 390 | (99.0) |
| Grade 3-4 AEs | 242 | (61.6) | 230 | (58.4) |
| Grade 5 AEs | 23 | (5.9) | 21 | (5.3) |
| Treatment-related AEs* | 371 | (94.4) | 376 | (95.4) |
| Treatment-related Grade 3-4 AEs | 219 | (55.7) | 188 | (47.7) |
| Treatment-related Grade 5 AEs | 11 | (2.8) | 9 | (2.3) |
| Serious AEs | 165 | (42.0) | 134 | (34.0) |
| Treatment-related serious AEs* | 100 | (25.4) | 76 | (19.3) |
| AEs leading to withdrawal from any treatment* | 128 | (32.6) | 98 | (24.9) |
| AEs leading to withdrawal from chemotherapy | 64 | (16.3) | 58 | (14.7) |

*Incidence of treatment-related adverse events (AEs), serious treatment-related AEs and AEs leading to withdrawal from any treatment are for any treatment.

### Example 7. Association Between Expression Levels of PD-L1, CXCL9, and IFNG and Clinical Response of Patients Having mUC to Treatment with Atezolizumab (MPDL3280A)

IMvigor211 (Clinical Trial ID No. NCT02302807) is a large, randomized, Phase III clinical trial that compared atezolizumab therapy to chemotherapy (taxanes or vinflunine) in platinum-treated metastatic urothelial carcinoma (mUC). The objective of this analysis was to assess clinical outcomes in subgroups defined by immune-score expression levels.

The IMvigor211 Trial enrolled patients with ≤2 prior lines of therapy for mUC whose disease had progressed during or following treatment with platinum-based chemotherapy (Powles et al., Lancet. pii: S0140-6736(17)33297-X, 2017). Patients were randomized 1:1 to receive atezolizumab 1200 mg or investigator's choice of chemotherapy (vinflunine, paclitaxel, or docetaxel), given intravenously every 3 weeks. Atezolizumab was administered until loss of clinical benefit, and chemotherapy was administered until RECIST v1.1 progressive disease.

Randomization was stratified by the following: the number of risk factors (time from prior chemotherapy <3 months; Eastern Cooperative Oncology Group Performance Status >0; hemoglobin <10 g/dL): 0 vs 1/2/3; the presence of liver metastases: yes vs no; investigator-pre-specified chemotherapy selection: vinflunine vs taxane.

The primary endpoint was overall survival (OS). Key secondary endpoints included objective response rate, duration of response and progression-free survival. Exploratory endpoints included the relationship between tumor immune specific- or disease-related biomarkers and efficacy. RNA sequencing was used to evaluate immune-score expression levels based on IFNG, CXCL9, and PD-L1.

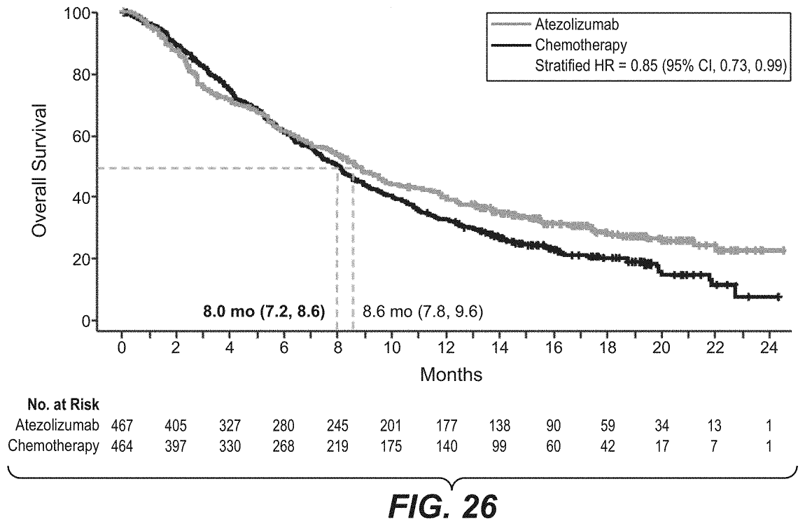

First, OS was analyzed in the ITT population (N=931). Results in the ITT population demonstrated improved OS in the atezolizumab treatment arm as compared to the chemotherapy treatment arm (FIG. 26).

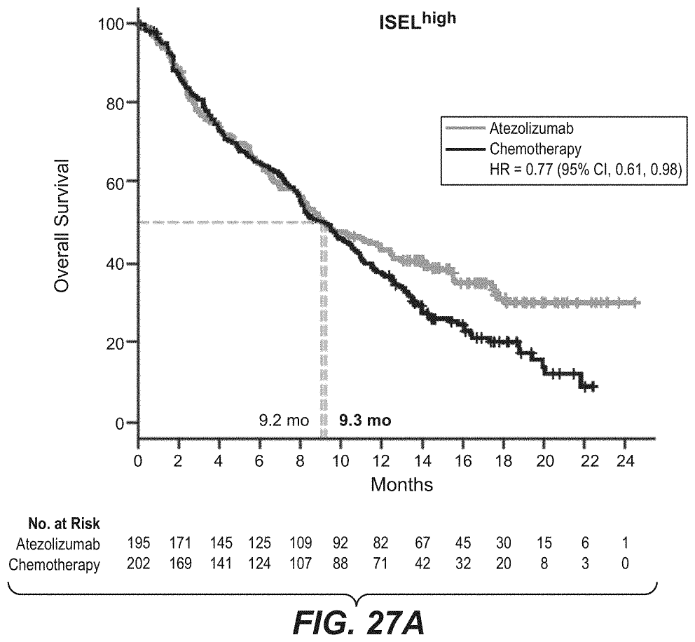

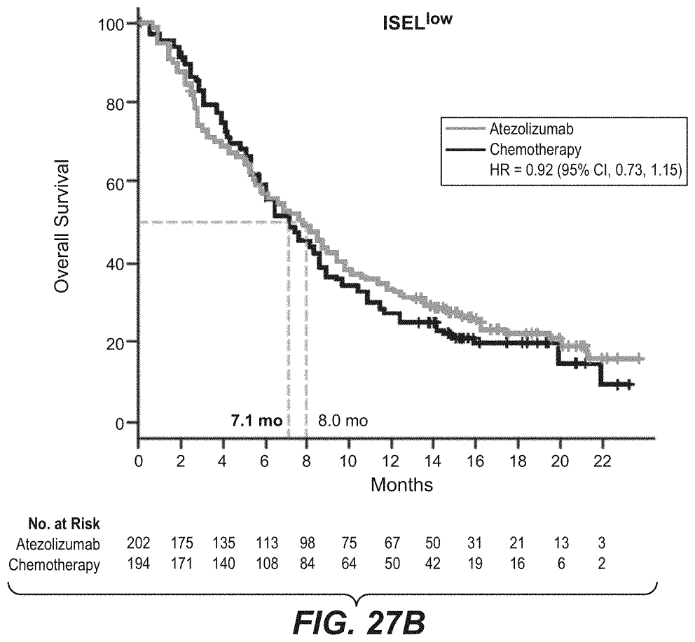

OS as a function of immune-score expression level was also evaluated (FIGS. 27A and 27B). The biomarker evaluable population included 793 patients. To evaluate whether PD-L1, CXCL9, and IFNG gene expression status was associated with patient response to atezolizumab (MPDL3280A) treatment, the immune-score expression level of PD-L1, CXCL9, and IFNG was assessed in tumor samples obtained from each patient. Tumor specimens obtained from the patients were categorized into high or low expression level subgroups based on the immune-score expression level of PD-L1, CXCL9, and IFNG relative to a cut-off value defined by the median immune-score expression level of the population analyzed. High immune-score expression levels (ISEL^{high}) were defined as PD-L1, CXCL9, and IFNG immune-score expression levels at or above the median cut-off. Low immune-score expression levels (ISEL^{low}) were defined as PD-L1, CXCL9, and IFNG immune-score expression levels below the median cut-off. As shown in FIGS. 27A and 27B, ISEL^{high} tumor status was associated with better prognosis in both arms, but in the ISEL^{high} population, separation of the Kaplan-Meier curves was observed.

### VII. OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of identifying an individual having a cancer who may benefit from a treatment comprising a PD-L1 binding antagonist, the method comprising determining the expression level of PD-L1, CXCL9, and IFNG in a sample from the individual, wherein an immune-score expression level of PD-L1, CXCL9, and IFNG in the sample that is above a reference immune-score expression level identifies the individual as one who may benefit from a treatment comprising a PD-L1 binding antagonist, wherein the reference immune-score expression level is an immune-score expression level of PD-L1, CXCL9, and IFNG in a reference population.

2. A method for selecting a therapy for an individual having a cancer, the method comprising determining the expression level of PD-L1, CXCL9, and IFNG in a sample from the individual, wherein an immune-score expression level of PD-L1, CXCL9, and IFNG in the sample that is above a reference immune-score expression level identifies the individual as one who may benefit from a treatment comprising a PD-L1 binding antagonist, wherein the reference immune-score expression level is an immune-score expression level of PD-L1, CXCL9, and IFNG in a reference population.

3. The method of claim 1, wherein the immune-score expression level of PD-L1, CXCL9, and IFNG in the sample is:
(a) above the reference immune-score expression level and the method further comprises administering to the individual an effective amount of a PD-L1 binding antagonist; or
(b) below the reference immune-score expression level, thereby identifying the individual as one who is less likely to benefit from a treatment comprising a PD-L1 binding antagonist, and the method further comprises administering to the individual an effective amount of an anti-cancer therapy other than, or in addition to, a PD-L1 binding antagonist.

4-6. (canceled)

7. A method of treating an individual having a cancer, the method comprising administering to the individual an effective amount of a PD-L1 binding antagonist, wherein prior to treatment the expression level of PD-L1, CXCL9, and IFNG in a sample from the individual has been determined and an immune-score expression level of PD-L1, CXCL9, and IFNG in the sample that is above a reference immune-score expression level has been determined, wherein the reference immune-score expression level is an immune-score expression level of PD-L1, CXCL9, and IFNG in a reference population.

8. The method of claim 7, wherein the immune-score expression level of PD-L1, CXCL9, and IFNG in the sample is in the top 80^{th} percentile of the immune-score expression level of PD-L1, CXCL9, and IFNG in the reference population.

9-10. (canceled)

11. The method of claim 7, wherein the reference population is a population of individuals having the cancer, the population of individuals consisting of a first subset of individuals who have been treated with a PD-L1 binding antagonist therapy and a second subset of individuals who have been treated with a non-PD-L1 binding antagonist therapy, wherein the non-PD-L1 binding antagonist therapy does not comprise a PD-L1 binding antagonist.

12. The method of claim 11, wherein the reference immune-score expression level significantly separates each of the first and second subsets of individuals based on a significant difference between an individual's responsiveness to treatment with the PD-L1 binding antagonist therapy and an individual's responsiveness to treatment with the non-PD-L1 binding antagonist therapy:
(a) above the reference immune-score expression level, wherein the individual's responsiveness to treatment with the PD-L1 binding antagonist therapy is significantly improved relative to the individual's responsiveness to treatment with the non-PD-L1 binding antagonist therapy; or
(b) below the reference immune-score expression level, wherein the individual's responsiveness to treatment with the non-PD-L1 binding antagonist therapy is significantly improved relative to the individual's responsiveness to treatment with the PD-L1 binding antagonist therapy.

13. (canceled)

14. The method of claim 12, wherein responsiveness to treatment is an increase in progression-free survival (PFS) or overall survival (OS).

15. (canceled)

16. The method of claim 7, wherein the immune-score expression level of PD-L1, CXCL9, and IFNG is:
(a) an average of the expression level of each of PD-L1, CXCL9, and IFNG;
(b) a median of the expression level of each of PD-L1, CXCL9, and IFNG; or
(c) a pre-assigned expression level of PD-L1, CXCL9, and IFNG.

17. The method of claim 16, wherein the average of the expression level of each of PD-L1, CXCL9, and IFNG is an average of a normalized expression level of each of PD-L1, CXCL9, and IFNG or the median of the expression level of each of PD-L1, CXCL9, and IFNG is a median of a normalized expression level of each of PD-L1, CXCL9, and IFNG.

18-19. (canceled)

20. The method of claim 17, wherein the normalized expression level of each of PD-L1, CXCL9, and IFNG is the expression level of each of PD-L1, CXCL9, and IFNG normalized to a reference gene.

21. (canceled)

22. A method of identifying an individual having a cancer who may benefit from a treatment comprising a PD-L1 binding antagonist, the method comprising determining the expression level of PD-L1, IFNG, GZMB, and CD8A in a sample from the individual, wherein an immune-score expression level of PD-L1, IFNG, GZMB, and CD8A in the sample that is above a reference immune-score expression level identifies the individual as one who may benefit from a treatment comprising a PD-L1 binding antagonist, wherein the reference immune-score expression level is an immune-score expression level of PD-L1, IFNG, GZMB, and CD8A in a reference population.

23. A method for selecting a therapy for an individual having a cancer, the method comprising determining the expression level of PD-L1, IFNG, GZMB, and CD8A in a sample from the individual, wherein an immune-score expression level of PD-L1, IFNG, GZMB, and CD8A in the sample that is above a reference immune-score expression level identifies the individual as one who may benefit from a treatment comprising a PD-L1 binding antagonist, wherein the reference immune-score expression level is an immune-score expression level of PD-L1, IFNG, GZMB, and CD8A in a reference population.

24. The method of claim 22 or 23, wherein the immune-score expression level of PD-L1, IFNG, GZMB, and CD8A in the sample is:
(a) above the reference immune-score expression level and the method further comprises administering to the individual an effective amount of a PD-L1 binding antagonist; or
(b) below the reference immune-score expression level, thereby identifying the individual as one who is less likely to benefit from a treatment comprising a PD-L1 binding antagonist, and the method further comprises administering to the individual an effective amount of an anti-cancer therapy other than, or in addition to, a PD-L1 binding antagonist.

25-27. (canceled)

28. A method of treating an individual having a cancer, the method comprising administering to the individual an effective amount of a PD-L1 binding antagonist, wherein prior to treatment the expression level of PD-L1, IFNG, GZMB, and CD8A in a sample from the individual has been determined and an immune-score expression level of PD-L1, IFNG, GZMB, and CD8A in the sample that is above a reference immune-score expression level has been determined, wherein the reference immune-score expression level is an immune-score expression level of PD-L1, IFNG, GZMB, and CD8A in a reference population.

29. The method of claim 28, wherein the immune-score expression level of PD-L1, IFNG, GZMB, and CD8A in the sample is in the top 80^{th} percentile of the immune-score expression level of PD-L1, IFNG, GZMB, and CD8A in the reference population.

30-31. (canceled)

32. The method of claim 28, wherein the reference population is a population of individuals having the cancer, the population of individuals consisting of a first subset of individuals who have been treated with a PD-L1 binding antagonist therapy and a second subset of individuals who have been treated with a non-PD-L1 binding antagonist therapy, wherein the non-PD-L1 binding antagonist therapy does not comprise a PD-L1 binding antagonist.

33. The method of claim 32, wherein the reference immune-score expression level significantly separates each of the first and second subsets of individuals based on a significant difference between an individual's responsiveness to treatment with the PD-L1 binding antagonist therapy and an individual's responsiveness to treatment with the non-PD-L1 binding antagonist therapy:
(a) above the reference immune-score expression level, wherein the individual's responsiveness to treatment with the PD-L1 binding antagonist therapy is significantly improved relative to the individual's responsiveness to treatment with the non-PD-L1 binding antagonist therapy; or
(b) below the reference immune-score expression level, wherein the individual's responsiveness to treatment with the non-PD-L1 binding antagonist therapy is significantly improved relative to the individual's responsiveness to treatment with the PD-L1 binding antagonist therapy.

34. (canceled)

35. The method of claim 33, wherein responsiveness to treatment is an increase in PFS or OS.

36. (canceled)

37. The method of claim 28, wherein the immune-score expression level of PD-L1, IFNG, GZMB, and CD8A is:
(a) an average of the expression level of each of PD-L1, IFNG, GZMB, and CD8A;
(b) a median of the expression level of each of PD-L1, IFNG, GZMB, and CD8A; or
(c) a pre-assigned expression level of PD-L1, IFNG, GZMB, and CD8A.

38. The method of claim 37, wherein the average expression level of each of PD-L1, IFNG, GZMB, and CD8A is an average of a normalized expression level of each of PD-L1, IFNG, GZMB, and CD8A or the median of the expression level of each of PD-L1, IFNG, GZMB, and CD8A is a median of a normalized expression level of each of PD-L1, IFNG, GZMB, and CD8A.

39-40. (canceled)

41. The method of claim 38, wherein the normalized expression level of each of PD-L1, IFNG, GZMB, and CD8A is the expression level of each of PD-L1, IFNG, GZMB, and CD8A normalized to a reference gene.

42. (canceled)

43. A method of identifying an individual having a cancer who may benefit from a treatment comprising a PD-L1 binding antagonist, the method comprising determining the expression level of PD-L1, IFNG, GZMB, CD8A, and PD-1 in a sample from the individual, wherein an immune-score expression level of PD-L1, IFNG, GZMB, CD8A, and PD-1 in the sample that is above a reference immune-score expression level identifies the individual as one who may benefit from a treatment comprising a PD-L1 binding antagonist, wherein the reference immune-score expression level is an immune-score expression level of PD-L1, IFNG, GZMB, CD8A, and PD-1 in a reference population.

44. A method for selecting a therapy for an individual having a cancer, the method comprising determining the expression level of PD-L1, IFNG, GZMB, CD8A, and PD-1 in a sample from the individual, wherein an immune-score expression level of PD-L1, IFNG, GZMB, CD8A, and PD-1 in the sample that is above a reference immune-score expression level identifies the individual as one who may benefit from a treatment comprising a PD-L1 binding antagonist, wherein the reference immune-score expression level is an immune-score expression level of PD-L1, IFNG, GZMB, CD8A, and PD-1 in a reference population.

45. The method of claim 43, or wherein the immune-score expression level of PD-L1, IFNG, GZMB, CD8A, and PD-1 in the sample is:
(a) above the reference immune-score expression level and the method further comprises administering to the individual an effective amount of a PD-L1 binding antagonist; or
(b) below the reference immune-score expression level, thereby identifying the individual as one who is less likely to benefit from a treatment comprising a PD-L1 binding antagonist, and the method further comprises administering to the individual an effective amount of an anti-cancer therapy other than, or in addition to, a PD-L1 binding antagonist.

46-48. (canceled)

49. A method of treating an individual having a cancer, the method comprising administering to the individual an effective amount of a PD-L1 binding antagonist, wherein prior to treatment the expression level of PD-L1, IFNG, GZMB, CD8A, and PD-1 in a sample from the individual has been determined and an immune-score expression level of PD-L1, IFNG, GZMB, CD8A, and PD-1 in the sample that is above a reference immune-score expression level has been determined, wherein the reference immune-score expression level is an immune-score expression level of PD-L1, IFNG, GZMB, CD8A, and PD-1 in a reference population.

50. The method of claim 49, wherein the immune-score expression level of PD-L1, IFNG, GZMB, CD8A, and PD-1 in the sample is in the top 80^{th} percentile of the immune-score expression level of PD-L1, IFNG, GZMB, CD8A, and PD-1 in the reference population.

51-52. (canceled)

53. The method of claim 49, wherein the reference population is a population of individuals having the cancer, the population of individuals consisting of a first subset of individuals who have been treated with a PD-L1 binding antagonist therapy and a second subset of individuals who have been treated with a non-PD-L1 binding antagonist therapy, wherein the non-PD-L1 binding antagonist therapy does not comprise a PD-L1 binding antagonist.

54. The method of claim 53, wherein the reference immune-score expression level significantly separates each of the first and second subsets of individuals based on a significant difference between an individual's responsiveness to treatment with the PD-L1 binding antagonist therapy and an individual's responsiveness to treatment with the non-PD-L1 binding antagonist therapy:
(a) above the reference immune-score expression level, wherein the individual's responsiveness to treatment with the PD-L1 binding antagonist therapy is significantly improved relative to the individual's responsiveness to treatment with the non-PD-L1 binding antagonist therapy; or
(b) below the reference immune-score expression level, wherein the individual's responsiveness to treatment with the non-PD-L1 binding antagonist therapy is significantly improved relative to the individual's responsiveness to treatment with the PD-L1 binding antagonist therapy.

55. (canceled)

56. The method of claim 54, wherein responsiveness to treatment is an increase in PFS or OS.

57. (canceled)

58. The method of claim 49, wherein the immune-score expression level of PD-L1, IFNG, GZMB, CD8A, and PD-1 is:
(a) an average of the expression level of each of PD-L1, IFNG, GZMB, CD8A, and PD-1;
(b) a median of the expression level of each of PD-L1, IFNG, GZMB, CD8A, and PD-1; or
(c) a pre-assigned expression level of PD-L1, IFNG, GZMB, CD8A, and PD-1.

59. The method of claim 58, wherein the average of the expression level of each of PD-L1, IFNG, GZMB, CD8A, and PD-1 is an average of a normalized expression level of each of PD-L1, IFNG, GZMB, CD8A, and PD-1 or the median of the expression level of each of PD-L1, IFNG, GZMB, CD8A, and PD-1 is a median of a normalized expression level of each of PD-L1, IFNG, GZMB, CD8A, and PD-1.

60-61. (canceled)

62. The method of claim 59, wherein the normalized expression level of each of PD-L1, IFNG, GZMB, CD8A, and PD-1 is the expression level of each of PD-L1, IFNG, GZMB, CD8A, and PD-1 normalized to a reference gene.

63-65. (canceled)

66. The method of claim 7, wherein benefit from the treatment comprising a PD-L1 binding antagonist is an increase in OS or PFS.

67-68. (canceled)

69. The method of claim 7, wherein the expression level is a nucleic acid expression level.

70. The method of claim 69, wherein the nucleic acid expression level is an mRNA expression level.

71. The method of claim 70, wherein the mRNA expression level is determined by RNA-seq, RT-qPCR, qPCR, multiplex qPCR or RT-qPCR, microarray analysis, SAGE, MassARRAY technique, ISH, or a combination thereof.

72-74. (canceled)

75. The method of claim 7, wherein the sample is a tissue sample, a cell sample, a whole blood sample, a plasma sample, a serum sample, or a combination thereof.

76. The method of claim 75, wherein the tissue sample is a tumor tissue sample.

77. The method of claim 76, wherein the tumor tissue sample comprises tumor cells, tumor-infiltrating immune cells, stromal cells, or a combination thereof.

78. The method of claim 76, wherein the tumor tissue sample is a formalin-fixed and paraffin-embedded (FFPE) sample, an archival sample, a fresh sample, or a frozen sample.

79. (canceled)

80. The method of claim 7, wherein the cancer is selected from the group consisting of a lung cancer, a kidney cancer, a bladder cancer, a breast cancer, a colorectal cancer, an ovarian cancer, a pancreatic cancer, a gastric carcinoma, an esophageal cancer, a mesothelioma, a melanoma, a head and neck cancer, a thyroid cancer, a sarcoma, a prostate cancer, a glioblastoma, a cervical cancer, a thymic carcinoma, a leukemia, a lymphoma, a myeloma, a mycosis fungoides, a merkel cell cancer, or a hematologic malignancy.

81. (canceled)

82. The method of claim 80, wherein:
(a) the lung cancer is a non-small cell lung cancer (NSCLC);
(b) the kidney cancer is a renal cell carcinoma (RCC);
(c) the bladder cancer is an urothelial bladder cancer (UBC); or
(d) the breast cancer is a triple negative breast cancer (TNBC).

83-85. (canceled)

86. The method of claim 7, wherein the PD-L1 binding antagonist inhibits the binding of PD-L1 to PD-1, the binding of PD-L1 to B7-1, or the binding of PD-L1 to both PD-1 and B7-1.

87. The method of claim 7, wherein the PD-L1 binding antagonist is an anti-PD-L1 antibody.

88. The method of claim 87, wherein the anti-PD-L1 antibody is selected from the group consisting of atezolizumab (MPDL3280A), MSB0010718C, MDX-1105, and MEDI4736.

89. The method of claim 87, wherein the anti-PD-L1 antibody comprises:
(a) the following hypervariable regions:
(i) an HVR-H1 sequence of GFTFSDSWIH (SEQ ID NO: 9);
(ii) an HVR-H2 sequence of AWISPYGGSTYYADSVKG (SEQ ID NO: 10);
(iii) an HVR-H3 sequence of RHWPGGFDY (SEQ ID NO: 11);
(iv) an HVR-L1 sequence of RASQDVSTAVA (SEQ ID NO: 12);
(v) an HVR-L2 sequence of SASFLYS (SEQ ID NO: 13); and
(vi) an HVR-L3 sequence of QQYLYHPAT (SEQ ID NO: 14); or
(b) (i) a heavy chain variable (VH) domain comprising an amino acid sequence having at least 90% sequence identity to the amino acid sequence of SEQ ID NO: 16; and (ii) a light chain variable (VL) domain comprising an amino acid sequence having at least 90% sequence identity to the amino acid sequence of SEQ ID NO: 17.

90-96. (canceled)

97. The method of claim 89, wherein the anti-PD-L1 antibody comprises:
(a) a VH domain comprising the amino acid sequence of SEQ ID NO: 16; and
(b) a VL domain comprising the amino acid sequence of SEQ ID NO: 17.

98. (canceled)

99. The method of claim 11, wherein the non-PD-L1 binding antagonist is an anti-neoplastic agent, a chemotherapeutic agent, a growth inhibitory agent, an anti-angiogenic agent, a radiation therapy, or a cytotoxic agent.

100. The method of claim 3, wherein the anti-cancer therapy is an anti-neoplastic agent, a chemotherapeutic agent, a growth inhibitory agent, an anti-angiogenic agent, a radiation therapy, or a cytotoxic agent.

101. The method of claim 7, wherein the individual has not been previously treated for the cancer.

102. The method of claim 101, wherein the individual has not been previously administered a PD-L1 binding antagonist.

103. The method of claim 7, wherein the treatment comprising a PD-L1 binding antagonist is a monotherapy or a combination therapy.

104. (canceled)

105. The method of claim 7, further comprising administering to the individual an effective amount of an additional therapeutic agent.

106. The method of claim 105, wherein the additional therapeutic agent is an anti-neoplastic agent, a chemotherapeutic agent, a growth inhibitory agent, an anti-angiogenic agent, a radiation therapy, a cytotoxic agent, or a combination thereof.

107. (canceled)

108. The method of claim 106, wherein:
(a) the chemotherapeutic agent is carboplatin; paclitaxel; or carboplatin and paclitaxel; or
(b) the anti-angiogenic agent is an anti-VEGF antibody.

109-114. (canceled)

115. The method of claim 108, wherein the anti-VEGF antibody is bevacizumab.

116. (canceled)

117. A kit for identifying an individual having a cancer who may benefit from a treatment comprising a PD-L1 binding antagonist, the kit comprising;
reagents for determining the expression level of PD-L1, CXCL9, and IFNG in a sample from the individual.

118. A kit for identifying an individual having a cancer who may benefit from a treatment comprising a PD-L1 binding antagonist, the kit comprising;
reagents for determining the expression level of PD-L1, IFNG, GZMB, and CD8A in a sample from the individual.

119. A kit for identifying an individual having a cancer who may benefit from a treatment comprising a PD-L1 binding antagonist, the kit comprising;
reagents for determining the expression level of PD-L1, IFNG, GZMB, CD8A, and PD-1 in a sample from the individual.

120-122. (canceled)
